(19) United States
(12) Patent Application Publication
(10) Pub. No.: US 2023/0234943 A1
(43) Pub. Date: Jul. 27, 2023

(54) TREX1 INHIBITORS AND USES THEREOF

(71) Applicant: Tempest Therapeutics, Inc., Brisbane, CA (US)

(72) Inventors: Thomas Walter DUBENSKY, JR., South San Francisco, CA (US); Ryan C. CLARK, South San Francisco, CA (US); Justin ERNST, San Diego, CA (US); Garrick K. PACKARD, San Diego, CA (US); Brett BUSCH, San Diego, CA (US); Joe Fred NAGAMIZO, San Diego, CA (US); Biswajit KALITA, Bangalore, Karnataka (IN); Athisayamani Jeyaraj DURAISWAMY, Bangalore, Karnataka (IN)

(21) Appl. No.: 18/002,557

(22) PCT Filed: Jun. 25, 2021
(86) PCT No.: PCT/US2021/039041

§ 371 Date: Dec. 20, 2022

Related U.S. Application Data
(60) Provisional application No. 63/044,705, filed on Jun. 26, 2020.

(51) Int. Cl. C07D 405/04; C07D 471/04; C07D 235/18; C07D 277/66; C07D 487/04; C07D 495/04; C07D 473/00; C07D 513/04; C07D 473/32; C07D 403/12; C07D 403/04; C07D 401/12; C07D 401/04; C07D 487/10; C07F 7/08; C07D 487/08; C07D 405/14; C07D 413/14; C07D 409/14; A61K 45/06; A61N 5/10

(52) U.S. Cl. CPC C07D 405/04; C07D 471/04; C07D 235/18; C07D 277/66; C07D 487/04; C07D 495/04; C07D 473/00; C07D 513/04; C07D 473/32; C07D 403/12; C07D 403/04; C07D 401/12; C07D 401/04; C07D 487/10; C07F 7/0812; C07D 487/08; C07D 405/14; C07D 413/14; C07D 409/14; A61K 45/06; A61N 5/10; A61N 2005/1098

(57) ABSTRACT

Described herein are compounds, methods of making such compounds, pharmaceutical compositions and medicaments comprising such compounds, and methods of using such compounds for inhibiting three prime repair exonuclease 1 (“TREX1”).

### CROSS-REFERENCE

This application claims the benefit of U.S. Provisional Application Ser. No. 63/044,705 filed Jun. 26, 2020 which is hereby incorporated by reference in its entirety.

### BACKGROUND OF THE INVENTION

The presence of tumor infiltrating T cells (“TILs”) is associated with improved clinical outcomes in multiple tumor types and also improves responses to immune checkpoint blockade therapies. Although T-cell responses to some tumors occur spontaneously, the majority of cancers are not naturally recognized by the immune system or have evolved multiple mechanisms of immune evasion. Preclinical and clinical data together have established a central role for type I interferons (“IFNs”) in linking innate and adaptive immune responses to mediate tumor rejection. Non-T cell-inflamed tumors in humans are deficient in type I IFNs. Thus, the development of therapeutic strategies for restoring type I IFN signaling is essential for expanding the number of patients who may be effectively treated with immunotherapy.

Innate immune sensing in the tumor microenvironment (“TME”) is a critical step in promoting spontaneous tumor-initiated T cell priming and subsequent TIL infiltration (Fuertes et al., J. Exp. Med. 2011, 208, 2005-2016). Transcriptional profiling analyses of melanoma patients has revealed that tumors containing infiltrating activated T cells are characterized by a type I IFN transcriptional signature (Harlin et al., Cancer Res. 2009, 69, 3077-3085). Studies in mice have demonstrated that type I IFN signaling plays a critical role in tumor-initiated T cell priming (Diamond et al., J Exp. Med. 2011, 208, 1989-2003; and Fuertes et al., J Exp. Med. 2011, 208, 2005-2016). Mice lacking the IFN-α/β receptor in dendritic cells (“DCs”) cannot reject immunogenic tumors and CD8α⁺ DCs from these mice are defective in antigen cross-presentation to CD8⁺ T cells. Furthermore, Baft3^{-/-} mice that lack the CD8α⁺ DC lineage lose the capacity to spontaneously prime tumor-specific CD8⁺ T cells (Fuertes et al., J. Exp. Med. 2011, 208, 2005-2016; and Hildner et al., Science 2008, 322, 1097-1100). These findings in humans and in mice indicate that the tumor-resident antigen presenting cell (“APC”) compartment is deficient/absent in non T cell-inflamed tumors. Thus, strategies to induce type I IFN signaling and APC activation in the TME to bridge the innate and adaptive immune responses may have therapeutic utility.

How the innate immune system is engaged by targeted ligands shapes the development of an adaptive response and is central to effective immunotherapy (Dubensky et al., Ther. Adv. Vaccines 2013, 1, 131-143; and Dubensky and Reed, Semin. Immunol. 2010, 22, 155-161). The design and development of ligands to activate innate immunity is guided by a fundamental understanding that conserved microbial structures known as Pathogen-Associated Molecular Patterns (“PAMPs”) are sensed by germ-line encoded host cell Pattern Recognition Receptors (“PRRs”), triggering a downstream signaling cascade resulting in the induction of cytokines and chemokines, and initiation of a specific adaptive immune response (Iwasaki and Medzhitov, Science 2010, 327, 291-295). Engagement of the innate immune system by PAMPs presented from an infectious agent or by cellular danger signals known as Danger Associated Molecular Patterns (“DAMPs”) shapes the development of the adaptive antigen-specific response. One objective in the design of innate immune activators is to select defined PAMPs, DAMPS, or synthetic molecules which activate designated PRRs and initiate a desired response. Innate immune ligands (agonists) such as monophosphoryl lipid A (“MPL”) and CpG are microbial-derived PAMPs recognized by Toll-like receptors (“TLRs”), a class of PRRs that signal through MyD88 and TRIF adaptor molecules and mediate induction of NF-kB dependent proinflammatory cytokines (Kawai and Akira, Nat. Immunol. 2010, 11, 373-384). While TLRs present on the cell surface (e.g., TLR-4) and endosomes (e.g., TLR-9) sense extracellular and vacuolar pathogens, the productive growth cycle of multiple pathogens including viruses and intracellular bacteria occurs in the cytosol. The compartmentalization of extracellular, vacuolar, and cytosolic PRRs has led to the hypothesis that the innate immune system can sense particular productively replicating pathogenic microbes by monitoring the cytosol (Vance et al., Science 2009, 323, 1208-1211). This provides a rationale for the use of agonists that activate PRRs comprising the cytosolic surveillance pathway and may be an effective strategy for the design of innate immune activators for cancer immunotherapy.

Nucleic acids from bacterial, viral, protozoan, and fungal pathogens are sensed by several distinct cytosolic signaling pathways. When activated, these individual pathways induce a characteristic cytokine profile, which in turn shapes the antigen (“Ag”)-specific immune response. For example, the nucleotide binding oligomerization domain (“NOD”)-like receptor (“NLR”) family, such as “absent in melanoma 2” (“AIM2”), senses cytosolic double-stranded (“ds”) DNA, triggering activation of the inflammasome and caspase-1 dependent production of IL-1β (Strowig et al., Nature 2012, 481, 278-286). The signaling cascade resulting from activation of the inflammasome stimulates priming of Th17-biased CD4⁺ T cell immunity, associated with protection against diverse pathogens, such as Streptococcus pneumoniae (Olliver et al., Infect. Immun. 2011, 79, 4210-4217).

Type I interferons (IFN-α, IFN-β) are the signature cytokines induced by two distinct TLR-independent cytosolic signaling pathways. In the first pathway, various forms of single-stranded and double-stranded (“ds”) RNA are sensed by RNA helicases, including retinoic acid-inducible gene I (“RIG-1”) and melanoma differentiation-associated gene 5 (“MDA-5”), and through the IFN-β promoter stimulator 1 (“IPS-1”) adaptor protein mediate phosphorylation of the IRF-3 transcription factor, leading to induction of IFN-β (Ireton and Gale, Viruses 2011, 3, 906-919). IPS-1^{-/-} deficient mice have increased susceptibility to infection with RNA viruses. Sensors that signal through the IPS-1 pathway are directly targeted for inactivation by various viral proteins, demonstrating a requirement of this cytosolic host defense pathway to control productive virus infection. Synthetic dsRNA, such as polyinosinic:polycytidylic acid (“poly (I:C)”) and poly ICLC, an analog that is formulated with poly L lysine to resist RNase digestion, is an agonist for both TLR3 and MDA5 pathways, is a powerful inducer of IFN-β, and is currently being evaluated in several diverse clinical settings (Caskey et al., J. Exp. Med. 2011, 208, 2357-2366).

Stimulator of Interferon Genes (“STING”) is the central mediator for the second cytosolic pathway that triggers type I interferon in response to sensing cytosolic double-stranded (“ds”) DNA from infectious pathogens or aberrant host cells (DAMPS) (Motwani, Nat. Rev. Genet. 2019, 20, 657-674, and Barber, Curr. Opin. Immunol. 2011, 23, 10-20). Alternatively known as TMEM173, MITA, ERIS, and MPYS, STING was discovered by Glen Barber and colleagues using cDNA expression cloning methods as a MyD88-independent host cell defense factor expressed in macrophages, dendritic cells, and fibroblasts, and was found to induce expression of IFN-β and NF-κB dependent pro-inflammatory cytokines in response to sensing cytoplasmic DNA (Ishikawa and Barber, Nature 2008, 455, 674-678). Significantly, and of particular relevance to the therapeutic modulation of STING, activation of this pathway occurs in response to sensing host cell DNA in the cytoplasm, originating from the nucleus or the mitochondria, both in a paracrine and autocrine fashion (Chen et al., Nat. Immunol. 2016, 17, 1142-1149).

Recent work has demonstrated that activation of the STING pathway in tumor-resident host APCs is required for induction of a spontaneous CD8⁺ T cell response against tumor-derived antigens in vivo (Woo et al., Immunity 2014, 41, 830-842; and Corrales et al., J Clin. Invest. 2016,126, 404-411). In addition, activation of this pathway and the subsequent production of IFN-β contributes to the anti-tumor effect of radiation, which can be potentiated with co-administration of a natural STING agonist (Deng et al., Immunity 2014, 41, 843-852). STING is a transmembrane protein localized to the endoplasmic reticulum that undergoes a conformational change in response to direct binding of cyclic dinucleotides (“CDNs”), resulting in a downstream signaling cascade involving TBK1 activation, IRF-3 phosphorylation, and production of IFN-β and other cytokines (Burdette et al., Nature 2011, 478, 515-518; Burdette and Vance, Nat. Immunol. 2013, 14, 19-26; and Ishikawa and Barber, Nature 2008, 455, 674-678). After CDN binding by STING, canonical NF-κB dependent cytokines are also induced (Chen et al., Nat. Immunol. 2016, 17, 1142-1149). IFN-β is the signature cytokine induced in response to STING activation, by either exogenous CDNs produced by bacterial infection, or through binding of a structurally distinct endogenous CDN produced by a host cyclic GMP-AMP synthetase (“cGAS”) in response to sensing cytosolic double-stranded DNA (“dsDNA”) (Ablasser et al., Nature 2013, 498, 380-384; Diner et al., Cell Rep. 2013, 3, 1355-1361; McWhirter et al., J. Exp. Med. 2009, 206, 1899-1911; Sun et al., Science 2013, 339, 786-791; Woodward et al., Science 2010, 328, 1703-1705; and Zhang et al., Mol. Cell 2013, 51, 226-235). IFNs stimulate expression of interferon-stimulated genes (“ISGs”), a key event that links host innate immunity to the initiation of adaptive immunity. These observations suggested that direct activation of the STING pathway in the TME with specific agonists might be an effective therapeutic strategy to promote broad tumor-initiated T cell priming against an individual's unique tumor antigen repertoire.

STING is expressed ubiquitously in both immune and somatic cells. Initial clinical approaches to target STING have primarily utilized intratumoral (“IT”) administration of synthetic modified CDNs, to avoid possible toxicity including a cytokine storm or cytokine release syndrome due to expression of high levels of pro-inflammatory cytokines such as IL-6 and TNF-α resulting from broad activation of STING with the systemic administration of potent ligands/agonists. As a single agent, IT injection of CDNs demonstrates potent anti-tumor effects in multiple syngeneic mouse tumor models without significant local or systemic toxicity. Direct IT injection of selected CDNs in established B16 melanoma, CT26 colon, and 4T1 breast carcinomas resulted in rapid and profound tumor regression and promoted lasting systemic antigen-specific T cell immunity (Sivick et al., Cell Rep. 2018, 25, 3074-3085; Corrales et al., Cell Rep. 2015, 11, 1018-1030; Foote et al., Cancer Immunol. Res. 2017, 5, 468-479; and Francica et al., Cancer Immunol. Res. 2018, 6, 422-433). In particular, these preclinical investigations demonstrated that tumor-specific CD8⁺ T cells primed locally in the draining lymph node serving the injected tumor could traffic to and cause the regression of distal non-injected tumors, supporting the scientific rationale for evaluation of STING agonists to treat patients with advanced metastatic cancers.

Distinct metastatic tumors are genetically diverse and have unique antigenic repertoires. In order to grow, proliferate, and spread, tumors evolve to avoid immune recognition via a process known as immunoediting. Silencing or deletion of genes encoding proteins required for antigen presentation can prevent presentation of particular antigens of major histocompatibility complex (“MHC”) class I and class II molecules, hindering recognition by antigen-specific cytolytic T cells and preventing tumor cell death (Mittal et al., Curr. Opin. Immunol. 2014, 27, 16-25). The immunoediting process is constant due to the genetic instability of tumor cells, such that the antigens presented by a given metastatic tumor in an individual with advanced cancer can be distinct from those presented by a distinct metastatic tumor lesion. The genetic heterogeneity in evolving progressing tumors means that a CD8⁺ T cell with specificity for a designated antigen expressed on one tumor cell, with said CD8⁺ T cell able to kill that tumor cell, may not recognize a separate and distinct tumor because its cognate antigen is not presented on that tumor cell. Implanted mouse tumor models, in comparison, lack genetic heterogeneity because these models are based on homogenous tumor cell lines that grow to lethality before immune selection. Thus, tumor-specific CD8⁺ T cells primed locally in the draining lymph node serving an injected tumor can traffic to and eradicate distal non-injected tumors. This observation in mice, which has been referred to as an abscopal effect, is an artificial model of human cancer because the identical tumor cell line, e.g., CT26 colorectal tumor cells, is implanted on opposite flanks of the mouse.

There is a need for broad priming of tumor antigen-specific CD4+ and CD8⁺ T cells in the lymph nodes that serve diverse metastatic tumors that are spread throughout the body of an affected individual with advanced cancer. Efficient systemic delivery of ligands to activate designated innate immune receptors selectively in the tumor microenvironment, but not broadly in extra-tumoral tissues where targeted immune receptors are expressed, is a desired therapeutic outcome. Such selective targeting of designated innate immune receptors in the TME is anticipated to induce desired IRF3- and NF-κB-dependent pro-inflammatory cytokines and chemokines that are required to recruit, activate, and initiate innate and adaptive immune cell populations, resulting in priming of tumor-specific T cell immunity. On the other hand, broad non-selective activation of innate immune receptors upon systemic delivery is not desired as high systemic levels of IRF3- and NF-κB-dependent pro-inflammatory cytokines and chemokines such as IFN-β, TNF-α, IFN-γ, IL-12p70, and IL-6 limits tolerability, can result in toxicity, and limits the effectiveness of priming tumor-specific immunity resulting from innate immune activation selectively in the TME. STING has been shown in mice to be a critical innate immune receptor for development of antigen-specific T cell immunity, and genetic mutations in STING result in a significant inflammatory disease in humans known as STING-associated vasculopathy with onset in infancy (“SAVI”), providing scientific rationale for targeting the STING pathway to initiate tumor-specific immunity (Fuertes et al., J. Exp. Med. 2011, 208, 2005-2016). However, because STING is expressed broadly in diverse immune cell and somatic cell populations, an efficient means to target activation of the STING pathway selectively in the TME with a systemically delivered (e.g., orally or intravenously) agent is highly desirable as a therapeutic approach to initiate tumor-antigen specific priming against diverse metastases and effective tumor eradication.

While STING is ubiquitously expressed in both immune and somatic cell populations, three prime repair exonuclease 1 (“TREX1”) is a 3′-5′ DNA exonuclease that maintains immune homeostasis by limiting activation of cGAS-STING in normal cells. TREX1 is induced by cytosolic DNA resulting from inflammation, DNA repair deficiency, chemotherapy, or radiotherapy. Severe human inflammatory diseases including Aicardi-Goutibres syndrome (“AGS”) and chilblain lupus are interferonopathies resulting from inactivating genetic mutations in TREX1, lead to increased levels of cytosolic dsDNA and chronic activation of the STING pathway. TREX1 is an upstream regulatory mediator of radiation-induced anti-tumor immunity, and the immunity induced by radiation is STING-dependent (Deng et al., Immunity 2014, 41, 843-852). Radiation dose is reversibly correlated with the induced level of IFN-β, the signature cytokine of activated STING. At high radiation dose levels, TREX1 is significantly induced at levels which substantially degrades cytosolic DNA, leading to lower levels of production of cGAMP by cGAS and correspondingly decreased activation of STING and induction of IFN-β. In contrast, hyperfractionated radiation (lower dose levels of radiation delivered over multiple doses) does not affect TREX1 levels and leads to significantly higher levels of IFN-β and development of effective anti-tumor immunity and tumor regression (Vanpouille-Box et al., Nature Comm. 2017, 8, 15618-15632). However, effective anti-tumor immunity and tumor regression can be optimally achieved when delivery of high-dose radiotherapy administered as a single dose and/or hypofractionated in conjunction with SBRT—to maximize tumor killing and dsDNA levels—is combined with an effective TREX-1 inhibitor. Genotoxic stress-mediated induction of TREX1 can also be achieved by DNA-modifying chemotherapeutic agents, including dsDNA crosslinking alkylating agents such as nimustine, carmustine, fotemustine, and topotecan (Tomicic et al., Biochimica et Biophysica Acta 2013, 1835, 11-27). Many advanced cancers exhibit deficient DNA repair, due to mutations in genes encoding proteins involved in various DNA repair pathways, leading to genomic plasticity and, consequently, increased tumor virulence. These mutations also result in increased levels of cytosolic DNA and correspondingly increased levels of TREX1, which in turn sufficiently degrades cytosolic DNA and diminishes the extent of activation of the cGAS-STING pathway. Thus, high levels of TREX1 expression facilitate evasion of immune recognition, and therapeutic intervention with agents that both increase the level of TME cytosolic dsDNA and inhibit TREX1 will result in profound activation of the cGAS-STING pathway and development of effective anti-tumor immunity.

### BRIEF SUMMARY OF THE INVENTION

Disclosed herein is a compound of Formula (I), or a pharmaceutically acceptable salt, solvate, tautomer, or stereoisomer thereof:

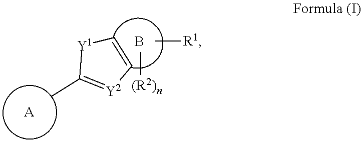

wherein:

### Ring A is

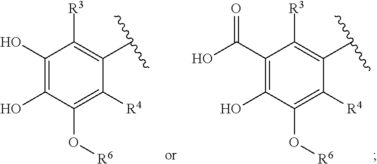

Ring B is phenyl or a 6-membered heteroaryl;
R¹ is hydrogen, deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more Ria;
each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
Y¹ is O, S, or NR^{Y1};
Y² is N or CR^{Y2}.
provided that when Y² is CR^{Y2}, Y¹ is not O;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a};
R^{Y2} is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y2a};
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R^{1a} is independently deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1b};
or two R^{1a} on the same carbon are taken together to form an oxo;
each R¹⁰ is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{10a};
each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{11a};
each R¹² and R¹³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a}.
or R¹² and R¹³ are taken together with the nitrogen atom to which they are attached to form a heterocycloalkyl optionally substituted with one or more R^{13a}.
each R^{Y1a}, R^{Y2a}, R^{2a}, R^{3a}, R^{4a}, R^{6a}, R^{10a}, R^{11a}, R^{12a}, R^{13a}, and R^{1b} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{Y2a}, two R^{2a}, two R^{3a}, two R^{4a}, two R^{5a}, two R^{6a}, two R^{10a}, two R^{11a}, two R^{12a}, two R^{13a}, and two R^{1b} on the same carbon are taken together to form an oxo, a cycloalkyl, or a heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; provided that the compound of Formula (I) is not

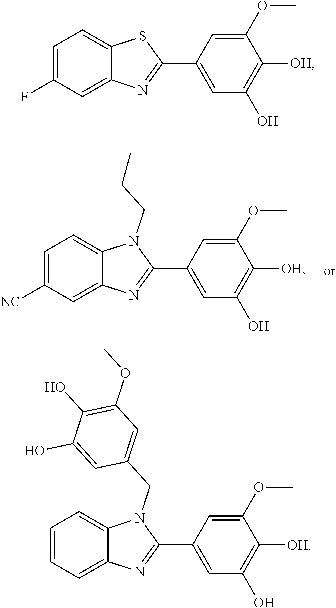 [SMILES: COc1cc(-c2nc3cc(F)ccc3s2)cc(O)c1O ; CCCn1c(-c2cc(O)c(O)c(OC)c2)nc2cc(C#N)ccc21 ; COc1cc(Cn2c(-c3cc(O)c(O)c(OC)c3)nc3ccccc32)cc(O)c1O]

Disclosed herein is a compound of Formula (II) or (III), or a pharmaceutically acceptable salt, solvate, tautomer, or stereoisomer thereof:

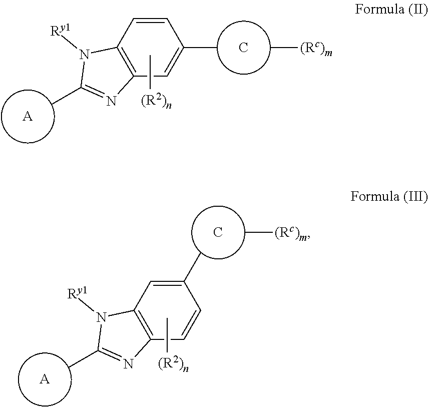

wherein:

### Ring A is

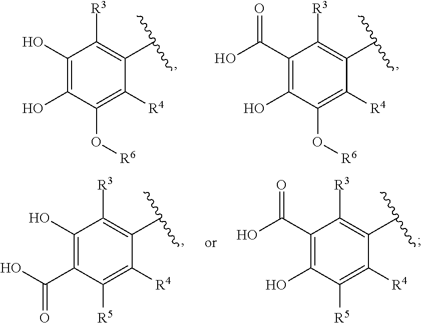

Ring C is cycloalkyl or heterocycloalkyl;
each R^{C} is independently deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{Ca};
or two R^{C} on the same carbon are taken together to form an oxo;
m is 1-3,
each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NRbC(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a}.
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or R⁴ and R⁵ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R¹⁰ is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{10a};
each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{11a};
each R¹² and R¹³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a}.
or R¹² and R¹³ are taken together with the nitrogen atom to which they are attached to form a heterocycloalkyl optionally substituted with one or more R^{13a}.
each R^{Y1a}, R^{2a}, R^{3a}, R^{4a}, R^{5a}, R^{6a}, R^{10a}, R^{11a}, R^{12a}, R^{13a}, and R^{Ca} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{2a}, two R^{3a}, two R^{4a}, two R^{5a}, two R^{6a}, two R^{10a}, two R^{11a}, two R^{12a}, two R^{13a}, and two R^{Ca} on the same carbon are taken together to form an oxo, a cycloalkyl, or heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

Disclosed herein is a compound of Formula (IV) and (V), or a pharmaceutically acceptable salt, solvate, tautomer, or stereoisomer thereof:

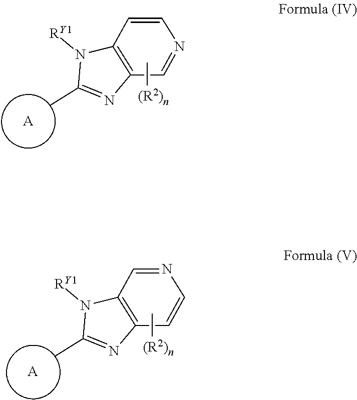

wherein:
Ring A is

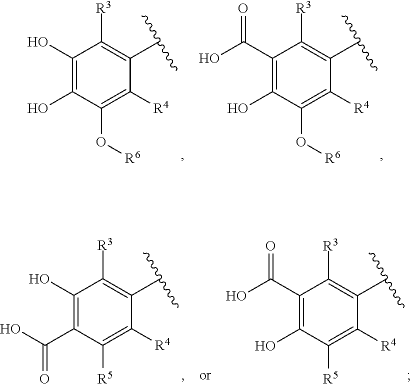

each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a}.
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or R⁴ and R⁵ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R^{Y1a}, R^{2a}, R^{3a}, R^{4a}, R^{5a}, R^{6a}, R^{10a}, R^{11a}, R^{12a}, R^{13a}, and R^{Ca} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{2a}, two R^{3a}, two R^{4a}, two R^{5a}, and two R^{6a} on the same carbon are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

Disclosed herein is a pharmaceutical composition comprising a compound disclosed herein, or a pharmaceutically acceptable salt, solvate, tautomer, or stereoisomer thereof, and a pharmaceutically acceptable excipient.

Disclosed herein is a method of treating cancer in a subject in need thereof, the method comprising administering a compound disclosed herein, or a pharmaceutically acceptable salt, solvate, tautomer, or stereoisomer thereof. In some embodiments, the cancer is characterized by a deficiency in one or more DNA repair pathways. In some embodiments, the DNA repair deficiency is a deficiency in the base excision repair (“BER”) pathway, the Fanconi anaemia-mediated repair (“FA”) pathway, the homologous recombination (“HR”) pathway, the nucleotide excision repair (“NER”) pathway, the non-homologous end joining (“NHEJ”) pathway, the mismatch repair (“MMR”) pathway, the RecQ-mediated repair (“RecQ”) pathway, or the double-stranded breaks (“DSB”) pathway. In some embodiments, the DNA repair deficiency is a deficiency in the homologous recombination (“HR”) pathway. In some embodiments, the DNA repair deficiency is a BRCA1 mutation. In some embodiments, the method further comprises administering a DNA repair inhibitor. In some embodiments, the DNA repair inhibitor is a poly ADP ribose polymerase (“PARP”) inhibitor. In some embodiments, the method further comprises administering an alkylating agent. In some embodiments, the alkylating agent is cyclophosphamide, chlormethine, uramustine, melphalan, chlorambucil, ifosfamide, bendamustine, carmustine, lomustine, nimustine, fotemustine, streptozocin, or busulfan. In some embodiments, the method further comprises administering a DNA damaging agent. In some embodiments, the DNA damaging agent is camptothecin, etoposide, oxaliplatin, cisplatin, or doxorubicin. In some embodiments, the compound is administered in conjunction with high-dose radiotherapy. In some embodiments, the high-dose radiotherapy is administered as a single dose and/or hypofractionated. In some embodiments, the compound is administered in conjunction with Stereotactic Body Radiation Therapy (SBRT).

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B. show how example 107 inhibits human TREX1 and prevents DNA degradation.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Oxo” refers to ═O.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₅ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₅ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₅ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₅ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl comprises 1 or 2 heteroatoms selected from nitrogen and oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₅ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl. Unless stated otherwise specifically in the specification, a heteroalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

### Compounds

Described herein are compounds that are useful in treating diseases associated with TREX1 and STING dysfunction. In some embodiments, the compounds disclosed herein are TREX1 inhibitors. In some embodiments, the compounds disclosed herein are reversible TREX1 inhibitors. In some embodiments, the compounds disclosed herein are reversible, non-competitive TREX1 inhibitors.

Disclosed herein is a compound of Formula (I), or a pharmaceutically acceptable salt, solvate, tautomer, or stereoisomer thereof:

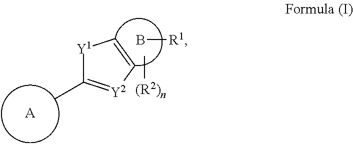

wherein:
Ring A is

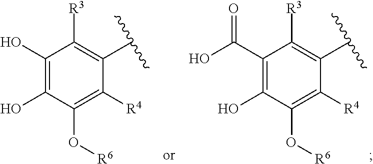

Ring B is phenyl or a 6-membered heteroaryl;
R¹ is hydrogen, deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more Ria;
each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
Y¹ is O, S, or NR^{Y1};
Y² is N or CR^{Y2}.
provided that when Y² is CR^{Y2}, Y¹ is not O;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a}.
R^{Y2} is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y2a};
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R^{1a} is independently deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1b};
or two R^{1a} on the same carbon are taken together to form an oxo;
each R¹⁰ is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{10a};
each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{11a};
each R¹² and R¹³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a};
or R¹² and R¹³ are taken together with the nitrogen atom to which they are attached to form a heterocycloalkyl optionally substituted with one or more R^{13a};
each R^{Y1a}, R^{Y2a}, R^{2a}, R^{3a}, R^{4a}, R^{6a}, R^{10a}, R^{11a}, R^{12a}, R^{13a}, and R^{1b} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{Y2a}, two R^{2a}, two R^{3a}, two R^{4a}, two R^{5a}, two R^{6a}, two R^{10a}, two R^{11a}, two R^{12a}, two R^{13a}, and two R^{1b} on the same carbon are taken together to form an oxo, a cycloalkyl, or a heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
   provided that the compound of Formula (I) is not

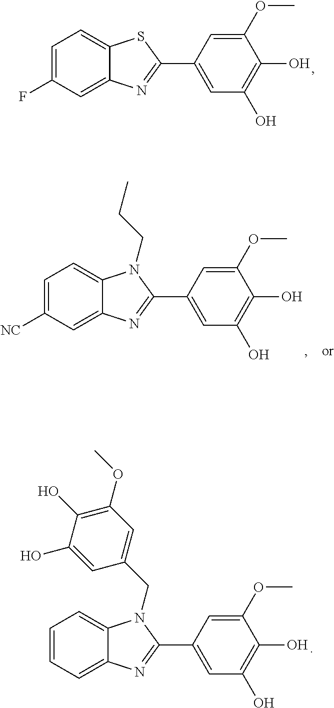 [SMILES: COc1cc(-c2nc3cc(F)ccc3s2)cc(O)c1O ; CCCn1c(-c2cc(O)c(O)c(OC)c2)nc2cc(C#N)ccc21 ; COc1cc(Cn2c(-c3cc(O)c(O)c(OC)c3)nc3ccccc32)cc(O)c1O]

In some embodiments of a compound of Formula (I), or a pharmaceutically acceptable salt, solvate, tautomer, or stereoisomer thereof, the compound is of Formula (Ia):

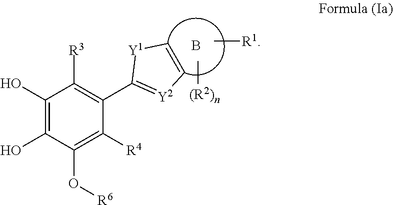

In some embodiments of a compound of Formula (I), or a pharmaceutically acceptable salt, solvate, tautomer, or stereoisomer thereof, the compound is of Formula (Ib)

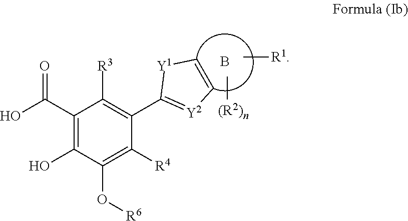

In some embodiments of a compound of Formula (I), (Ia), or (Ib), Ring B is phenyl. In some embodiments of a compound of Formula (I), (Ia), or (Ib), Ring B is pyridyl. In some embodiments of a compound of Formula (I), (Ia), or (Ib), Ring B is pyrimidyl. In some embodiments of a compound of Formula (I), (Ia), or (Ib), Ring B is pyrazinyl. In some embodiments of a compound of Formula (I), (Ia), or (Ib), Ring B is pyridazinyl.

In some embodiments of a compound of Formula (I), (Ia), or (Ib),

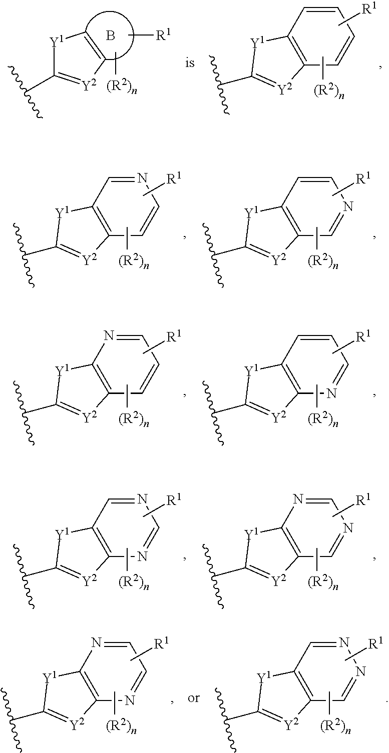

In some embodiments of a compound of Formula (I), (Ia), or (Ib),

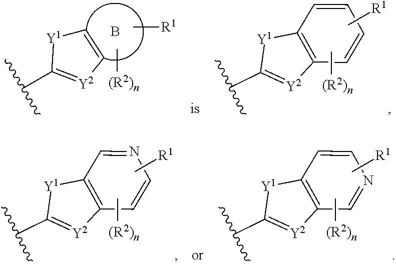

In some embodiments of a compound of Formula (I), (Ia), or (Ib),

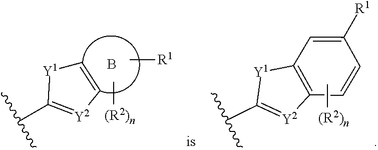

In some embodiments of a compound of Formula (I), (Ia), or (Ib),

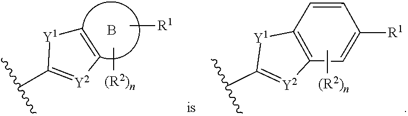

In some embodiments of a compound of Formula (I), (Ia), or (Ib),

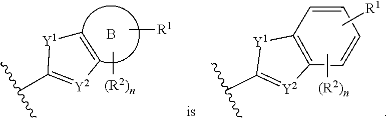

In some embodiments of a compound of Formula (I), (Ia), or (Ib),

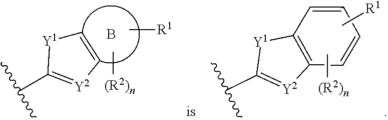

In some embodiments of a compound of Formula (I), (Ia), or (Ib), Y¹ is O. In some embodiments of a compound of Formula (I), (Ia), or (Ib), Y¹ is S.

In some embodiments of a compound of Formula (I), (Ia), or (Ib), Y¹ is NR^{Y1}. In some embodiments of a compound of Formula (I), (Ia), or (Ib), Y² is N. In some embodiments of a compound of Formula (I), (Ia), or (Ib), Y² is CR^{Y2}, provided that Y¹ is not O.

In some embodiments of a compound of Formula (I), (Ia), or (Ib), Y¹ is S and Y² is N. In some embodiments of a compound of Formula (I), (Ia), or (Ib), Y¹ is O and Y² is N. In some embodiments of a compound of Formula (I), (Ia), or (Ib), Y¹ is NRIW and Y² is N. In some embodiments of a compound of Formula (I), (Ia), or (Ib), Y¹ is NR^{Y1} and Y² is CR^{Y2}.

In some embodiments of a compound of Formula (I), (Ia), or (Ib), R^{Y1} is —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a}. In some embodiments of a compound of Formula (I), (Ia), or (Ib), R^{Y1} is C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{Y1a}. In some embodiments of a compound of Formula (I), (Ia), or (Ib), R^{Y1} is hydrogen, C₁-C₆alkyl, or heterocycloalkyl. In some embodiments of a compound of Formula (I), (Ia), or (Ib), R^{Y1} is hydrogen. In some embodiments of a compound of Formula (I), (Ia), or (Ib), R^{Y1} is heterocycloalkyl. In some embodiments of a compound of Formula (I), (Ia), or (Ib), R^{Y1} is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, or heterocycloalkyl; wherein the alkyl, and heterocycloalkyl is optionally substituted with one or more R^{Y1a}.

In some embodiments of a compound of Formula (I), (Ia), or (Ib), R^{Y1} is cycloalkyl or heterocycloalkyl; wherein the cycloalkyl and heterocycloalkyl is optionally substituted with one or more R^{Y1a}. In some embodiments of a compound of Formula (I), (Ia), or (Ib), R^{Y1} is cycloalkyl optionally substituted with one or more R^{Y1a}. In some embodiments of a compound of Formula (I), (Ia), or (Ib), R^{Y1} is a bicyclic cycloalkyl optionally substituted with one or more R^{Y1a}. In some embodiments of a compound of Formula (I), (Ia), or (Ib), R^{Y1} is heterocycloalkyl optionally substituted with one or more R^{Y1a}. In some embodiments of a compound of Formula (I), (Ia), or (Ib), R^{Y1} is

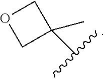 [SMILES: *C1(C)COC1]

In some embodiments of a compound of Formula (I), (Ia), or (Ib), R^{Y1} is

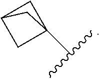 [SMILES: *C12CC(C1)C2]

In some embodiments of a compound of Formula (I), (Ia), or (Ib), the alkyl, alkenyl, alkynyl, cycloalkyl, heterocycloalkyl, aryl, and heteroaryl of R^{Y1} is optionally substituted with one, two, or three R^{Y1a}. In some embodiments of a compound of Formula (I), (Ia), or (Ib), the alkyl, alkenyl, alkynyl, cycloalkyl, heterocycloalkyl, aryl, and heteroaryl of R^{Y1} is optionally substituted with one or two R^{Y1a}. In some embodiments of a compound of Formula (I), (Ia), or (Ib), the alkyl, alkenyl, alkynyl, cycloalkyl, heterocycloalkyl, aryl, and heteroaryl of R^{Y1} is optionally substituted with one R^{Y1a}.

In some embodiments of a compound of Formula (I), (Ia), or (Ib), each R^{Y1a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{Y1a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{Y1a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{Y1a} is independently deuterium, halogen, C₁-C₆alkyl, or C₁-C₆haloalkyl. In some embodiments of a compound of Formula (I), (Ia), or (Ib), each R^{Y1a} is independently halogen or C₁-C₆alkyl. In some embodiments of a compound of Formula (I), (Ia), or (Ib), each R^{Y1a} is independently C₁-C₆alkyl.

In some embodiments of a compound of Formula (I), (Ia), or (Ib), R^{Y2} is hydrogen, deuterium, halogen, —CN, —OR^{b}, —NR^{c}R^{d}, C₁-C₆alkyl, C₁-C₆haloalkyl, C₁-C₆deuteroalkyl, cycloalkyl, heterocycloalkyl, aryl, or heteroaryl; wherein the alkyl, cycloalkyl, heterocycloalkyl, aryl, and heteroaryl is optionally substituted with one or more R^{Y2a}. In some embodiments of a compound of Formula (I), (Ia), or (Ib), R^{Y2} is hydrogen, halogen, C₁-C₆alkyl, or C₁-C₆haloalkyl. In some embodiments of a compound of Formula (I), (Ia), or (Ib), R^{Y2} is hydrogen.

In some embodiments of a compound of Formula (I), (Ia), or (Ib), the alkyl, alkenyl, alkynyl, cycloalkyl, heterocycloalkyl, aryl, and heteroaryl of R^{Y2} is optionally substituted with one, two, or three R^{Y2a}. In some embodiments of a compound of Formula (I), (Ia), or (Ib), the alkyl, alkenyl, alkynyl, cycloalkyl, heterocycloalkyl, aryl, and heteroaryl of R^{Y2} is optionally substituted with one or two R^{Y2a}. In some embodiments of a compound of Formula (I), (Ia), or (Ib), the alkyl, alkenyl, alkynyl, cycloalkyl, heterocycloalkyl, aryl, and heteroaryl of R^{Y2} is optionally substituted with one R^{Y2a}.

In some embodiments of a compound of Formula (I), (Ia), or (Ib), each R^{Y2a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{Y2a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{Y2a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{Y2a} is independently deuterium, halogen, C₁-C₆alkyl, or C₁-C₆haloalkyl. In some embodiments of a compound of Formula (I), (Ia), or (Ib), each R^{Y2a} is independently halogen or C₁-C₆alkyl.

In some embodiments of a compound of Formula (I), (Ia), or (Ib), R³ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{3a}. In some embodiments of a compound of Formula (I), (Ia), or (Ib), R³ is hydrogen, deuterium, halogen, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R^{3a}. In some embodiments of a compound of Formula (I), (Ia), or (Ib), R³ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl. In some embodiments of a compound of Formula (I), (Ia), or (Ib), R³ is hydrogen, halogen, C₁-C₆alkyl, or C₁-C₆haloalkyl. In some embodiments of a compound of Formula (I), (Ia), or (Ib), R³ is hydrogen or halogen. In some embodiments of a compound of Formula (I), (Ia), or (Ib), R³ is hydrogen. In some embodiments of a compound of Formula (I), (Ia), or (Ib), R³ is halogen, C₁-C₆alkyl, or C₁-C₆haloalkyl. In some embodiments of a compound of Formula (I), (Ia), or (Ib), R³ is halogen.

In some embodiments of a compound of Formula (I), (Ia), or (Ib), the alkyl, alkenyl, alkynyl, cycloalkyl, heterocycloalkyl, aryl, and heteroaryl of R³ is optionally substituted with one, two, or three R^{3a}. In some embodiments of a compound of Formula (I), (Ia), or (Ib), the alkyl, alkenyl, alkynyl, cycloalkyl, heterocycloalkyl, aryl, and heteroaryl of R³ is optionally substituted with one or two R^{3a}. In some embodiments of a compound of Formula (I), (Ia), or (Ib), the alkyl, alkenyl, alkynyl, cycloalkyl, heterocycloalkyl, aryl, and heteroaryl of R³ is optionally substituted with one R^{3a}.

In some embodiments of a compound of Formula (I), (Ia), or (Ib), each R^{3a} is independently deuterium, halogen, —CN, —OR^{b}, —NR^{c}R^{d}, —C(═O)R^{a}, —C(═O)OR^{b}, —C(═O)NRR, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{3a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{3a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{3a} is independently deuterium, halogen, C₁-C₆alkyl, or C₁-C₆haloalkyl. In some embodiments of a compound of Formula (I), (Ia), or (Ib), each R^{3a} is independently halogen or C₁-C₆alkyl.

In some embodiments of a compound of Formula (I), (Ia), or (Ib), R⁴ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{4a}. In some embodiments of a compound of Formula (I), (Ia), or (Ib), R⁴ is hydrogen, deuterium, halogen, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R^{4a}. In some embodiments of a compound of Formula (I), (Ia), or (Ib), R⁴ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl. In some embodiments of a compound of Formula (I), (Ia), or (Ib), R⁴ is hydrogen, halogen, C₁-C₆alkyl, or C₁-C₆haloalkyl. In some embodiments of a compound of Formula (I), (Ia), or (Ib), R⁴ is hydrogen or halogen. In some embodiments of a compound of Formula (I), (Ia), or (Ib), R⁴ is hydrogen. In some embodiments of a compound of Formula (I), (Ia), or (Ib), R⁴ is halogen, C₁-C₆alkyl, or C₁-C₆haloalkyl. In some embodiments of a compound of Formula (I), (Ia), or (Ib), R⁴ is halogen.

In some embodiments of a compound of Formula (I), (Ia), or (Ib), the alkyl, alkenyl, alkynyl, cycloalkyl, heterocycloalkyl, aryl, and heteroaryl of R⁴ is optionally substituted with one, two, or three R^{4a}. In some embodiments of a compound of Formula (I), (Ia), or (Ib), the alkyl, alkenyl, alkynyl, cycloalkyl, heterocycloalkyl, aryl, and heteroaryl of R⁴ is optionally substituted with one or two R^{4a}. In some embodiments of a compound of Formula (I), (Ia), or (Ib), the alkyl, alkenyl, alkynyl, cycloalkyl, heterocycloalkyl, aryl, and heteroaryl of R⁴ is optionally substituted with one R^{4a}.

In some embodiments of a compound of Formula (I), (Ia), or (Ib), each R^{4a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{4a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{4a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{4a} is independently deuterium, halogen, C₁-C₆alkyl, or C₁-C₆haloalkyl. In some embodiments of a compound of Formula (I), (Ia), or (Ib), each R^{4a} is independently halogen or C₁-C₆alkyl.

In some embodiments of a compound of Formula (I), (Ia), or (Ib), R⁶ is C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{6a}. In some embodiments of a compound of Formula (I), (Ia), or (Ib), R⁶ is C₁-C₆alkyl or C₁-C₆haloalkyl. In some embodiments of a compound of Formula (I), (Ia), or (Ib), R⁶ is C₁-C₆alkyl. In some embodiments of a compound of Formula (I), (Ia), or (Ib), R⁶ is methyl.

In some embodiments of a compound of Formula (I), (Ia), or (Ib), R⁴ and R⁶ are taken together to form a heterocycloalkyl. In some embodiments of a compound of Formula (I), (Ia), or (Ib), R⁴ and R⁶ are taken together to form a 5- or 6-membered heterocycloalkyl. In some embodiments of a compound of Formula (I), (Ia), or (Ib), R⁴ and R⁶ are taken together to form a 6-membered heterocycloalkyl.

In some embodiments of a compound of Formula (I), (Ia), or (Ib), the alkyl, alkenyl, alkynyl, cycloalkyl, heterocycloalkyl, aryl, and heteroaryl of R⁶ is optionally substituted with one, two, or three R^{6a}. In some embodiments of a compound of Formula (I), (Ia), or (Ib), the alkyl, alkenyl, alkynyl, cycloalkyl, heterocycloalkyl, aryl, and heteroaryl of R⁶ is optionally substituted with one or two R^{6a}. In some embodiments of a compound of Formula (I), (Ia), or (Ib), the alkyl, alkenyl, alkynyl, cycloalkyl, heterocycloalkyl, aryl, and heteroaryl of R⁶ is optionally substituted with one R^{6a}.

In some embodiments of a compound of Formula (I), (Ia), or (Ib), each R^{6a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{6a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{6a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{6a} is independently deuterium, halogen, C₁-C₆alkyl, or C₁-C₆haloalkyl. In some embodiments of a compound of Formula (I), (Ia), or (Ib), each R^{6a} is independently halogen or C₁-C₆alkyl.

In some embodiments of a compound of Formula (I), (Ia), or (Ib), each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{2a}.

In some embodiments of a compound of Formula (I), (Ia), or (Ib), each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl; wherein the alkyl is optionally substituted with one or more R^{2a}. In some embodiments of a compound of Formula (I), (Ia), or (Ib), each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl. In some embodiments of a compound of Formula (I), (Ia), or (Ib), each R² is independently hydrogen, deuterium, halogen, —CN, —OR^{b}, —NR^{c}R^{d}, C₁-C₆alkyl, C₁-C₆haloalkyl, or C₁-C₆deuteroalkyl. In some embodiments of a compound of Formula (I), (Ia), or (Ib), each R² is independently hydrogen, halogen, C₁-C₆alkyl, or C₁-C₆haloalkyl. In some embodiments of a compound of Formula (I), (Ia), or (Ib), each R² is independently hydrogen or halogen. In some embodiments of a compound of Formula (I), (Ia), or (Ib), each R² is hydrogen.

In some embodiments of a compound of Formula (I), (Ia), or (Ib), the alkyl, alkenyl, alkynyl, cycloalkyl, heterocycloalkyl, aryl, and heteroaryl of R² is optionally substituted with one, two, or three R^{2a}. In some embodiments of a compound of Formula (I), (Ia), or (Ib), the alkyl, alkenyl, alkynyl, cycloalkyl, heterocycloalkyl, aryl, and heteroaryl of R² is optionally substituted with one or two R^{2a}. In some embodiments of a compound of Formula (I), (Ia), or (Ib), the alkyl, alkenyl, alkynyl, cycloalkyl, heterocycloalkyl, aryl, and heteroaryl of R² is optionally substituted with one R^{2a}.

In some embodiments of a compound of Formula (I), (Ia), or (Ib), each R^{2a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{2a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{2a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{2a} is independently deuterium, halogen, C₁-C₆alkyl, or C₁-C₆haloalkyl. In some embodiments of a compound of Formula (I), (Ia), or (Ib), each R^{2a} is independently halogen or C₁-C₆alkyl.

In some embodiments of a compound of Formula (I), (Ia), or (Ib), n is 1 or 2. In some embodiments of a compound of Formula (I), (Ia), or (Ib), n is 1. In some embodiments of a compound of Formula (I), (Ia), or (Ib), n is 2.

In some embodiments of a compound of Formula (I), (Ia), or (Ib), R¹ is hydrogen, deuterium, halogen, —CN, —OR¹¹, —NR¹²R¹³, —C(═O)R¹⁰, —C(═O)OR¹¹, —C(═O)NR¹²R¹³, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{1a}. In some embodiments of a compound of Formula (I), (Ia), or (Ib), R¹ is hydrogen, deuterium, halogen, —CN, —OR¹¹, —NR¹²R¹³, —C(═O)R¹⁰, —C(═O)OR¹¹, —C(═O)NR¹²R¹³, C₁-C₆alkyl, C₁-C₆haloalkyl, or C₁-C₆deuteroalkyl. In some embodiments of a compound of Formula (I), (Ia), or (Ib), R¹ is hydrogen, halogen, C₁-C₆alkyl, or C₁-C₆haloalkyl. In some embodiments of a compound of Formula (I), (Ia), or (Ib), R¹ is hydrogen or halogen. In some embodiments of a compound of Formula (I), (Ia), or (Ib), R¹ is hydrogen. In some embodiments of a compound of Formula (I), (Ia), or (Ib), R¹ is cycloalkyl, heterocycloalkyl, aryl, or heteroaryl; wherein the cycloalkyl, heterocycloalkyl, aryl, and heteroaryl is optionally substituted with one or more R^{1a}. In some embodiments of a compound of Formula (I), (Ia), or (Ib), R¹ is cycloalkyl optionally substituted with one or more R^{1a}. In some embodiments of a compound of Formula (I), (Ia), or (Ib), R¹ is heterocycloalkyl optionally substituted with one or more R^{1a}. In some embodiments of a compound of Formula (I), (Ia), or (Ib), R¹ is heterocycloalkyl optionally substituted with one or more R^{1a}; wherein the heterocycloalkyl is piperidinyl or piperazinyl. In some embodiments of a compound of Formula (I), (Ia), or (Ib), R¹ is piperidinyl optionally substituted with one or more R^{1a}. In some embodiments of a compound of Formula (I), (Ia), or (Ib), R¹ is piperazinyl optionally substituted with one or more R^{1a}.

In some embodiments of a compound of Formula (I), (Ia), or (Ib), R¹ is —NR¹²R¹³, —C(═O)NR¹²R¹³, or —NR¹¹C(═O)R¹⁰. In some embodiments of a compound of Formula (I), (Ia), or (Ib), R¹ is —C(═O)NR¹²R¹³ or —NR¹¹C(═O)R¹⁰. In some embodiments of a compound of Formula (I), (Ia), or (Ib), R¹ is —C(═O)NR¹²R¹³. In some embodiments of a compound of Formula (I), (Ia), or (Ib), R¹ is heteroaryl optionally substituted with one or more R^{1a}.

In some embodiments of a compound of Formula (I), (Ia), or (Ib), the alkyl, alkenyl, alkynyl, cycloalkyl, heterocycloalkyl, aryl, and heteroaryl of R¹ is optionally substituted with one, two, or three Ria. In some embodiments of a compound of Formula (I), (Ia), or (Ib), the alkyl, alkenyl, alkynyl, cycloalkyl, heterocycloalkyl, aryl, and heteroaryl of R¹ is optionally substituted with one or two Ria. In some embodiments of a compound of Formula (I), (Ia), or (Ib), the alkyl, alkenyl, alkynyl, cycloalkyl, heterocycloalkyl, aryl, and heteroaryl of R¹ is optionally substituted with one R^{1a}.

In some embodiments of a compound of Formula (I), (Ia), or (Ib), each Ria is independently deuterium, halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, —C(═O)OR¹¹, —C(═O)NR¹²R¹³, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more R^{1b}; or two R^{1a} on the same carbon are taken together to form an oxo. In some embodiments of a compound of Formula (I), (Ia), or (Ib), each Ria is independently deuterium, halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, —C(═O)OR¹¹, —C(═O)NR¹²R¹³, C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is independently optionally substituted with one or more R^{1b}; or two R^{1a} on the same carbon are taken together to form an oxo. In some embodiments of a compound of Formula (I), (Ia), or (Ib), each R^{1a} is independently halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, C₁-C₆alkyl, C₁-C₆haloalkyl, or heteroaryl; wherein each alkyl and heteroaryl is independently optionally substituted with one or more R^{1b}; or two R^{1a} on the same carbon are taken together to form an oxo. In some embodiments of a compound of Formula (I), (Ia), or (Ib), each R^{1a} is independently —C(═O)R^{c}.

In some embodiments of a compound of Formula (I), (Ia), or (Ib), the alkyl, alkenyl, alkynyl, cycloalkyl, heterocycloalkyl, aryl, and heteroaryl of R^{1a} is optionally substituted with one, two, or three R^{1b}. In some embodiments of a compound of Formula (I), (Ia), or (Ib), the alkyl, alkenyl, alkynyl, cycloalkyl, heterocycloalkyl, aryl, and heteroaryl of R^{1a} is optionally substituted with one or two R^{1b}. In some embodiments of a compound of Formula (I), (Ia), or (Ib), the alkyl, alkenyl, alkynyl, cycloalkyl, heterocycloalkyl, aryl, and heteroaryl of R^{1a} is optionally substituted with one R^{1b}.

In some embodiments of a compound of Formula (I), (Ia), or (Ib), each R^{1b} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{1b} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{1b} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{1b} is independently deuterium, halogen, C₁-C₆alkyl, or C₁-C₆haloalkyl. In some embodiments of a compound of Formula (I), (Ia), or (Ib), each R^{1b} is independently halogen or C₁-C₆alkyl.

In some embodiments of a compound of Formula (I), (Ia), or (Ib), each R¹⁰ is independently C₁-C₆alkyl, C₂-C₆alkenyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, alkenyl, cycloalkyl, heterocycloalkyl, aryl, and heteroaryl is independently optionally substituted with one or more R^{10a}. In some embodiments of a compound of Formula (I), (Ia), or (Ib), each R¹⁰ is independently C₁-C₆alkyl, C₂-C₆alkenyl, C₁-C₆aminoalkyl, aryl, or heteroaryl; wherein each alkyl, alkenyl, aryl, and heteroaryl is independently optionally substituted with one or more R^{10a}. In some embodiments of a compound of Formula (I), (Ia), or (Ib), each R¹⁰ is independently aryl optionally substituted with one or more R^{10a}.

In some embodiments of a compound of Formula (I), (Ia), or (Ib), the alkyl, alkenyl, alkynyl, cycloalkyl, heterocycloalkyl, aryl, and heteroaryl of R¹⁰ is optionally substituted with one, two, or three R^{10a}. In some embodiments of a compound of Formula (I), (Ia), or (Ib), the alkyl, alkenyl, alkynyl, cycloalkyl, heterocycloalkyl, aryl, and heteroaryl of R¹⁰ is optionally substituted with one or two R^{10a}. In some embodiments of a compound of Formula (I), (Ia), or (Ib), the alkyl, alkenyl, alkynyl, cycloalkyl, heterocycloalkyl, aryl, and heteroaryl of R¹⁰ is optionally substituted with one R^{10a}.

In some embodiments of a compound of Formula (I), (Ia), or (Ib), each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (I), (Ia), or (Ib), each R^{10a} is independently halogen, —OR^{b}, —C(═O)OR^{b}, or C₁-C₆alkyl. In some embodiments of a compound of Formula (I), (Ia), or (Ib), each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{10a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{10a} is independently deuterium, halogen, C₁-C₆alkyl, or C₁-C₆haloalkyl. In some embodiments of a compound of Formula (I), (Ia), or (Ib), each R^{10a} is independently halogen or C₁-C₆alkyl.

In some embodiments of a compound of Formula (I), (Ia), or (Ib), each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkynyl, cycloalkyl, heterocycloalkyl, aryl, and heteroaryl is independently optionally substituted with one or more R^{1a}. In some embodiments of a compound of Formula (I), (Ia), or (Ib), each R¹¹ is independently hydrogen, C₁-C₆alkyl, C₁-C₆haloalkyl, C₁-C₆deuteroalkyl, cycloalkyl, heterocycloalkyl, aryl, or heteroaryl; wherein each alkynyl, cycloalkyl, heterocycloalkyl, aryl, and heteroaryl is independently optionally substituted with one or more R^{1a}. In some embodiments of a compound of Formula (I), (Ia), or (Ib), each R¹¹ is independently aryl optionally substituted with one or more R^{11a}.

In some embodiments of a compound of Formula (I), (Ia), or (Ib), the alkyl, alkenyl, alkynyl, cycloalkyl, heterocycloalkyl, aryl, and heteroaryl of R¹¹ is optionally substituted with one, two, or three R^{1a}. In some embodiments of a compound of Formula (I), (Ia), or (Ib), the alkyl, alkenyl, alkynyl, cycloalkyl, heterocycloalkyl, aryl, and heteroaryl of R¹¹ is optionally substituted with one or two R^{1a}. In some embodiments of a compound of Formula (I), (Ia), or (Ib), the alkyl, alkenyl, alkynyl, cycloalkyl, heterocycloalkyl, aryl, and heteroaryl of R¹¹ is optionally substituted with one R^{11a}.

In some embodiments of a compound of Formula (I), (Ia), or (Ib), each R^{11a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (I), (Ia), or (Ib), each R^{11a} is independently halogen, —OR^{b}, —C(═O)OR^{b}, or C₁-C₆alkyl. In some embodiments of a compound of Formula (I), (Ia), or (Ib), each R^{11a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{11a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{11a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{11a} is independently deuterium, halogen, C₁-C₆alkyl, or C₁-C₆haloalkyl. In some embodiments of a compound of Formula (I), (Ia), or (Ib), each R^{11a} is independently halogen or C₁-C₆alkyl.

In some embodiments of a compound of Formula (I), (Ia), or (Ib), each R¹² and R¹³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a}.

In some embodiments of a compound of Formula (I), (Ia), or (Ib), the alkyl, alkenyl, alkynyl, cycloalkyl, heterocycloalkyl, aryl, and heteroaryl of R¹² is optionally substituted with one, two, or three R^{12a}. In some embodiments of a compound of Formula (I), (Ia), or (Ib), the alkyl, alkenyl, alkynyl, cycloalkyl, heterocycloalkyl, aryl, and heteroaryl of R¹² is optionally substituted with one or two R^{12a}. In some embodiments of a compound of Formula (I), (Ia), or (Ib), the alkyl, alkenyl, alkynyl, cycloalkyl, heterocycloalkyl, aryl, and heteroaryl of R¹² is optionally substituted with one R^{12a}.

In some embodiments of a compound of Formula (I), (Ia), or (Ib), each R^{12a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (I), (Ia), or (Ib), each R^{12a} is independently halogen, —OR^{b}, —C(═O)OR^{b}, or C₁-C₆alkyl. In some embodiments of a compound of Formula (I), (Ia), or (Ib), each R^{12a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{12a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{12a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{12a} is independently deuterium, halogen, C₁-C₆alkyl, or C₁-C₆haloalkyl. In some embodiments of a compound of Formula (I), (Ia), or (Ib), each R^{12a} is independently halogen or C₁-C₆alkyl.

In some embodiments of a compound of Formula (I), (Ia), or (Ib), R¹² and R¹³ are taken together with the nitrogen atom to which they are attached to form a heterocycloalkyl optionally substituted with one or more R^{13a}.

In some embodiments of a compound of Formula (I), (Ia), or (Ib), the heterocycloalkyl formed when R¹² and R¹³ are taken together is optionally substituted with one, two, or three R^{13a}. In some embodiments of a compound of Formula (I), (Ia), or (Ib), the heterocycloalkyl formed when R¹² and R¹³ are taken together is optionally substituted with one or two R^{13a}. In some embodiments of a compound of Formula (I), (Ia), or (Ib), the heterocycloalkyl formed when R¹² and R¹³ are taken together is optionally substituted with one R^{13a}.

In some embodiments of a compound of Formula (I), (Ia), or (Ib), each R^{13a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (I), (Ia), or (Ib), each R^{13a} is independently halogen, —OR^{b}, —C(═O)OR^{b}, or C₁-C₆alkyl. In some embodiments of a compound of Formula (I), (Ia), or (Ib), each R^{13a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{13a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia), or (Ib), each R^{13a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia), or (Ib), each R^{13a} is independently deuterium, halogen, C₁-C₆alkyl, or C₁-C₆haloalkyl. In some embodiments of a compound of Formula (I), (Ia), or (Ib), each R^{13a} is independently halogen or C₁-C₆alkyl.

Disclosed herein is a compound of Formula (II) or (III), or a pharmaceutically acceptable salt, solvate, tautomer, or stereoisomer thereof:

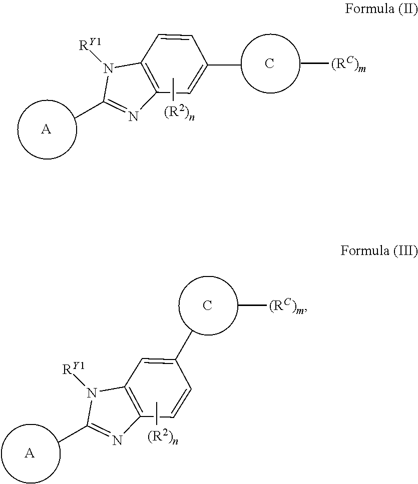

wherein:
Ring A is

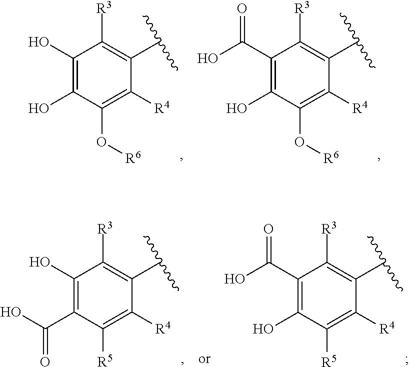

Ring C is cycloalkyl or heterocycloalkyl;
each R^{C} is independently deuterium, halogen, —CN, —OR¹¹, —SR¹¹, —S(═O)R¹⁰, —S(═O)₂R^{c}, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{Ca};
or two R^{C} on the same carbon are taken together to form an oxo;
m is 1-3,
each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a};
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or R⁴ and R⁵ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R¹⁰ is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{10a};
each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{11a};
each R¹² and R³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a}.
or R¹² and R¹³ are taken together with the nitrogen atom to which they are attached to form a heterocycloalkyl optionally substituted with one or more R^{13a}.
each R^{Y1a}, R^{2a}, R^{3a}, R^{4a}, R^{5a}, R^{6a}, R^{10a}, Ru^{11a}, R^{12a}, R^{13a}, and R^{Ca} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{2a}, two R^{3a}, two R^{4a}, two R^{5a}, two R^{6a}, two R^{10a}, two R^{11a}, two R^{12a}, two R^{13a}, and two R^{Ca} on the same carbon are taken together to form an oxo, a cycloalkyl, or heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

In some embodiments of a compound of Formula (II), or a pharmaceutically acceptable salt, solvate, tautomer, or stereoisomer thereof, the compound is of Formula (IIa):

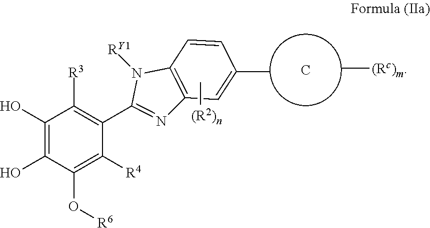

In some embodiments of a compound of Formula (II), or a pharmaceutically acceptable salt, solvate, tautomer, or stereoisomer thereof, the compound is of Formula (IIb):

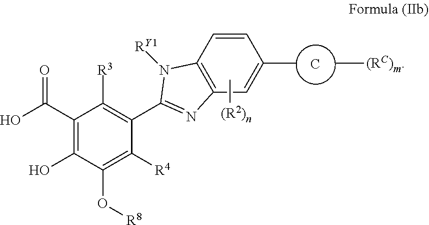

In some embodiments of a compound of Formula (II), or a pharmaceutically acceptable salt, solvate, tautomer, or stereoisomer thereof, the compound is of Formula (IIc):

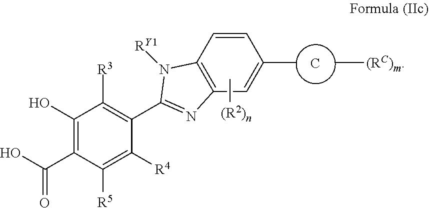

In some embodiments of a compound of Formula (II), or a pharmaceutically acceptable salt, solvate, tautomer, or stereoisomer thereof, the compound is of Formula (IId):

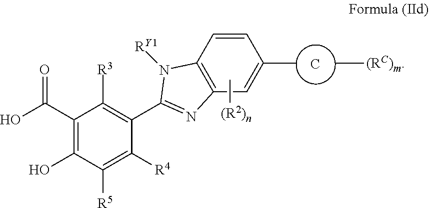

In some embodiments of a compound of Formula (III), or a pharmaceutically acceptable salt, solvate, tautomer, or stereoisomer thereof, the compound is of Formula (IIIa):

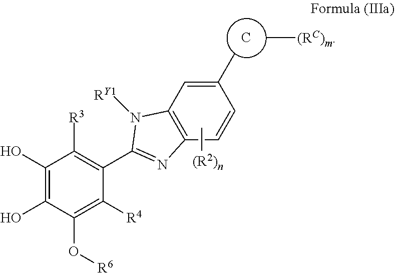

In some embodiments of a compound of Formula (III), or a pharmaceutically acceptable salt, solvate, tautomer, or stereoisomer thereof, the compound is of Formula (IIIb):

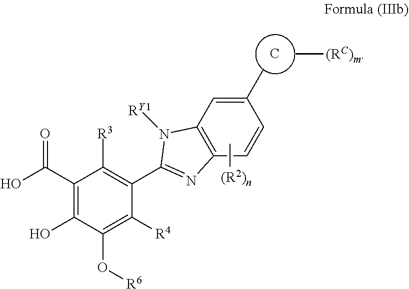

In some embodiments of a compound of Formula (III), or a pharmaceutically acceptable salt, solvate, tautomer, or stereoisomer thereof, the compound is of Formula (IIIc):

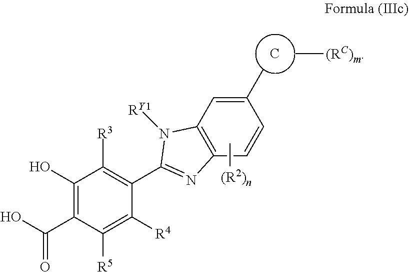

In some embodiments of a compound of Formula (III), or a pharmaceutically acceptable salt, solvate, tautomer, or stereoisomer thereof, the compound is of Formula (IIId):

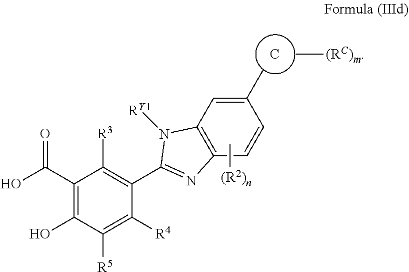

In some embodiments of a compound of Formula (II), (IIa)-(IId), (III), or (IIIa)-(IIId), Ring C is heterocycloalkyl. In some embodiments of a compound of Formula (II), (IIa)-(IId), (III), or (IIIa)-(IIId), Ring C is C₂-C₆ heterocycloalkyl. In some embodiments of a compound of Formula (II), (IIa)-(IId), (III), or (IIIa)-(IIId), Ring C is C₂-C₆ heterocycloalkyl with 1 or 2 heteroatoms selected from N, 0, or S. In some embodiments of a compound of Formula (II), (IIa)-(IId), (III), or (IIIa)-(IIId), Ring C is piperidinyl. In some embodiments of a compound of Formula (II), (IIa)-(IId), (III), or (IIIa)-(IIId), Ring C is piperazinyl.

In some embodiments of a compound of Formula (II), (IIa)-(IId), (III), or (IIIa)-(IIId), Ring C is cycloalkyl.

In some embodiments of a compound of Formula (II), (IIa)-(IId), (III), or (IIIa)-(IIId), each R^{C} is independently deuterium, halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, —C(═O)OR¹¹, —C(═O)NR¹²R¹³, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more R^{Ca}; or two R^{C} on the same carbon are taken together to form an oxo. In some embodiments of a compound of Formula (II), (IIa)-(IId), (III), or (IIIa)-(IIId), each R^{C} is independently deuterium, halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, —C(═O)OR¹¹, —C(═O)NR¹²R¹³, C₁-C₆alkyl, C₁-C₆haloalkyl, cycloalkyl, heterocycloalkyl, aryl, or heteroaryl; wherein each alkyl, cycloalkyl, heterocycloalkyl, aryl, and heteroaryl is independently optionally substituted with one or more R^{Ca}; or two R_{C} on the same carbon are taken together to form an oxo. In some embodiments of a compound of Formula (II), (IIa)-(IId), (III), or (IIIa)-(IIId), each R^{Ca} is independently —CN, —OR¹¹, —S(═O)₂R¹⁰, —NR¹²R¹³, —NHS(═O)₂R¹⁰, —S(═O)₂NR¹²R¹³, —C(═O)R¹⁰, C₁-C₆alkyl, C₁-C₆haloalkyl, or heteroaryl; wherein each alkyl and heteroaryl is independently optionally substituted with one or more R^{Ca}; or two R_{C} on the same carbon are taken together to form an oxo. In some embodiments of a compound of Formula (II), (IIa)-(IId), (III), or (IIIa)-(IIId), each R_{C} is independently —C(═O)R¹⁰.

In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R^{C} is optionally substituted with one, two, or three R^{Ca}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R_{C} is optionally substituted with one or two R^{Ca}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R^{C} is optionally substituted with one R^{Ca}.

In some embodiments of a compound of Formula (II), (IIa)-(IId), (III), or (IIIa)-(IIId), each R^{Ca} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{Ca} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), or (IIIa)-(IIId), each R^{Ca} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{Ca} is independently deuterium, halogen, C₁-C₆alkyl, or C₁-C₆haloalkyl. In some embodiments of a compound of Formula (II), (IIa)-(IId), (III), or (IIIa)-(IIId), each R^{Ca} is independently halogen or C₁-C₆alkyl.

In some embodiments of a compound of Formula (II), (IIa)-(IId), (III), or (IIIa)-(IIId), each R¹⁰ is independently C₁-C₆alkyl, C₂-C₆alkenyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, alkenyl, cycloalkyl, heterocycloalkyl, aryl, and heteroaryl is independently optionally substituted with one or more R^{10a}. In some embodiments of a compound of Formula (II), (IIa)-(IId), (III), or (IIIa)-(IIId), each R¹⁰ is independently C₁-C₆alkyl, C₂-C₆alkenyl, C₁-C₆aminoalkyl, aryl, or heteroaryl; wherein each alkyl, alkenyl, aryl, and heteroaryl is independently optionally substituted with one or more R^{10a}. In some embodiments of a compound of Formula (II), (IIa)-(IId), (III), or (IIIa)-(IIId), each R¹⁰ is independently aryl optionally substituted with one or more R^{10a}.

In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹⁰ is optionally substituted with one, two, or three R^{10a}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹⁰ is optionally substituted with one or two R^{10a}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹⁰ is optionally substituted with one R^{10a}.

In some embodiments of a compound of Formula (II), (IIa)-(IId), (III), or (IIIa)-(IIId), each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (II), (IIa)-(IId), (III), or (IIIa)-(IIId), each R^{10a} is independently halogen, —OR^{b}, —C(═O)OR^{b}, or C₁-C₆alkyl. In some embodiments of a compound of Formula (II), (IIa)-(IId), (III), or (IIIa)-(IIId), each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), or (IIIa)-(IIId), each R^{10a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{10a} is independently deuterium, halogen, C₁-C₆alkyl, or C₁-C₆haloalkyl. In some embodiments of a compound of Formula (II), (IIa)-(IId), (III), or (IIIa)-(IIId), each R^{10a} is independently halogen or C₁-C₆alkyl.

In some embodiments of a compound of Formula (II), (IIa)-(IId), (III), or (IIIa)-(IIId), each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkynyl, cycloalkyl, heterocycloalkyl, aryl, and heteroaryl is independently optionally substituted with one or more R^{11a}. In some embodiments of a compound of Formula (II), (IIa)-(IId), (III), or (IIIa)-(IIId), each R¹¹ is independently hydrogen, C₁-C₆alkyl, C₁-C₆haloalkyl, C₁-C₆deuteroalkyl, cycloalkyl, heterocycloalkyl, aryl, or heteroaryl; wherein each alkynyl, cycloalkyl, heterocycloalkyl, aryl, and heteroaryl is independently optionally substituted with one or more R^{11a}. In some embodiments of a compound of Formula (II), (IIa)-(IId), (III), or (IIIa)-(IIId), each R¹¹ is independently aryl optionally substituted with one or more R^{11a}.

In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹¹ is optionally substituted with one, two, or three R^{1a}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹¹ is optionally substituted with one or two R^{11a}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹¹ is optionally substituted with one R^{11a}.

In some embodiments of a compound of Formula (II), (IIa)-(IId), (III), or (IIIa)-(IIId), each R^{11a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (II), (IIa)-(IId), (III), or (IIIa)-(IIId), each R^{11a} is independently halogen, —OR^{b}, —C(═O)OR^{b}, or C₁-C₆alkyl. In some embodiments of a compound of Formula (II), (IIa)-(IId), (III), or (IIIa)-(IIId), each R^{11a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{11a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), or (IIIa)-(IIId), each R^{11a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{11a} is independently deuterium, halogen, C₁-C₆alkyl, or C₁-C₆haloalkyl. In some embodiments of a compound of Formula (II), (IIa)-(IId), (III), or (IIIa)-(IIId), each R^{11a} is independently halogen or C₁-C₆alkyl.

In some embodiments of a compound of Formula (II), (IIa)-(IId), (III), or (IIIa)-(IIId), each R¹² and R¹³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a}.

In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹² is optionally substituted with one, two, or three R^{12a}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹² is optionally substituted with one or two R^{12a}. In some embodiments of a compound of Formula (II), (IIa)-(IId), (III), or (IIIa)-(IIId), the alkyl, alkenyl, alkynyl, cycloalkyl, heterocycloalkyl, aryl, and heteroaryl of R¹² is optionally substituted with one R^{12a}.

In some embodiments of a compound of Formula (II), (IIa)-(IId), (III), or (IIIa)-(IIId), each R^{12a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (II), (IIa)-(IId), (III), or (IIIa)-(IIId), each R^{12a} is independently halogen, —OR^{b}, —C(═O)OR^{b}, or C₁-C₆alkyl. In some embodiments of a compound of Formula (II), (IIa)-(IId), (III), or (IIIa)-(IIId), each R^{12a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{12a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), or (IIIa)-(IIId), each R^{12a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{12a} is independently deuterium, halogen, C₁-C₆alkyl, or C₁-C₆haloalkyl. In some embodiments of a compound of Formula (II), (IIa)-(IId), (III), or (IIIa)-(IIId), each R^{12a} is independently halogen or C₁-C₆alkyl.

In some embodiments of a compound of Formula (II), (IIa)-(IId), (III), or (IIIa)-(IIId), R¹² and R¹³ are taken together with the nitrogen atom to which they are attached to form a heterocycloalkyl optionally substituted with one or more R^{13a}.

In some embodiments of a compound of Formula (II), (IIa)-(IId), (III), or (IIIa)-(IIId), the heterocycloalkyl formed when R¹² and R³ are taken together is optionally substituted with one, two, or three R^{13a}. In some embodiments of a compound of Formula (II), (IIa)-(IId), (III), or (IIIa)-(IIId), the heterocycloalkyl formed when R¹² and R³ are taken together is optionally substituted with one or two R^{13a}. In some embodiments of a compound of Formula (II), (IIa)-(IId), (III), or (IIIa)-(IIId), the heterocycloalkyl formed when R¹² and R³ are taken together is optionally substituted with one R^{13a}.

In some embodiments of a compound of Formula (II), (IIa)-(IId), (III), or (IIIa)-(IIId), each R^{13a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (II), (IIa)-(IId), (III), or (IIIa)-(IIId), each R^{13a} is independently halogen, —OR^{b}, —C(═O)OR^{b}, or C₁-C₆alkyl. In some embodiments of a compound of Formula (II), (IIa)-(IId), (III), or (IIIa)-(IIId), each R^{13a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{13a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), or (IIIa)-(IIId), each R^{13a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), or (IIIa)-(IIId), each R^{13a} is independently deuterium, halogen, C₁-C₆alkyl, or C₁-C₆haloalkyl. In some embodiments of a compound of Formula (II), (IIa)-(IId), (III), or (IIIa)-(IIId), each R^{13a} is independently halogen or C₁-C₆alkyl.

In some embodiments of a compound of Formula (II), (IIa)-(IId), (III), or (IIIa)-(IIId), m is 1 or 2. In some embodiments of a compound of Formula (II), (IIa)-(IId), (III), or (IIIa)-(IIId), m is 1. In some embodiments of a compound of Formula (II), (IIa)-(IId), or (III), (IIIa)-(IIId), m is 2.

Disclosed herein is a compound of Formula (IV) and (V), or a pharmaceutically acceptable salt, solvate, tautomer, or stereoisomer thereof:

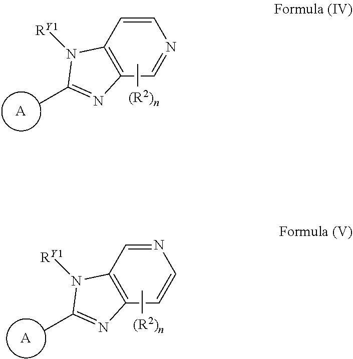

wherein:
Ring A is

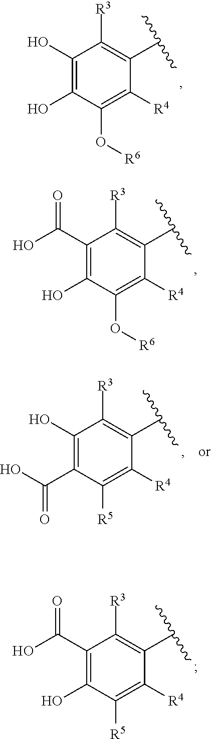

each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1a};
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or R⁴ and R⁵ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R^{Y1a}, R^{2a}, R^{3a}, R^{4a}, R^{5a}, R^{6a}, R^{10a}, R^{11a}, R^{12a}, R^{13a}, and R^{Ca} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR_{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{2a}, two R^{3a}, two R^{4a}, two R^{5a}, and two R^{6a} on the same carbon are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

In some embodiments of a compound of Formula (IV), or a pharmaceutically acceptable salt, solvate, tautomer, or stereoisomer thereof, the compound is of Formula (IVa):

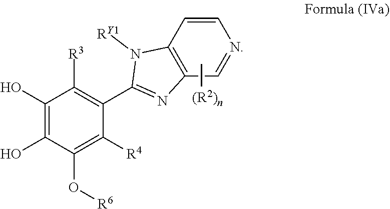

In some embodiments of a compound of Formula (IV), or a pharmaceutically acceptable salt, solvate, tautomer, or stereoisomer thereof, the compound is of Formula (IVb):

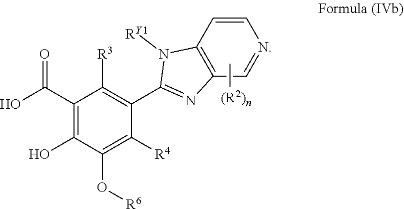

In some embodiments of a compound of Formula (IV), or a pharmaceutically acceptable salt, solvate, tautomer, or stereoisomer thereof, the compound is of Formula (IVc):

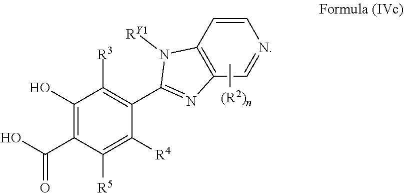

In some embodiments of a compound of Formula (IV), or a pharmaceutically acceptable salt, solvate, tautomer, or stereoisomer thereof, the compound is of Formula (IVd):

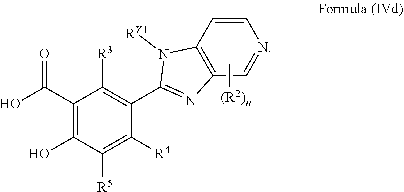

In some embodiments of a compound of Formula (V), or a pharmaceutically acceptable salt, solvate, tautomer, or stereoisomer thereof, the compound is of Formula (Va):

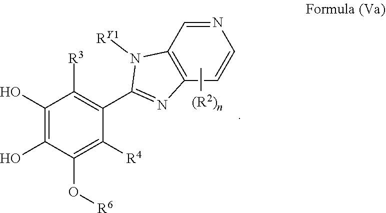

In some embodiments of a compound of Formula (V), or a pharmaceutically acceptable salt, solvate, tautomer, or stereoisomer thereof, the compound is of Formula (Vb):

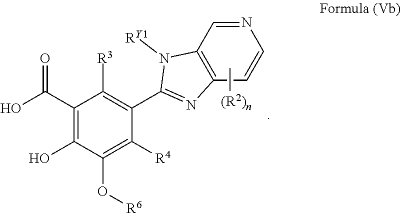

In some embodiments of a compound of Formula (V), or a pharmaceutically acceptable salt, solvate, tautomer, or stereoisomer thereof, the compound is of Formula (Vc):

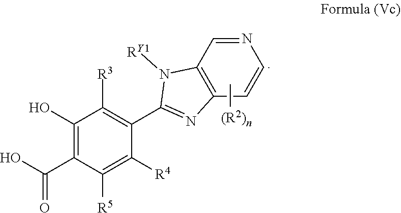

In some embodiments of a compound of Formula (V), or a pharmaceutically acceptable salt, solvate, tautomer, or stereoisomer thereof, the compound is of Formula (Vd):

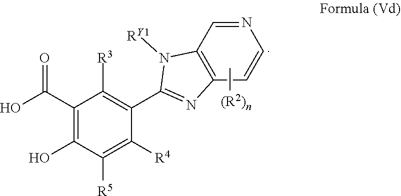

In some embodiments of a compound of Formula (II), (IIa)-(IId), (III), (IIIa)-(IIId), (IV), (IVa)-(IVd), (V), or (Va)-(Vd), R^{Y1} is —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a}. In some embodiments of a compound of Formula (II), (IIa)-(IId), (III), (IIIa)-(IIId), (IV), (IVa)-(IVd), (V), or (Va)-(Vd), R^{Y1} is C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{Y1a}. In some embodiments of a compound of Formula (II), (IIa)-(IId), (III), (IIIa)-(IIId), (IV), (IVa)-(IVd), (V), or (Va)-(Vd), R^{Y1} is hydrogen, C₁-C₆alkyl, or heterocycloalkyl. In some embodiments of a compound of Formula (II), (IIa)-(IId), (III), (IIIa)-(IIId), (IV), (IVa)-(IVd), (V), or (Va)-(Vd), R^{Y1} is hydrogen. In some embodiments of a compound of Formula (II), (IIa)-(IId), (III), (IIIa)-(IIId), (IV), (IVa)-(IVd), (V), or (Va)-(Vd), R^{Y1} is heterocycloalkyl. In some embodiments of a compound of Formula (II), (IIa)-(IId), (III), (IIIa)-(IIId), (IV), (IVa)-(IVd), (V), or (Va)-(Vd), R^{Y1} is hydrogen, —C(═O)R^{a}, C₁-C₆alkyl, C₁-C₆haloalkyl, C₁-C₆deuteroalkyl, C₁-C₆hydroxyalkyl, C₁-C₆aminoalkyl, or heterocycloalkyl; wherein the alkyl, and heterocycloalkyl is optionally substituted with one or more R^{Y1a}.

In some embodiments of a compound of Formula (II), (IIa)-(IId), (III), (IIIa)-(IIId), (IV), (IVa)-(IVd), (V), or (Va)-(Vd), R^{Y1} is cycloalkyl or heterocycloalkyl; wherein the cycloalkyl and heterocycloalkyl is optionally substituted with one or more R^{Y1a}. In some embodiments of a compound of Formula (II), (IIa)-(IId), (III), (IIIa)-(IIId), (IV), (IVa)-(IVd), (V), or (Va)-(Vd), R^{Y1} is cycloalkyl optionally substituted with one or more R^{Y1a}. In some embodiments of a compound of Formula (II), (IIa)-(IId), (III), (IIIa)-(IIId), (IV), (IVa)-(IVd), (V), or (Va)-(Vd), R^{Y1} is a bicyclic cycloalkyl optionally substituted with one or more R^{Y1a}. In some embodiments of a compound of Formula (II), (IIa)-(IId), (III), (IIIa)-(IIId), (IV), (IVa)-(IVd), (V), or (Va)-(Vd), R^{Y1} is heterocycloalkyl optionally substituted with one or more R^{Y1a}. In some embodiments of a compound of Formula (I), (Ia), or (Ib), R^{Y1} is

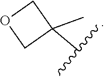 [SMILES: *C1(C)COC1]

In some embodiments of a compound of Formula (I), (Ia), or (Ib), R^{Y1} is

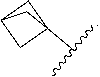 [SMILES: *C12CC(C1)C2]

In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R^{Y1} is optionally substituted with one, two, or three R^{Y1a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R^{Y1} is optionally substituted with one or two R^{Y1a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R^{Y1} is optionally substituted with one R^{Y1a}.

In some embodiments of a compound of Formula (II), (IIa)-(IId), (III), (IIIa)-(IIId), (IV), (IVa)-(IVd), (V), or (Va)-(Vd), each R^{Y1a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{Y1a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), (IIIa)-(IIId), (IV), (IVa)-(IVd), (V), or (Va)-(Vd), each R^{Y1a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{Y1a} is independently deuterium, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), each R^{Y1a} is independently halogen or C₁-C₆alkyl. In some embodiments of a compound of Formula (II), (IIa)-(IId), (III), (IIIa)-(IIId), (IV), (IVa)-(IVd), (V), or (Va)-(Vd), each R^{Y1a} is independently C₁-C₆alkyl.

In some embodiments of a compound of Formula (II), (IIa)-(IId), (III), (IIIa)-(IIId), (IV), (IVa)-(IVd), (V), or (Va)-(Vd), R³ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{3a}. In some embodiments of a compound of Formula (II), (IIa)-(IId), (III), (IIIa)-(IIId), (IV), (IVa)-(IVd), (V), or (Va)-(Vd), R³ is hydrogen, deuterium, halogen, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R^{3a}. In some embodiments of a compound of Formula (II), (IIa)-(IId), (III), (IIIa)-(IIId), (IV), (IVa)-(IVd), (V), or (Va)-(Vd), R³ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl. In some embodiments of a compound of Formula (II), (IIa)-(IId), (III), (IIIa)-(IIId), (IV), (IVa)-(IVd), (V), or (Va)-(Vd), R³ is hydrogen, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), R³ is hydrogen or halogen. In some embodiments of a compound of Formula (II), (IIa)-(IId), (III), (IIIa)-(IIId), (IV), (IVa)-(IVd), (V), or (Va)-(Vd), R³ is hydrogen. In some embodiments of a compound of Formula (II), (IIa)-(IId), (III), (IIIa)-(IIId), (IV), (IVa)-(IVd), (V), or (Va)-(Vd), R³ is halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), R³ is halogen.

In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R³ is optionally substituted with one, two, or three R^{3a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R³ is optionally substituted with one or two R^{3a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R³ is optionally substituted with one R^{3a}.

In some embodiments of a compound of Formula (II), (IIa)-(IId), (III), (IIIa)-(IIId), (IV), (IVa)-(IVd), (V), or (Va)-(Vd), each R^{3a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{3a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), (IIIa)-(IIId), (IV), (IVa)-(IVd), (V), or (Va)-(Vd), each R^{3a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{3a} is independently deuterium, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), each R^{3a} is independently halogen or C₁-C₆alkyl.

In some embodiments of a compound of Formula (II), (IIa)-(IId), (III), (IIIa)-(IIId), (IV), (IVa)-(IVd), (V), or (Va)-(Vd), R⁴ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{4a}. In some embodiments of a compound of Formula (II), (IIa)-(IId), (III), (IIIa)-(IIId), (IV), (IVa)-(IVd), (V), or (Va)-(Vd), R⁴ is hydrogen, deuterium, halogen, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R^{4a}. In some embodiments of a compound of Formula (II), (IIa)-(IId), (III), (IIIa)-(IIId), (IV), (IVa)-(IVd), (V), or (Va)-(Vd), R⁴ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl. In some embodiments of a compound of Formula (II), (IIa)-(IId), (III), (IIIa)-(IIId), (IV), (IVa)-(IVd), (V), or (Va)-(Vd), R⁴ is hydrogen, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), R⁴ is hydrogen or halogen. In some embodiments of a compound of Formula (II), (IIa)-(IId), (III), (IIIa)-(IIId), (IV), (IVa)-(IVd), (V), or (Va)-(Vd), R⁴ is hydrogen. In some embodiments of a compound of Formula (II), (IIa)-(IId), (III), (IIIa)-(IIId), (IV), (IVa)-(IVd), (V), or (Va)-(Vd), R⁴ is halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), R⁴ is halogen.

In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R⁴ is optionally substituted with one, two, or three R^{4a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R⁴ is optionally substituted with one or two R^{4a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R⁴ is optionally substituted with one R^{4a}.

In some embodiments of a compound of Formula (II), (IIa)-(IId), (III), (IIIa)-(IIId), (IV), (IVa)-(IVd), (V), or (Va)-(Vd), each R^{4a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{4a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), (IIIa)-(IIId), (IV), (IVa)-(IVd), (V), or (Va)-(Vd), each R^{4a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{4a} is independently deuterium, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), each R^{4a} is independently halogen or C₁-C₆alkyl.

In some embodiments of a compound of Formula (II), (IIc), (IId), (III), (IIIc), (IIId), (IV), (IVc), (IVd), (V), (Vc), or (Vd), R is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{1a}. In some embodiments of a compound of Formula (II), (IIc), (IId), (III), (IIIc), (IIId), (IV), (IVc), (IVd), (V), (Vc), or (Vd), R⁵ is hydrogen, deuterium, halogen, C₁-C₆alkyl, C₁-C₆haloalkyl, cycloalkyl, heterocycloalkyl, aryl, or heteroaryl; wherein the alkyl, cycloalkyl, heterocycloalkyl, aryl, and heteroaryl is optionally substituted with one or more R^{5a}. In some embodiments of a compound of Formula (II), (IIc), (IId), (III), (IIIc), (IIId), (IV), (IVc), (IVd), (V), (Vc), or (Vd), R is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl. In some embodiments of a compound of Formula (II), (IIc), (IId), (III), (IIIc), (IIId), (IV), (IVc), (IVd), (V), (Vc), or (Vd), R⁵ is hydrogen, halogen, C₁-C₆alkyl, or C₁-C₆haloalkyl. In some embodiments of a compound of Formula (II), (IIc), (IId), (III), (IIIc), (IIId), (IV), (IVc), (IVd), (V), (Vc), or (Vd), R⁵ is hydrogen or halogen. In some embodiments of a compound of Formula (II), (IIc), (IId), (III), (IIIc), (IIId), (IV), (IVc), (IVd), (V), (Vc), or (Vd), R⁵ is hydrogen.

In some embodiments of a compound of Formula (II), (IIc), (IId), (III), (IIIc), (IIId), (IV), (IVc), (IVd), (V), (Vc), or (Vd), the alkyl, alkenyl, alkynyl, cycloalkyl, heterocycloalkyl, aryl, and heteroaryl of R⁵ is optionally substituted with one, two, or three R^{5a}. In some embodiments of a compound of Formula (II), (IIc), (IId), (III), (IIIc), (IIId), (IV), (IVc), (IVd), (V), (Vc), or (Vd), the alkyl, alkenyl, alkynyl, cycloalkyl, heterocycloalkyl, aryl, and heteroaryl of R⁵ is optionally substituted with one or two R^{5a}. In some embodiments of a compound of Formula (II), (IIc), (IId), (III), (IIIc), (IIId), (IV), (IVc), (IVd), (V), (Vc), or (Vd), the alkyl, alkenyl, alkynyl, cycloalkyl, heterocycloalkyl, aryl, and heteroaryl of R⁵ is optionally substituted with one R^{5a}.

In some embodiments of a compound of Formula (II), (IIc), (IId), (III), (IIIc), (IIId), (IV), (IVc), (IVd), (V), (Vc), or (Vd), each R^{5a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIc), (IId), (III), (IIIc), (IIId), (IV), (IVc), (IVd), (V), (Vc), or (Vd), each R^{5a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIc), (IId), (III), (IIIc), (IIId), (IV), (IVc), (IVd), (V), (Vc), or (Vd), each R^{5a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIc), (IId), (III), (IIIc), (IIId), (IV), (IVc), (IVd), (V), (Vc), or (Vd), each R^{5a} is independently deuterium, halogen, C₁-C₆alkyl, or C₁-C₆haloalkyl. In some embodiments of a compound of Formula (II), (IIc), (IId), (III), (IIIc), (IIId), (IV), (IVc), (IVd), (V), (Vc), or (Vd), each R^{5a} is independently halogen or C₁-C₆alkyl.

In some embodiments of a compound of Formula (II), (IIa)-(IId), (III), (IIIa)-(IIId), (IV), (IVa)-(IVd), (V), or (Va)-(Vd), R⁶ is C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{6a}. In some embodiments of a compound of Formula (II), (IIa)-(IId), (III), (IIIa)-(IIId), (IV), (IVa)-(IVd), (V), or (Va)-(Vd), R⁶ is C₁-C₆alkyl or C₁-C₆haloalkyl. In some embodiments of a compound of Formula (II), (IIa)-(IId), (III), (IIIa)-(IIId), (IV), (IVa)-(IVd), (V), or (Va)-(Vd), R⁶ is C₁-C₆alkyl. In some embodiments of a compound of Formula (II), (IIa)-(IId), (III), (IIIa)-(IIId), (IV), (IVa)-(IVd), (V), or (Va)-(Vd), R⁶ is methyl.

In some embodiments of a compound of Formula (II), (IIa)-(IId), (III), (IIIa)-(IIId), (IV), (IVa)-(IVd), (V), or (Va)-(Vd), R⁴ and R⁶ are taken together to form a heterocycloalkyl. In some embodiments of a compound of Formula (II), (IIa)-(IId), (III), (IIIa)-(IIId), (IV), (IVa)-(IVd), (V), or (Va)-(Vd), R⁴ and R⁶ are taken together to form a 5- or 6-membered heterocycloalkyl.

In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R⁶ is optionally substituted with one, two, or three R^{6a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R⁶ is optionally substituted with one or two R^{6a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R⁶ is optionally substituted with one R^{6a}.

In some embodiments of a compound of Formula (II), (IIa)-(IId), (III), (IIIa)-(IIId), (IV), (IVa)-(IVd), (V), or (Va)-(Vd), each R^{6a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{6a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), (IIIa)-(IIId), (IV), (IVa)-(IVd), (V), or (Va)-(Vd), each R^{6a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{6a} is independently deuterium, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), each R^{6a} is independently halogen or C₁-C₆alkyl.

In some embodiments of a compound of Formula (II), (IIa)-(IId), (III), (IIIa)-(IIId), (IV), (IVa)-(IVd), (V), or (Va)-(Vd), each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{2a}.

In some embodiments of a compound of Formula (II), (IIa)-(IId), (III), (IIIa)-(IIId), (IV), (IVa)-(IVd), (V), or (Va)-(Vd), each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl; wherein the alkyl is optionally substituted with one or more R^{2a}. In some embodiments of a compound of Formula (II), (IIa)-(IId), (III), (IIIa)-(IIId), (IV), (IVa)-(IVd), (V), or (Va)-(Vd), each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl. In some embodiments of a compound of Formula (II), (IIa)-(IId), (III), (IIIa)-(IIId), (IV), (IVa)-(IVd), (V), or (Va)-(Vd), each R² is independently hydrogen, deuterium, halogen, —CN, —OR^{b}, —NR^{c}R^{d}, C₁-C₆alkyl, C₁-C₆haloalkyl, or C₁-C₆deuteroalkyl. In some embodiments of a compound of Formula (II), (IIa)-(IId), (III), (IIIa)-(IIId), (IV), (IVa)-(IVd), (V), or (Va)-(Vd), each R² is independently hydrogen, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), each R² is independently hydrogen or halogen. In some embodiments of a compound of Formula (II), (IIa)-(IId), (III), (IIIa)-(IIId), (IV), (IVa)-(IVd), (V), or (Va)-(Vd), each R² is hydrogen.

In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R² is optionally substituted with one, two, or three R^{2a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R² is optionally substituted with one or two R^{2a}. In some embodiments of a compound of Formula (II), (IIa)-(IId), (III), (IIIa)-(IIId), (IV), (IVa)-(IVd), (V), or (Va)-(Vd), the alkyl, alkenyl, alkynyl, cycloalkyl, heterocycloalkyl, aryl, and heteroaryl of R² is optionally substituted with one R^{2a}.

In some embodiments of a compound of Formula (II), (IIa)-(IId), (III), (IIIa)-(IIId), (IV), (IVa)-(IVd), (V), or (Va)-(Vd), each R^{2a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{2a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (II), (IIa)-(IId), (III), (IIIa)-(IIId), (IV), (IVa)-(IVd), (V), or (Va)-(Vd), each R^{2a} is independently deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (II), (IIa)-(IId), (III), (IIIa)-(IIId), (IV), (IVa)-(IVd), (V), or (Va)-(Vd), each R^{2a} is independently deuterium, halogen, C₁-C₆alkyl, or C₁-C₆haloalkyl. In some embodiments of a compound of Formula (II), (IIa)-(IId), (III), (IIIa)-(IIId), (IV), (IVa)-(IVd), (V), or (Va)-(Vd), each R^{2a} is independently halogen or C₁-C₆alkyl.

In some embodiments of a compound of Formula (II), (IIa)-(IId), (III), (IIIa)-(IIId), (IV), (IVa)-(IVd), (V), or (Va)-(Vd), n is 1 or 2. In some embodiments of a compound of Formula (II), (IIa)-(IId), (III), (IIIa)-(IIId), (IV), (IVa)-(IVd), (V), or (Va)-(Vd), n is 1. In some embodiments of a compound of Formula (II), (IIa)-(IId), (III), (IIIa)-(IIId), (IV), (IVa)-(IVd), (V), or (Va)-(Vd), n is 2.

In some embodiments of a compound of Formula (I), (Ia), (Ib), (II), (IIa)-(IId), (III), (IIIa)-(IIId), (IV), (IVa)-(IVd), (V), or (Va)-(Vd), each R^{a} is independently C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), (II), (IIa)-(IId), (III), (IIIa)-(IIId), (IV), (IVa)-(IVd), (V), or (Va)-(Vd), each R^{a} is independently C₁-C₆alkyl, C₁-C₆haloalkyl, cycloalkyl, or heterocycloalkyl; wherein each alkyl, cycloalkyl, and heterocycloalkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), (II), (IIa)-(IId), (III), (IIIa)-(IIId), (IV), (IVa)-(IVd), (V), or (Va)-(Vd), each R^{a} is independently C₁-C₆alkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

In some embodiments of a compound of Formula (I), (Ia), (Ib), (II), (IIa)-(IId), (III), (IIIa)-(IIId), (IV), (IVa)-(IVd), (V), or (Va)-(Vd), each R^{b} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), (II), (IIa)-(IId), (III), (IIIa)-(IIId), (IV), (IVa)-(IVd), (V), or (Va)-(Vd), each R^{b} is independently hydrogen, C₁-C₆alkyl, C₁-C₆haloalkyl, cycloalkyl, or heterocycloalkyl; wherein each alkyl, cycloalkyl, and heterocycloalkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), (II), (IIa)-(IId), (III), (IIIa)-(IIId), (IV), (IVa)-(IVd), (V), or (Va)-(Vd), each R^{b} is independently hydrogen or C₁-C₆alkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), (II), (IIa)-(IId), (III), (IIIa)-(IIId), (IV), (IVa)-(IVd), (V), or (Va)-(Vd), each R^{b} is hydrogen.

In some embodiments of a compound of Formula (I), (Ia), (Ib), (II), (IIa)-(IId), (III), (IIIa)-(IIId), (IV), (IVa)-(IVd), (V), or (Va)-(Vd), each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), (II), (IIa)-(IId), (III), (IIIa)-(IIId), (IV), (IVa)-(IVd), (V), or (Va)-(Vd), each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. In some embodiments of a compound of Formula (I), (Ia), (Ib), (II), (IIa)-(IId), (III), (IIIa)-(IIId), (IV), (IVa)-(IVd), (V), or (Va)-(Vd), each R^{c} and R^{d} is independently hydrogen or C₁-C₆alkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl. each R^{c} and R^{d} is hydrogen.

In some embodiments of a compound of Formula (I), (Ia), (Ib), (II), (IIa)-(IId), (III), (IIIa)-(IIId), (IV), (IVa)-(IVd), (V), or (Va)-(Vd), R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

For conciseness, “or a pharmaceutically acceptable salt, solvate, tautomer, or stereoisomer thereof” was not repeated in the embodiments above but a person of skill in the art will understand that all embodiments above apply to compounds of Formula (I), (Ia), (Ib), (II), (IIa)-(IId), (III), (IIIa)-(IIId), (IV), (IVa)-(IVd), (V), or (Va)-(Vd), or a pharmaceutically acceptable salt, solvate, tautomer, or stereoisomer thereof.

In some embodiments, the compound of Formula (I), (Ia), (Ib), (II), (IIa)-(IId), (III), (IIIa)-(IIId), (IV), (IVa)-(IVd), (V), or (Va)-(Vd), or a pharmaceutically acceptable salt, solvate, tautomer, or stereoisomer thereof, is selected from a compound found in table 1:

TABLE 1

| Ex. | | | LC-MS |
|---|---|---|---|
| # | Structure | Chemical Name | data |
| 1 | 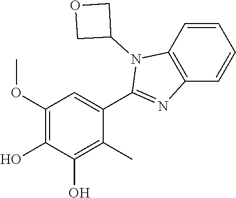 [SMILES: COc1cc(-c2nc3ccccc3n2C2COC2)c(C)c(O)c1O] | 6-methoxy-3-methyl-4- (1-(oxetan-3-yl)-1H- benzo[d]imidazol-2- yl)benzene-1,2-diol | 327.1 [M + 1]⁺ |
| 2 | 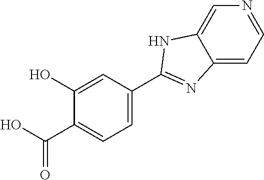 [SMILES: O=C(O)c1ccc(-c2nc3ccncc3[nH]2)cc1O] | 2-hydroxy-4-(3H- imidazo[4,5-c]pyridin-2- yl)benzoic acid | 256.1 [M + 1]⁺ |
| 3 | 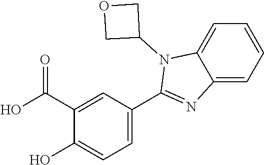 [SMILES: O=C(O)c1cc(-c2nc3ccccc3n2C2COC2)ccc1O] | 2-hydroxy-5-(1-(oxetan- 3-yl)-1H- benzo[d]imidazol-2- yl)benzoic acid | 311.3 [M + 1]⁺ |
| 4 | 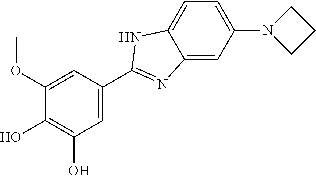 [SMILES: COc1cc(-c2nc3cc(N4CCC4)ccc3[nH]2)cc(O)c1O] | 5-(5-(azetidin-1-yl)-1H- benzo[d]imidazol-2-yl)- 3-methoxybenzene-1,2- diol | 312.2 [M + 1]⁺ |
| 5 | 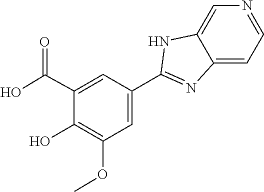 [SMILES: COc1cc(-c2nc3ccncc3[nH]2)cc(C(=O)O)c1O] | 2-hydroxy-5-(3H- imidazo[4,5-c]pyridin-2- yl)-3-methoxybenzoic acid | 286.1 [M + 1]⁺ |
| 6 | 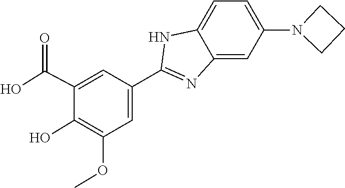 [SMILES: COc1cc(-c2nc3cc(N4CCC4)ccc3[nH]2)cc(C(=O)O)c1O] | 5-(5-(azetidin-1-yl)-1H- benzo[d]imidazol-2-yl)- 2-hydroxy-3- methoxybenzoic acid | 340.1 [M + 1]⁺ |
| 7 | 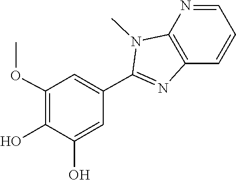 [SMILES: COc1cc(-c2nc3cccnc3n2C)cc(O)c1O] | 3-methoxy-5-(3-methyl- 3H-imidazo[4,5- b]pyridin-2-yl)benzene- 1,2-diol | 272.2 [M + 1]⁺ |
| 8 | 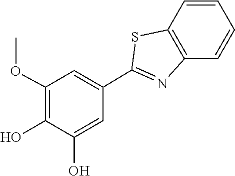 [SMILES: COc1cc(-c2nc3ccccc3s2)cc(O)c1O] | 5-(benzo[d]thiazol-2-yl)- 3-methoxybenzene-1,2- diol | 274.1 [M + 1]⁺ |
| 9 | 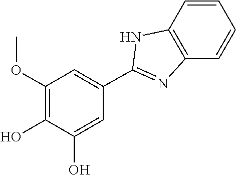 [SMILES: COc1cc(-c2nc3ccccc3[nH]2)cc(O)c1O] | 5-(1H-benzo[d]imidazol- 2-yl)-3- methoxybenzene-1,2- diol | 257.2 [M + 1]⁺ |
| 10 | 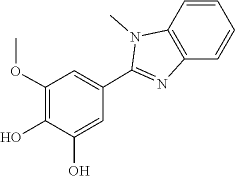 [SMILES: COc1cc(-c2nc3ccccc3n2C)cc(O)c1O] | 3-methoxy-5-(1-methyl- 1H-benzo[d]imidazol-2- yl)benzene-1,2-diol | 271.0 [M + 1]⁺ |
| 11 | 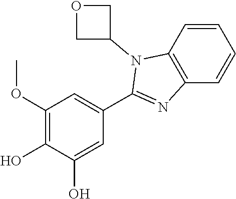 [SMILES: COc1cc(-c2nc3ccccc3n2C2COC2)cc(O)c1O] | 3-methoxy-5-(1-(oxetan- 3-yl)-1H- benzo[d]imidazol-2- yl)benzene-1,2-diol | 313.2 [M + 1]⁺ |
| 12 | 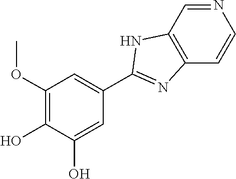 [SMILES: COc1cc(-c2nc3ccncc3[nH]2)cc(O)c1O] | 5-(3H-imidazo[4,5- c]pyridin-2-yl)-3- methoxybenzene-1,2- diol | 258.1 [M + 1]⁺ |
| 13 | 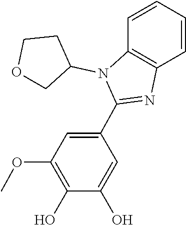 [SMILES: COc1cc(-c2nc3ccccc3n2C2CCOC2)cc(O)c1O] | 3-methoxy-5-(1- (tetrahydrofuran-3-yl)- 1H-benzo[d]imidazol-2- yl)benzene-1,2-diol | 327.2 [M + 1]⁺ |
| 14 | 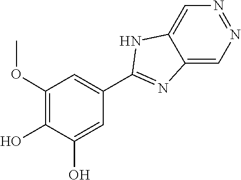 [SMILES: COc1cc(-c2nc3cnncc3[nH]2)cc(O)c1O] | 5-(1H-imidazo[4,5- d]pyridazin-2-yl)-3- methoxybenzene-1,2- diol | 259.2 [M + 1]⁺ |
| 15 | 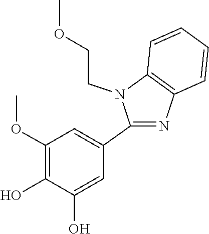 [SMILES: COCCn1c(-c2cc(O)c(O)c(OC)c2)nc2ccccc21] | 3-methoxy-5-(1-(2- methoxyethyl)-1H- benzo[d]imidazol-2- yl)benzene-1,2-diol | 315.2 [M + 1]⁺ |
| 16 | 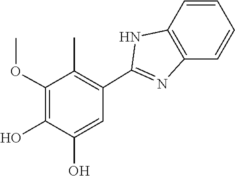 [SMILES: COc1c(C)c(-c2nc3ccccc3[nH]2)cc(O)c1O] | 5-(1H-benzo[d]imidazol- 2-yl)-3-methoxy-4- methylbenzene-1,2-diol | 271.1 [M + 1]⁺ |
| 17 | 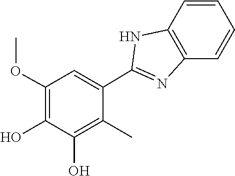 [SMILES: COc1cc(-c2nc3ccccc3[nH]2)c(C)c(O)c1O] | 4-(1H-benzo[d]imidazol- 2-yl)-6-methoxy-3- methylbenzene-1,2-diol | 271.2 [M + 1]⁺ |
| 18 | 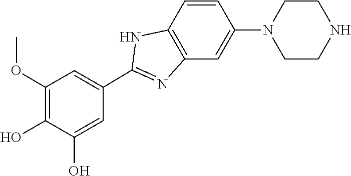 [SMILES: COc1cc(-c2nc3cc(N4CCNCC4)ccc3[nH]2)cc(O)c1O] | 3-methoxy-5-(5- (piperazin-1-yl)-1H- benzo[d]imidazol-2- yl)benzene-1,2-diol | 341.2 [M + 1]⁺ |
| 19 | 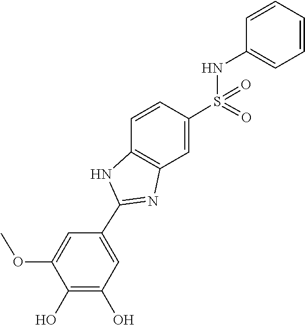 [SMILES: COc1cc(-c2nc3cc(S(=O)(=O)Nc4ccccc4)ccc3[nH]2)cc(O)c1O] | 2-(3,4-dihydroxy-5- methoxyphenyl)-N- phenyl-1H- benzo[d]imidazole-5- sulfonamide | 412.1 [M + 1]⁺ |
| 20 | 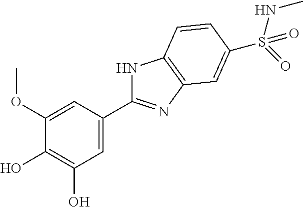 [SMILES: CNS(=O)(=O)c1ccc2[nH]c(-c3cc(O)c(O)c(OC)c3)nc2c1] | 2-(3,4-dihydroxy-5- methoxyphenyl)-N- methyl-1H- benzo[d]imidazole-5- sulfonamide | 350.1 [M + 1]⁺ |
| 21 | 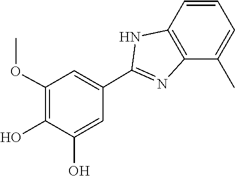 [SMILES: COc1cc(-c2nc3c(C)cccc3[nH]2)cc(O)c1O] | 3-methoxy-5-(4-methyl- 1H-benzo[d]imidazol-2- yl)benzene-1,2-diol | 271.2 [M + 1]⁺ |
| 22 | 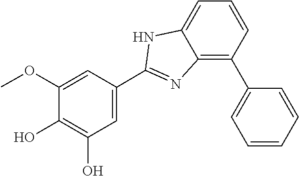 [SMILES: COc1cc(-c2nc3c(-c4ccccc4)cccc3[nH]2)cc(O)c1O] | 3-methoxy-5-(4-phenyl- 1H-benzo[d]imidazol-2- yl)benzene-1,2-diol | 333.3 [M + 1]⁺ |
| 23 | 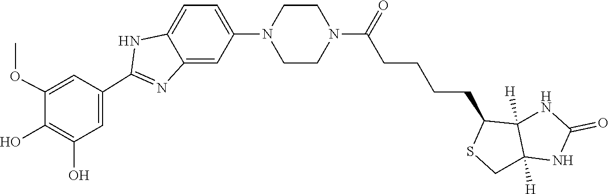 [SMILES: COc1cc(-c2nc3cc(N4CCN(C(=O)CCCC[C@@H]5SC[C@@H]6NC(=O)N[C@@H]65)CC4)ccc3[nH]2)cc(O)c1O] | (3aS,4S,6aR)-4-(5-(4-(2- (3,4-dihydroxy-5- methoxyphenyl)-1H- benzo[d]imidazol-5- yl)piperazin-1-yl)-5- oxopentyl)tetrahydro- 1H-thieno[3,4- d]imidazol-2(3H)-one | 567.2 [M + 1]⁺ |
| 24 | 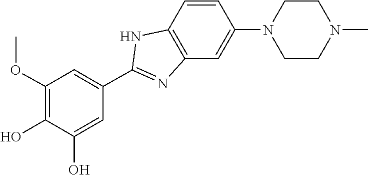 [SMILES: COc1cc(-c2nc3cc(N4CCN(C)CC4)ccc3[nH]2)cc(O)c1O] | 3-methoxy-5-(5-(4- methylpiperazin-1-yl)- 1H-benzo[d]imidazol-2- yl)benzene-1,2-diol | 355.0 [M + 1]⁺ |
| 25 | 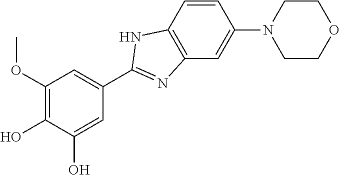 [SMILES: COc1cc(-c2nc3cc(N4CCOCC4)ccc3[nH]2)cc(O)c1O] | 3-methoxy-5-(5- morpholino-1H- benzo[d]imidazol-2- yl)benzene-1,2-diol | 342.1 [M + 1]⁺ |
| 26 | 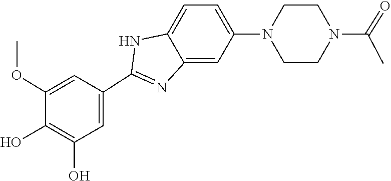 [SMILES: COc1cc(-c2nc3cc(N4CCN(C(C)=O)CC4)ccc3[nH]2)cc(O)c1O] | 1-(4-(2-(3,4-dihydroxy- 5-methoxyphenyl)-1H- benzo[d]imidazol-5- yl)piperazin-1- yl)ethanone | 383.2 [M + 1]⁺ |
| 27 | 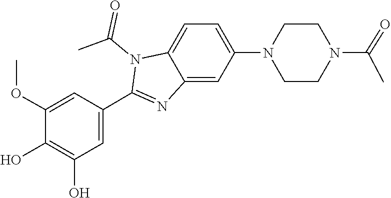 [SMILES: COc1cc(-c2nc3cc(N4CCN(C(C)=O)CC4)ccc3n2C(C)=O)cc(O)c1O] | 1-(4-(1-acetyl-2-(3,4- dihydroxy-5- methoxyphenyl)-1H- benzo[d]imidazol-5- yl)piperazin-1- yl)ethanone | 425.2 [M + 1]⁺ |
| 28 | 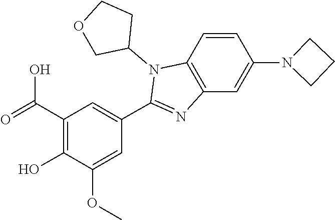 [SMILES: COc1cc(-c2nc3cc(N4CCC4)ccc3n2C2CCOC2)cc(C(=O)O)c1O] | 5-(5-(azetidin-1-yl)-1- (tetrahydrofuran-3-yl)- 1H-benzo[d]imidazol-2- yl)-2-hydroxy-3- methoxybenzoic acid | 410.4 [M + 1]⁺ |
| 29 | 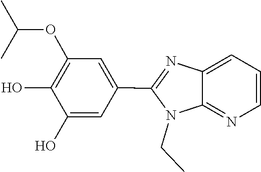 [SMILES: CCn1c(-c2cc(O)c(O)c(OC(C)C)c2)nc2cccnc21] | 5-(3-ethyl-3H- imidazo[4,5-b]pyridin-2- yl)-3- isopropoxybenzene-1,2- diol | 314.1 [M + 1]⁺ |
| 30 | 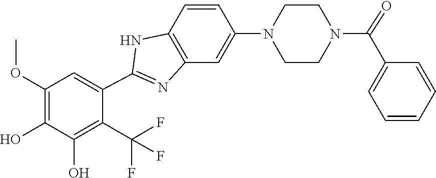 [SMILES: COc1cc(-c2nc3cc(N4CCN(C(=O)c5ccccc5)CC4)ccc3[nH]2)c(C(F)(F)F)c(O)c1O] | (4-(2-(3,4-dihydroxy-5- methoxy-2- (trifluoromethyl)phenyl)- 1H-benzo[d]imidazol-5- yl)piperazin-1- yl)(phenyl)methanone | 513.1 [M + 1]⁺ |
| 31 | 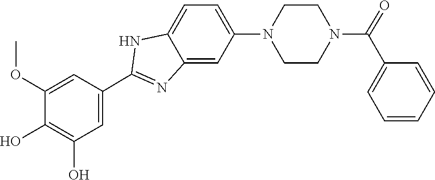 [SMILES: COc1cc(-c2nc3cc(N4CCN(C(=O)c5ccccc5)CC4)ccc3[nH]2)cc(O)c1O] | (4-(2-(3,4-dihydroxy-5- methoxyphenyl)-1H- benzo[d]imidazol-5- yl)piperazin-1- yl)(phenyl)methanone | 445.2 [M + 1]⁺ |
| 32 | 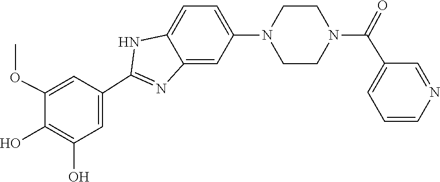 [SMILES: COc1cc(-c2nc3cc(N4CCN(C(=O)c5cccnc5)CC4)ccc3[nH]2)cc(O)c1O] | (4-(2-(3,4-dihydroxy-5- methoxyphenyl)-1H- benzo[d]imidazol-5- yl)piperazin-1- yl)(pyridin-3- yl)methanone | 446.2 [M + 1]⁺ |
| 33 | 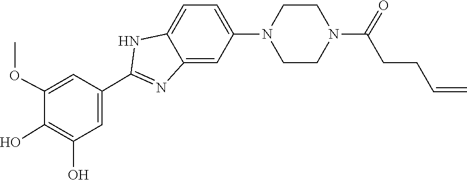 [SMILES: C=CCCC(=O)N1CCN(c2ccc3[nH]c(-c4cc(O)c(O)c(OC)c4)nc3c2)CC1] | 1-(4-(2-(3,4-dihydroxy- 5-methoxyphenyl)-1H- benzo[d]imidazol-5- yl)piperazin-1-yl)pent-4- en-1-one | 423.2 [M + 1]⁺ |
| 34 | 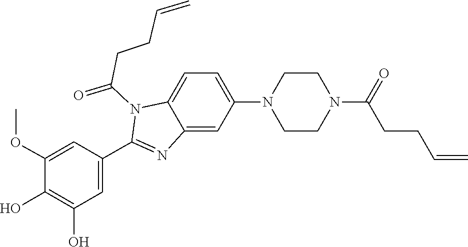 [SMILES: C=CCCC(=O)N1CCN(c2ccc3c(c2)nc(-c2cc(O)c(O)c(OC)c2)n3C(=O)CCC=C)CC1] | 1-(4-(2-(3,4-dihydroxy- 5-methoxyphenyl)-1- (pent-4-enoyl)-1H- benzo[d]imidazol-5- yl)piperazin-1-yl)pent-4- en-1-one | 505.2 [M + 1]⁺ |
| 35 | 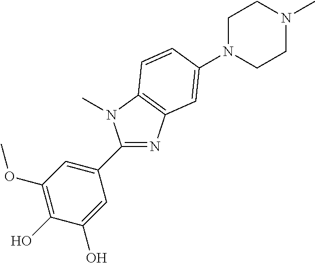 | 3-methoxy-5-(1-methyl- 5-(4-methylpiperazin-1- yl)-1H- benzo[d]imidazol-2- yl)benzene-1,2-diol | 369.2 [M + 1]⁺ |
| 36 | 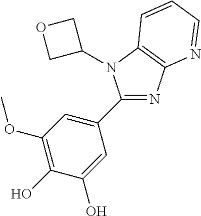 [SMILES: COc1cc(-c2nc3ncccc3n2C2COC2)cc(O)c1O] | 3-methoxy-5-(1-(oxetan- 3-yl)-1H-imidazo[4,5- b]pyridin-2-yl)benzene- 1,2-diol | 314.0 [M + 1]⁺ |
| 37 | 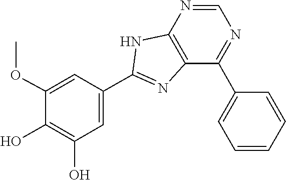 [SMILES: COc1cc(-c2nc3c(-c4ccccc4)ncnc3[nH]2)cc(O)c1O] | 3-methoxy-5-(6-phenyl- 9H-purin-8-yl)benzene- 1,2-diol | 335.1 [M + 1]⁺ |
| 38 | 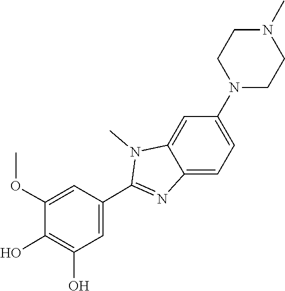 [SMILES: COc1cc(-c2nc3ccc(N4CCN(C)CC4)cc3n2C)cc(O)c1O] | 3-methoxy-5-(1-methyl- 6-(4-methylpiperazin-1- yl)-1H- benzo[d]imidazol-2- yl)benzene-1,2-diol | 369.2 [M + 1]⁺ |
| 39 | 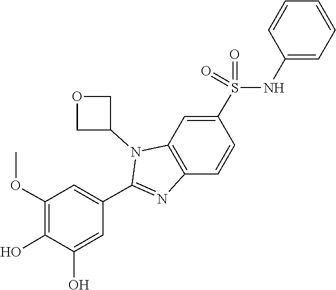 [SMILES: COc1cc(-c2nc3ccc(S(=O)(=O)Nc4ccccc4)cc3n2C2COC2)cc(O)c1O] | 2-(3,4-dihydroxy-5- methoxyphenyl)-1- (oxetan-3-yl)-N-phenyl- 1H-benzo[d]imidazole- 6-sulfonamide | 468.2 [M + 1]⁺ |
| 40 | 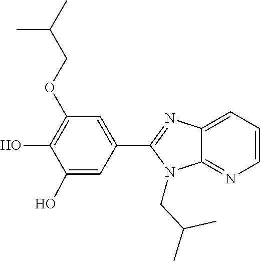 [SMILES: CC(C)COc1cc(-c2nc3cccnc3n2CC(C)C)cc(O)c1O] | 3-isobutoxy-5-(3- isobutyl-3H- imidazo[4,5-b]pyridin-2- yl)benzene-1,2-diol | 356.2 [M + 1]⁺ |
| 41 | 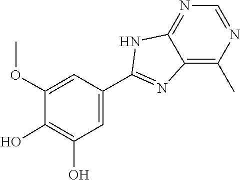 [SMILES: COc1cc(-c2nc3c(C)ncnc3[nH]2)cc(O)c1O] | 3-methoxy-5-(6-methyl- 9H-purin-8-yl)benzene- 1,2-diol | 273.0 [M + 1]⁺ |
| 42 | 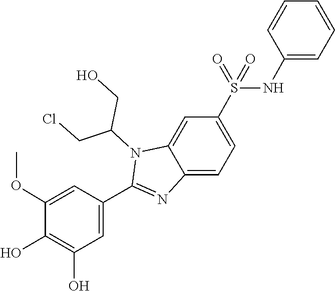 [SMILES: COc1cc(-c2nc3ccc(S(=O)(=O)Nc4ccccc4)cc3n2C(CO)CCl)cc(O)c1O] | 1-(1-chloro-3- hydroxypropan-2-yl)-2- (3,4-dihydroxy-5- methoxyphenyl)-N- phenyl-1H- benzo[d]imidazole-6- sulfonamide | 504.1 [M + 1]⁺ |
| 43 | 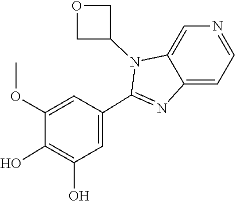 [SMILES: COc1cc(-c2nc3ccncc3n2C2COC2)cc(O)c1O] | 3-methoxy-5-(3-(oxetan- 3-yl)-3H-imidazo[4,5- c]pyridin-2-yl)benzene- 1,2-diol | 314.2 [M + 1]⁺ |
| 44 | 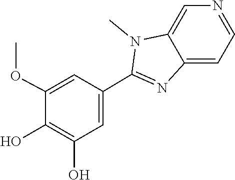 [SMILES: COc1cc(-c2nc3ccncc3n2C)cc(O)c1O] | 3-methoxy-5-(3-methyl- 3H-imidazo[4,5- c]pyridin-2-yl)benzene- 1,2-diol | 272.1 [M + 1]⁺ |
| 45 | 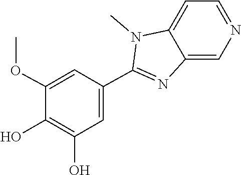 [SMILES: COc1cc(-c2nc3cnccc3n2C)cc(O)c1O] | 3-methoxy-5-(1-methyl- 1H-imidazo[4,5- c]pyridin-2-yl)benzene- 1,2-diol | 272.1 [M + 1]⁺ |
| 46 | 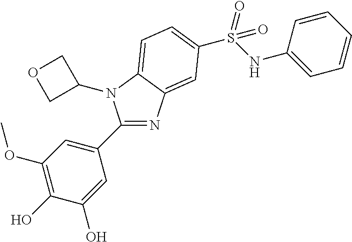 [SMILES: COc1cc(-c2nc3cc(S(=O)(=O)Nc4ccccc4)ccc3n2C2COC2)cc(O)c1O] | 2-(3,4-dihydroxy-5- methoxyphenyl)-1- (oxetan-3-yl)-N-phenyl- 1H-benzo[d]imidazole- 5-sulfonamide | 468.2 [M + 1]⁺ |
| 47 | 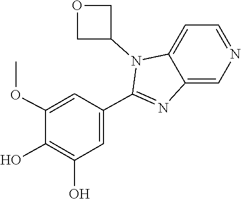 [SMILES: COc1cc(-c2nc3cnccc3n2C2COC2)cc(O)c1O] | 3-methoxy-5-(1-(oxetan- 3-yl)-1H-imidazo[4,5- c]pyridin-2-yl)benzene- 1,2-diol | 314.1 [M + 1]⁺ |
| 48 | 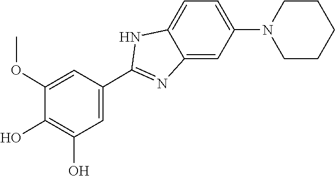 [SMILES: COc1cc(-c2nc3cc(N4CCCCC4)ccc3[nH]2)cc(O)c1O] | 3-methoxy-5-(5- (piperidin-1-yl)-1H- benzo[d]imidazol-2- yl)benzene-1,2-diol | 340.1 [M + 1]⁺ |
| 49 | 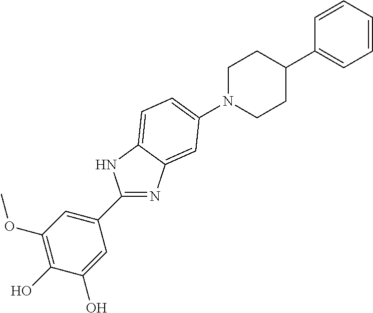 [SMILES: COc1cc(-c2nc3cc(N4CCC(c5ccccc5)CC4)ccc3[nH]2)cc(O)c1O] | 3-methoxy-5-(5-(4- phenylpiperidin-1-yl)- 1H-benzo[d]imidazol-2- yl)benzene-1,2-diol | 416.2 [M + 1]⁺ |
| 50 | 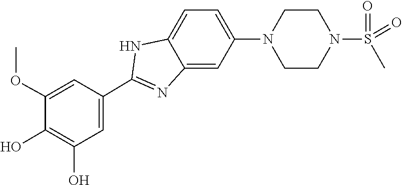 [SMILES: COc1cc(-c2nc3cc(N4CCN(S(C)(=O)=O)CC4)ccc3[nH]2)cc(O)c1O] | 3-methoxy-5-(5-(4- (methylsulfonyl)piperazin- 1-yl)-1H- benzo[d]imidazol-2- yl)benzene-1,2-diol | 419.2 [M + 1]⁺ |
| 51 | 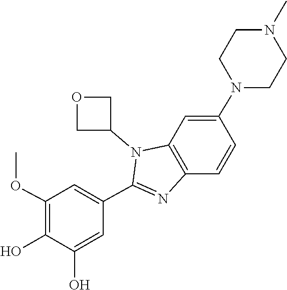 [SMILES: COc1cc(-c2nc3ccc(N4CCN(C)CC4)cc3n2C2COC2)cc(O)c1O] | 3-methoxy-5-(6-(4- methylpiperazin-1-yl)-1- (oxetan-3-yl)-1H- benzo[d]imidazol-2- yl)benzene-1,2-diol | 411.3 [M + 1]⁺ |
| 52 | 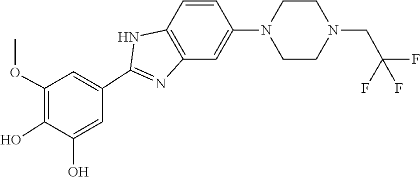 [SMILES: COc1cc(-c2nc3cc(N4CCN(CC(F)(F)F)CC4)ccc3[nH]2)cc(O)c1O] | 3-methoxy-5-(5-(4- (2,2,2- trifluoroethyl)piperazin- 1-yl)-1H- benzo[d]imidazol-2- yl)benzene-1,2-diol | 423.2 [M + 1]⁺ |
| 53 | 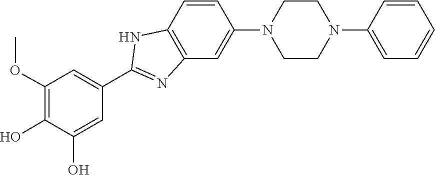 [SMILES: COc1cc(-c2nc3cc(N4CCN(c5ccccc5)CC4)ccc3[nH]2)cc(O)c1O] | 3-methoxy-5-(5-(4- phenylpiperazin-1-yl)- 1H-benzo[d]imidazol-2- yl)benzene-1,2-diol | 417.2 [M + 1]⁺ |
| 54 | 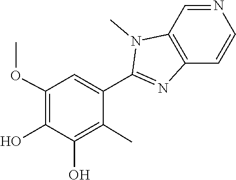 [SMILES: COc1cc(-c2nc3ccncc3n2C)c(C)c(O)c1O] | 6-methoxy-3-methyl-4- (3-methyl-3H- imidazo[4,5-c]pyridin-2- yl)benzene-1,2-diol | 286.4 [M + 1]⁺ |
| 55 | 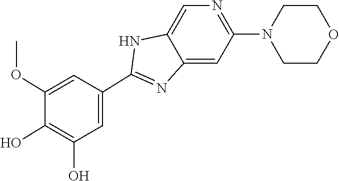 [SMILES: COc1cc(-c2nc3cc(N4CCOCC4)ncc3[nH]2)cc(O)c1O] | 3-methoxy-5-(6- morpholino-3H- imidazo[4,5-c]pyridin-2- yl)benzene-1,2-diol | 343.1 [M + 1]⁺ |
| 56 | 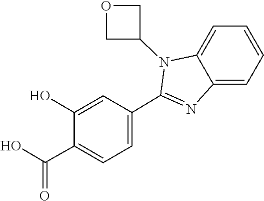 [SMILES: O=C(O)c1ccc(-c2nc3ccccc3n2C2COC2)cc1O] | 2-hydroxy-4-(1-(oxetan- 3-yl)-1H- benzo[d]imidazol-2- yl)benzoic acid | 311.1 [M + 1]⁺ |
| 57 | 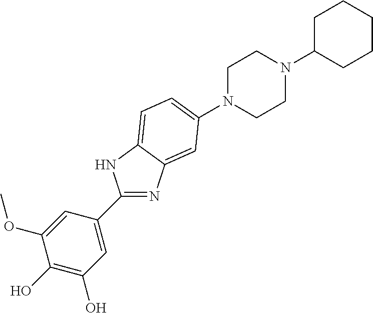 [SMILES: COc1cc(-c2nc3cc(N4CCN(C5CCCCC5)CC4)ccc3[nH]2)cc(O)c1O] | 5-(5-(4- cyclohexylpiperazin-1- yl)-1H- benzo[d]imidazol-2-yl)- 3-methoxybenzene-1,2- diol | 423.2 [M + 1]⁺ |
| 58 | 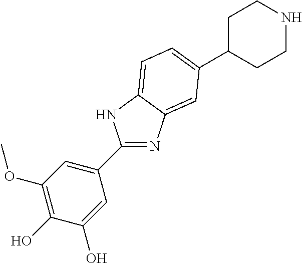 [SMILES: COc1cc(-c2nc3cc(C4CCNCC4)ccc3[nH]2)cc(O)c1O] | 3-methoxy-5-(5- (piperidin-4-yl)-1H- benzo[d]imidazol-2- yl)benzene-1,2-diol | 340.2 [M + 1]⁺ |
| 59 | 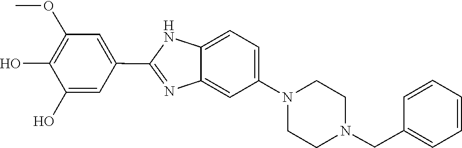 [SMILES: COc1cc(-c2nc3cc(N4CCN(Cc5ccccc5)CC4)ccc3[nH]2)cc(O)c1O] | 5-(5-(4-benzylpiperazin- 1-yl)-1H- benzo[d]imidazol-2-yl)- 3-methoxybenzene-1,2- diol | 431.2 [M + 1]⁺ |
| 60 | 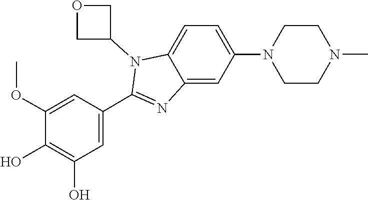 [SMILES: COc1cc(-c2nc3cc(N4CCN(C)CC4)ccc3n2C2COC2)cc(O)c1O] | 3-methoxy-5-(5-(4- methylpiperazin-1-yl)-1- (oxetan-3-yl)-1H- benzo[d]imidazol-2- yl)benzene-1,2-diol | 411.2 [M + 1]⁺ |
| 61 | 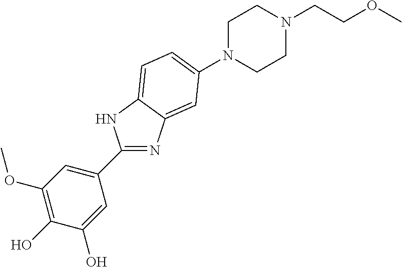 | 3-methoxy-5-(5-(4-(2- methoxyethyl)piperazin- 1-yl)-1H- benzo[d]imidazol-2- yl)benzene-1,2-diol | 399.2 [M + 1]⁺ |
| 62 | 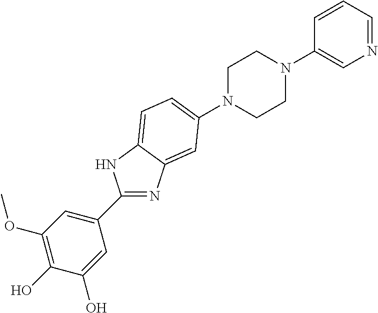 [SMILES: COc1cc(-c2nc3cc(N4CCN(c5cccnc5)CC4)ccc3[nH]2)cc(O)c1O] | 3-methoxy-5-(5-(4- (pyridin-3-yl)piperazin- 1-yl)-1H- benzo[d]imidazol-2- yl)benzene-1,2-diol | 418.2 [M + 1]⁺ |
| 63 | 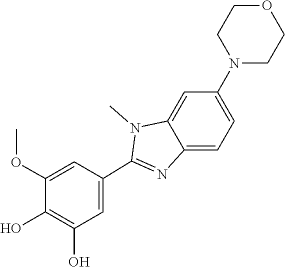 [SMILES: COc1cc(-c2nc3ccc(N4CCOCC4)cc3n2C)cc(O)c1O] | 3-methoxy-5-(1-methyl- 6-morpholino-1H- benzo[d]imidazol-2- yl)benzene-1,2-diol | 356.2 [M + 1]⁺ |
| 64 | 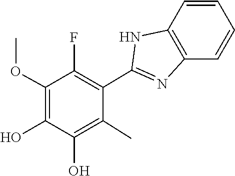 [SMILES: COc1c(O)c(O)c(C)c(-c2nc3ccccc3[nH]2)c1F] | 4-(1H-benzo[d]imidazol- 2-yl)-5-fluoro-6- methoxy-3- methylbenzene-1,2-diol | 289.1 [M + 1]⁺ |
| 65 | 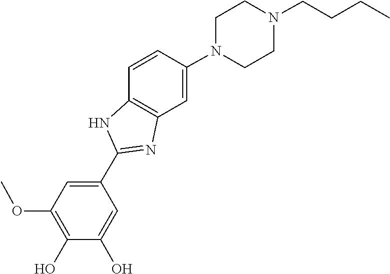 [SMILES: CCCCN1CCN(c2ccc3[nH]c(-c4cc(O)c(O)c(OC)c4)nc3c2)CC1] | 5-(5-(4-butylpiperazin-1- yl)-1H- benzo[d]imidazol-2-yl)- 3-methoxybenzene-1,2- diol | 397.3 [M + 1]⁺ |
| 66 | 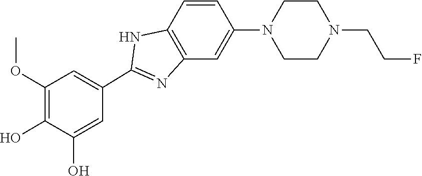 [SMILES: COc1cc(-c2nc3cc(N4CCN(CCF)CC4)ccc3[nH]2)cc(O)c1O] | 5-(5-(4-(2- fluoroethyl)piperazin-1- yl)-1H- benzo[d]imidazol-2-yl)- 3-methoxybenzene-1,2- diol | 387.2 [M + 1]⁺ |
| 67 | 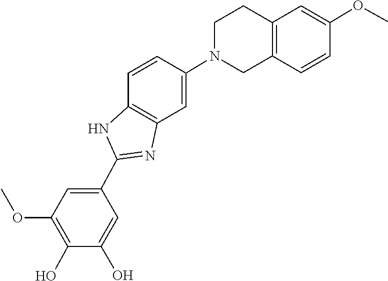 | 3-methoxy-5-(5-(6- methoxy-3,4- dihydroisoquinolin- 2(1H)-yl)-1H- benzo[d]imidazol-2- yl)benzene-1,2-diol | 418.4 [M + 1]⁺ |
| 68 | 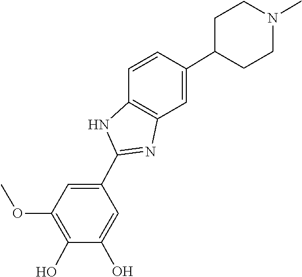 | 3-methoxy-5-(5-(1- methylpiperidin-4-yl)- 1H-benzo[d]imidazol-2- yl)benzene-1,2-diol | 354.2 [M + 1]⁺ |
| 69 | 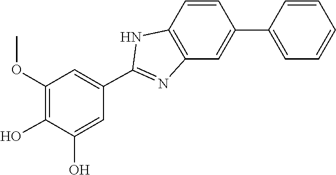 [SMILES: COc1cc(-c2nc3cc(-c4ccccc4)ccc3[nH]2)cc(O)c1O] | 3-methoxy-5-(5-phenyl- 1H-benzo[d]imidazol-2- yl)benzene-1,2-diol | 333.3 [M + 1]⁺ |
| 70 | 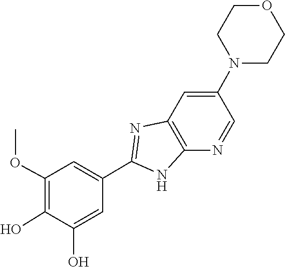 [SMILES: COc1cc(-c2nc3cc(N4CCOCC4)cnc3[nH]2)cc(O)c1O] | 3-methoxy-5-(6- morpholino-3H- imidazo[4,5-b]pyridin-2- yl)benzene-1,2-diol | 343.2 [M + 1]⁺ |
| 71 | 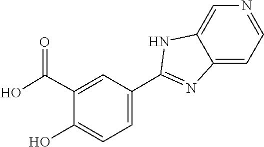 [SMILES: O=C(O)c1cc(-c2nc3ccncc3[nH]2)ccc1O] | 2-hydroxy-5-(3H- imidazo[4,5-c]pyridin-2- yl)benzoic acid | 256.1 [M + 1]⁺ |
| 72 | 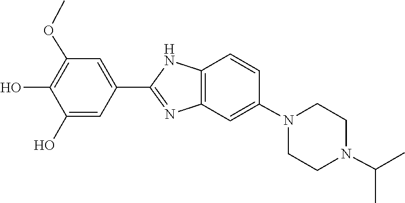 [SMILES: COc1cc(-c2nc3cc(N4CCN(C(C)C)CC4)ccc3[nH]2)cc(O)c1O] | 5-(5-(4- isopropylpiperazin-1-yl)- 1H-benzo[d]imidazol-2- yl)-3-methoxybenzene- 1,2-diol | 383.2 [M + 1]⁺ |
| 73 | 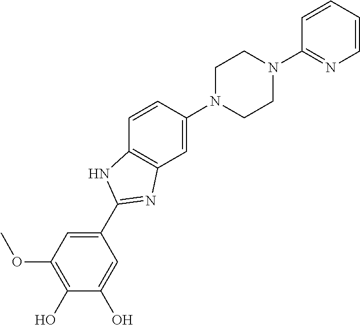 [SMILES: COc1cc(-c2nc3cc(N4CCN(c5ccccn5)CC4)ccc3[nH]2)cc(O)c1O] | 3-methoxy-5-(5-(4- (pyridin-2-yl)piperazin- 1-yl)-1H- benzo[d]imidazol-2- yl)benzene-1,2-diol | 418.2 [M + 1]⁺ |
| 74 | 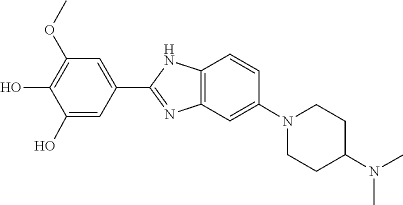 [SMILES: COc1cc(-c2nc3cc(N4CCC(N(C)C)CC4)ccc3[nH]2)cc(O)c1O] | 5-(5-(4- (dimethylamino)piperidin- 1-yl)-1H- benzo[d]imidazol-2-yl)- 3-methoxybenzene-1,2- diol | 383.2 [M + 1]⁺ |
| 75 | 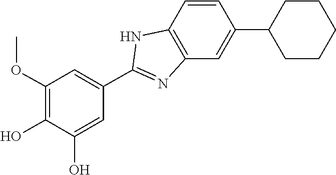 [SMILES: COc1cc(-c2nc3cc(C4CCCCC4)ccc3[nH]2)cc(O)c1O] | 5-(5-cyclohexyl-1H- benzo[d]imidazol-2-yl)- 3-methoxybenzene-1,2- diol | 339.2 [M + 1]⁺ |
| 76 | 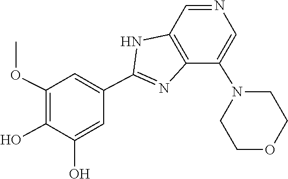 [SMILES: COc1cc(-c2nc3c(N4CCOCC4)cncc3[nH]2)cc(O)c1O] | 3-methoxy-5-(7- morpholino-3H- imidazo[4,5-c]pyridin-2- yl)benzene-1,2-diol | 343.2 [M + 1]⁺ |
| 77 | 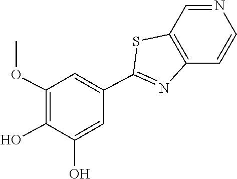 [SMILES: COc1cc(-c2nc3ccncc3s2)cc(O)c1O] | 3-methoxy-5- (thiazolo[5,4-c]pyridin- 2-yl)benzene-1,2-diol | 275.1 [M + 1]⁺ |
| 78 | 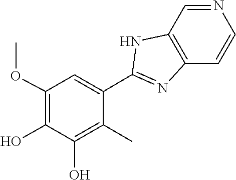 [SMILES: COc1cc(-c2nc3ccncc3[nH]2)c(C)c(O)c1O] | 4-(3H-imidazo[4,5- c]pyridin-2-yl)-6- methoxy-3- methylbenzene-1,2-diol | 272.1 [M + 1]⁺ |
| 79 | 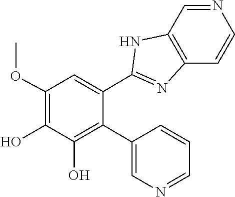 [SMILES: COc1cc(-c2nc3ccncc3[nH]2)c(-c2cccnc2)c(O)c1O] | 4-(3H-imidazo[4,5- c]pyridin-2-yl)-6- methoxy-3-(pyridin-3- yl)benzene-1,2-diol | 335.1 [M + 1]⁺ |
| 80 | 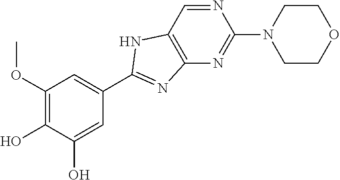 [SMILES: COc1cc(-c2nc3nc(N4CCOCC4)ncc3[nH]2)cc(O)c1O] | 3-methoxy-5-(2- morpholino-7H-purin-8- yl)benzene-1,2-diol | 344.2 [M + 1]⁺ |
| 81 | 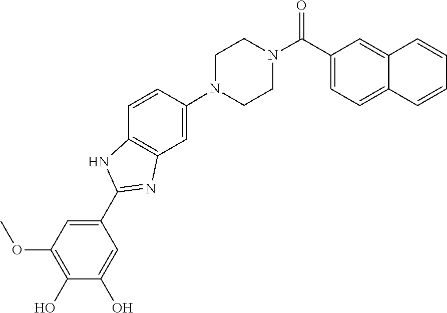 [SMILES: COc1cc(-c2nc3cc(N4CCN(C(=O)c5ccc6ccccc6c5)CC4)ccc3[nH]2)cc(O)c1O] | (4-(2-(3,4-dihydroxy-5- methoxyphenyl)-1H- benzo[d]imidazol-5- yl)piperazin-1- yl)(naphthalen-2- yl)methanone | 495.2 [M + 1]⁺ |
| 82 | 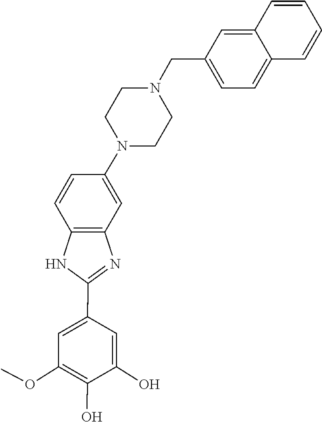 [SMILES: COc1cc(-c2nc3cc(N4CCN(Cc5ccc6ccccc6c5)CC4)ccc3[nH]2)cc(O)c1O] | 3-methoxy-5-(5-(4- (naphthalen-2- ylmethyl)piperazin-1- yl)-1H- benzo[d]imidazol-2- yl)benzene-1,2-diol | 481.2 [M + 1]⁺ |
| 83 | 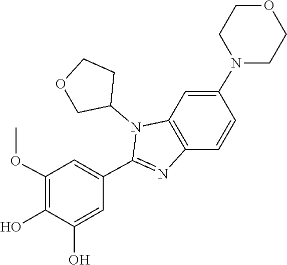 [SMILES: COc1cc(-c2nc3ccc(N4CCOCC4)cc3n2C2CCOC2)cc(O)c1O] | 3-methoxy-5-(6- morpholino-1- (tetrahydrofuran-3-yl)- 1H-benzo[d]imidazol-2- yl)benzene-1,2-diol | 412.2 [M + 1]⁺ |
| 84 | 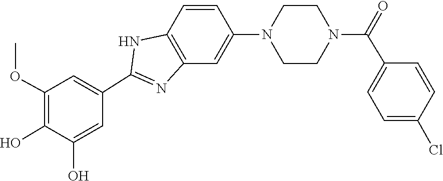 [SMILES: COc1cc(-c2nc3cc(N4CCN(C(=O)c5ccc(Cl)cc5)CC4)ccc3[nH]2)cc(O)c1O] | (4-chlorophenyl)(4-(2- (3,4-dihydroxy-5- methoxyphenyl)-1H- benzo[d]imidazol-5- yl)piperazin-1- yl)methanone | 479.2 [M + 1]⁺ |
| 85 | 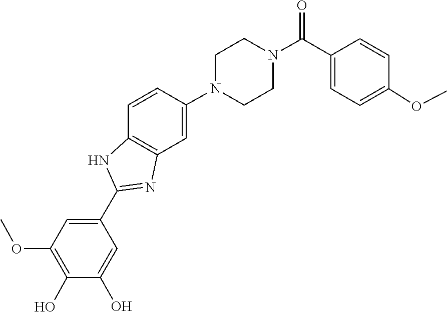 | (4-(2-(3,4-dihydroxy-5- methoxyphenyl)-1H- benzo[d]imidazol-5- yl)piperazin-1-yl)(4- methoxyphenyl)methanone | 475.2 [M + 1]⁺ |
| 86 | 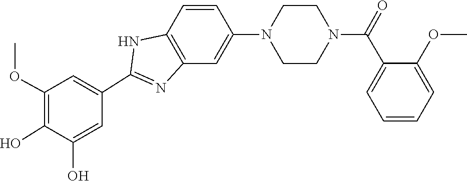 [SMILES: COc1ccccc1C(=O)N1CCN(c2ccc3[nH]c(-c4cc(O)c(O)c(OC)c4)nc3c2)CC1] | (4-(2-(3,4-dihydroxy-5- methoxyphenyl)-1H- benzo[d]imidazol-5- yl)piperazin-1-yl)(2- methoxyphenyl)methanone | 475.3 [M + 1]⁺ |
| 87 | 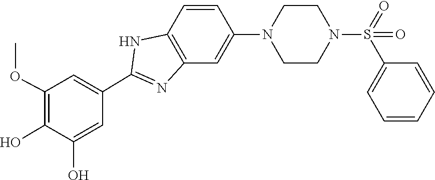 [SMILES: COc1cc(-c2nc3cc(N4CCN(S(=O)(=O)c5ccccc5)CC4)ccc3[nH]2)cc(O)c1O] | 3-methoxy-5-(5-(4- (phenylsulfonyl)piperazin- 1-yl)-1H- benzo[d]imidazol-2- yl)benzene-1,2-diol | 481.2 [M + 1]⁺ |
| 88 | 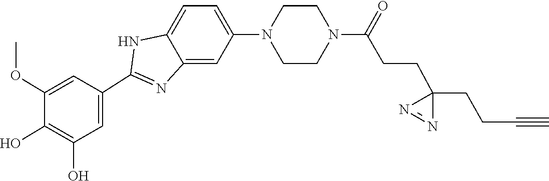 [SMILES: C#CCCC1(CCC(=O)N2CCN(c3ccc4[nH]c(-c5cc(O)c(O)c(OC)c5)nc4c3)CC2)N=N1] | 3-(3-(but-3-yn-1-yl)-3H- diazirin-3-yl)-1-(4-(2- (3,4-dihydroxy-5- methoxyphenyl)-1H- benzo[d]imidazol-5- yl)piperazin-1- yl)propan-1-one | 489.5 [M + 1]⁺ |
| 89 | 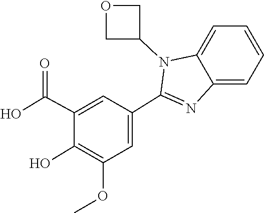 [SMILES: COc1cc(-c2nc3ccccc3n2C2COC2)cc(C(=O)O)c1O] | 2-hydroxy-3-methoxy-5- (1-(oxetan-3-yl)-1H- benzo[d]imidazol-2- yl)benzoic acid | 341.1 [M + 1]⁺ |
| 90 | 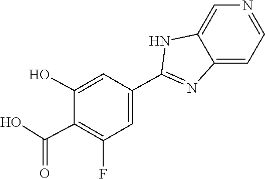 [SMILES: O=C(O)c1c(O)cc(-c2nc3ccncc3[nH]2)cc1F] | 2-fluoro-6-hydroxy-4- (3H-imidazo[4,5- c]pyridin-2-yl)benzoic acid | 274.0 [M + 1]⁺ |
| 91 | 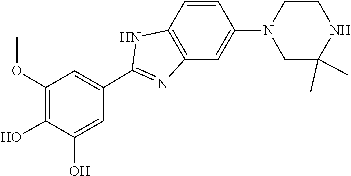 [SMILES: COc1cc(-c2nc3cc(N4CCNC(C)(C)C4)ccc3[nH]2)cc(O)c1O] | 5-(5-(3,3- dimethylpiperazin-1-yl)- 1H-benzo[d]imidazol-2- yl)-3-methoxybenzene- 1,2-diol | 369.3 [M + 1]⁺ |
| 92 | 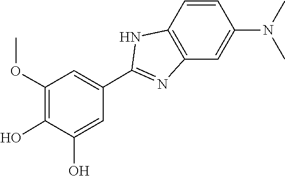 [SMILES: COc1cc(-c2nc3cc(N(C)C)ccc3[nH]2)cc(O)c1O] | 5-(5-(dimethylamino)- 1H-benzo[d]imidazol-2- yl)-3-methoxybenzene- 1,2-diol | 300.2 [M + 1]⁺ |
| 93 | 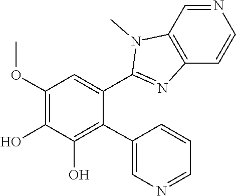 [SMILES: COc1cc(-c2nc3ccncc3n2C)c(-c2cccnc2)c(O)c1O] | 6-methoxy-4-(3-methyl- 3H-imidazo[4,5- c]pyridin-2-yl)-3- (pyridin-3-yl)benzene- 1,2-diol | 349.1 [M + 1]⁺ |
| 94 | 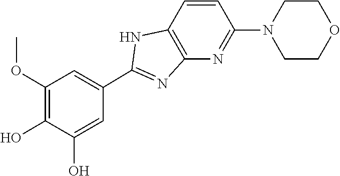 [SMILES: COc1cc(-c2nc3nc(N4CCOCC4)ccc3[nH]2)cc(O)c1O] | 3-methoxy-5-(5- morpholino-1H- imidazo[4,5-b]pyridin-2- yl)benzene-1,2-diol | 343.1 [M + 1]⁺ |
| 95 | 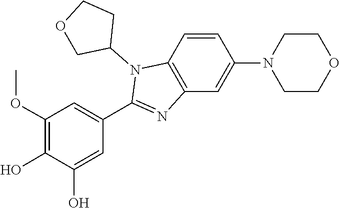 [SMILES: COc1cc(-c2nc3cc(N4CCOCC4)ccc3n2C2CCOC2)cc(O)c1O] | 3-methoxy-5-(5- morpholino-1- (tetrahydrofuran-3-yl)- 1H-benzo[d]imidazol-2- yl)benzene-1,2-diol | 412.2 [M + 1]⁺ |
| 96 | 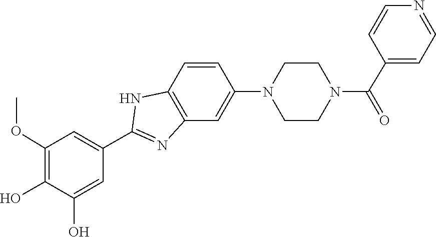 [SMILES: COc1cc(-c2nc3cc(N4CCN(C(=O)c5ccncc5)CC4)ccc3[nH]2)cc(O)c1O] | (4-(2-(3,4-dihydroxy-5- methoxyphenyl)-1H- benzo[d]imidazol-5- yl)piperazin-1- yl)(pyridin-4- yl)methanone | 446.2 [M + 1]⁺ |
| 97 | 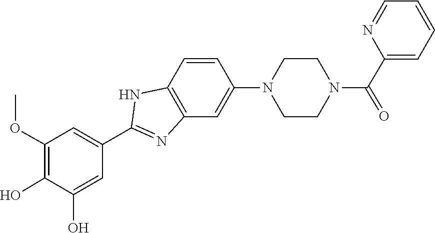 [SMILES: COc1cc(-c2nc3cc(N4CCN(C(=O)c5ccccn5)CC4)ccc3[nH]2)cc(O)c1O] | (4-(2-(3,4-dihydroxy-5- methoxyphenyl)-1H- benzo[d]imidazol-5- yl)piperazin-1- yl)(pyridin-2- yl)methanone | 446.2 [M + 1]⁺ |
| 98 | 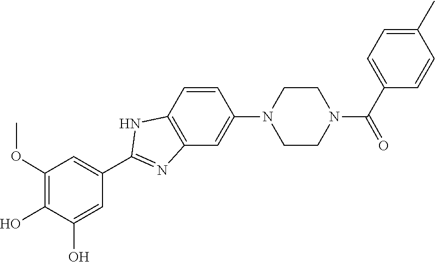 [SMILES: COc1cc(-c2nc3cc(N4CCN(C(=O)c5ccc(C)cc5)CC4)ccc3[nH]2)cc(O)c1O] | (4-(2-(3,4-dihydroxy-5- methoxyphenyl)-1H- benzo[d]imidazol-5- yl)piperazin-1-yl)(p- tolyl)methanone | 459.3 [M + 1]⁺ |
| 99 | 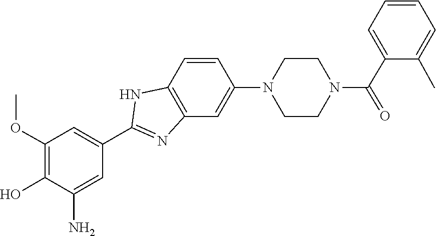 [SMILES: COc1cc(-c2nc3cc(N4CCN(C(=O)c5ccccc5C)CC4)ccc3[nH]2)cc(N)c1O] | (4-(2-(3,4-dihydroxy-5- methoxyphenyl)-1H- benzo[d]imidazol-5- yl)piperazin-1-yl)(o- tolyl)methanone | 459.3 [M + 1]⁺ |
| 100 | 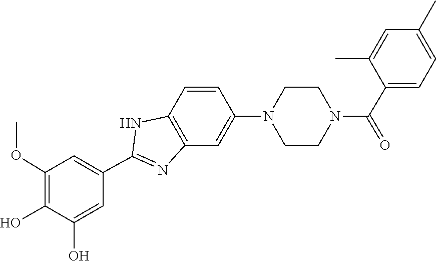 [SMILES: COc1cc(-c2nc3cc(N4CCN(C(=O)c5ccc(C)cc5C)CC4)ccc3[nH]2)cc(O)c1O] | (4-(2-(3,4-dihydroxy-5- methoxyphenyl)-1H- benzo[d]imidazol-5- yl)piperazin-1-yl)(2,4- dimethylphenyl)methanone | 473.3 [M + 1]⁺ |
| 101 | 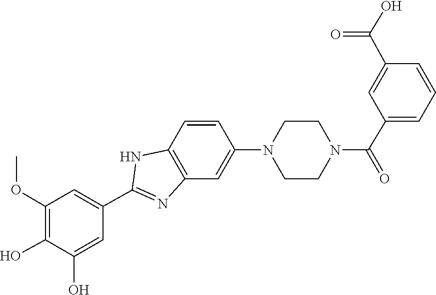 [SMILES: COc1cc(-c2nc3cc(N4CCN(C(=O)c5cccc(C(=O)O)c5)CC4)ccc3[nH]2)cc(O)c1O] | 3-(4-(2-(3,4-dihydroxy- 5-methoxyphenyl)-1H- benzo[d]imidazol-5- yl)piperazine-1- carbonyl)benzoic acid | 489.3 [M + 1]⁺ |
| 102 | 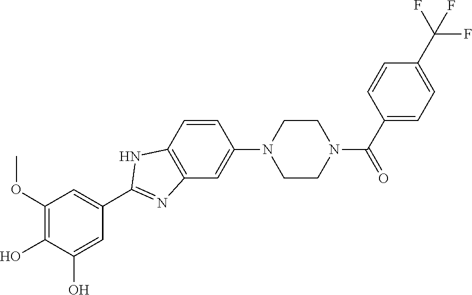 [SMILES: COc1cc(-c2nc3cc(N4CCN(C(=O)c5ccc(C(F)(F)F)cc5)CC4)ccc3[nH]2)cc(O)c1O] | (4-(2-(3,4-dihydroxy-5- methoxyphenyl)-1H- benzo[d]imidazol-5- yl)piperazin-1-yl)(4- (trifluoromethyl)phenyl) methanone | 513.2 [M + 1]⁺ |
| 103 | 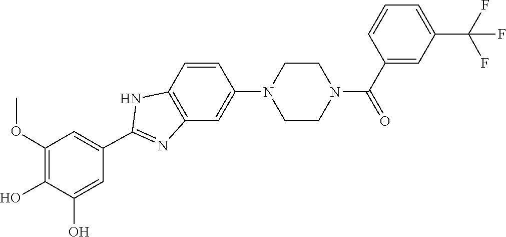 [SMILES: COc1cc(-c2nc3cc(N4CCN(C(=O)c5cccc(C(F)(F)F)c5)CC4)ccc3[nH]2)cc(O)c1O] | (4-(2-(3,4-dihydroxy-5- methoxyphenyl)-1H- benzo[d]imidazol-5- yl)piperazin-1-yl)(3- (trifluoromethyl)phenyl) methanone | 513.3 [M + 1]⁺ |
| 104 | 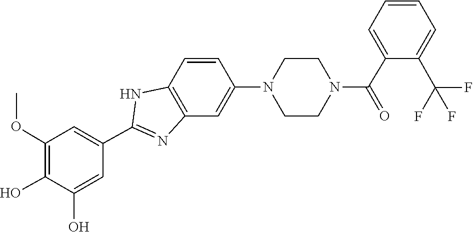 [SMILES: COc1cc(-c2nc3cc(N4CCN(C(=O)c5ccccc5C(F)(F)F)CC4)ccc3[nH]2)cc(O)c1O] | (4-(2-(3,4-dihydroxy-5- methoxyphenyl)-1H- benzo[d]imidazol-5- yl)piperazin-1-yl)(2- (trifluoromethyl)phenyl) methanone | 513.3 [M + 1]⁺ |
| 105 | 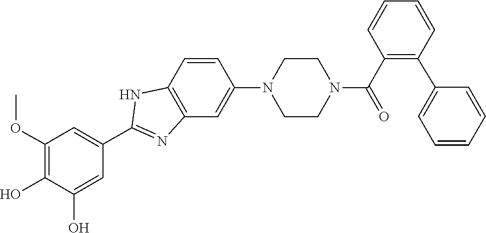 [SMILES: COc1cc(-c2nc3cc(N4CCN(C(=O)c5ccccc5-c5ccccc5)CC4)ccc3[nH]2)cc(O)c1O] | [1,1′-biphenyl]-2-yl(4- (2-(3,4-dihydroxy-5- methoxyphenyl)-1H- benzo[d]imidazol-5- yl)piperazin-1- yl)methanone | 521.3 [M + 1]⁺ |
| 106 | 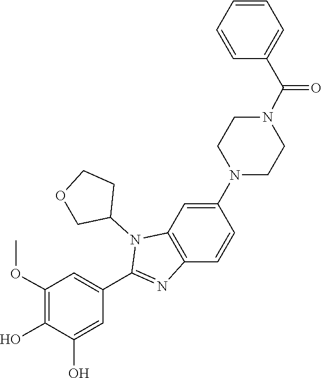 [SMILES: COc1cc(-c2nc3ccc(N4CCN(C(=O)c5ccccc5)CC4)cc3n2C2CCOC2)cc(O)c1O] | (4-(2-(3,4-dihydroxy-5- methoxyphenyl)-1- (tetrahydrofuran-3-yl)- 1H-benzo[d]imidazol-6- yl)piperazin-1- yl)(phenyl)methanone | 515.3 [M + 1]⁺ |
| 107 | 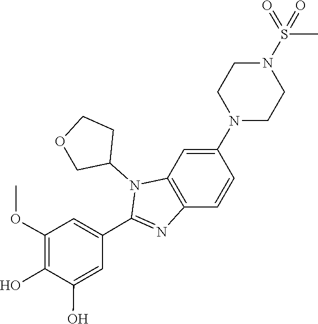 [SMILES: COc1cc(-c2nc3ccc(N4CCN(S(C)(=O)=O)CC4)cc3n2C2CCOC2)cc(O)c1O] | 3-methoxy-5-(6-(4- (methylsulfonyl)piperazin- 1-yl)-1- (tetrahydrofuran-3-yl)- 1H-benzo[d]imidazol-2- yl)benzene-1,2-diol | 489.2 [M + 1]⁺ |
| 108 | 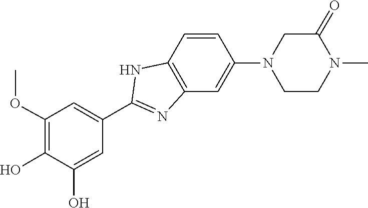 [SMILES: COc1cc(-c2nc3cc(N4CCN(C)C(=O)C4)ccc3[nH]2)cc(O)c1O] | 4-(2-(3,4-dihydroxy-5- methoxyphenyl)-1H- benzo[d]imidazol-5-yl)- 1-methylpiperazin-2-one | 367.4 [M − 1]⁻ |
| 109 | 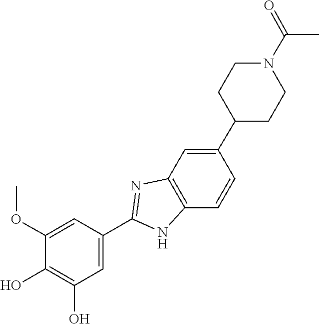 [SMILES: COc1cc(-c2nc3cc(C4CCN(C(C)=O)CC4)ccc3[nH]2)cc(O)c1O] | 1-(4-(2-(3,4-dihydroxy- 5-methoxyphenyl)-1H- benzo[d]imidazol-5- yl)piperidin-1- yl)ethanone | 382.2 [M + 1]⁺ |
| 110 | 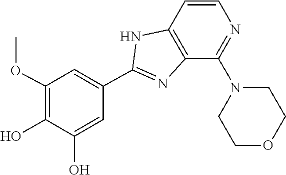 [SMILES: COc1cc(-c2nc3c(N4CCOCC4)nccc3[nH]2)cc(O)c1O] | 3-methoxy-5-(4- morpholino-1H- imidazo[4,5-c]pyridin-2- yl)benzene-1,2-diol | 343.1 [M + 1]⁺ |
| 111 | 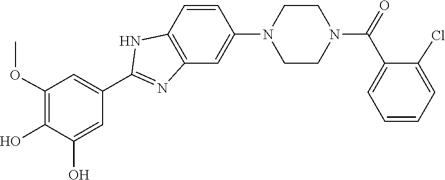 [SMILES: COc1cc(-c2nc3cc(N4CCN(C(=O)c5ccccc5Cl)CC4)ccc3[nH]2)cc(O)c1O] | (2-chlorophenyl)(4-(2- (3,4-dihydroxy-5- methoxyphenyl)-1H- benzo[d]imidazol-5- yl)piperazin-1- yl)methanone | 479.2 [M + 1]⁺ |
| 112 | 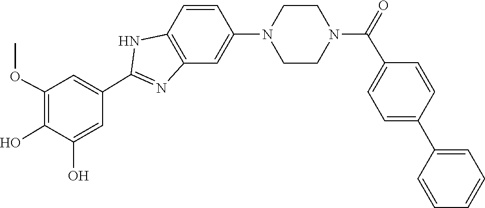 [SMILES: COc1cc(-c2nc3cc(N4CCN(C(=O)c5ccc(-c6ccccc6)cc5)CC4)ccc3[nH]2)cc(O)c1O] | [1,1′-biphenyl]-4-yl(4- (2-(3,4-dihydroxy-5- methoxyphenyl)-1H- benzo[d]imidazol-5- yl)piperazin-1- yl)methanone | 519.4 [M − 1]⁻ |
| 113 | 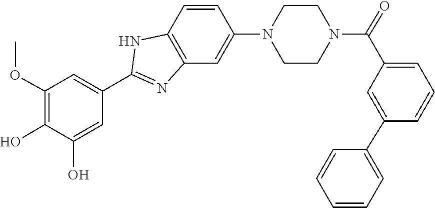 [SMILES: COc1cc(-c2nc3cc(N4CCN(C(=O)c5cccc(-c6ccccc6)c5)CC4)ccc3[nH]2)cc(O)c1O] | [1,1′-biphenyl]-3-yl(4- (2-(3,4-dihydroxy-5- methoxyphenyl)-1H- benzo[d]imidazol-5- yl)piperazin-1- yl)methanone | 521.3 [M + 1]⁺ |
| 114 | 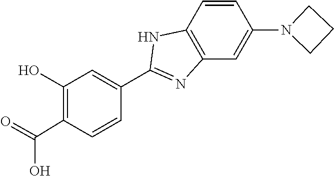 [SMILES: O=C(O)c1ccc(-c2nc3cc(N4CCC4)ccc3[nH]2)cc1O] | 4-(5-(azetidin-1-yl)-1H- benzo[d]imidazol-2-yl)- 2-hydroxybenzoic acid | 310.0 [M + 1]⁺ |
| 115 | 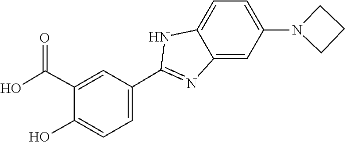 [SMILES: O=C(O)c1cc(-c2nc3cc(N4CCC4)ccc3[nH]2)ccc1O] | 5-(5-(azetidin-1-yl)-1H- benzo[d]imidazol-2-yl)- 2-hydroxybenzoic acid | 310.0 [M + 1]⁺ |
| 116 | 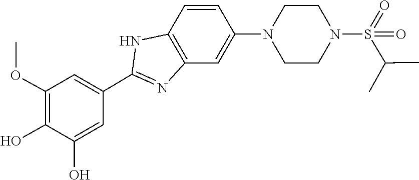 [SMILES: COc1cc(-c2nc3cc(N4CCN(S(=O)(=O)C(C)C)CC4)ccc3[nH]2)cc(O)c1O] | 5-(5-(4- (isopropylsulfonyl) piperazin-1-yl)-1H- benzo[d]imidazol-2-yl)- 3-methoxybenzene-1,2- diol | 447.1 [M + 1]⁺ |
| 117 | 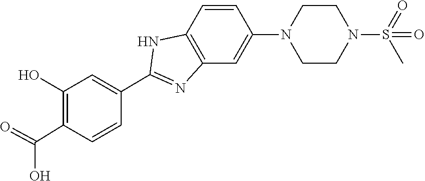 [SMILES: CS(=O)(=O)N1CCN(c2ccc3[nH]c(-c4ccc(C(=O)O)c(O)c4)nc3c2)CC1] | 2-hydroxy-4-(5-(4- (methylsulfonyl) piperazin-1-yl)-1H- benzo[d]imidazol-2- yl)benzoic acid | 417.1 [M + 1]⁺ |
| 118 | 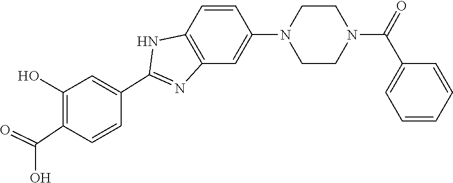 [SMILES: O=C(O)c1ccc(-c2nc3cc(N4CCN(C(=O)c5ccccc5)CC4)ccc3[nH]2)cc1O] | 4-(5-(4- benzoylpiperazin-1-yl)- 1H-benzo[d]imidazol-2- yl)-2-hydroxybenzoic acid | 443.1 [M + 1]⁺ |
| 119 | 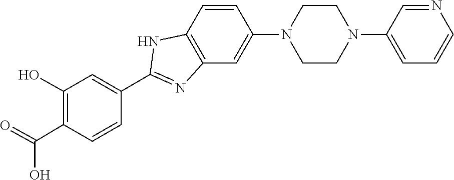 [SMILES: O=C(O)c1ccc(-c2nc3cc(N4CCN(c5cccnc5)CC4)ccc3[nH]2)cc1O] | 2-hydroxy-4-(5-(4- (pyridin-3-yl)piperazin- 1-yl)-1H- benzo[d]imidazol-2- yl)benzoic acid | 416.1 [M + 1]⁺ |
| 120 | 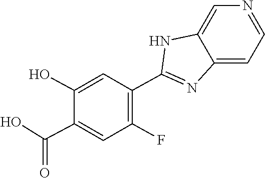 [SMILES: O=C(O)c1cc(F)c(-c2nc3ccncc3[nH]2)cc1O] | 5-fluoro-2-hydroxy-4- (3H-imidazo[4,5- c]pyridin-2-yl)benzoic acid | 274.1 [M + 1]⁺ |
| 121 | 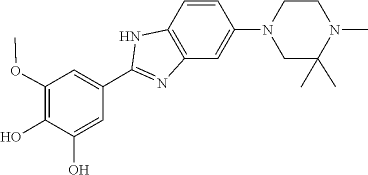 | 3-methoxy-5-(5-(3,3,4- trimethylpiperazin-1-yl)- 1H-benzo[d]imidazol-2- yl)benzene-1,2-diol | 383.3 [M + 1]⁺ |
| 122 | 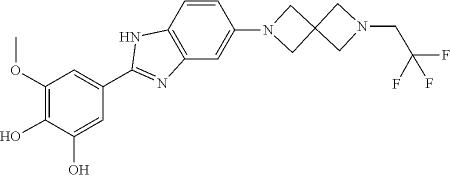 [SMILES: COc1cc(-c2nc3cc(N4CC5(CN(CC(F)(F)F)C5)C4)ccc3[nH]2)cc(O)c1O] | 3-methoxy-5-(5-(6- (2,2,2-trifluoroethyl)- 2,6- diazaspiro[3.3]heptan-2- yl)-1H- benzo[d]imidazol-2- yl)benzene-1,2-diol | 435.2 [M + 1]⁺ |
| 123 | 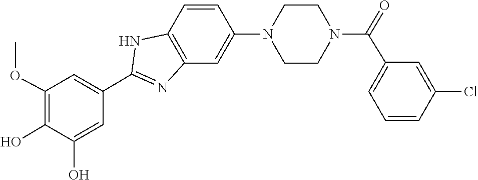 [SMILES: COc1cc(-c2nc3cc(N4CCN(C(=O)c5cccc(Cl)c5)CC4)ccc3[nH]2)cc(O)c1O] | (3-chlorophenyl)(4-(2- (3,4-dihydroxy-5- methoxyphenyl)-1H- benzo[d]imidazol-5- yl)piperazin-1- yl)methanone | 479.3 [M + 1]⁺ |
| 124 | 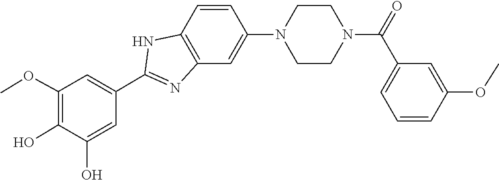 | (4-(2-(3,4-dihydroxy-5- methoxyphenyl)-1H- benzo[d]imidazol-5- yl)piperazin-1-yl)(3- methoxyphenyl)methanone | 475.3 [M + 1]⁺ |
| 125 | 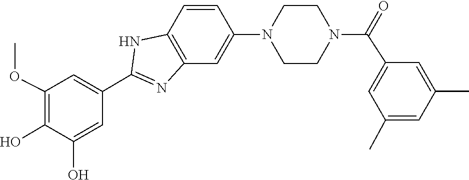 [SMILES: COc1cc(-c2nc3cc(N4CCN(C(=O)c5cc(C)cc(C)c5)CC4)ccc3[nH]2)cc(O)c1O] | (4-(2-(3,4-dihydroxy-5- methoxyphenyl)-1H- benzo[d]imidazol-5- yl)piperazin-1-yl)(3,5- dimethylphenyl)methanone | 473.3 [M + 1]⁺ |
| 126 | 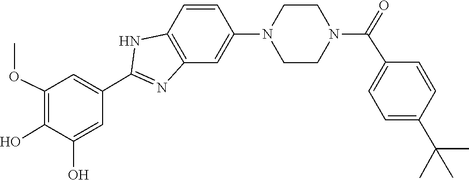 [SMILES: COc1cc(-c2nc3cc(N4CCN(C(=O)c5ccc(C(C)(C)C)cc5)CC4)ccc3[nH]2)cc(O)c1O] | (4-(tert-butyl)phenyl)(4- (2-(3,4-dihydroxy-5- methoxyphenyl)-1H- benzo[d]imidazol-5- yl)piperazin-1- yl)methanone | 501.3 [M + 1]⁺ |
| 127 | 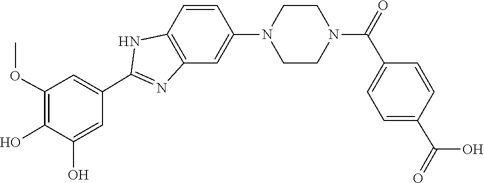 [SMILES: COc1cc(-c2nc3cc(N4CCN(C(=O)c5ccc(C(=O)O)cc5)CC4)ccc3[nH]2)cc(O)c1O] | 4-(4-(2-(3,4-dihydroxy- 5-methoxyphenyl)-1H- benzo[d]imidazol-5- yl)piperazine-1- carbonyl)benzoic acid | 489.3 [M + 1]⁺ |
| 128 | 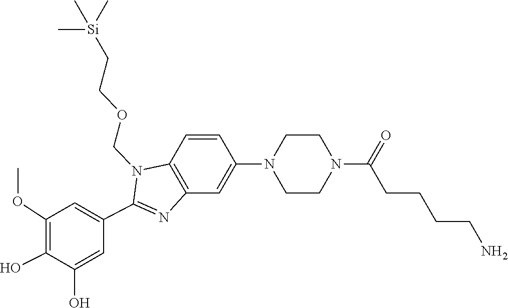 [SMILES: COc1cc(-c2nc3cc(N4CCN(C(=O)CCCCN)CC4)ccc3n2COCC[Si](C)(C)C)cc(O)c1O] | 5-amino-1-(4-(2-(3,4- dihydroxy-5- methoxyphenyl)-1-((2- (trimethylsilyl)ethoxy) methyl)-1H- benzo[d]imidazol-5- yl)piperazin-1-yl)pentan- 1-one | 570.4 [M + 1]⁺ |
| 129 | 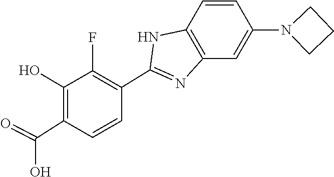 [SMILES: O=C(O)c1ccc(-c2nc3cc(N4CCC4)ccc3[nH]2)c(F)c1O] | 4-(5-(azetidin-1-yl)-1H- benzo[d]imidazol-2-yl)- 3-fluoro-2- hydroxybenzoic acid | 328.1 [M + 1]⁺ |
| 130 | 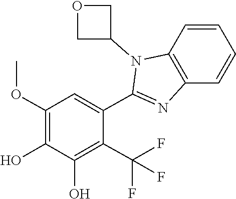 [SMILES: COc1cc(-c2nc3ccccc3n2C2COC2)c(C(F)(F)F)c(O)c1O] | 6-methoxy-4-(1-(oxetan- 3-yl)-1H- benzo[d]imidazol-2-yl)- 3- (trifluoromethyl)benzene- 1,2-diol | 381.0 [M + 1]⁺ |
| 131 | 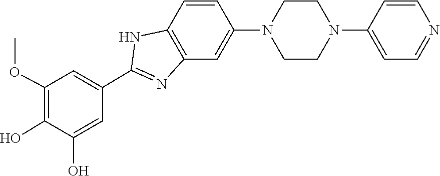 [SMILES: COc1cc(-c2nc3cc(N4CCN(c5ccncc5)CC4)ccc3[nH]2)cc(O)c1O] | 3-methoxy-5-(5-(4- (pyridin-4-yl)piperazin- 1-yl)-1H- benzo[d]imidazol-2- yl)benzene-1,2-diol | 418.3 [M + 1]⁺ |
| 132 | 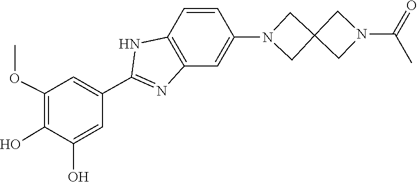 [SMILES: COc1cc(-c2nc3cc(N4CC5(CN(C(C)=O)C5)C4)ccc3[nH]2)cc(O)c1O] | 1-(6-(2-(3,4-dihydroxy- 5-methoxyphenyl)-1H- benzo[d]imidazol-5-yl)- 2,6- diazaspiro[3.3]heptan-2- yl)ethan-1-one | 395.2 [M + 1]⁺ |
| 133 | 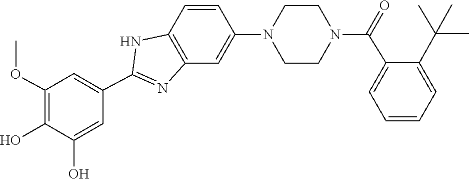 [SMILES: COc1cc(-c2nc3cc(N4CCN(C(=O)c5ccccc5C(C)(C)C)CC4)ccc3[nH]2)cc(O)c1O] | (2-(tert-butyl)phenyl)(4- (2-(3,4-dihydroxy-5- methoxyphenyl)-1H- benzo[d]imidazol-5- yl)piperazin-1- yl)methanone | 501.4 [M + 1]⁺ |
| 134 | 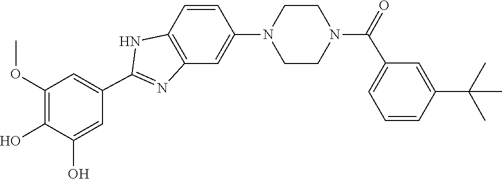 [SMILES: COc1cc(-c2nc3cc(N4CCN(C(=O)c5cccc(C(C)(C)C)c5)CC4)ccc3[nH]2)cc(O)c1O] | (3-(tert-butyl)phenyl)(4- (2-(3,4-dihydroxy-5- methoxyphenyl)-1H- benzo[d]imidazol-5- yl)piperazin-1- yl)methanone | 501.4 [M + 1]⁺ |
| 135 | 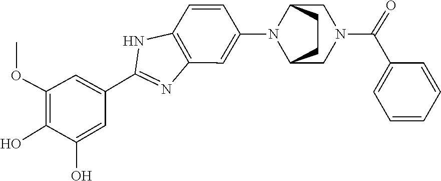 [SMILES: COc1cc(-c2nc3cc(N4[C@@H]5CC[C@H]4CN(C(=O)c4ccccc4)C5)ccc3[nH]2)cc(O)c1O] | ((1R,5S)-8-(2-(3,4- dihydroxy-5- methoxyphenyl)-1H- benzo[d]imidazol-5-yl)- 3,8- diazabicyclo[3.2.1]octan- 3-yl)(phenyl)methanone | 471.3 [M + 1]⁺ |
| 136 | 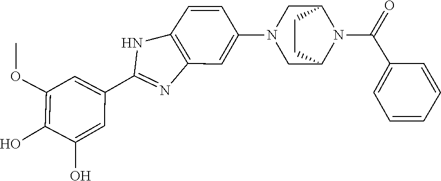 [SMILES: COc1cc(-c2nc3cc(N4C[C@H]5CC[C@@H](C4)N5C(=O)c4ccccc4)ccc3[nH]2)cc(O)c1O] | (1R,5S)-3-(2-(3,4- dihydroxy-5- methoxyphenyl)-1H- benzo[d]imidazol-5-yl)- 3,8- diazabicyclo[3.2.1]octan- 8-yl)(phenyl)methanone | 471.2 [M + 1]⁺ |
| 137 | 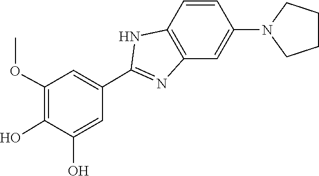 [SMILES: COc1cc(-c2nc3cc(N4CCCC4)ccc3[nH]2)cc(O)c1O] | 3-methoxy-5-(5- (pyrrolidin-1-yl)-1H- benzo[d]imidazol-2- yl)benzene-1,2-diol | 326.2 [M + 1]⁺ |
| 138 | 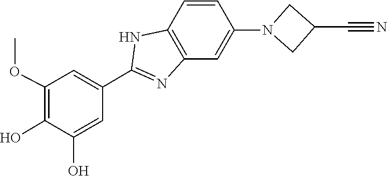 [SMILES: COc1cc(-c2nc3cc(N4CC(C#N)C4)ccc3[nH]2)cc(O)c1O] | 1-(2-(3,4-dihydroxy-5- methoxyphenyl)-1H- benzo[d]imidazol-5- yl)azetidine-3- carbonitrile | 337.1 [M + 1]⁺ |
| 139 | 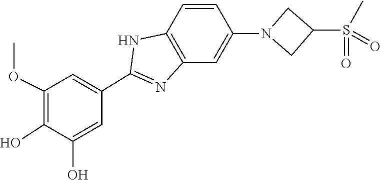 [SMILES: COc1cc(-c2nc3cc(N4CC(S(C)(=O)=O)C4)ccc3[nH]2)cc(O)c1O] | 3-methoxy-5-(5-(3- (methylsulfonyl)azetidin- 1-yl)-1H- benzo[d]imidazol-2- yl)benzene-1,2-diol | 390.2 [M + 1]⁺ |
| 140 | 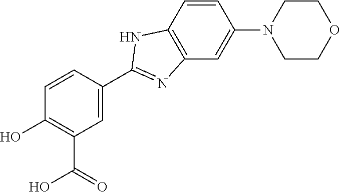 [SMILES: O=C(O)c1cc(-c2nc3cc(N4CCOCC4)ccc3[nH]2)ccc1O] | 2-hydroxy-5-(5- morpholino-1H- benzo[d]imidazol-2- yl)benzoic acid | 340.1 [M + 1]⁺ |
| 141 | 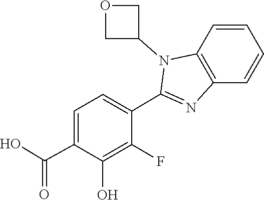 [SMILES: O=C(O)c1ccc(-c2nc3ccccc3n2C2COC2)c(F)c1O] | 3-fluoro-2-hydroxy-4-(1- (oxetan-3-yl)-1H- benzo[d]imidazol-2- yl)benzoic acid | 329.0 [M + 1]⁺ |
| 142 | 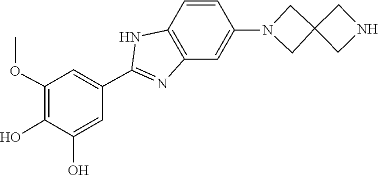 [SMILES: COc1cc(-c2nc3cc(N4CC5(CNC5)C4)ccc3[nH]2)cc(O)c1O] | 5-(5-(2,6- diazaspiro[3.3]heptan-2- yl)-1H- benzo[d]imidazol-2-yl)- 3-methoxybenzene-1,2- diol | 353.2 [M + 1]⁺ |
| 143 | 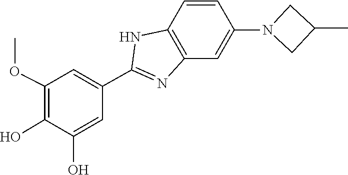 [SMILES: COc1cc(-c2nc3cc(N4CC(C)C4)ccc3[nH]2)cc(O)c1O] | 3-methoxy-5-(5-(3- methylazetidin-1-yl)-1H- benzo[d]imidazol-2- yl)benzene-1,2-diol | 326.2 [M + 1]⁺ |
| 144 | 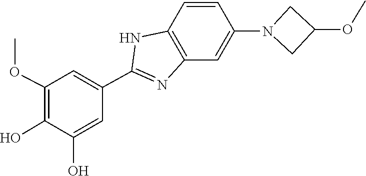 [SMILES: COc1cc(-c2nc3cc(N4CC(OC)C4)ccc3[nH]2)cc(O)c1O] | 3-methoxy-5-(5-(3- methoxyazetidin-1-yl)- 1H-benzo[d]imidazol-2- yl)benzene-1,2-diol | 342.2 [M + 1]⁺ |
| 145 | 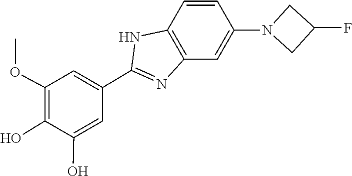 [SMILES: COc1cc(-c2nc3cc(N4CC(F)C4)ccc3[nH]2)cc(O)c1O] | 5-(5-(3-fluoroazetidin-1- yl)-1H- benzo[d]imidazol-2-yl)- 3-methoxybenzene-1,2- diol | 330.2 [M + 1]⁺ |
| 146 | 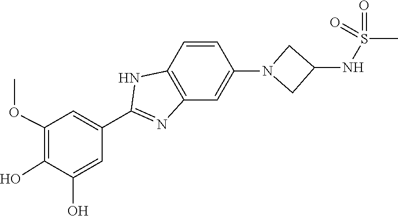 [SMILES: COc1cc(-c2nc3cc(N4CC(NS(C)(=O)=O)C4)ccc3[nH]2)cc(O)c1O] | N-(1-(2-(3,4-dihydroxy- 5-methoxyphenyl)-1H- benzo[d]imidazol-5- yl)azetidin-3- yl)methanesulfonamide | 405.2 [M + 1]⁺ |
| 147 | 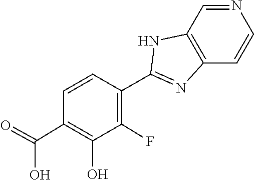 [SMILES: O=C(O)c1ccc(-c2nc3ccncc3[nH]2)c(F)c1O] | 3-fluoro-2-hydroxy-4- (3H-imidazo[4,5- c]pyridin-2-yl)benzoic acid | 274.0 [M + 1]⁺ |
| 148 | 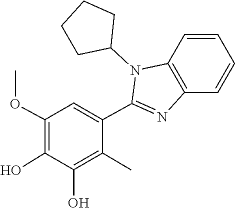 [SMILES: COc1cc(-c2nc3ccccc3n2C2CCCC2)c(C)c(O)c1O] | 4-(1-cyclopentyl-1H- benzo[d]imidazol-2-yl)- 6-methoxy-3- methylbenzene-1,2-diol | 339.2 [M + 1]⁺ |
| 149 | 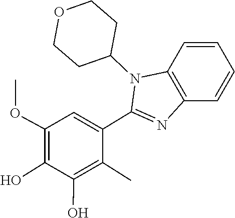 [SMILES: COc1cc(-c2nc3ccccc3n2C2CCOCC2)c(C)c(O)c1O] | 6-methoxy-3-methyl-4- (1-(tetrahydro-2H-pyran- 4-yl)-1H- benzo[d]imidazol-2- yl)benzene-1,2-diol | 355.2 [M + 1]⁺ |
| 150 | 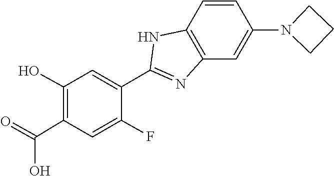 [SMILES: O=C(O)c1cc(F)c(-c2nc3cc(N4CCC4)ccc3[nH]2)cc1O] | 4-(5-(azetidin-1-yl)-1H- benzo[d]imidazol-2-yl)- 5-fluoro-2- hydroxybenzoic acid | 328.0 [M + 1]⁺ |
| 151 | 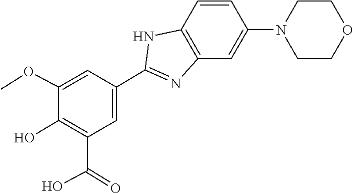 [SMILES: COc1cc(-c2nc3cc(N4CCOCC4)ccc3[nH]2)cc(C(=O)O)c1O] | 2-hydroxy-3-methoxy-5- (5-morpholino-1H- benzo[d]imidazol-2- yl)benzoic acid | 370.1 [M + 1]⁺ |
| 152 | 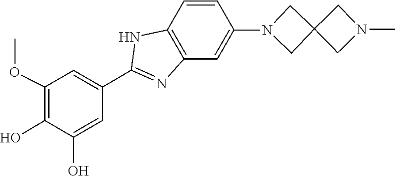 [SMILES: COc1cc(-c2nc3cc(N4CC5(CN(C)C5)C4)ccc3[nH]2)cc(O)c1O] | 3-methoxy-5-(5-(6- methyl-2,6- diazaspiro[3.3]heptan-2- yl)-1H- benzo[d]imidazol-2- yl)benzene-1,2-diol | 367.3 [M + 1]⁺ |
| 153 | 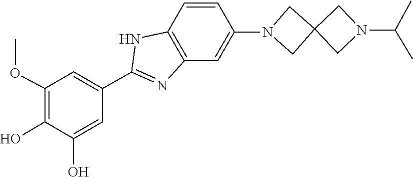 [SMILES: COc1cc(-c2nc3cc(N4CC5(C4)CN(C(C)C)C5)ccc3[nH]2)cc(O)c1O] | 5-(5-(6-isopropyl-2,6- diazaspiro[3.3]heptan-2- yl)-1H- benzo[d]imidazol-2-yl)- 3-methoxybenzene-1,2- diol | 395.2 [M + 1]⁺ |
| 154 | 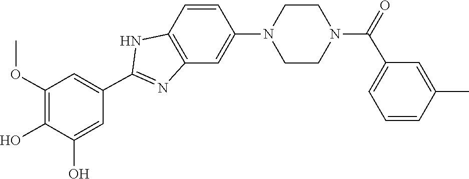 [SMILES: COc1cc(-c2nc3cc(N4CCN(C(=O)c5cccc(C)c5)CC4)ccc3[nH]2)cc(O)c1O] | (4-(2-(3,4-dihydroxy-5- methoxyphenyl)-1H- benzo[d]imidazol-5- yl)piperazin-1-yl)(m- tolyl)methanone | 459.3 [M + 1]⁺ |
| 155 | 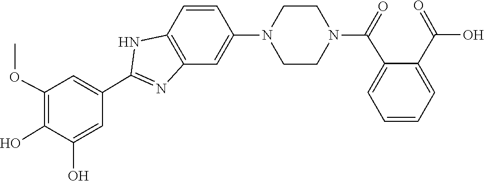 [SMILES: COc1cc(-c2nc3cc(N4CCN(C(=O)c5ccccc5C(=O)O)CC4)ccc3[nH]2)cc(O)c1O] | 2-(4-(2-(3,4-dihydroxy- 5-methoxyphenyl)-1H- benzo[d]imidazol-5- yl)piperazine-1- carbonyl)benzoic acid | 489.3 [M + 1]⁺ |
| 156 | 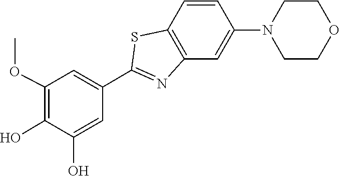 [SMILES: COc1cc(-c2nc3cc(N4CCOCC4)ccc3s2)cc(O)c1O] | 3-methoxy-5-(5- morpholinobenzo[d] thiazol-2-yl)benzene- 1,2-diol | 359.2 [M + 1]⁺ |
| 157 | 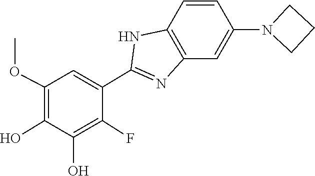 [SMILES: COc1cc(-c2nc3cc(N4CCC4)ccc3[nH]2)c(F)c(O)c1O] | 4-(5-(azetidin-1-yl)-1H- benzo[d]imidazol-2-yl)- 3-fluoro-6- methoxybenzene-1,2- diol | 330.2 [M + 1]⁺ |
| 158 | 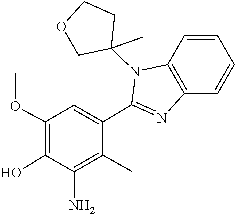 [SMILES: COc1cc(-c2nc3ccccc3n2C2(C)CCOC2)c(C)c(N)c1O] | 6-methoxy-3-methyl-4- (1-(3- methyltetrahydrofuran- 3-yl)-1H- benzo[d]imidazol-2- yl)benzene-1,2-diol | 355.2 [M + 1]⁺ |
| 159 | 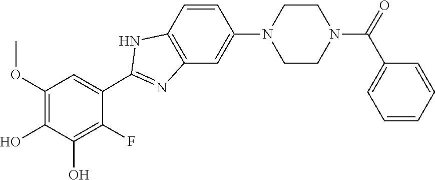 [SMILES: COc1cc(-c2nc3cc(N4CCN(C(=O)c5ccccc5)CC4)ccc3[nH]2)c(F)c(O)c1O] | (4-(2-(2-fluoro-3,4- dihydroxy-5- methoxyphenyl)-1H- benzo[d]imidazol-5- yl)piperazin-1- yl)(phenyl)methanone | 463.1 [M + 1]⁺ |
| 160 | 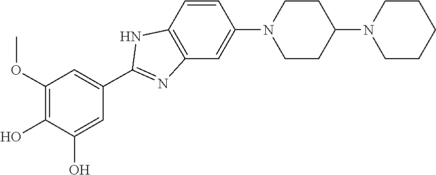 [SMILES: COc1cc(-c2nc3cc(N4CCC(N5CCCCC5)CC4)ccc3[nH]2)cc(O)c1O] | 5-(5-([1,4′-bipiperidin]- 1′-yl)-1H- benzo[d]imidazol-2-yl)- 3-methoxybenzene-1,2- diol | 423.3 [M + 1]⁺ |
| 161 | 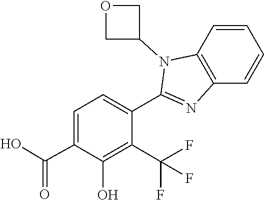 [SMILES: O=C(O)c1ccc(-c2nc3ccccc3n2C2COC2)c(C(F)(F)F)c1O] | 2-hydroxy-4-(1-(oxetan- 3-yl)-1H- benzo[d]imidazol-2-yl)- 3- (trifluoromethyl)benzoic acid | 379.2 [M + 1]⁺ |
| 162 | 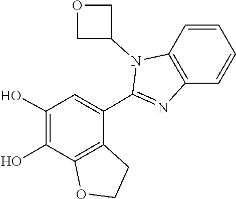 [SMILES: Oc1cc(-c2nc3ccccc3n2C2COC2)c2c(c1O)OCC2] | 4-(1-(oxetan-3-yl)-1H- benzo[d]imidazol-2-yl)- 2,3-dihydrobenzofuran- 6,7-diol | 325.2 [M + 1]⁺ |
| 163 | 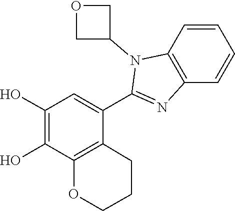 [SMILES: Oc1cc(-c2nc3ccccc3n2C2COC2)c2c(c1O)OCCC2] | 5-(1-(oxetan-3-yl)-1H- benzo[d]imidazol-2- yl)chroman-7,8-diol | 339.2 [M + 1]⁺ |
| 164 | 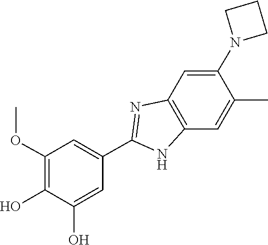 [SMILES: COc1cc(-c2nc3cc(N4CCC4)c(C)cc3[nH]2)cc(O)c1O] | 5-(5-(azetidin-1-yl)-6- methyl-1H- benzo[d]imidazol-2-yl)- 3-methoxybenzene-1,2- diol | 326.2 [M + 1]⁺ |
| 165 | 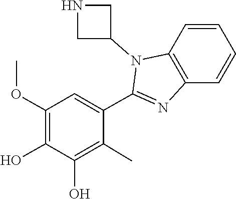 [SMILES: COc1cc(-c2nc3ccccc3n2C2CNC2)c(C)c(O)c1O] | 4-(1-(azetidin-3-yl)-1H- benzo[d]imidazol-2-yl)- 6-methoxy-3- methylbenzene-1,2-diol | 326.2 [M + 1]⁺ |
| 166 | 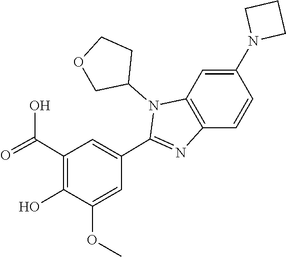 [SMILES: COc1cc(-c2nc3ccc(N4CCC4)cc3n2C2CCOC2)cc(C(=O)O)c1O] | 5-(6-(azetidin-1-yl)-1- (tetrahydrofuran-3-yl)- 1H-benzo[d]imidazol-2- yl)-2-hydroxy-3- methoxybenzoic acid | 410.2 [M + 1]⁺ |
| 167 | 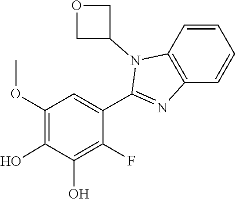 [SMILES: COc1cc(-c2nc3ccccc3n2C2COC2)c(F)c(O)c1O] | 3-fluoro-6-methoxy-4- (1-(oxetan-3-yl)-1H- benzo[d]imidazol-2- yl)benzene-1,2-diol | 331.0 [M + 1]⁺ |
| 168 | 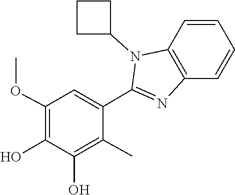 [SMILES: COc1cc(-c2nc3ccccc3n2C2CCC2)c(C)c(O)c1O] | 4-(1-cyclobutyl-1H- benzo[d]imidazol-2-yl)- 6-methoxy-3- methylbenzene-1,2-diol | 325.3 [M + 1]⁺ |
| 169 | 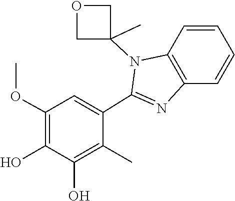 [SMILES: COc1cc(-c2nc3ccccc3n2C2(C)COC2)c(C)c(O)c1O] | 6-methoxy-3-methyl-4- (1-(3-methyloxetan-3- yl)-1H- benzo[d]imidazol-2- yl)benzene-1,2-diol | 341.3 [M + 1]⁺ |
| 170 | 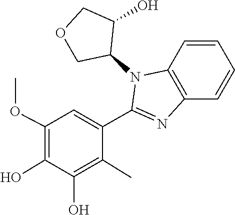 [SMILES: COc1cc(-c2nc3ccccc3n2[C@H]2COC[C@@H]2O)c(C)c(O)c1O] | 4-(1-((3S,4R)-4- hydroxytetrahydrofuran- 3-yl)-1H- benzo[d]imidazol-2-yl)- 6-methoxy-3- methylbenzene-1,2-diol | 357.3 [M + 1]⁺ |
| 171 | 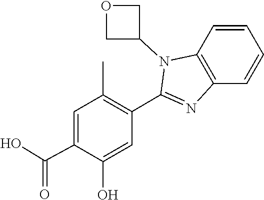 [SMILES: Cc1cc(C(=O)O)c(O)cc1-c1nc2ccccc2n1C1COC1] | 2-hydroxy-5-methyl-4- (1-(oxetan-3-yl)-1H- benzo[d]imidazol-2- yl)benzoic acid | 325.2 [M + 1]⁺ |
| 172 | 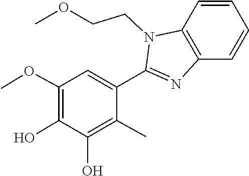 [SMILES: COCCn1c(-c2cc(OC)c(O)c(O)c2C)nc2ccccc21] | 6-methoxy-4-(1-(2- methoxyethyl)-1H- benzo[d]imidazol-2-yl)- 3-methylbenzene-1,2- diol | 329.2 [M + 1]+ |
| 173 | 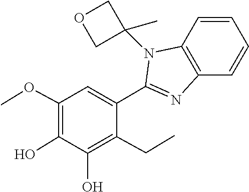 [SMILES: CCc1c(-c2nc3ccccc3n2C2(C)COC2)cc(OC)c(O)c1O] | 3-ethyl-6-methoxy-4-(1- (3-methyloxetan-3-yl)- 1H-benzo[d]imidazol-2- yl)benzene-1,2-diol | 355.3 [M + 1]+ |
| 174 | 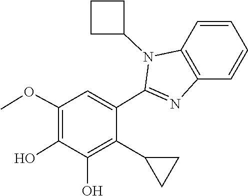 [SMILES: COc1cc(-c2nc3ccccc3n2C2CCC2)c(C2CC2)c(O)c1O] | 4-(1-cyclobutyl-1H- benzo[d]imidazol-2-yl)- 3-cyclopropyl-6- methoxybenzene-1,2- diol | 351.3 [M + 1]+ |
| 175 | 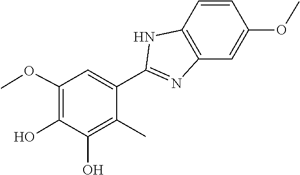 | 6-methoxy-4-(5- methoxy-1H- benzo[d]imidazol-2-yl)- 3-methylbenzene-1,2- diol | 301.2 [M + 1]+ |
| 176 | 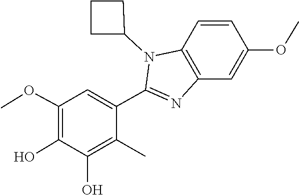 | 4-(1-cyclobutyl-5- methoxy-1H- benzo[d]imidazol-2-yl)- 6-methoxy-3- methylbenzene-1,2-diol | 355.3 [M + 1]+ |
| 177 | 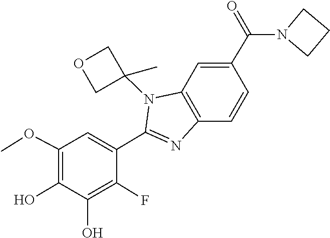 [SMILES: COc1cc(-c2nc3ccc(C(=O)N4CCC4)cc3n2C2(C)COC2)c(F)c(O)c1O] | 4-[6-(azetidine-1- carbonyl)-1-(3- methyloxetan-3-yl)-1H- 1,3-benzodiazol-2-yl]-3- fluoro-6- methoxybenzene-1,2- diol | 428.2 [M + 1]+ |
| 178 | 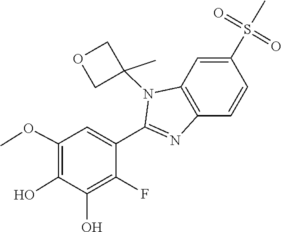 [SMILES: COc1cc(-c2nc3ccc(S(C)(=O)=O)cc3n2C2(C)COC2)c(F)c(O)c1O] | 3-fluoro-6-methoxy-4- (1-(3-methyloxetan-3- yl)-6-(methylsulfonyl)- 1H-benzo[d]imidazol-2- yl)benzene-1,2-diol | 423.1 [M + 1]+ |
| 179 | 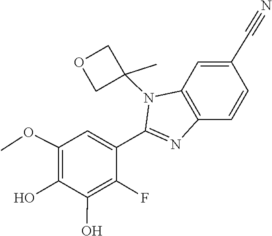 [SMILES: COc1cc(-c2nc3ccc(C#N)cc3n2C2(C)COC2)c(F)c(O)c1O] | 2-(2-fluoro-3,4- dihydroxy-5- methoxyphenyl)-1-(3- methyloxetan-3-yl)-1H- 1,3-benzodiazole-6- carbonitrile | 370.1 [M + 1]+ |
| 180 | 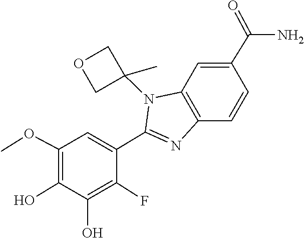 [SMILES: COc1cc(-c2nc3ccc(C(N)=O)cc3n2C2(C)COC2)c(F)c(O)c1O] | 2-(2-fluoro-3,4- dihydroxy-5- methoxyphenyl)-1-(3- methyloxetan-3-yl)-1H- benzo[d]imidazole-6- carboxamide | 388.2 [M + 1]+ |
| 181 | 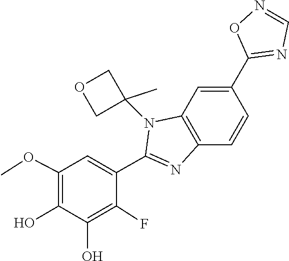 [SMILES: COc1cc(-c2nc3ccc(-c4ncno4)cc3n2C2(C)COC2)c(F)c(O)c1O] | 3-fluoro-6-methoxy-4- (1-(3-methyloxetan-3- yl)-6-(1,2,4-oxadiazol-5- yl)-1H- benzo[d]imidazol-2- yl)benzene-1,2-diol | 413.1 [M + 1]+ |
| 182 | 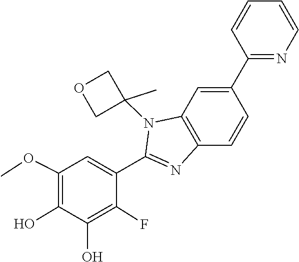 [SMILES: COc1cc(-c2nc3ccc(-c4ccccn4)cc3n2C2(C)COC2)c(F)c(O)c1O] | 3-fluoro-6-methoxy-4- (1-(3-methyloxetan-3- yl)-6-(pyridin-2-yl)-1H- benzo[d]imidazol-2- yl)benzene-1,2-diol | 422.2 [M + 1]+ |
| 183 | 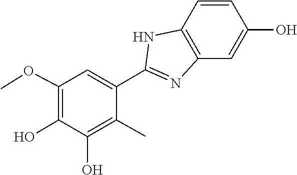 [SMILES: COc1cc(-c2nc3cc(O)ccc3[nH]2)c(C)c(O)c1O] | 4-(5-hydroxy-1H-1,3- benzodiazol-2-yl)-6- methoxy-3- methylbenzene-1,2-diol | 287.2 [M + 1]+ |
| 184 | 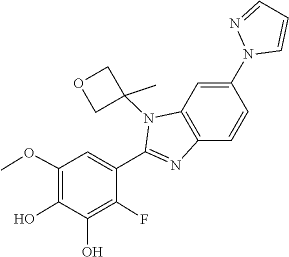 [SMILES: COc1cc(-c2nc3ccc(-n4cccn4)cc3n2C2(C)COC2)c(F)c(O)c1O] | 3-fluoro-6-methoxy-4- (1-(3-methyloxetan-3- yl)-6-(1H-pyrazol-1-yl)- 1H-benzo[d]imidazol-2- yl)benzene-1,2-diol | 411.2 [M + 1]+ |
| 185 | 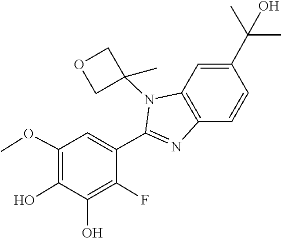 [SMILES: COc1cc(-c2nc3ccc(C(C)(C)O)cc3n2C2(C)COC2)c(F)c(O)c1O] | 3-fluoro-4-(6-(2- hydroxypropan-2-yl)-1- (3-methyloxetan-3-yl)- 1H-benzo[d]imidazol-2- yl)-6-methoxybenzene- 1,2-diol | 403.2 [M + 1]+ |
| 186 | 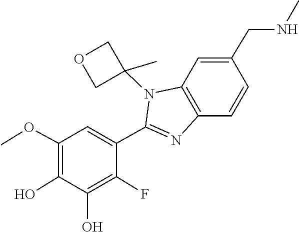 | 3-fluoro-6-methoxy-4- {6- [(methylamino)methyl]- 1-(3-methyloxetan-3-yl)- 1H-1,3-benzodiazol-2- yl}benzene-1,2-diol | 388.2 [M + 1]+ |
| 187 | 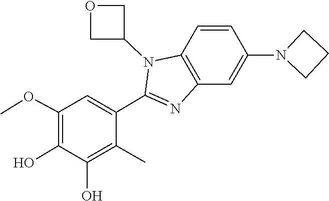 [SMILES: COc1cc(-c2nc3cc(N4CCC4)ccc3n2C2COC2)c(C)c(O)c1O] | 4-(5-(azetidin-1-yl)-1- (oxetan-3-yl)-1H- benzo[d]imidazol-2-yl)- 6-methoxy-3- methylbenzene-1,2-diol | 382.2 [M + 1]+ |
| 188 | 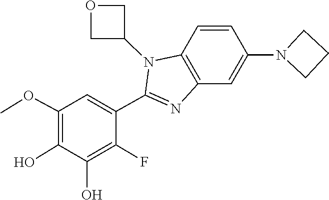 [SMILES: COc1cc(-c2nc3cc(N4CCC4)ccc3n2C2COC2)c(F)c(O)c1O] | 4-(5-(azetidin-1-yl)-1- (oxetan-3-yl)-1H- benzo[d]imidazol-2-yl)- 3-fluoro-6- methoxybenzene-1,2- diol | 386.1 [M + 1]+ |
| 189 | 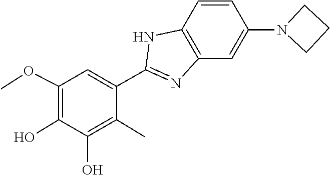 [SMILES: COc1cc(-c2nc3cc(N4CCC4)ccc3[nH]2)c(C)c(O)c1O] | 4-(5-(azetidin-1-yl)-1H- benzo[d]imidazol-2-yl)- 6-methoxy-3- methylbenzene-1,2-diol | 326.1 [M + 1]+ |
| 190 | 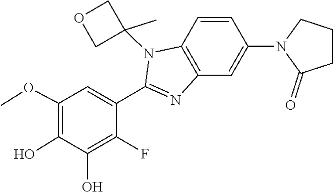 [SMILES: COc1cc(-c2nc3cc(N4CCCC4=O)ccc3n2C2(C)COC2)c(F)c(O)c1O] | 1-(2-(2-fluoro-3,4- dihydroxy-5- methoxyphenyl)-1-(3- methyloxetan-3-yl)-1H- benzo[d]imidazol-5- yl)pyrrolidin-2-one | 428.2 [M + 1]+ |
| 191 | 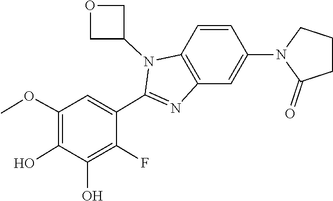 [SMILES: COc1cc(-c2nc3cc(N4CCCC4=O)ccc3n2C2COC2)c(F)c(O)c1O] | 1-(2-(2-fluoro-3,4- dihydroxy-5- methoxyphenyl)-1- (oxetan-3-yl)-1H- benzo[d]imidazol-5- yl)pyrrolidin-2-one | 414.2 [M + 1]+ |
| 192 | 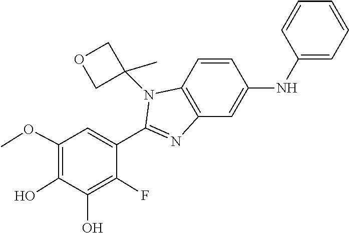 [SMILES: COc1cc(-c2nc3cc(Nc4ccccc4)ccc3n2C2(C)COC2)c(F)c(O)c1O] | 3-fluoro-6-methoxy-4- (1-(3-methyloxetan-3- yl)-5-(phenylamino)-1H- benzo[d]imidazol-2- yl)benzene-1,2-diol | 436.3 [M + 1]+ |
| 193 | 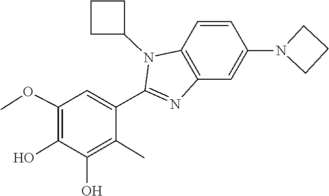 [SMILES: COc1cc(-c2nc3cc(N4CCC4)ccc3n2C2CCC2)c(C)c(O)c1O] | 4-(5-(azetidin-1-yl)-1- cyclobutyl-1H- benzo[d]imidazol-2-yl)- 6-methoxy-3- methylbenzene-1,2-diol | 380.2 [M + 1]+ |
| 194 | 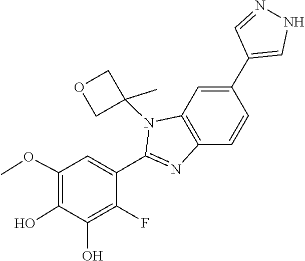 [SMILES: COc1cc(-c2nc3ccc(-c4cn[nH]c4)cc3n2C2(C)COC2)c(F)c(O)c1O] | 3-fluoro-6-methoxy-4- (1-(3-methyloxetan-3- yl)-6-(1H-pyrazol-4-yl)- 1H-benzo[d]imidazol-2- yl)benzene-1,2-diol | 411.2 [M + 1]+ |
| 195 | 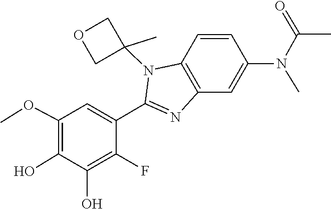 [SMILES: COc1cc(-c2nc3cc(N(C)C(C)=O)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5-yl)- N-methylacetamide | 416.1 [M + 1]+ |
| 196 | 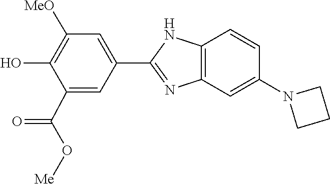 [SMILES: COC(=O)c1cc(-c2nc3cc(N4CCC4)ccc3[nH]2)cc(OC)c1O] | methyl 5-(5-(azetidin-1- yl)-1H- benzo[d]imidazol-2-yl)- 2-hydroxy-3- methoxybenzoate | 354.1 [M + 1]+ |
| 197 | 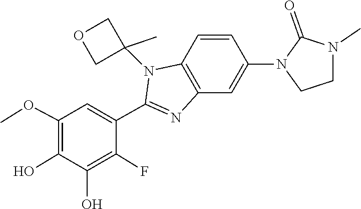 | 1-(2-(2-fluoro-3,4- dihydroxy-5- methoxyphenyl)-1-(3- methyloxetan-3-yl)-1H- benzo[d]imidazol-5-yl)- 3-methylimidazolidin-2- one | 443.1 [M + 1]+ |
| 198 | 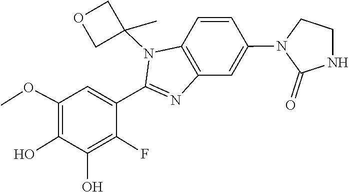 [SMILES: COc1cc(-c2nc3cc(N4CCNC4=O)ccc3n2C2(C)COC2)c(F)c(O)c1O] | 1-(2-(2-fluoro-3,4- dihydroxy-5- methoxyphenyl)-1-(3- methyloxetan-3-yl)-1H- benzo[d]imidazol-5- yl)imidazolidin-2-one | 429.2 [M + 1]+ |
| 199 | 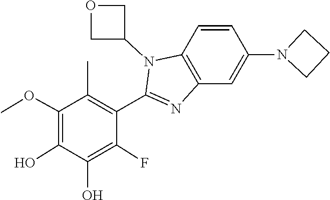 [SMILES: COc1c(C)c(-c2nc3cc(N4CCC4)ccc3n2C2COC2)c(F)c(O)c1O] | 4-(5-(azetidin-1-yl)-1- (oxetan-3-yl)-1H- benzo[d]imidazol-2-yl)- 3-fluoro-6-methoxy-5- methylbenzene-1,2-diol | 400.1 [M + 1]+ |
| 200 | 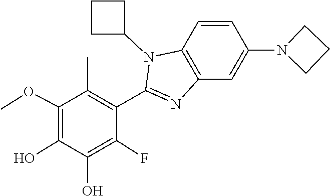 [SMILES: COc1c(C)c(-c2nc3cc(N4CCC4)ccc3n2C2CCC2)c(F)c(O)c1O] | 4-(5-(azetidin-1-yl)-1- cyclobutyl-1H- benzo[d]imidazol-2-yl)- 3-fluoro-6-methoxy-5- methylbenzene-1,2-diol | 398.1 [M + 1]+ |
| 201 | 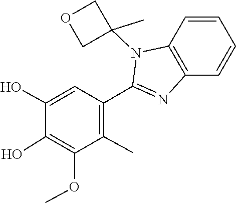 [SMILES: COc1c(C)c(-c2nc3ccccc3n2C2(C)COC2)cc(O)c1O] | 3-methoxy-4-methyl-5- (1-(3-methyloxetan-3- yl)-1H- benzo[d]imidazol-2- yl)benzene-1,2-diol | 341.1 [M + 1]+ |
| 202 | 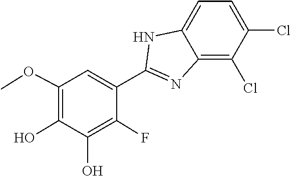 [SMILES: COc1cc(-c2nc3c(Cl)c(Cl)ccc3[nH]2)c(F)c(O)c1O] | 4-(4,5-dichloro-1H- benzo[d]imidazol-2-yl)- 3-fluoro-6- methoxybenzene-1,2- diol | 343.0 [M + 1]+ |
| 203 | 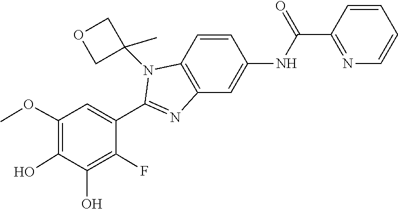 [SMILES: COc1cc(-c2nc3cc(NC(=O)c4ccccn4)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5- yl)picolinamide | 465.0 [M + 1]+ |
| 204 | 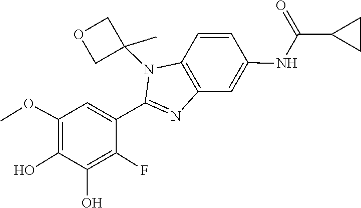 [SMILES: COc1cc(-c2nc3cc(NC(=O)C4CC4)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5- yl)cyclopropanecarbox- amide | 428.2 [M + 1]+ |
| 205 | 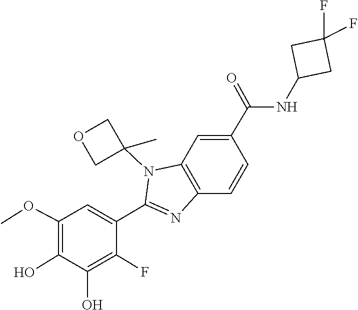 [SMILES: COc1cc(-c2nc3ccc(C(=O)NC4CC(F)(F)C4)cc3n2C2(C)COC2)c(F)c(O)c1O] | N-(3,3- difluorocyclobutyl)-2-(2- fluoro-3,4-dihydroxy-5- methoxyphenyl)-1-(3- methyloxetan-3-yl)-1H- benzo[d]imidazole-6- carboxamide | 478.0 [M + 1]+ |
| 206 | 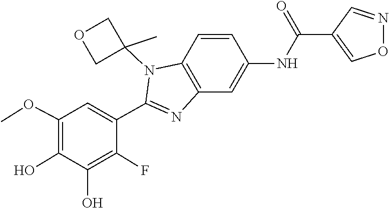 [SMILES: COc1cc(-c2nc3cc(NC(=O)c4cnoc4)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5- yl)isoxazole-4- carboxamide | 455.3 [M + 1]+ |
| 207 | 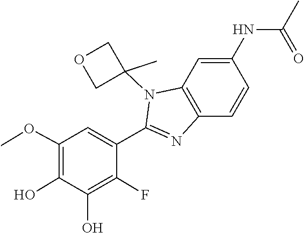 [SMILES: COc1cc(-c2nc3ccc(NC(C)=O)cc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-6- yl)acetamide | 402.1 [M + 1]+ |
| 209 | 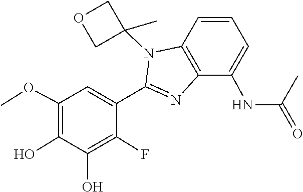 [SMILES: COc1cc(-c2nc3c(NC(C)=O)cccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-4- yl)acetamide | 402.1 [M + 1]+ |
| 210 | 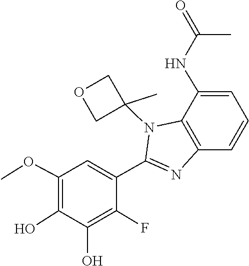 [SMILES: COc1cc(-c2nc3cccc(NC(C)=O)c3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-7- yl)acetamide | 402.3 [M + 1]+ |
| 211 | 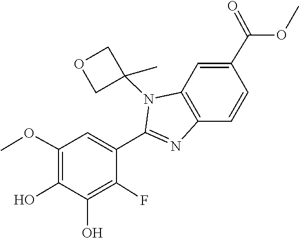 | methyl 2-(2-fluoro-3,4- dihydroxy-5- methoxyphenyl)-1-(3- methyloxetan-3-yl)-1H- benzo[d]imidazole-6- carboxylate | 403.3 [M + 1]+ |
| 212 | 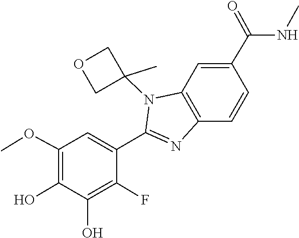 | 2-(2-fluoro-3,4- dihydroxy-5- methoxyphenyl)-N- methyl-1-(3- methyloxetan-3-yl)-1H- benzo[d]imidazole-6- carboxamide | 402.0 [M + 1]+ |
| 213 | 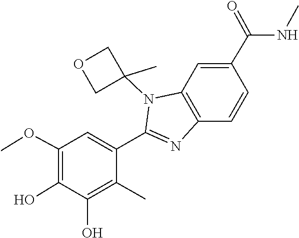 | 2-(3,4-dihydroxy-5- methoxy-2- methylphenyl)-N- methyl-1-(3- methyloxetan-3-yl)-1H- benzo[d]imidazole-6- carboxamide | 398.3 [M + 1]+ |
| 214 | 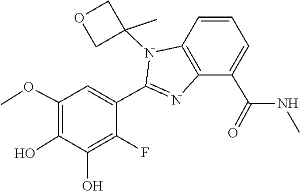 | 2-(2-fluoro-3,4- dihydroxy-5- methoxyphenyl)-N- methyl-1-(3- methyloxetan-3-yl)-1H- benzo[d]imidazole-4- carboxamide | 402.3 [M + 1]+ |
| 215 | 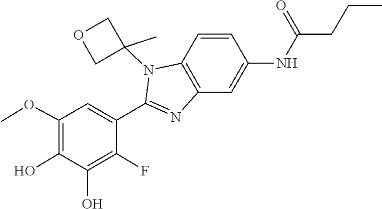 [SMILES: CCCC(=O)Nc1ccc2c(c1)nc(-c1cc(OC)c(O)c(O)c1F)n2C1(C)COC1] | N-(2-(2-fluoro-3,4- dihydroxy-5- methoxyphenyl)-1-(3- methyloxetan-3-yl)-1H- benzo[d]imidazol-5- yl)butyramide | 430.0 [M + 1]+ |
| 216 | 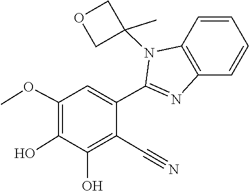 [SMILES: COc1cc(-c2nc3ccccc3n2C2(C)COC2)c(C#N)c(O)c1O] | 2,3-dihydroxy-4- methoxy-6-(1-(3- methyloxetan-3-yl)-1H- benzo[d]imidazol-2- yl)benzonitrile | 352.3 [M + 1]+ |
| 217 | 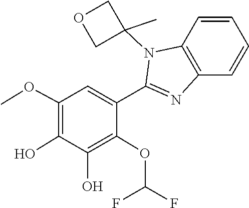 | 3-(difluoromethoxy)-6- methoxy-4-(1-(3- methyloxetan-3-yl)-1H- benzo[d]imidazol-2- yl)benzene-1,2-diol | 393.3 [M + 1]+ |
| 218 | 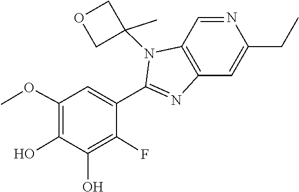 [SMILES: CCc1cc2nc(-c3cc(OC)c(O)c(O)c3F)n(C3(C)COC3)c2cn1] | 4-(6-ethyl-3-(3- methyloxetan-3-yl)-3H- imidazo[4,5-c]pyridin-2- yl)-3-fluoro-6- methoxybenzene-1,2- diol | 374.3 [M + 1]+ |
| 219 | 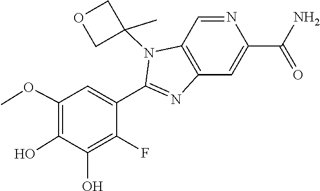 [SMILES: COc1cc(-c2nc3cc(C(N)=O)ncc3n2C2(C)COC2)c(F)c(O)c1O] | 2-(2-fluoro-3,4- dihydroxy-5- methoxyphenyl)-3-(3- methyloxetan-3-yl)-3H- imidazo[4,5-c]pyridine- 6-carboxamide | 389.3 [M + 1]+ |
| 220 | 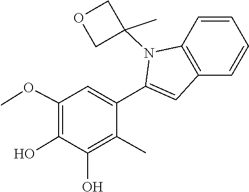 [SMILES: COc1cc(-c2cc3ccccc3n2C2(C)COC2)c(C)c(O)c1O] | 6-methoxy-3-methyl-4- (1-(3-methyloxetan-3- yl)-1H-indol-2- yl)benzene-1,2-diol | 338.0 [M + 1]+ |
| 221 | 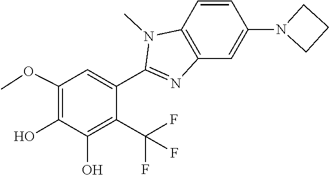 [SMILES: COc1cc(-c2nc3cc(N4CCC4)ccc3n2C)c(C(F)(F)F)c(O)c1O] | 4-(5-(azetidin-1-yl)-1- methyl-1H- benzo[d]imidazol-2-yl)- 6-methoxy-3- (trifluoromethyl)benzene- 1,2-diol | 394.0 [M + 1]+ |
| 222 | 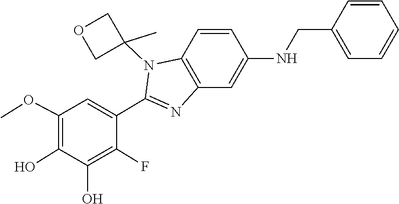 [SMILES: COc1cc(-c2nc3cc(NCc4ccccc4)ccc3n2C2(C)COC2)c(F)c(O)c1O] | 4-(5-(benzylamino)-1- (3-methyloxetan-3-yl)- 1H-benzo[d]imidazol-2- yl)-3-fluoro-6- methoxybenzene-1,2- diol | 450.4 [M + 1]+ |
| 223 | 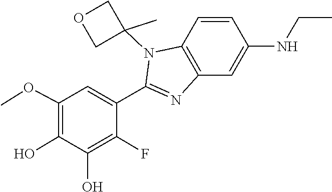 [SMILES: CCNc1ccc2c(c1)nc(-c1cc(OC)c(O)c(O)c1F)n2C1(C)COC1] | 4-(5-(ethylamino)-1-(3- methyloxetan-3-yl)-1H- benzo[d]imidazol-2-yl)- 3-fluoro-6- methoxybenzene-1,2- diol | 388.1 [M + 1]+ |
| 224 | 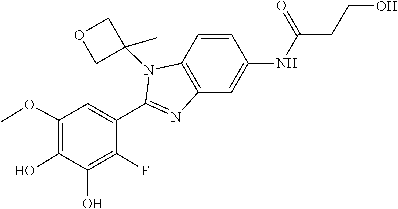 [SMILES: COc1cc(-c2nc3cc(NC(=O)CCO)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5-yl)- 3-hydroxypropanamide | 432.1 [M + 1]+ |
| 225 | 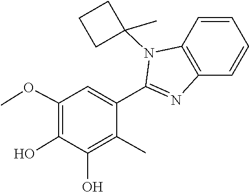 [SMILES: COc1cc(-c2nc3ccccc3n2C2(C)CCC2)c(C)c(O)c1O] | 6-methoxy-3-methyl-4- (1-(1-methylcyclobutyl)- 1H-benzo[d]imidazol-2- yl)benzene-1,2-diol | 339.1 [M + 1]+ |
| 226 | 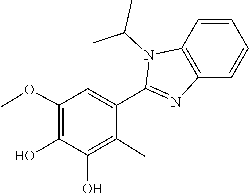 [SMILES: COc1cc(-c2nc3ccccc3n2C(C)C)c(C)c(O)c1O] | 4-(1-isopropyl-1H- benzo[d]imidazol-2-yl)- 6-methoxy-3- methylbenzene-1,2-diol | 313.1 [M + 1]+ |
| 227 | 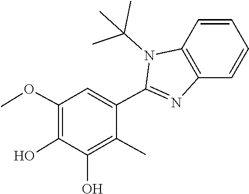 [SMILES: COc1cc(-c2nc3ccccc3n2C(C)(C)C)c(C)c(O)c1O] | 4-(1-(tert-butyl)-1H- benzo[d]imidazol-2-yl)- 6-methoxy-3- methylbenzene-1,2-diol | 327.1 [M + 1]+ |
| 228 | 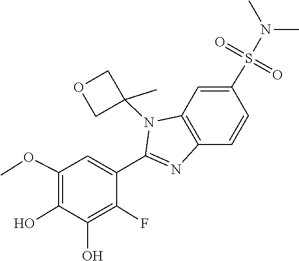 | 2-(2-fluoro-3,4- dihydroxy-5- methoxyphenyl)-N,N- dimethyl-1-(3- methyloxetan-3-yl)-1H- benzo[d]imidazole-6- sulfonamide | 452.1 [M + 1]+ |
| 229 | 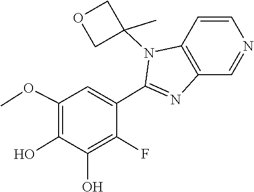 [SMILES: COc1cc(-c2nc3cnccc3n2C2(C)COC2)c(F)c(O)c1O] | 3-fluoro-6-methoxy-4- (1-(3-methyloxetan-3- yl)-1H-imidazo[4,5- c]pyridin-2-yl)benzene- 1,2-diol | 346.2 [M + 1]+ |
| 230 | 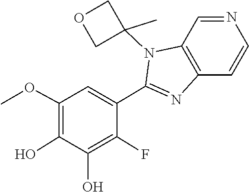 [SMILES: COc1cc(-c2nc3ccncc3n2C2(C)COC2)c(F)c(O)c1O] | 3-fluoro-6-methoxy-4- (3-(3-methyloxetan-3- yl)-3H-imidazo[4,5- c]pyridin-2-yl)benzene- 1,2-diol | 345.9 [M + 1]+ |
| 231 | 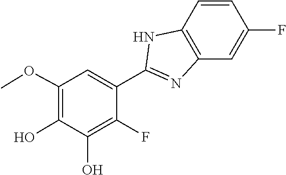 [SMILES: COc1cc(-c2nc3cc(F)ccc3[nH]2)c(F)c(O)c1O] | 3-fluoro-4-(5-fluoro-1H- benzo[d]imidazol-2-yl)- 6-methoxybenzene-1,2- diol | 293.0 [M + 1]+ |
| 232 | 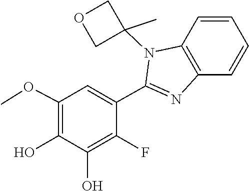 [SMILES: COc1cc(-c2nc3ccccc3n2C2(C)COC2)c(F)c(O)c1O] | 3-fluoro-6-methoxy-4- (1-(3-methyloxetan-3- yl)-1H- benzo[d]imidazol-2- yl)benzene-1,2-diol | 345.3 [M + 1]+ |
| 233 | 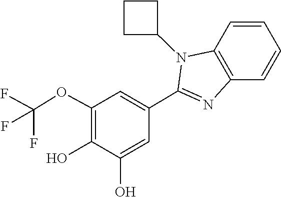 [SMILES: Oc1cc(-c2nc3ccccc3n2C2CCC2)cc(OC(F)(F)F)c1O] | 5-(1-cyclobutyl-1H- benzo[d]imidazol-2-yl)- 3- (trifluoromethoxy)benzene- 1,2-diol | 365.1 [M + 1]+ |
| 234 | 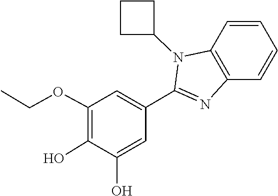 [SMILES: CCOc1cc(-c2nc3ccccc3n2C2CCC2)cc(O)c1O] | 5-(1-cyclobutyl-1H- benzo[d]imidazol-2-yl)- 3-ethoxybenzene-1,2- diol | 325.1 [M + 1]+ |
| 235 | 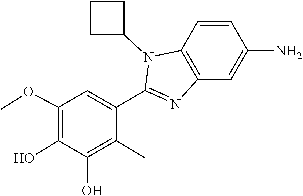 [SMILES: COc1cc(-c2nc3cc(N)ccc3n2C2CCC2)c(C)c(O)c1O] | 4-(5-amino-1- cyclobutyl-1H- benzo[d]imidazol-2-yl)- 6-methoxy-3- methylbenzene-1,2-diol | 340.1 [M + 1]+ |
| 236 | 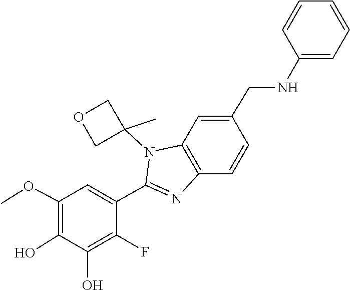 [SMILES: COc1cc(-c2nc3ccc(CNc4ccccc4)cc3n2C2(C)COC2)c(F)c(O)c1O] | 3-fluoro-6-methoxy-4- (1-(3-methyloxetan-3- yl)-6- ((phenylamino)methyl)- 1H-benzo[d]imidazol-2- yl)benzene-1,2-diol | 450.5 [M + 1]+ |
| 237 | 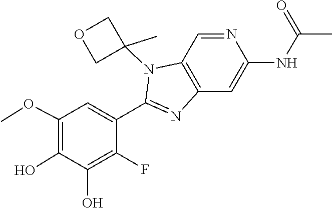 [SMILES: COc1cc(-c2nc3cc(NC(C)=O)ncc3n2C2(C)COC2)c(F)c(O)c1O] | N-(2-(2-fluoro-3,4- dihydroxy-5- methoxyphenyl)-3-(3- methyloxetan-3-yl)-3H- imidazo[4,5-c]pyridin-6- yl)acetamide | 403.4 [M + 1]+ |
| 238 | 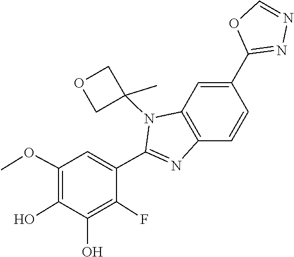 [SMILES: COc1cc(-c2nc3ccc(-c4nnco4)cc3n2C2(C)COC2)c(F)c(O)c1O] | 3-fluoro-6-methoxy-4- (1-(3-methyloxetan-3- yl)-6-(1,3,4-oxadiazol-2- yl)-1H- benzo[d]imidazol-2- yl)benzene-1,2-diol | 413.3 [M + 1]+ |
| 239 | 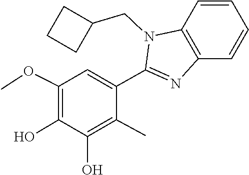 [SMILES: COc1cc(-c2nc3ccccc3n2CC2CCC2)c(C)c(O)c1O] | 4-(1-(cyclobutylmethyl)- 1H-benzo[d]imidazol-2- yl)-6-methoxy-3- methylbenzene-1,2-diol | 339.2 [M + 1]+ |
| 241 | 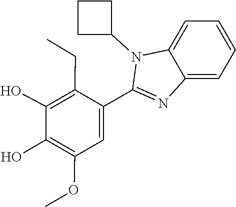 [SMILES: CCc1c(-c2nc3ccccc3n2C2CCC2)cc(OC)c(O)c1O] | 4-(1-cyclobutyl-1H- benzo[d]imidazol-2-yl)- 3-ethyl-6- methoxybenzene-1,2- diol | 339.2 [M + 1]+ |
| 242 | 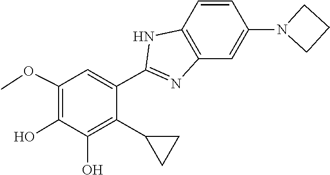 [SMILES: COc1cc(-c2nc3cc(N4CCC4)ccc3[nH]2)c(C2CC2)c(O)c1O] | 4-(5-(azetidin-1-yl)-1H- benzo[d]imidazol-2-yl)- 3-cyclopropyl-6- methoxybenzene-1,2- diol | 352.3 [M + 1]+ |
| 243 | 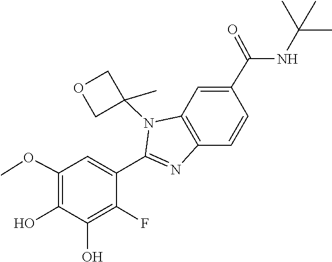 [SMILES: COc1cc(-c2nc3ccc(C(=O)NC(C)(C)C)cc3n2C2(C)COC2)c(F)c(O)c1O] | N-tert-butyl-2-(2-fluoro- 3,4-dihydroxy-5- methoxyphenyl)-1-(3- methyloxetan-3-yl)-1H- 1,3-benzodiazole-6- carboxamide | 444.2 [M + 1]+ |
| 244 | 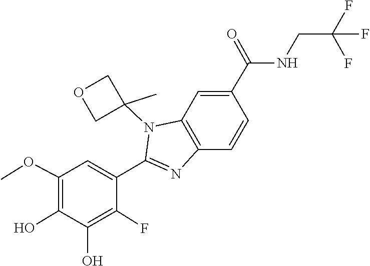 [SMILES: COc1cc(-c2nc3ccc(C(=O)NCC(F)(F)F)cc3n2C2(C)COC2)c(F)c(O)c1O] | 2-(2-fluoro-3,4- dihydroxy-5- methoxyphenyl)-1-(3- methyloxetan-3-yl)-N- (2,2,2-trifluoroethyl)- 1H-1,3-benzodiazole-6- carboxamide | 470.1 [M + 1]+ |
| 245 | 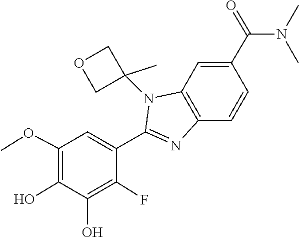 | 2-(2-fluoro-3,4- dihydroxy-5- methoxyphenyl)-N,N- dimethyl-1-(3- methyloxetan-3-yl)-1H- benzo[d]imidazole-6- carboxamide | 416.2 [M + 1]+ |
| 246 | 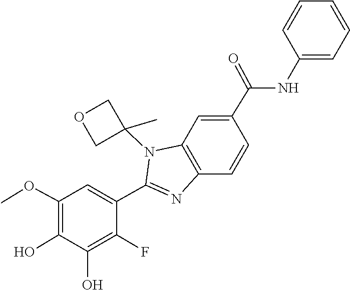 [SMILES: COc1cc(-c2nc3ccc(C(=O)Nc4ccccc4)cc3n2C2(C)COC2)c(F)c(O)c1O] | 2-(2-fluoro-3,4- dihydroxy-5- methoxyphenyl)-1-(3- methyloxetan-3-yl)-N- phenyl-1H- benzo[d]imidazole-6- carboxamide | 464.2 [M + 1]+ |
| 247 | 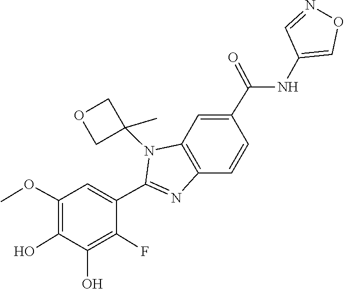 [SMILES: COc1cc(-c2nc3ccc(C(=O)Nc4cnoc4)cc3n2C2(C)COC2)c(F)c(O)c1O] | 2-(2-fluoro-3,4- dihydroxy-5- methoxyphenyl)-N- (isoxazol-4-yl)-1-(3- methyloxetan-3-yl)-1H- benzo[d]imidazole-6- carboxamide | 455.2 [M + 1]+ |
| 248 | 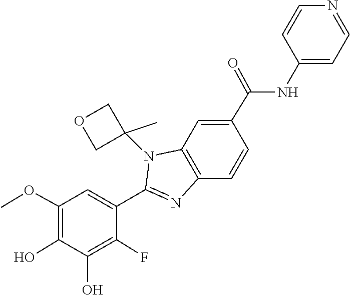 [SMILES: COc1cc(-c2nc3ccc(C(=O)Nc4ccncc4)cc3n2C2(C)COC2)c(F)c(O)c1O] | 2-(2-fluoro-3,4- dihydroxy-5- methoxyphenyl)-1-(3- methyloxetan-3-yl)-N- (pyridin-4-yl)-1H-1,3- benzodiazole-6- carboxamide | 465.2 [M + 1]+ |
| 249 | 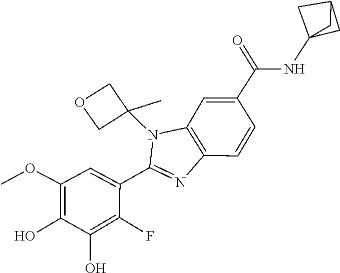 [SMILES: COc1cc(-c2nc3ccc(C(=O)NC45CC(C4)C5)cc3n2C2(C)COC2)c(F)c(O)c1O] | N-(bicyclo[1.1.1]pentan- 1-yl)-2-(2-fluoro-3,4- dihydroxy-5- methoxyphenyl)-1-(3- methyloxetan-3-yl)-1H- benzo[d]imidazole-6- carboxamide | 454.2 [M + 1]+ |
| 250 | 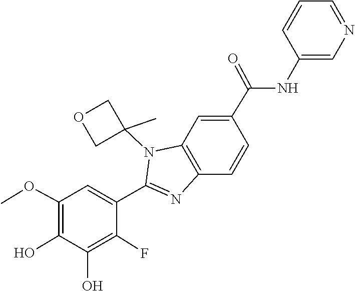 [SMILES: COc1cc(-c2nc3ccc(C(=O)Nc4cccnc4)cc3n2C2(C)COC2)c(F)c(O)c1O] | 2-(2-fluoro-3,4- dihydroxy-5- methoxyphenyl)-1-(3- methyloxetan-3-yl)-N- (pyridin-3-yl)-1H-1,3- benzodiazole-6- carboxamide | 465.1 [M + 1]+ |
| 251 | 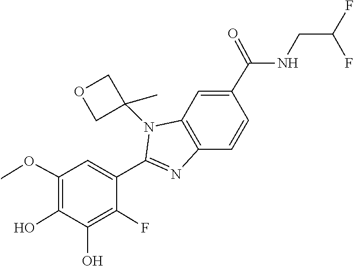 [SMILES: COc1cc(-c2nc3ccc(C(=O)NCC(F)F)cc3n2C2(C)COC2)c(F)c(O)c1O] | N-(2,2-difluoroethyl)-2- (2-fluoro-3,4-dihydroxy- 5-methoxyphenyl)-1-(3- methyloxetan-3-yl)-1H- 1,3-benzodiazole-6- carboxamide | 452.1 [M + 1]+ |
| 252 | 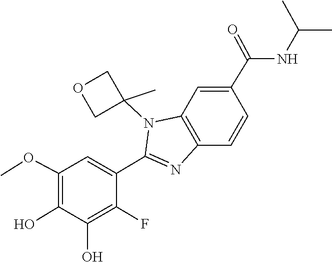 [SMILES: COc1cc(-c2nc3ccc(C(=O)NC(C)C)cc3n2C2(C)COC2)c(F)c(O)c1O] | 2-(2-fluoro-3,4- dihydroxy-5- methoxyphenyl)-N- isopropyl-1-(3- methyloxetan-3-yl)-1H- benzo[d]imidazole-6- carboxamide | 430.2 [M + 1]+ |
| 253 | 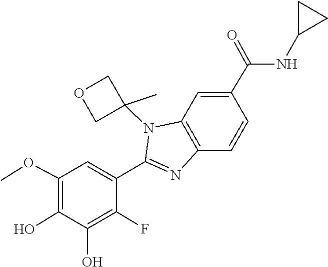 [SMILES: COc1cc(-c2nc3ccc(C(=O)NC4CC4)cc3n2C2(C)COC2)c(F)c(O)c1O] | N-cyclopropyl-2-(2- fluoro-3,4-dihydroxy-5- methoxyphenyl)-1-(3- methyloxetan-3-yl)-1H- benzo[d]imidazole-6- carboxamide | 428.2 [M + 1]+ |
| 254 | 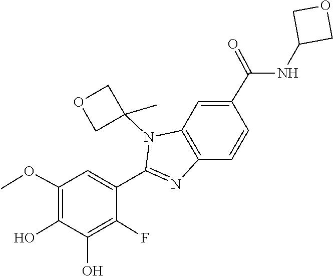 [SMILES: COc1cc(-c2nc3ccc(C(=O)NC4COC4)cc3n2C2(C)COC2)c(F)c(O)c1O] | 2-(2-fluoro-3,4- dihydroxy-5- methoxyphenyl)-1-(3- methyloxetan-3-yl)-N- (oxetan-3-yl)-1H- benzo[d]imidazole-6- carboxamide | 444.2 [M + 1]+ |
| 255 | 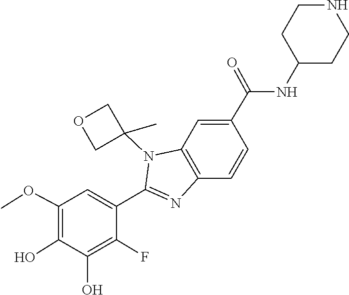 [SMILES: COc1cc(-c2nc3ccc(C(=O)NC4CCNCC4)cc3n2C2(C)COC2)c(F)c(O)c1O] | 2-(2-fluoro-3,4- dihydroxy-5- methoxyphenyl)-1-(3- methyloxetan-3-yl)-N- (piperidin-4-yl)-1H- benzo[d]imidazole-6- carboxamide | 469.1 [M − 1]− |
| 256 | 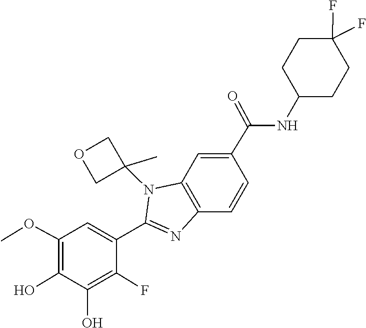 [SMILES: COc1cc(-c2nc3ccc(C(=O)NC4CCC(F)(F)CC4)cc3n2C2(C)COC2)c(F)c(O)c1O] | N-(4,4- difluorocyclohexyl)-2- (2-fluoro-3,4-dihydroxy- 5-methoxyphenyl)-1-(3- methyloxetan-3-yl)-1H- benzo[d]imidazole-6- carboxamide | 506.1 [M + 1]+ |
| 257 | 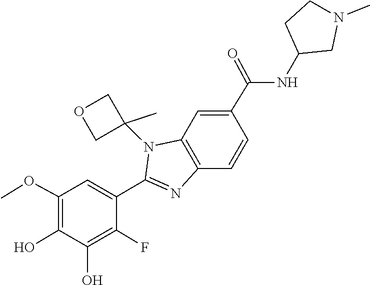 | 2-(2-fluoro-3,4- dihydroxy-5- methoxyphenyl)-1-(3- methyloxetan-3-yl)-N- (1-methylpyrrolidin-3- yl)-1H- benzo[d]imidazole-6- carboxamide | 471.2 [M + 1]+ |
| 258 | 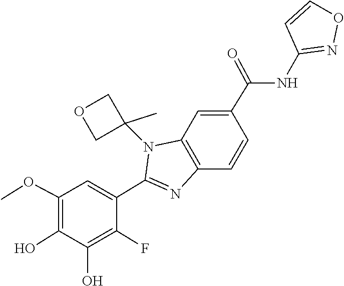 [SMILES: COc1cc(-c2nc3ccc(C(=O)Nc4ccon4)cc3n2C2(C)COC2)c(F)c(O)c1O] | 2-(2-fluoro-3,4- dihydroxy-5- methoxyphenyl)-1-(3- methyloxetan-3-yl)-N- (1,2-oxazol-3-yl)-1H- 1,3-benzodiazole-6- carboxamide | 455.2 [M + 1]+ |
| 259 | 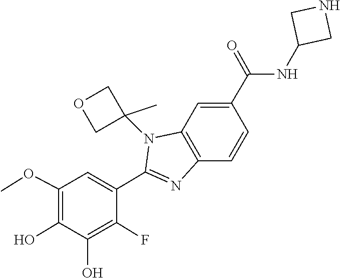 [SMILES: COc1cc(-c2nc3ccc(C(=O)NC4CNC4)cc3n2C2(C)COC2)c(F)c(O)c1O] | N-(azetidin-3-yl)-2-(2- fluoro-3,4-dihydroxy-5- methoxyphenyl)-1-(3- methyloxetan-3-yl)-1H- 1,3-benzodiazole-6- carboxamide | 443.2 [M + 1]+ |
| 260 | 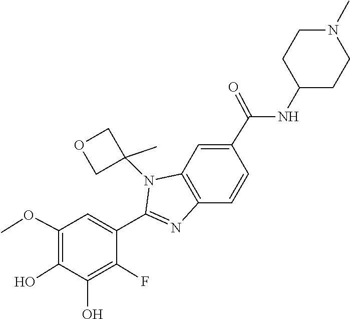 [SMILES: COc1cc(-c2nc3ccc(C(=O)NC4CCN(C)CC4)cc3n2C2(C)COC2)c(F)c(O)c1O] | 2-(2-fluoro-3,4- dihydroxy-5- methoxyphenyl)-1-(3- methyloxetan-3-yl)-N- (1-methylpiperidin-4- yl)-1H-1,3- benzodiazole-6- carboxamide | 483.1 [M − 1]− |
| 261 | 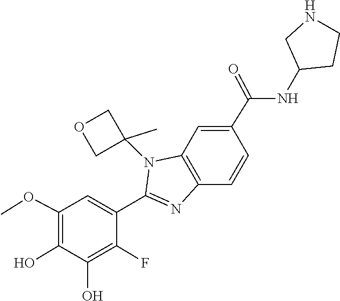 [SMILES: COc1cc(-c2nc3ccc(C(=O)NC4CCNC4)cc3n2C2(C)COC2)c(F)c(O)c1O] | 2-(2-fluoro-3,4- dihydroxy-5- methoxyphenyl)-1-(3- methyloxetan-3-yl)-N- (pyrrolidin-3-yl)-1H- benzo[d]imidazole-6- carboxamide | 455.2 [M − 1]− |
| 262 | 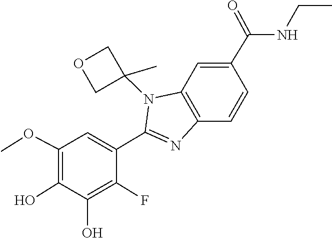 [SMILES: CCNC(=O)c1ccc2nc(-c3cc(OC)c(O)c(O)c3F)n(C3(C)COC3)c2c1] | N-ethyl-2-(2-fluoro-3,4- dihydroxy-5- methoxyphenyl)-1-(3- methyloxetan-3-yl)-1H- benzo[d]imidazole-6- carboxamide | 416.3 [M + 1]+ |
| 263 | 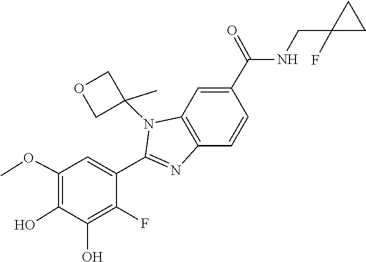 [SMILES: COc1cc(-c2nc3ccc(C(=O)NCC4(F)CC4)cc3n2C2(C)COC2)c(F)c(O)c1O] | 2-(2-fluoro-3,4- dihydroxy-5- methoxyphenyl)-N-[(1- fluorocyclopropyl)methyl]- 1-(3-methyloxetan-3- yl)-1H-1,3- benzodiazole-6- carboxamide. | 460.2 [M + 1]+ |
| 264 | 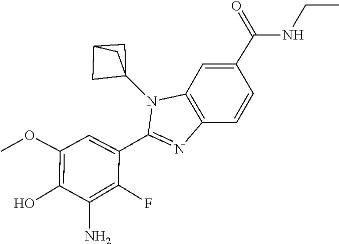 [SMILES: CCNC(=O)c1ccc2nc(-c3cc(OC)c(O)c(N)c3F)n(C34CC(C3)C4)c2c1] | 1-(bicyclo[1.1.1]pentan- 1-yl)-N-ethyl-2-(2- fluoro-3,4-dihydroxy-5- methoxyphenyl)-1H- benzo[d]imidazole-6- carboxamide | 412.2 [M + 1]+ |
| 265 | 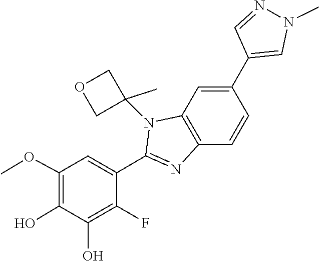 | 3-fluoro-6-methoxy-4- (6-(1-methyl-1H- pyrazol-4-yl)-1-(3- methyloxetan-3-yl)-1H- benzo[d]imidazol-2- yl)benzene-1,2-diol | 425.1 [M + 1]+ |
| 266 | 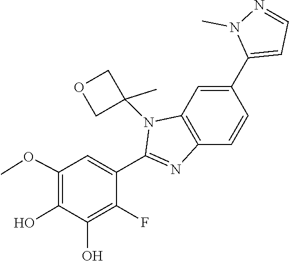 [SMILES: COc1cc(-c2nc3ccc(-c4ccnn4C)cc3n2C2(C)COC2)c(F)c(O)c1O] | 3-fluoro-6-methoxy-4- (6-(1-methyl-1H- pyrazol-5-yl)-1-(3- methyloxetan-3-yl)-1H- benzo[d]imidazol-2- yl)benzene-1,2-diol | 425.2 [M + 1]+ |
| 267 | 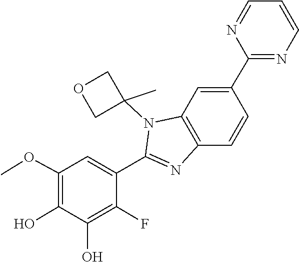 [SMILES: COc1cc(-c2nc3ccc(-c4ncccn4)cc3n2C2(C)COC2)c(F)c(O)c1O] | 3-fluoro-6-methoxy-4- (1-(3-methyloxetan-3- yl)-6-(pyrimidin-2-yl)- 1H-benzo[d]imidazol-2- yl)benzene-1,2-diol | 423.1 [M + 1]+ |
| 268 | 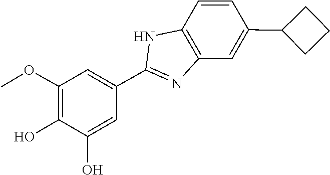 [SMILES: COc1cc(-c2nc3cc(C4CCC4)ccc3[nH]2)cc(O)c1O] | 5-(5-cyclobutyl-1H- benzo[d]imidazol-2-yl)- 3-methoxybenzene-1,2- diol | 311.2 [M + 1]+ |
| 269 | 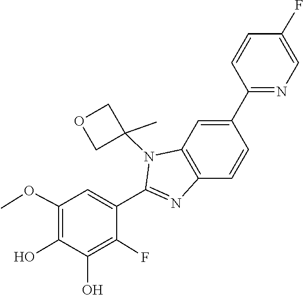 [SMILES: COc1cc(-c2nc3ccc(-c4ccc(F)cn4)cc3n2C2(C)COC2)c(F)c(O)c1O] | 3-fluoro-4-(6-(5- fluoropyridin-2-yl)-1-(3- methyloxetan-3-yl)-1H- benzo[d]imidazol-2-yl)- 6-methoxybenzene-1,2- diol | 440.1 [M + 1]+ |
| 270 | 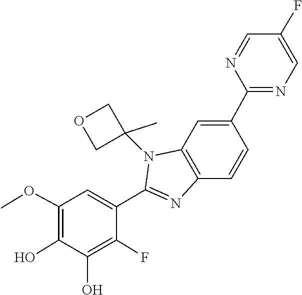 [SMILES: COc1cc(-c2nc3ccc(-c4ncc(F)cn4)cc3n2C2(C)COC2)c(F)c(O)c1O] | 3-fluoro-4-(6-(5- fluoropyrimidin-2-yl)-1- (3-methyloxetan-3-yl)- 1H-benzo[d]imidazol-2- yl)-6-methoxybenzene- 1,2-diol | 441.1 [M + 1]+ |
| 271 | 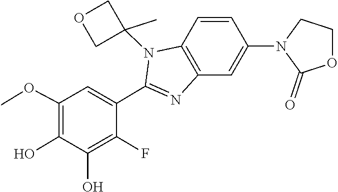 [SMILES: COc1cc(-c2nc3cc(N4CCOC4=O)ccc3n2C2(C)COC2)c(F)c(O)c1O] | 3-(2-(2-fluoro-3,4- dihydroxy-5- methoxyphenyl)-1-(3- methyloxetan-3-yl)-1H- benzo[d]imidazol-5- yl)oxazolidin-2-one | 430.1 [M + 1]+ |
| 272 | 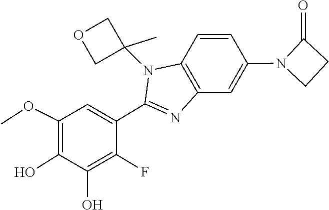 [SMILES: COc1cc(-c2nc3cc(N4CCC4=O)ccc3n2C2(C)COC2)c(F)c(O)c1O] | 1-(2-(2-fluoro-3,4- dihydroxy-5- methoxyphenyl)-1-(3- methyloxetan-3-yl)-1H- benzo[d]imidazol-5- yl)azetidin-2-one | 414.1 [M + 1]+ |
| 273 | 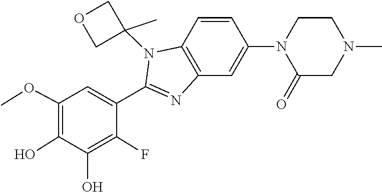 | 1-(2-(2-fluoro-3,4- dihydroxy-5- methoxyphenyl)-1-(3- methyloxetan-3-yl)-1H- benzo[d]imidazol-5-yl)- 4-methylpiperazin-2-one | 457.4 [M + 1]+ |
| 274 | 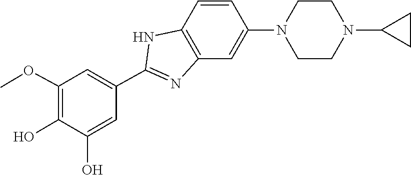 [SMILES: COc1cc(-c2nc3cc(N4CCN(C5CC5)CC4)ccc3[nH]2)cc(O)c1O] | 5-(5-(4- cyclopropylpiperazin-1- yl)-1H- benzo[d]imidazol-2-yl)- 3-methoxybenzene-1,2- diol | 381.3 [M + 1]+ |
| 275 | 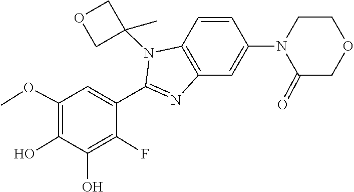 [SMILES: COc1cc(-c2nc3cc(N4CCOCC4=O)ccc3n2C2(C)COC2)c(F)c(O)c1O] | 4-(2-(2-fluoro-3,4- dihydroxy-5- methoxyphenyl)-1-(3- methyloxetan-3-yl)-1H- benzo[d]imidazol-5- yl)morpholin-3-one | 444.2 [M + 1]+ |
| 276 | 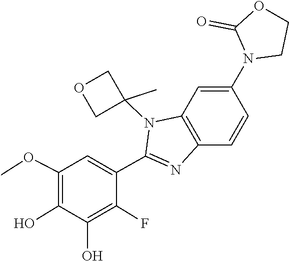 [SMILES: COc1cc(-c2nc3ccc(N4CCOC4=O)cc3n2C2(C)COC2)c(F)c(O)c1O] | 3-(2-(2-fluoro-3,4- dihydroxy-5- methoxyphenyl)-1-(3- methyloxetan-3-yl)-1H- benzo[d]imidazol-6- yl)oxazolidin-2-one | 430.1 [M + 1]+ |
| 277 | 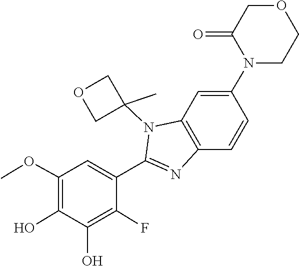 [SMILES: COc1cc(-c2nc3ccc(N4CCOCC4=O)cc3n2C2(C)COC2)c(F)c(O)c1O] | 4-(2-(2-fluoro-3,4- dihydroxy-5- methoxyphenyl)-1-(3- methyloxetan-3-yl)-1H- benzo[d]imidazol-6- yl)morpholin-3-one | 444.2 [M + 1]+ |
| 278 | 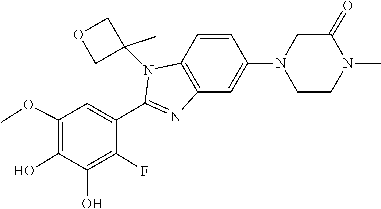 | 4-(2-(2-fluoro-3,4- dihydroxy-5- methoxyphenyl)-1-(3- methyloxetan-3-yl)-1H- benzo[d]imidazol-5-yl)- 1-methylpiperazin-2-one | 457.5 [M + 1]+ |
| 279 | 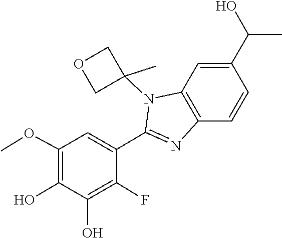 [SMILES: COc1cc(-c2nc3ccc(C(C)O)cc3n2C2(C)COC2)c(F)c(O)c1O] | 3-fluoro-4-(6-(1- hydroxyethyl)-1-(3- methyloxetan-3-yl)-1H- benzo[d]imidazol-2-yl)- 6-methoxybenzene-1,2- diol | 389.0 [M + 1]+ |
| 280 | 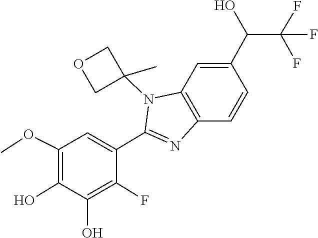 [SMILES: COc1cc(-c2nc3ccc(C(O)C(F)(F)F)cc3n2C2(C)COC2)c(F)c(O)c1O] | 3-fluoro-6-methoxy-4- (1-(3-methyloxetan-3- yl)-6-(2,2,2-trifluoro-1- hydroxyethyl)-1H- benzo[d]imidazol-2- yl)benzene-1,2-diol | 443.3 [M + 1]+ |
| 281 | 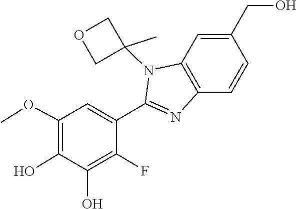 [SMILES: COc1cc(-c2nc3ccc(CO)cc3n2C2(C)COC2)c(F)c(O)c1O] | 3-fluoro-4-(6- (hydroxymethyl)-1-(3- methyloxetan-3-yl)-1H- benzo[d]imidazol-2-yl)- 6-methoxybenzene-1,2- diol | 375.3 [M + 1]+ |
| 282 | 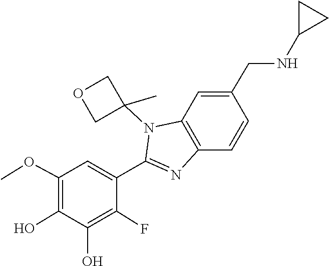 [SMILES: COc1cc(-c2nc3ccc(CNC4CC4)cc3n2C2(C)COC2)c(F)c(O)c1O] | 4-(6- ((cyclopropylamino)methyl)- 1-(3-methyloxetan- 3-yl)-1H- benzo[d]imidazol-2-yl)- 3-fluoro-6- methoxybenzene-1,2- diol | 414.4 [M + 1]+ |
| 283 | 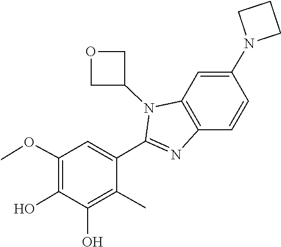 [SMILES: COc1cc(-c2nc3ccc(N4CCC4)cc3n2C2COC2)c(C)c(O)c1O] | 4-(6-(azetidin-1-yl)-1- (oxetan-3-yl)-1H- benzo[d]imidazol-2-yl)- 6-methoxy-3- methylbenzene-1,2-diol | 382.2 [M + 1]+ |
| 284 | 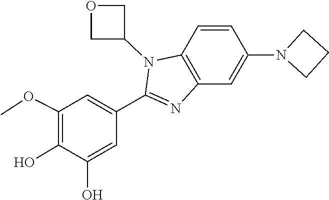 [SMILES: COc1cc(-c2nc3cc(N4CCC4)ccc3n2C2COC2)cc(O)c1O] | 5-(5-(azetidin-1-yl)-1- (oxetan-3-yl)-1H- benzo[d]imidazol-2-yl)- 3-methoxybenzene-1,2- diol | 368.2 [M + 1]+ |
| 285 | 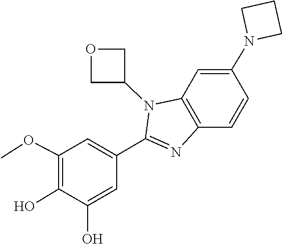 [SMILES: COc1cc(-c2nc3ccc(N4CCC4)cc3n2C2COC2)cc(O)c1O] | 5-(6-(azetidin-1-yl)-1- (oxetan-3-yl)-1H- benzo[d]imidazol-2-yl)- 3-methoxybenzene-1,2- diol | 368.2 [M + 1]+ |
| 286 | 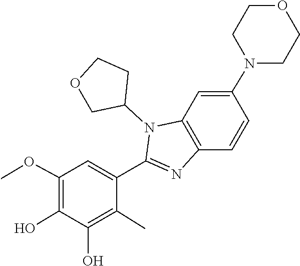 [SMILES: COc1cc(-c2nc3ccc(N4CCOCC4)cc3n2C2CCOC2)c(C)c(O)c1O] | 6-methoxy-3-methyl-4- (6-morpholino-1- (tetrahydrofuran-3-yl)- 1H-benzo[d]imidazol-2- yl)benzene-1,2-diol | 426.3 [M + 1]+ |
| 287 | 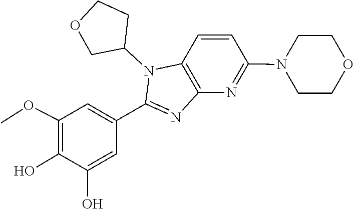 [SMILES: COc1cc(-c2nc3nc(N4CCOCC4)ccc3n2C2CCOC2)cc(O)c1O] | 3-methoxy-5-(5- morpholino-1- (tetrahydrofuran-3-yl)- 1H-imidazo[4,5- b]pyridin-2-yl)benzene- 1,2-diol | 413.3 [M + 1]+ |
| 288 | 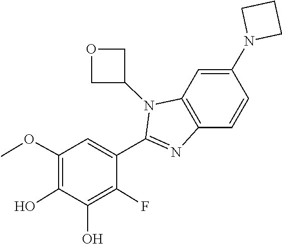 [SMILES: COc1cc(-c2nc3ccc(N4CCC4)cc3n2C2COC2)c(F)c(O)c1O] | 4-(6-(azetidin-1-yl)-1- (oxetan-3-yl)-1H- benzo[d]imidazol-2-yl)- 3-fluoro-6- methoxybenzene-1,2- diol | 386.1 [M + 1]+ |
| 289 | 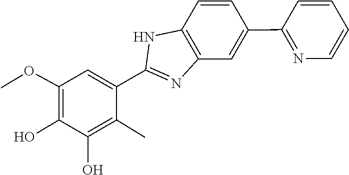 [SMILES: COc1cc(-c2nc3cc(-c4ccccn4)ccc3[nH]2)c(C)c(O)c1O] | 6-methoxy-3-methyl-4- [5-(pyridin-2-yl)-1H-1,3- benzodiazol-2- yl]benzene-1,2-diol | 348.2 [M + 1]+ |
| 290 | 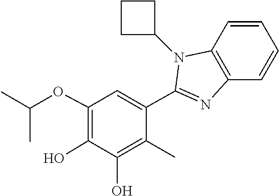 [SMILES: Cc1c(-c2nc3ccccc3n2C2CCC2)cc(OC(C)C)c(O)c1O] | 4-(1-cyclobutyl-1H-1,3- benzodiazol-2-yl)-3- methyl-6-(propan-2- yloxy)benzene-1,2-diol | 353.0 [M + 1]+ |
| 291 | 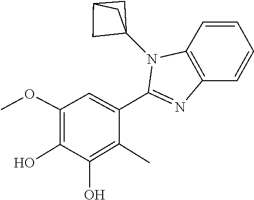 [SMILES: COc1cc(-c2nc3ccccc3n2C23CC(C2)C3)c(C)c(O)c1O] | 4-(1- (bicyclo[1.1.1]pentan-1- yl)-1H- benzo[d]imidazol-2-yl)- 6-methoxy-3- methylbenzene-1,2-diol | 337.4 [M + 1]+ |
| 292 | 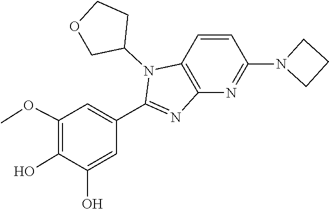 [SMILES: COc1cc(-c2nc3nc(N4CCC4)ccc3n2C2CCOC2)cc(O)c1O] | 5-(5-(azetidin-1-yl)-1- (tetrahydrofuran-3-yl)- 1H-imidazo[4,5- b]pyridin-2-yl)-3- methoxybenzene-1,2- diol | 383.3 [M + 1]+ |
| 293 | 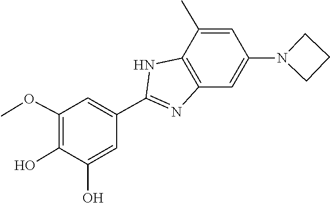 [SMILES: COc1cc(-c2nc3cc(N4CCC4)cc(C)c3[nH]2)cc(O)c1O] | 5-(5-(azetidin-1-yl)-7- methyl-1H- benzo[d]imidazol-2-yl)- 3-methoxybenzene-1,2- diol | 326.2 [M + 1]+ |
| 294 | 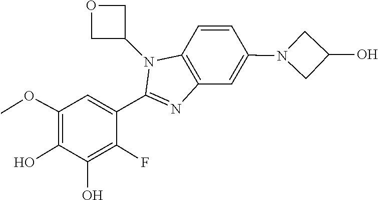 [SMILES: COc1cc(-c2nc3cc(N4CC(O)C4)ccc3n2C2COC2)c(F)c(O)c1O] | 3-fluoro-4-(5-(3- hydroxyazetidin-1-yl)-1- (oxetan-3-yl)-1H- benzo[d]imidazol-2-yl)- 6-methoxybenzene-1,2- diol | 402.1 [M + 1]+ |
| 295 | 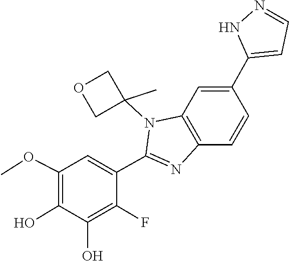 [SMILES: COc1cc(-c2nc3ccc(-c4ccn[nH]4)cc3n2C2(C)COC2)c(F)c(O)c1O] | 3-fluoro-6-methoxy-4- (1-(3-methyloxetan-3- yl)-6-(1H-pyrazol-5-yl)- 1H-benzo[d]imidazol-2- yl)benzene-1,2-diol | 411.2 [M + 1]+ |
| 296 | 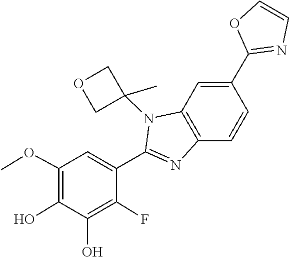 [SMILES: COc1cc(-c2nc3ccc(-c4ncco4)cc3n2C2(C)COC2)c(F)c(O)c1O] | 3-fluoro-6-methoxy-4- (1-(3-methyloxetan-3- yl)-6-(oxazol-2-yl)-1H- benzo[d]imidazol-2- yl)benzene-1,2-diol | 412.1 [M + 1]+ |
| 297 | 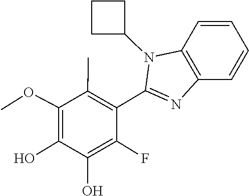 [SMILES: COc1c(C)c(-c2nc3ccccc3n2C2CCC2)c(F)c(O)c1O] | 4-(1-cyclobutyl-1H- benzo[d]imidazol-2-yl)- 3-fluoro-6-methoxy-5- methylbenzene-1,2-diol | 343.2 [M + 1]+ |
| 298 | 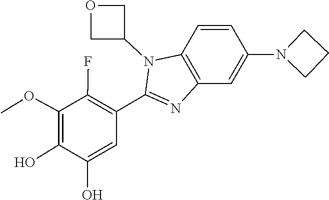 [SMILES: COc1c(O)c(O)cc(-c2nc3cc(N4CCC4)ccc3n2C2COC2)c1F] | 5-(5-(azetidin-1-yl)-1- (oxetan-3-yl)-1H- benzo[d]imidazol-2-yl)- 4-fluoro-3- methoxybenzene-1,2- diol | 386.2 [M + 1]+ |
| 299 | 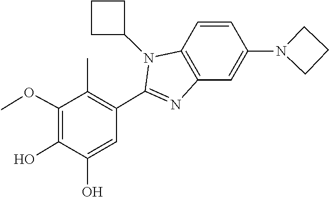 [SMILES: COc1c(C)c(-c2nc3cc(N4CCC4)ccc3n2C2CCC2)cc(O)c1O] | 5-(5-(azetidin-1-yl)-1- cyclobutyl-1H- benzo[d]imidazol-2-yl)- 3-methoxy-4- methylbenzene-1,2-diol | 380.2 [M + 1]+ |
| 300 | 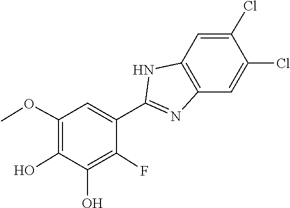 [SMILES: COc1cc(-c2nc3cc(Cl)c(Cl)cc3[nH]2)c(F)c(O)c1O] | 4-(5,6-dichloro-1H- benzo[d]imidazol-2-yl)- 3-fluoro-6- methoxybenzene-1,2- diol | 343.0 [M + 1]+ |
| 301 | 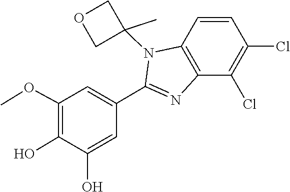 [SMILES: COc1cc(-c2nc3c(Cl)c(Cl)ccc3n2C2(C)COC2)cc(O)c1O] | 5-(4,5-dichloro-1-(3- methyloxetan-3-yl)-1H- benzo[d]imidazol-2-yl)- 3-methoxybenzene-1,2- diol | 394.9 [M + 1]+ |
| 302 | 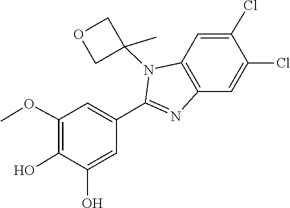 [SMILES: COc1cc(-c2nc3cc(Cl)c(Cl)cc3n2C2(C)COC2)cc(O)c1O] | 5-(5,6-dichloro-1-(3- methyloxetan-3-yl)-1H- benzo[d]imidazol-2-yl)- 3-methoxybenzene-1,2- diol | 394.9 [M + 1]+ |
| 303 | 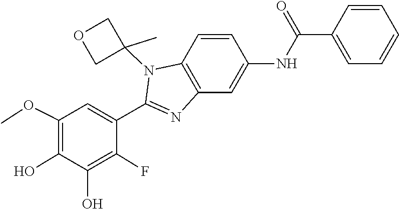 [SMILES: COc1cc(-c2nc3cc(NC(=O)c4ccccc4)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5- yl)benzamide | 464.3 [M + 1]+ |
| 304 | 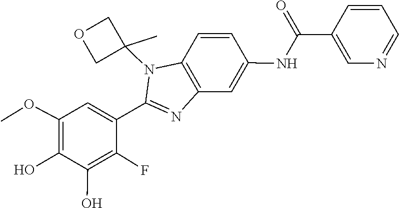 [SMILES: COc1cc(-c2nc3cc(NC(=O)c4cccnc4)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5- yl)nicotinamide | 465.3 [M + 1]+ |
| 305 | 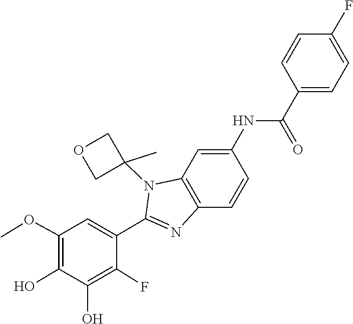 [SMILES: COc1cc(-c2nc3ccc(NC(=O)c4ccc(F)cc4)cc3n2C2(C)COC2)c(F)c(O)c1O] | 4-fluoro-N-(2-(2-fluoro- 3,4-dihydroxy-5- methoxyphenyl)-1-(3- methyloxetan-3-yl)-1H- benzo[d]imidazol-6- yl)benzamide | 482.3 [M + 1]+ |
| 306 | 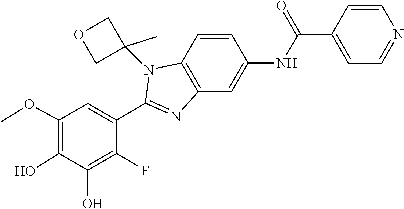 [SMILES: COc1cc(-c2nc3cc(NC(=O)c4ccncc4)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5- yl)isonicotinamide | 465.2 [M + 1]+ |
| 307 | 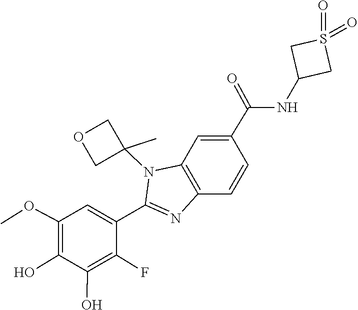 [SMILES: COc1cc(-c2nc3ccc(C(=O)NC4CS(=O)(=O)C4)cc3n2C2(C)COC2)c(F)c(O)c1O] | N-(1,1-dioxidothietan-3- yl)-2-(2-fluoro-3,4- dihydroxy-5- methoxyphenyl)-1-(3- methyloxetan-3-yl)-1H- benzo[d]imidazole-6- carboxamide | 492.3 [M + 1]+ |
| 308 | 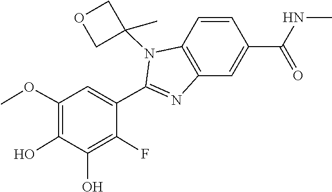 | 2-(2-fluoro-3,4- dihydroxy-5- methoxyphenyl)-N- methyl-1-(3- methyloxetan-3-yl)-1H- benzo[d]imidazole-5- carboxamide | 402.2 [M + 1]+ |
| 309 | 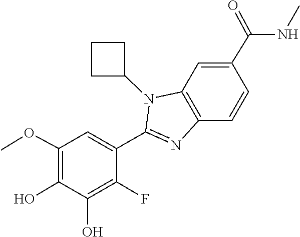 | 1-cyclobutyl-2-(2- fluoro-3,4-dihydroxy-5- methoxyphenyl)-N- methyl-1H- benzo[d]imidazole-6- carboxamide | 386.3 [M + 1]+ |
| 310 | 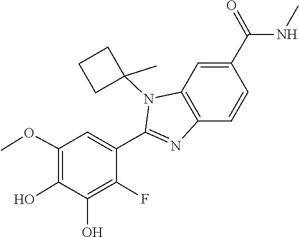 | 2-(2-fluoro-3,4- dihydroxy-5- methoxyphenyl)-N- methyl-1-(1- methylcyclobutyl)-1H- benzo[d]imidazole-6- carboxamide | 400.2 [M + 1]+ |
| 311 | 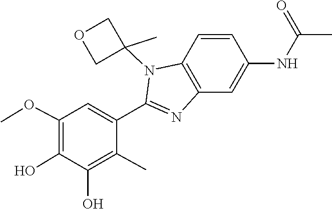 [SMILES: COc1cc(-c2nc3cc(NC(C)=O)ccc3n2C2(C)COC2)c(C)c(O)c1O] | N-(2-(3,4-dihydroxy-5- methoxy-2- methylphenyl)-1-(3- methyloxetan-3-yl)-1H- benzo[d]imidazol-5- yl)acetamide | 398.0 [M + 1]+ |
| 312 | 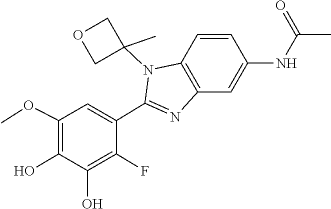 [SMILES: COc1cc(-c2nc3cc(NC(C)=O)ccc3n2C2(C)COC2)c(F)c(O)c1O] | N-(2-(2-fluoro-3,4- dihydroxy-5- methoxyphenyl)-1-(3- methyloxetan-3-yl)-1H- benzo[d]imidazol-5- yl)acetamide | 402.0 [M + 1]+ |
| 313 | 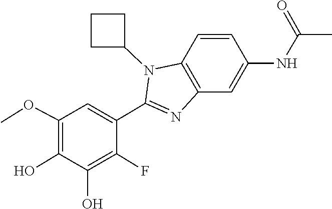 [SMILES: COc1cc(-c2nc3cc(NC(C)=O)ccc3n2C2CCC2)c(F)c(O)c1O] | N-(1-cyclobutyl-2-(2- fluoro-3,4-dihydroxy-5- methoxyphenyl)-1H- benzo[d]imidazol-5- yl)acetamide | 386.0 [M + 1]+ |
| 314 | 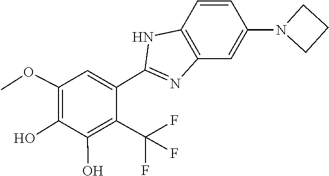 [SMILES: COc1cc(-c2nc3cc(N4CCC4)ccc3[nH]2)c(C(F)(F)F)c(O)c1O] | 4-(5-(azetidin-1-yl)-1H- benzo[d]imidazol-2-yl)- 6-methoxy-3- (trifluoromethyl)benzene- 1,2-diol | 380.1 [M + 1]+ |
| 314 | 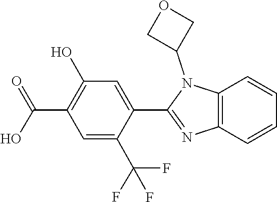 [SMILES: O=C(O)c1cc(C(F)(F)F)c(-c2nc3ccccc3n2C2COC2)cc1O] | 2-hydroxy-4-(1-(oxetan- 3-yl)-1H- benzo[d]imidazol-2-yl)- 5- (trifluoromethyl)benzoic acid | 379.16 [M + H]+ |
| 315 | 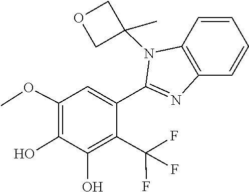 [SMILES: COc1cc(-c2nc3ccccc3n2C2(C)COC2)c(C(F)(F)F)c(O)c1O] | 6-methoxy-4-(1-(3- methyloxetan-3-yl)-1H- benzo[d]imidazol-2-yl)- 3- (trifluoromethyl)benzene- 1,2-diol | 395.3 [M + 1]+ |
| 316 | 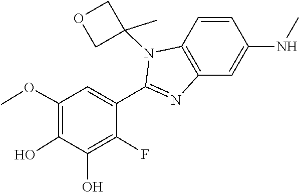 | 3-fluoro-6-methoxy-4- (5-(methylamino)-1-(3- methyloxetan-3-yl)-1H- benzo[d]imidazol-2- yl)benzene-1,2-diol | 374.2 [M + 1]+ |
| 317 | 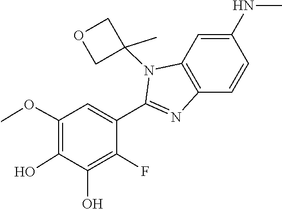 | 3-fluoro-6-methoxy-4- (6-(methylamino)-1-(3- methyloxetan-3-yl)-1H- benzo[d]imidazol-2- yl)benzene-1,2-diol | 374.3 [M + 1]+ |
| 318 | 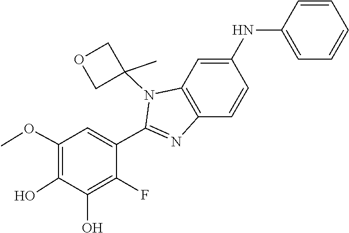 [SMILES: COc1cc(-c2nc3ccc(Nc4ccccc4)cc3n2C2(C)COC2)c(F)c(O)c1O] | 3-fluoro-6-methoxy-4- (1-(3-methyloxetan-3- yl)-6-(phenylamino)-1H- benzo[d]imidazol-2- yl)benzene-1,2-diol | 436.3 [M + 1]+ |
| 319 | 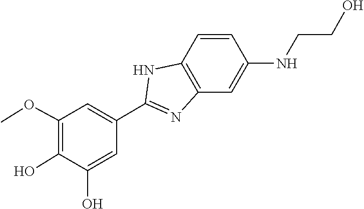 [SMILES: COc1cc(-c2nc3cc(NCCO)ccc3[nH]2)cc(O)c1O] | 5-(5-((2- hydroxyethyl)amino)- 1H-benzo[d]imidazol-2- yl)-3-methoxybenzene- 1,2-diol | 316.1 [M + 1]+ |
| 320 | 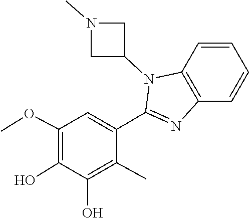 [SMILES: COc1cc(-c2nc3ccccc3n2C2CN(C)C2)c(C)c(O)c1O] | 6-methoxy-3-methyl-4- (1-(1-methylazetidin-3- yl)-1H- benzo[d]imidazol-2- yl)benzene-1,2-diol | 340.3 [M + 1]+ |
| 321 | 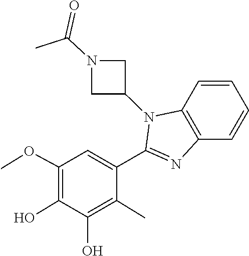 [SMILES: COc1cc(-c2nc3ccccc3n2C2CN(C(C)=O)C2)c(C)c(O)c1O] | 1-(3-(2-(3,4-dihydroxy- 5-methoxy-2- methylphenyl)-1H- benzo[d]imidazol-1- yl)azetidin-1-yl)ethan-1- one | 368.3 [M + 1]+ |
| 322 | 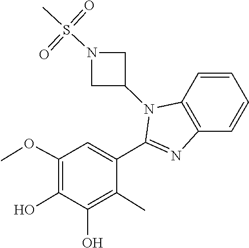 [SMILES: COc1cc(-c2nc3ccccc3n2C2CN(S(C)(=O)=O)C2)c(C)c(O)c1O] | 6-methoxy-3-methyl-4- (methylsulfonyl)azetidin- 3-yl)-1H- benzo[d]imidazol-2- yl)benzene-1,2-diol | 404.2 [M + 1]+ |
| 323 | 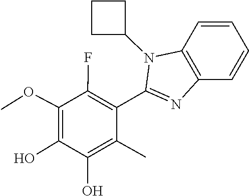 [SMILES: COc1c(O)c(O)c(C)c(-c2nc3ccccc3n2C2CCC2)c1F] | 4-(1-cyclobutyl-1H- benzo[d]imidazol-2-yl)- 5-fluoro-6-methoxy-3- methylbenzene-1,2-diol | 343.1 [M + 1]+ |
| 324 | 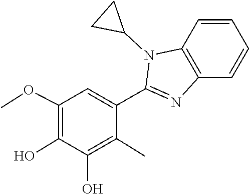 [SMILES: COc1cc(-c2nc3ccccc3n2C2CC2)c(C)c(O)c1O] | 4-(1-cyclopropyl-1H- benzo[d]imidazol-2-yl)- 6-methoxy-3- methylbenzene-1,2-diol | 311.1 [M + 1]+ |
| 325 | 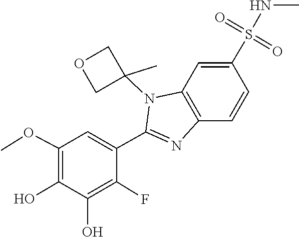 | 2-(2-fluoro-3,4- dihydroxy-5- methoxyphenyl)-N- methyl-1-(3- methyloxetan-3-yl)-1H- benzo[d]imidazole-6- sulfonamide | 438.1 [M + 1]+ |
| 326 | 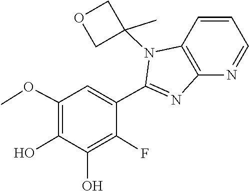 [SMILES: COc1cc(-c2nc3ncccc3n2C2(C)COC2)c(F)c(O)c1O] | 3-fluoro-6-methoxy-4- (1-(3-methyloxetan-3- yl)-1H-imidazo[4,5- b]pyridin-2-yl)benzene- 1,2-diol | 346.1 [M + 1]+ |
| 327 | 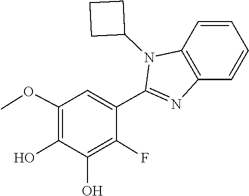 [SMILES: COc1cc(-c2nc3ccccc3n2C2CCC2)c(F)c(O)c1O] | 4-(1-cyclobutyl-1H- benzo[d]imidazol-2-yl)- 3-fluoro-6- methoxybenzene-1,2- diol | 329.1 [M + 1]+ |
| 328 | 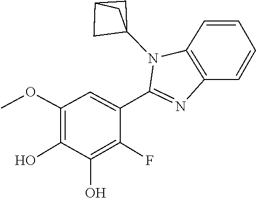 [SMILES: COc1cc(-c2nc3ccccc3n2C23CC(C2)C3)c(F)c(O)c1O] | 4-(1- (bicyclo[1.1.1]pentan-1- yl)-1H- benzo[d]imidazol-2-yl)- 3-fluoro-6- methoxybenzene-1,2- diol | 341.3 [M + 1]+ |
| 329 | 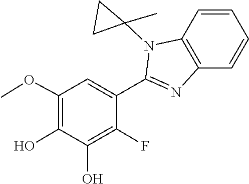 [SMILES: COc1cc(-c2nc3ccccc3n2C2(C)CC2)c(F)c(O)c1O] | 3-fluoro-6-methoxy-4- (1-(1- methylcyclopropyl)-1H- benzo[d]imidazol-2- yl)benzene-1,2-diol | 329.2 [M + 1]+ |
| 330 | 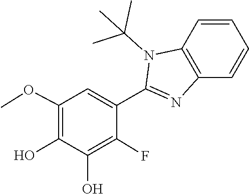 [SMILES: COc1cc(-c2nc3ccccc3n2C(C)(C)C)c(F)c(O)c1O] | 4-(1-(tert-butyl)-1H- benzo[d]imidazol-2-yl)- 3-fluoro-6- methoxybenzene-1,2- diol | 331.2 [M + 1]+ |
| 331 | 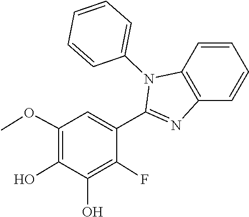 [SMILES: COc1cc(-c2nc3ccccc3n2-c2ccccc2)c(F)c(O)c1O] | 3-fluoro-6-methoxy-4- (l-phenyl-1H-1,3- benzodiazol-2- yl)benzene-1,2-diol | 351.1 [M + 1]+ |
| 332 | 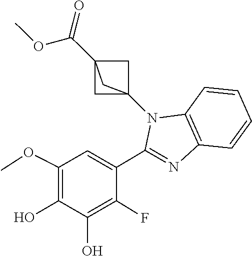 [SMILES: COC(=O)C12CC(n3c(-c4cc(OC)c(O)c(O)c4F)nc4ccccc43)(C1)C2] | methyl 3-(2-(2-fluoro- 3,4-dihydroxy-5- methoxyphenyl)-1H- benzo[d]imidazol-1- yl)bicyclo[1.1.1]pentane- 1-carboxylate | 399.4 [M + 1]+ |
| 333 | 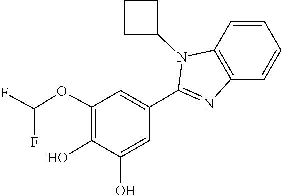 [SMILES: Oc1cc(-c2nc3ccccc3n2C2CCC2)cc(OC(F)F)c1O] | 5-(1-cyclobutyl-1H- benzo[d]imidazol-2-yl)- 3- (difluoromethoxy)benzene- 1,2-diol | 347.0 [M + 1]+ |
| 334 | 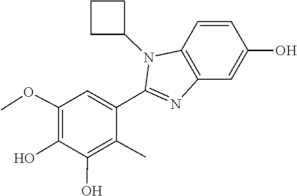 [SMILES: COc1cc(-c2nc3cc(O)ccc3n2C2CCC2)c(C)c(O)c1O] | 4-(1-cyclobutyl-5- hydroxy-1H- benzo[d]imidazol-2-yl)- 6-methoxy-3- methylbenzene-1,2-diol | 341.2 [M + 1]+ |
| 335 | 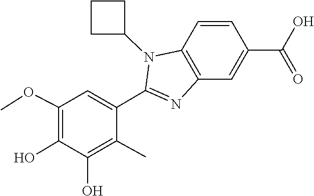 [SMILES: COc1cc(-c2nc3cc(C(=O)O)ccc3n2C2CCC2)c(C)c(O)c1O] | 1-cyclobutyl-2-(3,4- dihydroxy-5-methoxy-2- methylphenyl)-1H- benzo[d]imidazole-6- carboxylic acid | 369.2 [M + 1]+ |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C ¹⁴C¹⁵N, ¹⁸O, ¹⁷O, ³¹P ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, tautomer, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

In some embodiments, the unsubstituted benzimidazole in the compounds described herein exists as a tautomer as seen below:

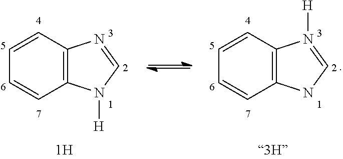

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, the compounds disclosed herein are prepared according to the procedure shown in Scheme 1.

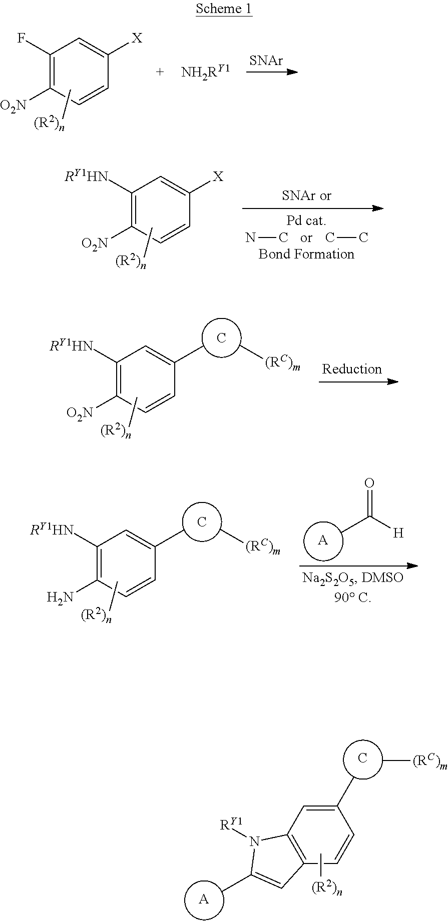

In some embodiments, the first step comprises a nucleophilic aromatic substitution (SNAr) reaction between an aryl halide and a nucleophilic amine. In some embodiments, the aryl halide is an aryl fluoride. In some embodiments, the SNAr reaction is accomplished with a base. In some embodiments, the base is triethylamine, potassium carbonate, or N,N-diisopropylethylamine. In some embodiments, the SNAr reaction is performed in a solvent with a high boiling point. In some embodiments, the solvent is isopropyl acetate, dimethylformamide, N-methylpyrrolidone, or 1,4-dioxane. In some embodiments, the SNAr reaction is accomplished with heating.

In some embodiments, the second step comprises a nucleophilic aromatic substitution (SNAr) reaction between an aryl halide and a nucleophilic amine. In some embodiments, the second step comprises a palladium-catalyzed cross-coupling reaction. In some embodiments, the second step comprises a palladium-catalyzed N—C bond-forming reaction. In some embodiments, the second step comprises a palladium-catalyzed C—C bond-forming reaction. In some embodiments, the second step comprises a Heck coupling. In some embodiments, the second step comprises a Sonogashira coupling. In some embodiments, the second step comprises a Negishi coupling. In some embodiments, the second step comprises a Stille coupling. In some embodiments, the second step comprises a Suzuki coupling. In some embodiments, the second step comprises a Stille coupling. In some embodiments, the second step comprises an Ullmann coupling. In some embodiments, the second step comprises a Stille coupling. In some embodiments, the second step comprises a Chan-Lam coupling. In some embodiments, the second step comprises a Stille coupling. In some embodiments, the second step comprises a Buchwald-Hartwig coupling. In some embodiments, the second step is optional.

In some embodiments, the third step is a reduction. In some embodiments, the reduction is a nitro to amine reduction. In some embodiments, the reduction is accomplished with gaseous hydrogen in the presence of a catalyst. In some embodiments, the catalyst is palladium on carbon. In some embodiments, the catalyst is palladium hydroxide. In some embodiments, the reduction is performed in methanol.

In some embodiments, the fourth step is a condensation reaction between a diamine and an aldehyde to provide a benzimidazole. In some embodiments, the condensation is accomplished with sodium metabisulfite. In some embodiments, the condensation is performed in dimethylsulfoxide. In some embodiments, the condensation is accomplished with heating.

In some embodiments, the compounds disclosed herein are prepared according to the procedure shown in Scheme 2.

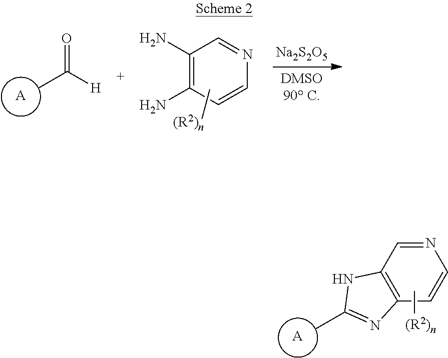

In some embodiments, the reaction is a condensation reaction between an aldehyde and a diamino pyridine to provide an imidazolopyridine. In some embodiments, the condensation is accomplished with sodium metabisulfite. In some embodiments, the condensation is performed in dimethylsulfoxide. In some embodiments, the condensation is accomplished with heating.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising a compound described herein, or a pharmaceutically acceptable salt, solvate, tautomer, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached.

### Methods of Treatment

The compounds disclosed herein, or pharmaceutically acceptable salts, solvates, or stereoisomers thereof, are useful for the inhibition of TREX1.

Provided herein are compounds that are inhibitors of TREX1 and are therefore useful for treating one or more disorders associated with the activity of TREX1 or mutants thereof.

Provided herein is a method of treating cancer in a subject in need thereof, the method comprising administering a compound disclosed herein, or a pharmaceutically acceptable salt, solvate, tautomer, or stereoisomer thereof. In some embodiments, the cancer is selected from non-Hodgkin lymphoma, Hodgkin lymphoma, squamous cell carcinoma, cancer of the head and neck, cholangiocarcinoma, hepatocellular carcinoma, bladder cancer, sarcoma, colon cancer, gastric cancer, thyroid cancer, lung cancer, leukemia, pancreatic cancer, melanoma, multiple myeloma, brain cancer, CNS cancer, renal cancer, prostate cancer, ovarian cancer, and breast cancer.

In some embodiments, the cancer is a solid tumor malignancy. In some embodiments, the solid tumor malignancy is bone cancer (for example, but not limited to, chondrosarcoma, Ewing's sarcoma, malignant fibrous histiocytoma of bone/osteosarcoma, osteosarcoma, or rhabdomyosarcoma), heart cancer, brain and nervous system cancer (for example, but not limited to, astrocytoma, brainstem glioma, pilocytic astrocytoma, ependymoma, primitive neuroectodermal tumor, cerebellar astrocytoma, cerebral astrocytoma, glioma, medulloblastoma, glioblastoma, neuroblastoma, oligodendroglioma, pineal astrocytoma, pituitary adenoma, or visual pathway and hypothalamic glioma), breast cancer (for example, but not limited to, invasive lobular carcinoma, tubular carcinoma, invasive cribriform carcinoma, medullary carcinoma, male breast cancer, phyllodes tumor, or inflammatory breast cancer), endocrine system cancer (for example, but not limited to, adrenocortical carcinoma, islet cell carcinoma (endocrine pancreas), multiple endocrine neoplasia syndrome, parathyroid cancer, pheochromocytoma, or thyroid cancer), eye cancer (for example, but not limited to, uveal melanoma or retinoblastoma), gastrointestinal cancer (for example, but not limited to, anal cancer, appendix cancer, cholangiocarcinoma, gastrointestinal carcinoid tumor, colon cancer, extrahepatic bile duct cancer, gallbladder cancer, gastric (stomach) cancer, gastrointestinal carcinoid tumor, gastrointestinal stromal tumor (gist), hepatocellular cancer, pancreatic cancer, or rectal cancer), genitourinary and gynecologic cancer (for example, but not limited to, bladder cancer, cervical cancer, endometrial cancer, extragonadal germ cell tumor, ovarian cancer, ovarian epithelial cancer (surface epithelial-stromal tumor), ovarian germ cell tumor, penile cancer, renal cell carcinoma, renal pelvis and ureter, transitional cell cancer, prostate cancer, testicular cancer, gestational trophoblastic tumor, ureter and renal pelvis, transitional cell cancer, urethral cancer, uterus cancer, uterine sarcoma, vaginal cancer, vulvar cancer, or Wilms tumor), head and neck cancer (for example, but not limited to, is esophageal cancer, nasopharyngeal carcinoma, oral cancer, oropharyngeal cancer, paranasal sinus and nasal cavity cancer, pharyngeal cancer, salivary gland cancer, or hypopharyngeal cancer), skin cancer (for example, but not limited to, basal cell carcinoma, squamous cell carcinoma, skin adnexal tumors (e.g. sebaceous carcinoma), melanoma, Merkel cell carcinoma, or sarcomas of primary cutaneous origin (e.g. dermatofibrosarcoma protuberans)), or thoracic and respiratory cancer (bronchial adenomas/carcinoid, small cell lung cancer, mesothelioma, non-small cell lung cancer, pleuropulmonary blastoma, laryngeal cancer, or thymoma and thymic carcinoma).

TREX1 is a component of the cellular DNA repair mechanism. Treatment of patients with cancers having DNA repair deficiencies (such as BRCA1 mutations) with DNA repair inhibitors (such as poly ADP ribose polymerase (“PARP”) inhibitors) is synergistic due to the dramatically reduced probability of developing resistance against two DNA repair insults; this approach is known as synthetic lethality. Hence, TREX1 inhibitors also possess potential utility as effective synthetic lethality partners in patients with cancers characterized by defective DNA repair.

In some embodiments, the DNA repair deficiency is a deficiency in the base excision repair (“BER”) pathway (such as a PolB mutation). In some embodiments, the DNA repair deficiency is a deficiency in the Fanconi anaemia-mediated repair (“FA”) pathway (such as an FANCA mutation). In some embodiments, the DNA repair deficiency is a deficiency in the homologous recombination (“HR”) pathway (such as a BRCA1 mutation). In some embodiments, the DNA repair deficiency is a deficiency in the nucleotide excision repair (“NER”) pathway (such as an XPA mutation). In some embodiments, the DNA repair deficiency is a deficiency in the non-homologous end joining (“NHEJ”) pathway (such as an MRE11 mutation). In some embodiments, the DNA repair deficiency is a deficiency in the mismatch repair (“MMR”) pathway (such as an hMSH2 mutation). In some embodiments, the DNA repair deficiency is a deficiency in the RecQ-mediated repair (“RecQ”) pathway (such as a BLM mutation). In some embodiments, the DNA repair deficiency is a deficiency in the double-stranded breaks (“DSB”) pathway (such as a POLQ mutation).

In some embodiments, the cancer is characterized by a deficiency in one or more DNA repair pathways. In some embodiments, the DNA repair deficiency is a deficiency in the base excision repair (“BER”) pathway, the Fanconi anaemia-mediated repair (“FA”) pathway, the homologous recombination (“HR”) pathway, the nucleotide excision repair (“NER”) pathway, the non-homologous end joining (“NHEJ”) pathway, the mismatch repair (“MMR”) pathway, the RecQ-mediated repair (“RecQ”) pathway, or the double-stranded breaks (“DSB”) pathway. In some embodiments, the DNA repair deficiency is a deficiency in the homologous recombination (“HR”) pathway. In some embodiments, the DNA repair deficiency is a BRCA1 mutation.

Provided herein is a method of treating cancer in a subject in need thereof, the method comprising administering a reversible, non-competitive TREX1 inhibitor. In some embodiments, the cancer is a solid tumor malignancy. In some embodiments, the cancer is selected from non-Hodgkin lymphoma, Hodgkin lymphoma, squamous cell carcinoma, cancer of the head and neck, cholangiocarcinoma, hepatocellular carcinoma, bladder cancer, sarcoma, colon cancer, gastric cancer, thyroid cancer, lung cancer, leukemia, pancreatic cancer, melanoma, multiple myeloma, brain cancer, CNS cancer, renal cancer, prostate cancer, ovarian cancer, and breast cancer.

Provided herein is a method of increasing type I interferon production in a subject in need thereof, the method comprising administering a reversible, non-competitive TREX1 inhibitor. In some embodiments, the increase in type I interferon production occurs in the tumor microenvironment. In some embodiments, the TREX1 inhibitor is administered systemically. In some embodiments, the TREX1 inhibitor comprises a compound disclosed herein, or a pharmaceutically acceptable salt, solvate, tautomer, or stereoisomer thereof.

### Combination Therapy

In certain instances, the compound described herein, or a pharmaceutically acceptable salt, solvate, tautomer, or stereoisomer thereof, is administered in combination with a second therapeutic agent.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, tautomer, or stereoisomer thereof, is co-administered with a second therapeutic agent, wherein the compound described herein, or a pharmaceutically acceptable salt, solvate, tautomer, or stereoisomer thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically effective dosages of the compounds disclosed herein will be utilized in formulating a pharmaceutical composition and/or in treatment regimens when the compounds disclosed herein are administered in combination with a second therapeutic agent. Therapeutically effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, tautomer, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, tautomer, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, tautomer, or stereoisomer thereof, as well as combination therapies, are administered before, during, or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, the compound described herein, or a pharmaceutically acceptable salt, solvate, tautomer, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

In some embodiments, the compound described herein, or a pharmaceutically acceptable salt, solvate, tautomer, or stereoisomer thereof, is administered in combination with a DNA repair inhibitor. In some embodiments, the DNA repair inhibitor is a poly ADP ribose polymerase (“PARP”) inhibitor. In some embodiments, the PARP inhibitor is olaparib, rucaparib, niraparib, talazoparib, veliparib, pamiparib, CEP 9722, or E7016.

In some embodiments, the compound described herein, or a pharmaceutically acceptable salt, solvate, tautomer, or stereoisomer thereof, is administered in combination with an alkylating agent. In some embodiments, the alkylating agent is cyclophosphamide, chlormethine, uramustine, melphalan, chlorambucil, ifosfamide, bendamustine, carmustine, lomustine, nimustine, fotemustine, streptozocin, or busulfan.

In some embodiments, the compound described herein, or a pharmaceutically acceptable salt, solvate, tautomer, or stereoisomer thereof, is administered in conjunction with high-dose radiotherapy administered as a single dose and/or hypofractionated.

In some embodiments, the compound described herein, or a pharmaceutically acceptable salt, solvate, tautomer, or stereoisomer thereof, is administered in conjunction with Stereotactic Body Radiation Therapy (SBRT).

### EXAMPLES

### Example 1: Synthesis of 6-methoxy-3-methyl-4-(1-(oxetan-3-yl)-1H-benzo[d]imidazol-2-yl)benzene-1,2-diol

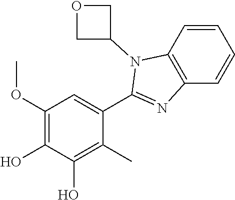 [SMILES: COc1cc(-c2nc3ccccc3n2C2COC2)c(C)c(O)c1O]

Step 1: A solution of 1-fluoro-2-nitrobenzene (1.00 g, 1.0 eq., 7.09 mmol), triethylamine (1.5 mL, 10.6 mmol) and oxetan-3-amine (514 mg, 7.04 mmol) in isopropanol (10 mL) was stirred at 90° C. for 1 hour. After cooling to room temperature, precipitated solids were filtered and dried to afford N-(2-nitrophenyl)oxetan-3-amine as a yellow solid.

Step 2: A mixture of 3-nitro-N-(oxetan-3-yl)pyridin-2-amine (700 mg, 3.59 mmol) and palladium on carbon (10% w/w, 450 mg, 3.59 mmol) in methanol (5.00 mL) was stirred under hydrogen gas (1 atm) for 16 hours. The reaction mass was filtered and the filtrate was concentrated under reduced pressure to afford N1-(oxetan-3-yl)benzene-1,2-diamine as a dark brown liquid which was immediately used for the next step without purification.

Step 3: To a stirred solution of 3,4-dihydroxy-5-methoxybenzaldehyde (5.00 g, 29.7 mmol) in acetic acid (50.0 mL) was added bromine (1.58 mL, 29.7 mmol) at 0° C. and the resulting mixture stirred at room temperature overnight. The reaction mixture was poured into chilled water; the precipitated solids were filtered and dried to afford 2-bromo-3,4-dihydroxy-5-methoxybenzaldehyde as off white solid.

Step 4: To a stirred solution of 2-bromo-3,4-dihydroxy-5-methoxybenzaldehyde (4.00 g, 16.2 mmol) in N,N-dimethylformamide (20.0 mL) was added sodium hydride (744 mg, 32.4 mmol) portion wise at 0° C.; after 15 min, benzyl bromide (4.95 mL, 40.5 mmol) was added dropwise at 0° C. and stirred the reaction mixture at room temperature for 3 hours. The reaction mass was quenched with chilled water (50 mL) and extracted with diethyl ether. The organic layer was washed with water, dried over sodium sulfate and concentrated. The crude compound was purified by silica gel chromatography with 10% ethyl acetate in n-hexanes as eluent to afford 3,4-bis(benzyloxy)-2-bromo-5-methoxybenzaldehyde as white solid.

Step 5: A mixture of 3,4-bis(benzyloxy)-2-bromo-5-methoxybenzaldehyde (500 mg, 1.17 mmol) and dipotassium carbonate (485 mg, 3 eq., 3.51 mmol) in methanol (5 mL) was degassed with argon for 10 minutes followed by addition of trimethyl-1,3,5,2,4,6-trioxatriborinane (367 mg, 2.93 mmol) and 1,1′-bis(diphenylphosphino)ferrocene]dichloropalladium(II) (128 mg, 176 μmol) at room temperature. The reaction mixture was then heated at 85° C. for 10 hours. After cooling to room temperature, the reaction mixture was filtered and concentrated to afford crude compound which was purified by silica gel chromatography with 12% ethyl acetate in n-hexanes as eluent to afford 3-(benzyloxy)-4-hydroxy-5-methoxy-2-methylbenzaldehyde as colorless liquid.

Step 6: A mixture of 3-(benzyloxy)-4-hydroxy-5-methoxy-2-methylbenzaldehyde (106 mg, 0.389 mmol), N1-(oxetan-3-yl)benzene-1,2-diamine (63.9 mg, 0.389 mmol) and sodium metabisulphite (111 mg, 0.584 mmol) in dimethylsulfoxide (5 mL) was heated and stirred at 85° C. for 16 hours. After cooling to room temperature, the reaction mixture was poured into chilled water; the precipitated solids were filtered and dried to afford 2-(benzyloxy)-6-methoxy-3-methyl-4-[1-(oxetan-3-yl)-1H-1,3-benzodiazol-2-yl]phenol as a yellow solid.

Step 7: A suspension of 2-(benzyloxy)-6-methoxy-3-methyl-4-[1-(oxetan-3-yl)-1H-1,3-benzodiazol-2-yl]phenol (100 mg, 0.25 mmol) and palladium hydroxide (10.0 mg, 0.07 mmol) in methanol (10 mL) was stirred under hydrogen pressure (1 atm) overnight. The reaction mixture was filtered, and the filtrate was concentrated. The crude compound was purified by preparative HPLC to afford 6-methoxy-3-methyl-4-(1-(oxetan-3-yl)-1H-benzo[d]imidazol-2-yl)benzene-1,2-diol as white solid. MS (ESI) m/z 327.14 [M+1]⁺.

### Example 2. Synthesis of 2-hydroxy-4-(3H-imidazo[4,5-c]pyridin-2-yl)benzoic acid

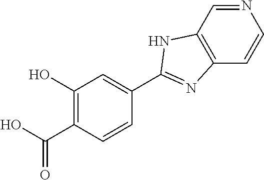 [SMILES: O=C(O)c1ccc(-c2nc3ccncc3[nH]2)cc1O]

4-Formyl-2-hydroxybenzoic acid (1.00 eq, 51.0 mg, 0.307 mmol), 3,4-diaminopyridine (1.00 eq, 33.5 mg, 0.307 mmol), sodium metabisulfite (0.500 eq, 29.2 mg, 0.153 mmol), and DMSO (0.700 mL) were combined in a sealed vial with a magnetic stirbar. The resulting mixture was stirred at 90° C. for 16 h. The reaction mixture was diluted with 1.5 mL of TFA to dissolve most of the reaction mixture. The crude mixture was diluted slightly with methanol, filtered, and purified via preparatory HPLC (5-25% acetonitrile in water with 0.1% TFA). Fractions containing the desired product were combined and lyophilized to dryness to afford 2-hydroxy-4-(3H-imidazo[4,5-c]pyridin-2-yl)benzoic acid as a white solid. MS m/z 256.1 [M+1]⁺.

### Example 3: Synthesis of 2-hydroxy-5-(1-(oxetan-3-yl)-1H-benzo[d]imidazol-2-yl)benzoic acid

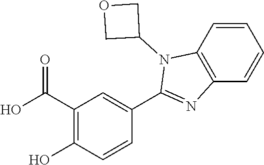 [SMILES: O=C(O)c1cc(-c2nc3ccccc3n2C2COC2)ccc1O]

Step 1: 1-Fluoro-2-nitrobenzene (1.00 eq, 386 mg, 2.74 mmol), oxetan-3-amine (1.00 eq, 200 mg, 2.74 mmol), potassium carbonate (1.30 eq, 492 mg, 3.56 mmol), and DMF (2.5 mL) were combined in a sealed vial with a magnetic stirbar. The resulting suspension was stirred at 80° C. for 16 h. The reaction mixture was diluted with EtOAc and washed with water. The aqueous layer was back-extracted with EtOAc and the combined organics were washed with brine twice, dried over sodium sulfate, filtered and evaporated to dryness. The crude was purified by silica gel chromatography (10-50% EtOAc in hexanes) to afford N-(2-nitrophenyl)oxetan-3-amine as an orange solid.

Step 2: Palladium on Carbon (0.0500 eq, 137 mg, 0.130 mmol) was added to a stirring solution of N-(2-nitrophenyl)oxetan-3-amine (1.00 eq, 503 mg, 2.59 mmol) in methanol (10 mL). The reaction was stirred under a bubbling stream of H₂ gas for 15 min and then under an atmosphere of H₂ gas for 16 h. The reaction mixture was filtered thru Celite and the filter cake washed with MeOH. The combined filtrates were evaporated to dryness to afford N1-(oxetan-3-yl)benzene-1,2-diamine as a dark oil.

Step 3: N1-(Oxetan-3-yl)benzene-1,2-diamine (1.00 eq, 39.0 mg, 0.238 mmol), 5-formylsalicylic acid (1.00 eq, 39.5 mg, 0.238 mmol), sodium metabisulfite (0.500 eq, 22.6 mg, 0.119 mmol), and DMSO (0.7000 mL) were combined in a sealed vial with a magnetic stirbar. The resulting mixture was stirred at 80° C. for 16 h. The reaction mixture was diluted with 1 mL TFA and 0.1 mL of MeOH, filtered, and purified via preparatory HPLC (10-35% acetonitrile in water with 0.1% TFA). Fractions containing the desired product were combined and lyophilized to dryness to afford 2-hydroxy-5-(1-(oxetan-3-yl)-1H-benzo[d]imidazol-2-yl)benzoic acid as an off-white solid. MS m/z 311.1 [M+1]⁺.

### Example 4: Synthesis of 5-(5-(azetidin-1-yl)-1H-benzo[d]imidazol-2-yl)-3-methoxybenzene-1,2-diol

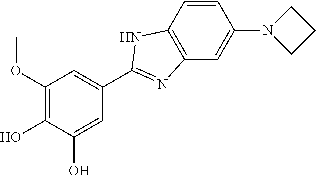 [SMILES: COc1cc(-c2nc3cc(N4CCC4)ccc3[nH]2)cc(O)c1O]

Step 1: To a stirred solution of 5-fluoro-2-nitroaniline (1.00 g, 6.41 mmol) in 1-methylpyrrolidin-2-one (10.0 mL), azetidine (366 mg, 6.41 mmol) and N-ethyldiisopropylamine (5.53 mL, 32.0 mmol) were added at room temperature and heated the reaction mass at 100° C. for 4 hours. After cooling to room temperature, it was slowly poured into chilled water; the precipitated solids were filtered, washed with diethyl ether (2×10 mL) and dried to afford 5-(azetidin-1-yl)-2-nitroaniline as a yellow solid.

Step 2: A mixture of 5-(azetidin-1-yl)-2-nitroaniline (250 mg, 1.29 mmol) and palladium on carbon (10% w/w, 250 mg, 1.17 mmol) in methanol (5.00 mL) was stirred at room temperature under 1 atmospheric hydrogen pressure for 16 hours. After completion, the reaction mixture was filtered and the filtrate was concentrated under reduced pressure to afford 4-(azetidin-1-yl)benzene-1,2-diamine as dark brown gum which was used immediately for the next step.

Step 3: A mixture of 4-(azetidin-1-yl)benzene-1,2-diamine (190 mg, 1.16 mmol), 3,4-dihydroxy-5-methoxybenzaldehyde (157 mg, 0.93 mmol) and sodium metabisulfite (443 mg, 2.33 mmol) in dimethyl sulfoxide (4.00 mL) was stirred at 85° C. for 16 hours. After cooling to room temperature, the reaction mixture was poured into brine (10 mL) and filtered the precipitated solids. The crude compound was purified by preparative HPLC to afford 5-(5-(azetidin-1-yl)-1H-benzo[d]imidazol-2-yl)-3-methoxybenzene-1,2-diol as a yellow solid. MS (ESI) m/z 312.16 [M+1]⁺.

### Example 5. Synthesis of 2-hydroxy-5-(3H-imidazo[4,5-c]pyridin-2-yl)-3-methoxybenzoic acid

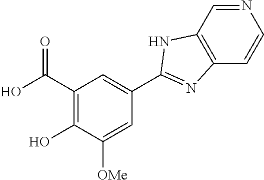 [SMILES: COc1cc(-c2nc3ccncc3[nH]2)cc(C(=O)O)c1O]

5-Carboxyvanillin (1.00 eq, 89.6 mg, 0.457 mmol), 3,4-diaminopyridine (1.00 eq, 49.8 mg, 0.457 mmol), sodium metabisulfite (0.500 eq, 43.4 mg, 0.228 mmol), and DMSO (1 mL) were combined in a sealed vial with a magnetic stirbar. The resulting mixture was stirred at 90° C. for 16 h. The reaction mixture was diluted with 5 mL of TFA. The crude mixture was diluted slightly with methanol, filtered, and purified via preparatory HPLC (5-30% acetonitrile in water with 0.1% TFA). Fractions containing the desired product were combined and lyophilized to dryness to afford 2-hydroxy-5-(3H-imidazo[4,5-c]pyridin-2-yl)-3-methoxy-benzoic acid; 2,2,2-trifluoroacetic acid as a light yellow solid. MS m/z 286.1 [M+1]⁺.

### Example 6. Synthesis of 5-(6-(azetidin-1-yl)-1H-benzo[d]imidazol-2-yl)-2-hydroxy-3-methoxybenzoic acid

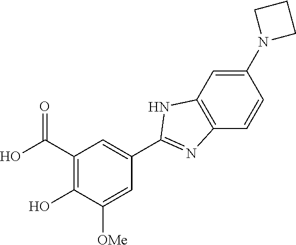 [SMILES: COc1cc(-c2nc3ccc(N4CCC4)cc3[nH]2)cc(C(=O)O)c1O]

Step 1: 5-Fluoro-2-nitroaniline (1.00 eq, 5.01 g, 32.1 mmol), potassium carbonate (1.20 eq, 5.32 g, 38.5 mmol), azetidine (1.20 eq, 2199 mg, 38.5 mmol), and 1,4-dioxane (20 mL) were combined in a sealable vessel with a stirbar, sealed, stirred, and heated at 90° C. with a heating block for 2 h. After cooling to room temperature, the reaction mixture was concentrated on the rotary evaporator with silica gel. The residue was purified via silica gel chromatography (0-60% ethyl acetate in dichloromethane) to afford the 5-(azetidin-1-yl)-2-nitroaniline as an orange solid.

Step 2: Palladium hydroxide (0.0300 eq, 54.5 mg, 0.0776 mmol) was added to a stirring suspension of 5-(azetidin-1-yl)-2-nitroaniline (1.00 eq, 500 mg, 2.59 mmol) in ethanol (10 mL) and methanol (2 mL). The reaction was stirred under a bubbling stream of H₂ gas for 15 min and then under an atmosphere of H₂ gas for 16 h. The reaction mixture was filtered thru Celite and the filter cake washed with MeOH. The combined filtrates were evaporated to dryness. The crude residue was dissolved in dichloromethane and purified via silica gel chromatography (0 to 20% MeOH in EtOAc) to afford 4-(azetidin-1-yl)benzene-1,2-diamine as a dark oil.

Step 3: 4-(Azetidin-1-yl)benzene-1,2-diamine (1.00 eq, 50.0 mg, 0.306 mmol), 5-Carboxyvanillin (1.00 eq, 60.1 mg, 0.306 mmol), sodium metabisulfite (0.500 eq, 29.1 mg, 0.153 mmol), and DMSO (1 mL) were combined in a sealed vial with a magnetic stirbar. The resulting mixture was stirred at 80° C. for 16 h. The reaction mixture was diluted with 0.4 mL TFA and 0.1 mL of MeOH, filtered, and purified via preparatory HPLC (10-35% acetonitrile in water with 0.1% TFA). Fractions containing the desired product were combined and lyophilized to dryness to afford 5-[6-(azetidin-1-yl)-1H-benzimidazol-2-yl]-2-hydroxy-3-methoxybenzoic acid as a yellow solid. MS m/z 340.1 [M+1]⁺.

### Example 7-171: Example 7-171 were Synthesized as Described in Examples 1-6. LC-MS Data is Found in Table 1

### Synthesis of 3,4-bis(benzyloxy)-2-fluoro-5-methoxybenzaldehyde

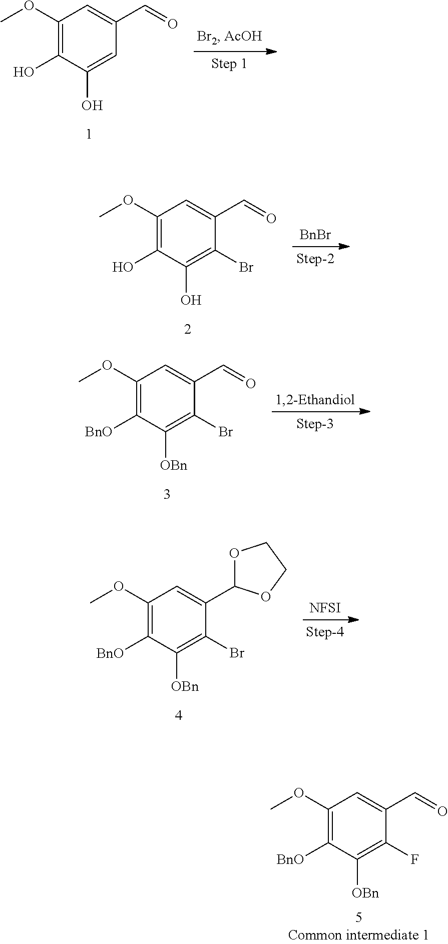

Step-1: To a solution of 3,4-dihydroxy-5-methoxybenzaldehyde (20.0 g, 59.5 mmol) in acetic acid (100 mL) and dibromine (5.94 mL, 118.94 mmol) diluted in acetic acid (20 mL) was added dropwise to the reaction mixture and stirred for 16 hr. After completion of the reaction, the reaction mixture was quenched with saturated solution of Sodium thiosulfate and stirred for 10 minute so that solid compound precipitate. After that reaction mixture filtered out and solid washed with ice cold water and dried under vacuum. The solid afford 2-bromo-3,4-dihydroxy-5-methoxybenzaldehyde (24.0 g, 97.15 mmol) as off white solid.

### Yield: 24.0 g, 81.68%

Step-2: To a solution of 2-bromo-3,4-dihydroxy-5-methoxybenzaldehyde (24.0 g, 97.1 mmol) in N,N-dimethylformamide (240 mL), (chloromethyl)benzene (33.54 mL, 3 eq., 291 mmol), dipotassium carbonate (40.3 g, 3 eq., 291 mmol) and potassium iodide (3.23 g, 0.2 eq., 19.4 mmol) were added to the reaction mixture and stirred and heated at 60° C. for 4 h. After completion of the reaction, the reaction mixture was filtered and the filtrate was concentrated under reduced pressure to get the crude. The crude purified by flash chromatography. The desired fractions were concentrated to afford 3,4-bis(benzyloxy)-2-bromo-5-methoxybenzaldehyde (30.0 g, 70.21 mmol) as off white solid.

### Yield: 30.0 g, (72.27%)

Step-3: To a solution of 3,4-bis(benzyloxy)-2-bromo-5-methoxybenzaldehyde (15.00 g, 35.1 mmol) in dry toluene (120.0 mL), ethane-1,2-diol (6.54 gm, 3 eq., 105.3 mmol) and Dry paratoluenesulfonic acid (604.49 mg, 0.1 eq., 3.51 mmol) were added to the reaction mixture was stirred and heated at 130° C. for 16 h. After completion of the reaction, the reaction mixture was concentrated under reduced pressure to get the crude. The crude purified by flash chromatography. The desired fractions were concentrated to afford 2-[3,4-bis(benzyloxy)-2-bromo-5-methoxyphenyl]-1,3-dioxolane (10.00 g, 21.22 mmol) as off white solid. Yield: 10.00 g, 54%

Step-4: To a Pre dried RBF 2-[3,4-bis(benzyloxy)-2-bromo-5-methoxyphenyl]-1,3-dioxolane (5.00 g, 6.38 mmol) was added in Tetrahydrofuran (50.0 mL) under inert atmosphere. The solution was cooled at −78° C. and n-Butyllithium (7.64 mL, 1.8 eq., 19.09 mmol) was added dropwise. The reaction mixture stirred for 30 minutes at −78° C. N-(benzenesulfonyl)-N-fluorobenzenesulfonamide (5.35 g, 1.6 eq., 16.97 mmol) in tetrahydrofuran (10 ml) was added dropwise at −78° C. and stirred for 1 h. After completion, the reaction mixture quenched with 100 mL of 6 N hydrochloric acid solution (stirred for 2 h), extracted with ethyl acetate. The combined organic layers dried over anhydrous sodium sulphate, filtered and concentrated to obtain crude. The crude was purified by flash chromatography to afford 3,4-bis(benzyloxy)-2-fluoro-5-methoxybenzaldehyde (2.00 g, 5.46 mmol) as light yellow solid. Yield: 2.00 g, 51.46%

NMR Data: 1HNMR (400 MHz, DMSO-d6): δ 10.12 (s, 1H), 7.48-7.33 (m, 11H), 7.16 (d, J=6.0 Hz, 1H), 5.15 (s, 2H), 5.07 (s, 2H), 3.86 (s, 3H).

### Example 172: Synthesis of 6-methoxy-4-[1-(2-methoxyethyl)-1H-1,3-benzodiazol-2-yl]-3-methylbenzene-1,2-diol

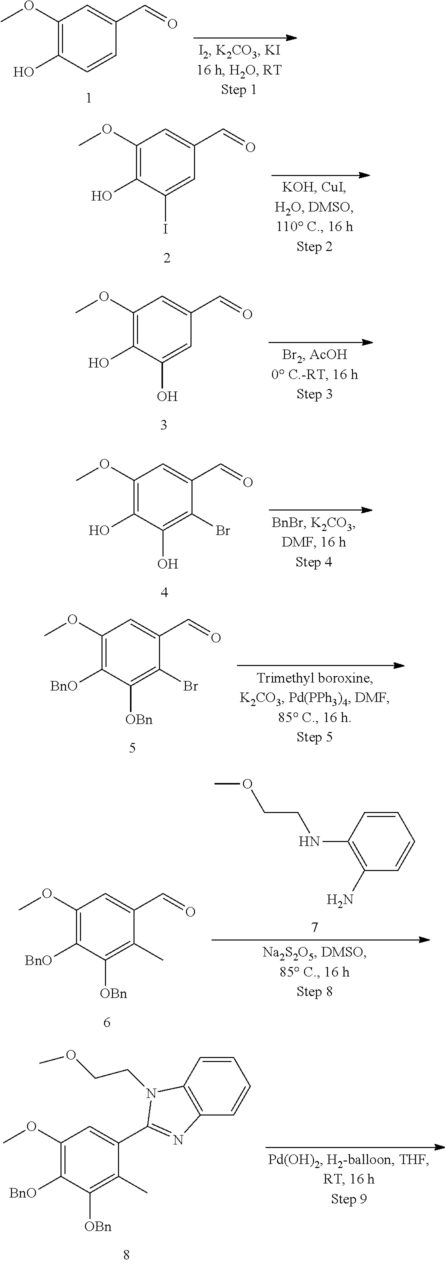

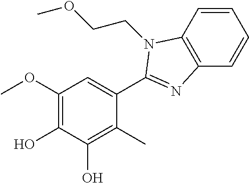 [SMILES: COCCn1c(-c2cc(OC)c(O)c(O)c2C)nc2ccccc21]

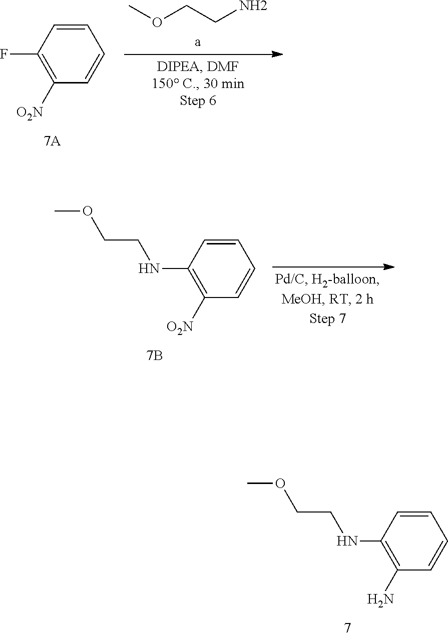

### Step 1: 4-Hydroxy-3-Iodo-5-Methoxybenzaldehyde

To a stirred solution of 4-hydroxy-3-methoxybenzaldehyde (10 g, 65.7 mmol, 1 eq) in water (150 mL), dipotassium carbonate (13.6 g, 98.6 mmol, 1.5 eq) was added at room temperature. Then potassium iodide (12 g, 72.3 mmol, 1.1 eq) and iodine (12.5 g, 98.6 mmol, 1.5 eq) was added three portions over 30 min. The reaction mixture was stirred for 16 hours at room temperature. The reaction mixture was quenched with saturated Na₂S₂O₃ solution, precipitated solids were filtered and washed with a saturated Na₂S₂O₃ solution and H₂O to afford 4-hydroxy-3-iodo-5-methoxybenzaldehyde as a light brown solid. LCMS (ESI)m/z 279.0 [M+H]⁺

### Step 2: 3,4-dihydroxy-5-methoxybenzaldehyde

To a solution of 4-hydroxy-3-iodo-5-methoxybenzaldehyde (5 g, 18.0 mmol, 1 eq) in DMSO (4 mL) was added potassium hydroxide (10.1 g, 180 mmol, 10 eq) in water (24 mL) and diiodocopper (0.571 g, 1.80 mmol, 0.1 eq) in autoclave apparatus. The reaction mixture was stirred at 110° C. for 16 hours. After cooling to room temperature, poured into water and acidified with conc HCl up to pH˜1.0. The mixture was extracted with ethyl acetate twice. The combined organic phase was dried over sodium sulfate and concentrated. The crude compound was purified by flash chromatography to afford 3,4-dihydroxy-5-methoxybenzaldehyde as a yellow solid. LC-MS (ES) m/z: 169.0 [M+H]⁺

### Step 3: 2-bromo-3,4-dihydroxy-5-methoxybenzaldehyde

To a stirred solution of 3,4-dihydroxy-5-methoxybenzaldehyde (2.6 g, 15.5 mmol, 1 eq.) in acetic acid (10 mL) was added dibromine (2.72 mL, 17.0 mmol, 1.1 eq.) at 0° C. and stirred at room temperature for 16 hours. The progress of the reaction was monitored by TLC (30% Ethyl acetate in Hexane). After completion of the reaction, the reaction mixture was quenched with saturated sodium thiosulfate and stirred for 10 minutes, precipitated solids were filtered and washed with ice cold water and dried to afford 2-bromo-3,4-dihydroxy-5-methoxybenzaldehyde as an off white solid. LCMS (ESI)m/z 247.0[M]⁺& 249.0[M+2H]⁺

### Step 4: 3,4-bis(benzyloxy)-2-bromo-5-methoxybenzaldehyde

To a stirred solution of 2-bromo-3,4-dihydroxy-5-methoxybenzaldehyde (2.5 g, 10.1 mmol, 1 eq.) in N, N-dimethylformamide (10 mL) was added dipotassium carbonate (4.20 g, 30.4 mmol, 3 eq.) at room temperature. After stirring for 10 minutes, (bromomethyl)benzene (3.61 mL, 30.4 mmol, 3 eq.) was added and stirred the reaction mixture at room temperature for 16 hours. The reaction mixture was quenched with chilled water and extracted with ethyl acetate, dried over sodium sulfate and concentrated. The crude was purified by flash chromatography using 5-10% ethyl acetate in hexane as eluent to afford 3,4-bis(benzyloxy)-2-bromo-5-methoxybenzaldehyde (2.26 g, 52.27% Yield) as light yellow liquid. LCMS (ESI)m/z 427.3[M+H]⁺ & 429 [M+2H]⁺

### Step 5: 3,4-bis(benzyloxy)-5-methoxy-2-methylbenzaldehyde

To a stirred solution of 3,4-bis(benzyloxy)-2-bromo-5-methoxybenzaldehyde (1.26 g, 2.95 mmol, 1 eq.) in N, N-dimethylformamide (4 mL) was added dipotassium carbonate (1.22 g, 8.85 mmol, 3 eq.) followed by trimethyl-1,3,5,2,4,6-trioxatriborinane (0.925 g, 7.37 mmol, 2.5 eq.) in sealed tube purged with argon for 5 min. Then tetrakis(triphenylphosphane) palladium (0.682 g, 0.590 mmol, 0.2 eq.) was added to the reaction mixture and stirred at 85° C. for 16 hours, progress of the reaction was monitored by TLC (10% Ethyl acetate in Hexane). After completion of the reaction, reaction mixture was filtered in celite bed and the filtrate was extracted with ethyl acetate, washed with water &brine solution, dried over sodium sulfate and filtered and concentrated. The crude was purified by flash chromatography using 1%-10% ethyl acetate in hexane as eluent to afford 3,4-bis(benzyloxy)-5-methoxy-2-methylbenzaldehyde (0.5 g, 46.79% Yield) as a light brown liquid. LCMS (ESI)m/z 363.0 [M+H]⁺

### Step 6: N-(2-methoxyethyl)-2-nitroaniline

To a stirred solution of 1-fluoro-2-nitrobenzene (0.5 g, 0.003 mmol, 1 eq.) in 1-methylpyrrolidin-2-one (2 mL) was added ethylbis(propan-2-yl)amine (1.79 mL, 10.3 mmol, 2.9 eq.) added and 2-methoxyethan-1-amine (0.266 g, 0.003 mmol, 1 eq.) and the reaction mixture was stirred at 150° C. for 30 min in microwave. Progress of the reaction was monitored by TLC (10% Ethyl acetate in Hexane). After completion of the reaction, reaction mixture was diluted with water extracted with ethyl acetate. Combined organic layer was washed with brine, dried over anhydrous sodium sulphate and concentrated. The crude was purified by flash column chromatography with 5%-10% ethyl acetate in hexane as eluent to afford N-(2-methoxyethyl)-2-nitroaniline (0.6 g, 86.3% Yield) as a color less liquid. LCMS (ESI)m/z 197.1 [M+H]⁺

### Step 7: N1-(2-methoxyethyl)benzene-1,2-diamine

A suspension of N-(2-methoxyethyl)-2-nitroaniline (0.3 g, 1.53 mmol, 1 eq) and 10% palladium on carbon (0.75 g, 0.705 mmol, 0.4 eq) in methanol (6 mL) was stirred at room temperature under hydrogen pressure (1 atm) for 3 hours, The reaction was monitored by TLC (50% Ethyl acetate in Hexane). After completion of the reaction, reaction mixture was filtered and the filtrate was concentrated under reduced pressure to afford N1-(2-methoxyethyl)benzene-1,2-diamine (0.250 g, Crude) as a light pink solid. The obtained crude was forwarded to next step without purification. LCMS (ESI)m/z 167.1 [M+H]⁺

### Step 8: 2-[3,4-bis(benzyloxy)-5-methoxy-2-methylphenyl]-1-(2-methoxyethyl)-1H-1,3-benzodiazole

A mixture of 3,4-bis(benzyloxy)-5-methoxy-2-methylbenzaldehyde (0.150 g, 0.414 mmol, 1 eq) N1-(2-methoxyethyl) benzene-1,2-diamine (0.103 g, 0.621 mmol, 1.5 eq) and sodium metabisulphite (0.118 g, 0.621, 1.5 eq) in dimethylsulfoxide (3 mL) was stirred at 85° C. for 12 hours. The progress of the reaction was monitored by TLC (30% Ethyl acetate in Hexane). After completion of the reaction, reaction mixture was diluted with Ethyl acetate and washed with water and brine, dried over sodium sulfate and filtered and concentrated. The crude was purified by flash column chromatography with 15%-20% ethyl acetate in hexane to afford 2-[3,4-bis(benzyloxy)-5-methoxy-2-methylphenyl]-1-(2-methoxyethyl)-1H-1,3-benzodiazole (0.2 g, 95% Yield) as colorless liquid. LCMS (ESI)m/z 509.3 [M+H]⁺

### Step 9: 6-methoxy-4-[1-(2-methoxyethyl)-1H-1,3-benzodiazol-2-yl]-3-methylbenzene-1,2-diol; acetic acid

To a stirred solution of 2-[3,4-bis(benzyloxy)-5-methoxy-2-methylphenyl]-1-(2-methoxyethyl)-1H-1,3-benzodiazole (0.2 g, 0.393 mmol, 1 eq) in THF (10 mL) was added Pd(OH)₂ on Carbon (0.06 g) was stirred under hydrogen pressure for 12 hours at room temperature. The reaction was monitored by TLC. After completing the starting material reaction mixture was filtered and the filtrate was concentrated under pressure to get crude compound. The crude compound was purified by preparative HPLC to afford 6-methoxy-4-[1-(2-methoxyethyl)-1H-1,3-benzodiazol-2-yl]-3-methylbenzene-1,2-diol; acetic acid. (12 mg, 7.8% Yield) as a white solid. ¹H NMR (400 MHz, DMSO-d₆): δ ppm 1.87 (s, 3H), 3.04 (s, 3H), 3.49 (s, 2H), 3.75 (s, 3H), 4.19 (s, 2H), 6.53 (s, 1H), 7.20-7.23 (m, 3H), 7.61 (m, 2H), 8.60 (s, 1H), 8.89 (s, 1H). LCMS (ESI)m/z 329.2 [M+H]+

### Example 173: Synthesis of 3-ethyl-6-methoxy-4-[1-(3-methyloxetan-3-yl)-1H-1,3-benzodiazol-2-yl]benzene-1,2-diol

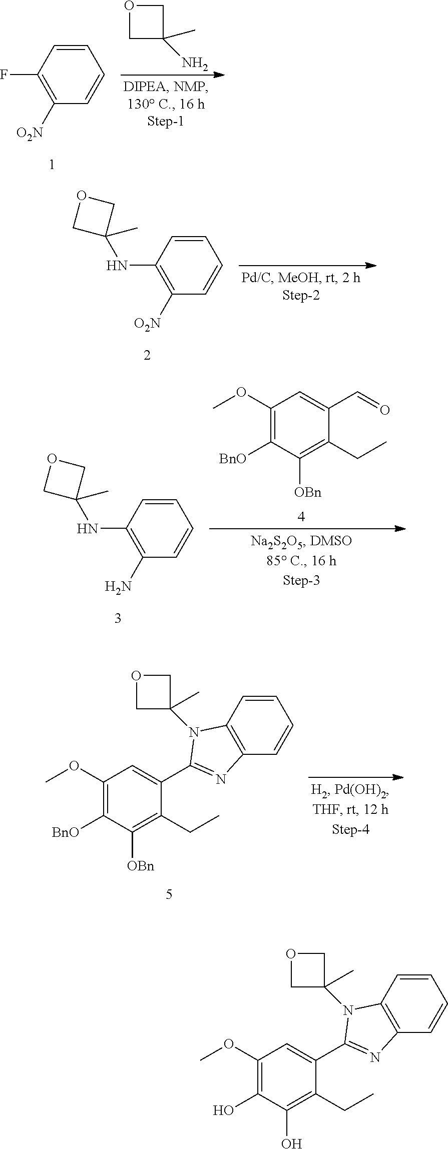

### Step 1: 3-methyl-N-(2-nitrophenyl)oxetan-3-amine

To a stirred solution of 1-fluoro-2-nitrobenzene (1 g, 7.09 mmol, 1 eq) in 1-methylpyrrolidin-2-one (5 mL) was added ethylbis(propan-2-yl) amine (2.47 mL, 14.2 mmol, 2 eq) and methyloxetan-3-amine (1.54 g, 17.7 mmol, 2.5 eq). The reaction mixture was heated to 130° C. for 16 hours. The progress of the reaction was monitored by TLC (10% Ethyl acetate in Hexane). After completion of the reaction, reaction mixture was diluted with Ethyl acetate and washed with water and brine, dried over sodium sulfate and filtered and concentrated. The crude was purified by flash column chromatography with 5-10% ethyl acetate in n-hexane as eluent to afford 3-methyl-N-(2-nitrophenyl) oxetan-3-amine (1 g, 67.7% Yield) as a light brown liquid. LCMS (ESI)m/z 209.1 [M+H]⁺

### Step 2: N1-(3-methyloxetan-3-yl) benzene-1,2-diamine

A suspension of 3-methyl-N-(2-nitrophenyl) oxetan-3-amine (0.22 g, 1.06 mmol, 1 eq) and 10% palladium on carbon (0.04 g) in methanol (4 mL) was stirred at room temperature under hydrogen balloon for 3 hours. The reaction was monitored by TLC (30% Ethyl acetate in Hexane). After completion of the reaction, reaction mixture was filtered, filtrate was concentrated to afford N1-(3-methyloxetan-3-yl)benzene-1,2-diamine (0.120 g, Crude) as light pink solid. The crude was forwarded to next step without purification. LCMS (ESI)m/z 179.1 [M+H]+

### Step 3: 2-[3,4-bis(benzyloxy)-2-ethyl-5-methoxyphenyl]-1-(3-methyloxetan-3-yl)-1H-1,3-benzodiazole

A mixture of 3,4-bis(benzyloxy)-2-ethyl-5-methoxybenzaldehyde (0.160 g, 0.425 mmol, 1 eq), N1-(3-methyloxetan-3-yl) benzene-1,2-diamine (0.114 g, 0.638 mmol, 1.5 eq) and sodium metabisulphite (0.121 g, 0.638, 1.5 eq) in DMSO (4 mL) was stirred at 85° C. for 12 hours. The progress of the reaction was monitored by TLC (30% Ethyl acetate in Hexane). After completion of the reaction, reaction mixture was diluted with ethyl acetate and washed with water and brine, dried over sodium sulfate and concentrated. The crude was purified by flash column chromatography with 15-20% ethyl acetate in hexane to afford 2-[3,4-bis(benzyloxy)-2-ethyl-5-methoxyphenyl]-1-(3-methyloxetan-3-yl)-1H-1,3-benzodiazole (0.130 g, 57.21% Yield) as a brown liquid. LCMS (ESI)m/z 553.3 [M+H]⁺

### Step 4: 3-ethyl-6-methoxy-4-[1-(3-methyloxetan-3-yl)-1H-1,3-benzodiazol-2-yl] benzene-1,2-diol; acetic acid

A suspension of 2-[3,4-bis(benzyloxy)-2-ethyl-5-methoxyphenyl]-1-(3-methyloxetan-3-yl)-1H-1,3-benzodiazole (0.1 g, 0.187 mmol, 1 eq) and Pd(OH)₂ on Carbon (0.04 g) in THF (5 mL) was stirred under hydrogen pressure (1 atm) for 12 hours. The reaction was monitored by TLC. After completing the starting material reaction mixture was filtered through celite bed washing with THF. The filtrate was concentrated. The crude compound was purified by preparative HPLC to afford 3-ethyl-6-methoxy-4-[1-(3-methyloxetan-3-yl)-1H-1,3-benzodiazol-2-yl]benzene-1,2-diol; acetic acid (0.02 g, 26% Yield) as a white solid. ¹H NMR (400 MHz, DMSO-d₆): δ ppm 1.04 (t, J=8, 3H), 1.90 (s, 1H), 2.26 (s, 3H), 2.33 (s, 1H), 2.78 (bs, 1H), 3.92 (bs, 3H), 4.44 (bs, 1H), 4.71 (bs, 1H), 4.83 (bs, 2H), 6.40 (s, 1H), 7.22 (s, 3H), 7.68 (s, 1H), 8.66 (bs, 1H), 8.92 (bs, 1H). LCMS (ESI)m/z 355.2 [M+H]+

### Example 174: Synthesis of 6-methoxy-4-[1-(2-methoxyethyl)-1H-1,3-benzodiazol-2-yl]-3-methylbenzene-1,2-diol

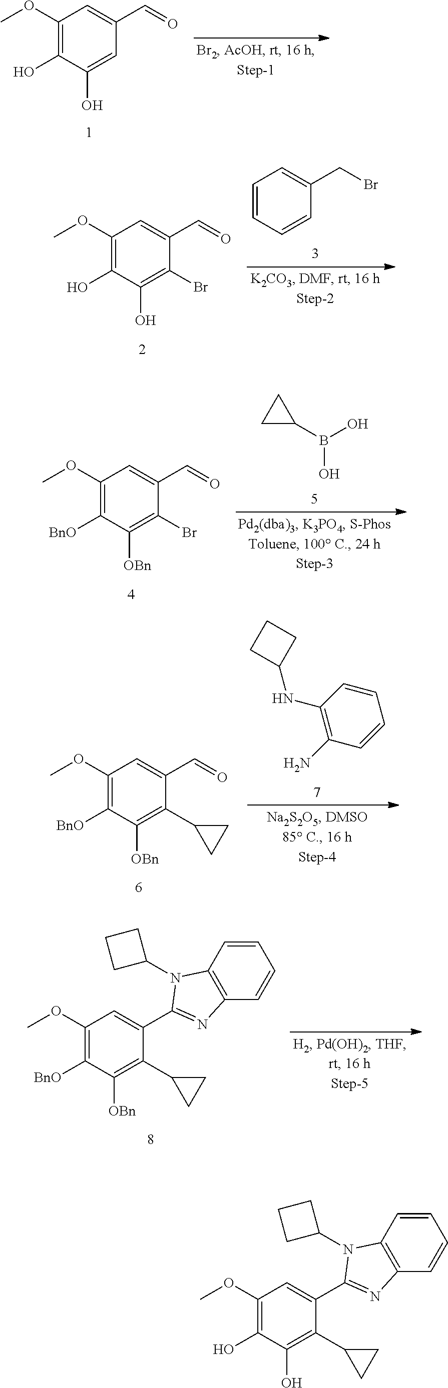

### Step 1: 2-bromo-3,4-dihydroxy-5-methoxybenzaldehyde

To a stirred solution of 3,4-dihydroxy-5-methoxybenzaldehyde (2.00 g, 11.9 mmol) in acetic acid (20.0 mL) was added bromine (674 μL, 1.1 eq., 13.1 mmol) at 0° C. and the resulting mixture was stirred at room temperature for 16 hours. The reaction mixture was quenched with saturated sodium thiosulfate solution, precipitated solids were filtered and washed with ice cold water (25 mL) and n-pentane (25 mL), dried to afford to afford 2-bromo-3,4-dihydroxy-5-methoxybenzaldehyde as grey colored solid (1 g, 34% Yield) as off white solid. LCMS (ESI)m/z 247 [M]⁺, 249.0 [M+2H]+

### Step 2: 3,4-bis(benzyloxy)-2-bromo-5-methoxybenzaldehyde

To a stirred solution of 2-bromo-3,4-dihydroxy-5-methoxybenzaldehyde (1.00 g, 4.05 mmol) in N,N-dimethylformamide (15.0 mL) was added dipotassium carbonate (1.68 g, 3 eq., 12.1 mmol) at 0° C. and stirred for 10 min, (bromomethyl)benzene (1.44 mL, 3 eq., 12.1 mmol) was added and the resulting mixture was stirred at room temperature for 16 hours. The reaction mass was quenched with chilled water (50 mL) and extracted with ethyl acetate (2×50 mL). The organic layer was washed with water, dried over sodium sulfate and concentrated. The crude was purified by flash column chromatography with 5-10% ethyl acetate in heptane as eluent. to afford 3,4-bis(benzyloxy)-2-bromo-5-methoxybenzaldehyde (0.93 g, 53.7% Yield) as an off white solid. LCMS (ESI)m/z 427 [M]⁺, 429.0 [M+2H]+

### Step 3: 3,4-bis(benzyloxy)-2-cyclopropyl-5-methoxybenzaldehyde

To a stirred solution of 3,4-bis(benzyloxy)-2-bromo-5-methoxybenzaldehyde (280 mg, 655 μmol) in toluene (10.0 mL) was added 3,4-bis(benzyloxy)-2-cyclopropyl-5-methoxybenzaldehyde (720 mg, 1.85 mmol) and tripotassium phosphate (278 mg, 2 eq., 1.31 mmol) and degassed with argon for 10 min. Then dicyclohexyl({2′,6′-dimethoxy-[1,1′-biphenyl]-2-yl})phosphane (53.8 mg, 0.2 eq., 131 μmol) and tris((1E,4E)-1,5-diphenylpenta-1,4-dien-3-one) dipalladium (60.0 mg, 0.1 eq., 65.5 μmol) was added and the resulting mixture was stirred at 100° C. in a sealed tube for 24 hours. Cooled to room temperature and filtered through celite. The bed was washed with ethyl acetate and the filtrate was concentrated. The crude was purified by flash column chromatography with 10% ethyl acetate in heptane as eluent to afford 3,4-bis(benzyloxy)-2-cyclopropyl-5-methoxybenzaldehyde (0.72 g, Crude). as a yellow oil. LCMS (ESI)m/z 388.9 [M+H]⁺.

### Step 4: 2-(3,4-bis(benzyloxy)-2-cyclopropyl-5-methoxyphenyl)-1-cyclobutyl-1H-benzo[d]imidazole

A mixture of 3,4-bis(benzyloxy)-2-cyclopropyl-5-methoxybenzaldehyde (720 mg, 1.85 mmol) N1-cyclobutylbenzene-1,2-diamine (451 mg, 1.5 eq., 2.78 mmol) and sodium metabisulphite (528 mg, 1.5 eq., 2.78 mmol) in DMSO (15.0 mL) was heated at 85° C. for 16 hours. Cooled to room temperature, water (10 mL) was added and extracted with ethyl acetate (2×50 mL). The combined organic extract was washed with water (15 mL), brine solution (10 mL) and dried over sodium sulphate and concentrated. The crude was purified by column chromatography using 70% ethyl acetate in heptane as eluent to afford 2-(3,4-bis(benzyloxy)-2-cyclopropyl-5-methoxyphenyl)-1-cyclobutyl-1H-benzo[d]imidazole as a thick brown oil. LCMS (ESI)m/z 531.3 [M+H]+

### Step 5: 4-(1-cyclobutyl-1H-benzo[d]imidazol-2-yl)-3-cyclopropyl-6-methoxybenzene-1,2-diol

A suspension of 2-[3,4-bis(benzyloxy)-2-cyclopropyl-5-methoxyphenyl]-1-cyclobutyl-1H-1,3-benzodiazole (180 mg, 149 μmol) and palladium(2+) hydroxide (12.0 mg, 98.0 μmol) in THF (15.0 mL) was stirred under hydrogen pressure (1 atm) at room temperature for 16 hours. The reaction mixture was filtered through celite, filtrate was concentrated. The crude was purified by preparative HPLC to afford 4-(1-cyclobutyl-1H-benzo[d]imidazol-2-yl)-3-cyclopropyl-6-methoxybenzene-1,2-diol (0.022 g, 42% Yield) as an off white solid. ¹H NMR (400 MHz, DMSO-d₆): δ ppm 0.14 (s, 1H), 0.27 (s, 2H), 0.58 (d, J=8.0 Hz, 1H), 1.57 (t, J=6.8 Hz, 1H), 1.81-1.74 (m, 1H), 1.90 (d, J=9.2 Hz, 1H), 2.16 (d, J=4.4 Hz, 1H), 2.67 (t, J=13.6 Hz, 2H), 2.86-2.78 (m, 1H), 3.76 (s, 3H), 4.80 (t, J=8.4 Hz, 1H), 6.43 (s, 1H), 7.28-7.20 (m, 2H), 7.64 (d, J=7.2 Hz, 1H), 7.85 (d, J=7.6 Hz, 1H), 8.45 (bs, 1H), 8.87 (bs, 1H). LCMS (ESI)m/z 351.3 (M+H)+. Melting point: 209° C.

### Example 175: Synthesis of 6-methoxy-4-(5-methoxy-1H-benzo[d]imidazol-2-yl)-3-methylbenzene-1,2-diol

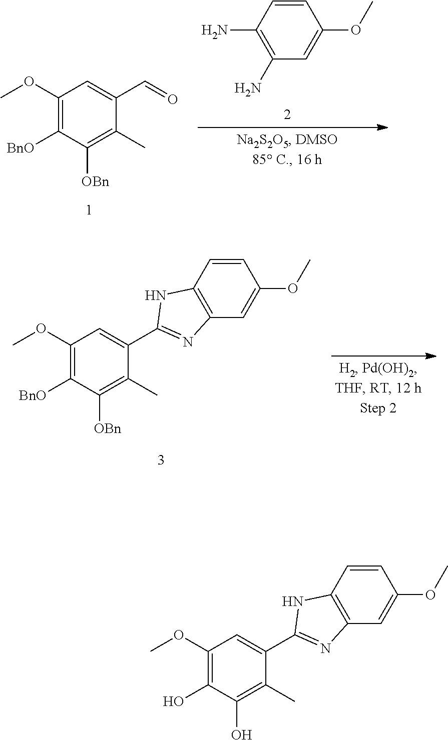

### Step 1: 2-(3,4-bis(benzyloxy)-5-methoxy-2-methylphenyl)-5-methoxy-1H-benzo [d] imidazole

A mixture of 3,4-bis(benzyloxy)-5-methoxy-2-methylbenzaldehyde (0.110 g, 0.304 mmol, 1.0 equiv), 4-methoxybenzene-1,2-diamine (0.052 g, 0.364 mmol, 1.2 equiv) and sodium metabisulphite (0.087 g, 0.455 mmol, 1.5 equiv) in DMSO (5.0 mL) was heated at 85° C. for 16 hours. The reaction mixture was diluted with water (15 mL), extracted with ethyl acetate (2×20 mL). Combined organic layer was washed with brine (15 mL), dried over anhydrous sodium sulphate and concentrated. The crude was purified by flash column chromatography with 15-20% ethyl acetate in hexane as eluent to afford 2-[3,4-bis(benzyloxy)-5-methoxy-2-methylphenyl]-5-methoxy-1H-1,3-benzodiazole (0.120 g, mixture) as brown semisolid. LCMS (ESI)m/z 481.2 [M+H]⁺.

### Step 2: 6-methoxy-4-(5-methoxy-1H-benzo[d]imidazol-2-yl)-3-methylbenzene-1,2-diol

A suspension of 2-[3,4-bis(benzyloxy)-5-methoxy-2-methylphenyl]-5-methoxy-1H-1,3-benzodiazole (0.120 g, 0.250 mmol, 1.0 eq) and 10% palladium hydroxide (12 mg) in THF (6 mL) was stirred under hydrogen pressure (1 atm) for 12 hours. The reaction mixture was filtered, and the filtrate was concentrated. The Crude was purified by preparative HPLC method to afford 6-methoxy-4-(5-methoxy-1H-benzo[d]imidazol-2-yl)-3-methylbenzene-1,2-diol (7 mg, 9.3% Yield) as a dark brown solid. ¹H NMR (400 MHz, DMSO-d₆): δ ppm 3.32-3.33 (m, 3H), 6.75-6.78 (m, 2H), 6.80 (s, 1H), 6.93 (s, 1H), 7.32-7.34 (m, 1H), 7.47-7.49 (m, 1H), 8.61 (bs, 2H), 12.15 (s, 1H). LCMS (ESI)m/z 301.2 [M+H]+ Melting point: 249.1° C.

### Example 176: Synthesis of 6-methoxy-4-[1-(2-methoxyethyl)-1H-1,3-benzodiazol-2-yl]-3-methylbenzene-1,2-diol-acetic acid

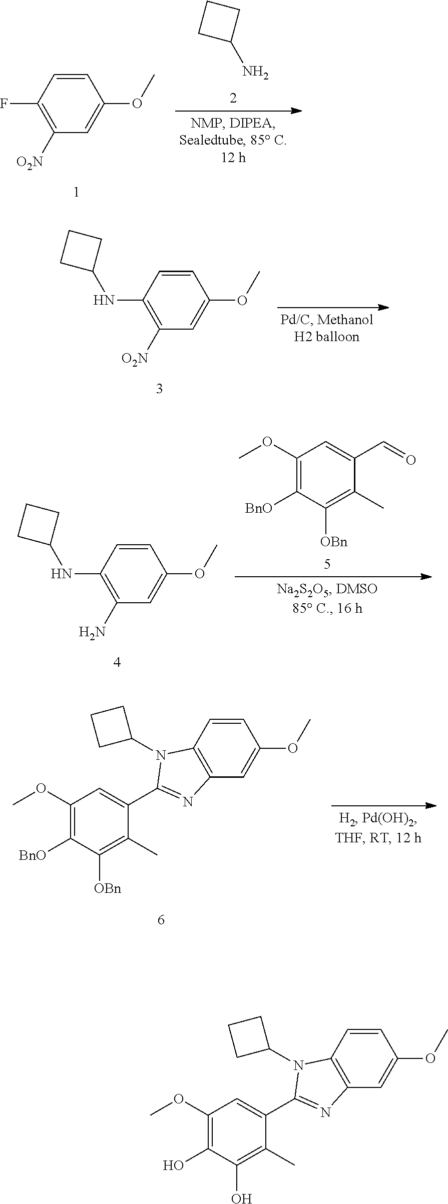

### Step 1: N-cyclobutyl-4-methoxy-2-nitroaniline

A solution of 1-fluoro-4-methoxy-2-nitrobenzene (1 g, 5.84 mmol, 1 eq) in 1-methyl pyrrolidin-2-one (12 mL), ethylbis(propan-2-yl)amine (3.24 mL, 17.5 mmol, 3 eq) and cyclobutamine (1.3 mL, 14.6 mmol, 2.5 eq) was heated at 80° C. for 12 hours in a seal tube. The reaction mixture was cool to room temperature and diluted with ethyl acetate (50 mL). Organic layer was washed with water (25 mL), brine (25 mL), dried over anhydrous sodium sulfate. Concentrated. The crude was purified by flash column chromatography with 5-10% ethyl acetate in hexane as eluent to afford N-cyclobutyl-4-methoxy-2-nitroaniline (1.1 g, 84.7% Yield) as dark red solid. LCMS (ESI)m/z 222.9 [M+H]+

### Step 2: N1-cyclobutyl-4-methoxybenzene-1,2-diamine

A suspension of N-cyclobutyl-4-methoxy-2-nitroaniline (0.350 g, 1.57 mmol, 1.0 eq) and 10% palladium on carbon in methanol (5 mL) was stirred under hydrogen pressure for 2.5 hours at room temperature. The reaction mixture was filtered and the filtrate was concentrated to afford N1-cyclobutyl-4-methoxybenzene-1,2-diamine (0.250 g, crude) as a brown solid. LCMS (ESI)m/z 193.2 [M+H]⁺.

### Step 3: 2-[3,4-bis(benzyloxy)-5-methoxy-2-methylphenyl]-1-cyclobutyl-5-methoxy-1H-1,3-benzodiazole

A mixture of 3,4-bis(benzyloxy)-5-methoxy-2-methylbenzaldehyde (0.110 g, 0.304 mmol, 1.0 equiv) and N1-cyclobutyl-4-methoxybenzene-1,2-diamine (0.070 g, 0.364 mmol, 1.2 equiv) and sodium metabisulphite (0.087 g, 0.455 mmol, 1.5 equiv) in DMSO (6.0 mL) was heated at 85° C. for 16 hours. The reaction mixture was diluted with water (15 mL), extracted with ethyl acetate (2×20 mL). Combined organic layer was washed with brine (15 mL), dried over anhydrous sodium sulphate and concentrated. The crude was purified by flash column chromatography as eluent to afford 2-[3,4-bis(benzyloxy)-5-methoxy-2-methylphenyl]-1-cyclobutyl-5-methoxy-1H-1,3-benzodiazole (0.140 g, mixture) as brown semi solid. LCMS (ESI)m/z 535.3 [M+H]⁺.

### Step 4: 4-(1-cyclobutyl-5-methoxy-1H-1,3-benzodiazol-2-yl)-6-methoxy-3-methylbenzene-1,2-diol

A suspension of 2-[3,4-bis(benzyloxy)-5-methoxy-2-methylphenyl]-1-cyclobutyl-5-methoxy-1H-1,3-benzodiazole (0.140 g, 0.262 mmol, 1.0 eq) and 10% palladium hydroxide (14 mg) in THF (6 mL) was stirred under hydrogen pressure (1 atm) for 12 hours. The reaction mixture was filtered and the filtrate was concentrated. The crude was purified by preparative HPLC to afford 4-(1-cyclobutyl-5-methoxy-1H-1,3-benzodiazol-2-yl)-6-methoxy-3-methylbenzene-1,2-diol (4 mg, 4% Yield) as an off white solid. ¹H NMR (400 MHz, DMSO-d₆): δ ppm 1.70-1.86 (m, 4H), 2.24-2.25 (m, 2H), 2.65-2.70 (m, 2H), 3.58-3.80 (m, 6H), 4.65-4.69 (m, 1H), 6.43 (s, 1H), 6.88-6.90 (m, 1H), 7.18 (s, 1H), 7.71-7.73 (m, 1H), 8.72 (s, 2H). LCMS (ESI)m/z 355.3[M+H]⁺.

### Example 177: Synthesis of 6-methoxy-4-[1-(2-methoxyethyl)-1H-1,3-benzodiazol-2-yl]-3-methylbenzene-1,2-diol

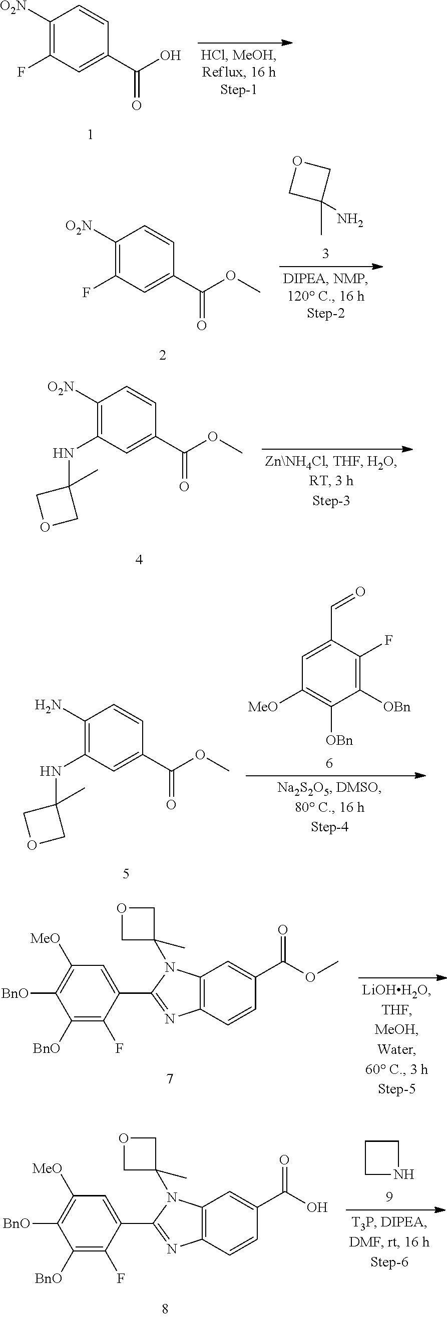

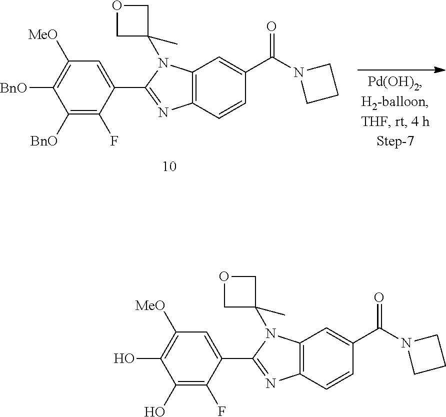 [SMILES: COc1cc(-c2nc3ccc(C(=O)N4CCC4)cc3n2C2(C)COC2)c(F)c(OCc2ccccc2)c1OCc1ccccc1 ; COc1cc(-c2nc3ccc(C(=O)N4CCC4)cc3n2C2(C)COC2)c(F)c(O)c1O]

### Step 1: Methyl 3-fluoro-4-nitrobenzoate

To a stirred solution of 3-fluoro-4-nitrobenzoic acid (10.0 g, 54.0 mmol) in methanol (100 mL) was added hydrogen chloride (6.00 mL) in 0° C. The mixture was heated at 80° C. for 16 hours. The mixture was monitored by TLC (40% Ethyl acetate in Hexane) and LCMS. After completing the starting material, the mixture was concentrated under reduced pressure. The reaction mixture was basified with saturated sodium bicarbonate up to pH-8 and extracted with ethyl acetate, washed with brine, dried over sodium sulfate and concentrated to afford methyl 3-fluoro-4-nitrobenzoate (9.50 g, 88% Yield) as an off white solid. LCMS (ESI)m/z 200 [M+H]+

### Step 2: methyl 3-[(3-methyloxetan-3-yl) amino]-4-nitrobenzoate

To a stirred solution of methyl 3-fluoro-4-nitrobenzoate (2.30 g, 11.5 mmol) in 1-methylpyrrolidin-2-one (20.0 mL) was added ethylbis(propan-2-yl) amine (6.04 mL, 3 eq., 34.6 mmol) and 3-methyloxetan-3-amine (1.01 g, 1 eq., 11.5 mmol) at room temperature. The reaction mixture was heated at 120° C. for 16 hours. The reaction mixture was quenched with water and extracted in ethyl acetate, washed with brine, dried over sodium sulfate and concentrated. The crude product was purified by flash chromatography to afford methyl 3-[(3-methyloxetan-3-yl) amino]-4-nitrobenzoate (1.30 g, 42.27% Yield) as a yellow solid. LCMS (ESI)m/z 267.1 [M+H]+

### Step 3: Methyl 4-amino-3-[(3-methyloxetan-3-yl) amino] benzoate

To a stirred solution of methyl 3-[(3-methyloxetan-3-yl) amino]-4-nitrobenzoate (1.30 g, 4.88 mmol) in methanol (20.0 mL) was added ammonium chloride (1.31 g, 5 eq., 24.4 mmol) followed by zinc (3.19 g, 10 eq., 48.8 mmol) at 0° C. The reaction mixture stirred at room temperature for 16 hours. The progress of the reaction mixture was monitored by TLC. After completion of the starting material, the reaction mixture was dissolved in water and extracted with ethyl acetate, washed with brine, dried over sodium sulfate and concentrated to afford methyl 4-amino-3-[(3-methyloxetan-3-yl) amino]benzoate (1.00 g, Crude) as a yellow color. The crude was taken in next step without purification. LCMS (ESI)m/z 237.1 [M+H]+

### Step 4: Methyl 2-[3,4-bis(benzyloxy)-2-fluoro-5-methoxyphenyl]-1-(3-methyloxetan-3-yl)-1H-1,3-benzodiazole-6-carboxylate

A mixture of solution of 3,4-bis(benzyloxy)-2-fluoro-5-methoxybenzaldehyde (100 mg, 0.273 mmol), methyl 4-amino-3-[(3-methyloxetan-3-yl) amino] benzoate (77.4 mg, 1.2 eq., 0.328 mmol) and sodium metabisulphite (77.8 mg, 1.5 eq., 0.409 mmol) in DMSO (5.00 mL) was heated at 80° C. for 3 hours. The reaction mixture was diluted with water, extracted with ethyl acetate, washed with brine, dried over anhydrous sodium sulphate and concentrated. The crude was purified by flash column chromatography with 45-50% ethyl acetate in hexane to afford methyl 2-[3,4-bis(benzyloxy)-2-fluoro-5-methoxyphenyl]-1-(3-methyloxetan-3-yl)-1H-1,3-benzodiazole-6-carboxylate (135 mg, 80% Yield) as yellow semi solid. LCMS (ESI)m/z 583.2[M+1]+

### Step 5: 2-[3,4-bis(benzyloxy)-2-fluoro-5-methoxyphenyl]-1-(3-methyloxetan-3-yl)-1H-1,3-benzodiazole-6-carboxylic acid

To a stirred solution of methyl 2-[3,4-bis(benzyloxy)-2-fluoro-5-methoxyphenyl]-1-(3-methyloxetan-3-yl)-1H-1,3-benzodiazole-6-carboxylate (180 mg, 0.309 mmol) in THF (10.0 mL) and methanol (10.0 mL) was added lithium hydrate hydroxide (38.9 mg, 3 eq., 0.927 mmol) in water (10.0 mL) at room temperature. The reaction mixture was heated at 60° C. for 3 hours. The reaction mixture was concentrated and then diluted with water and acidified with saturated citric acid solution, then extracted with ethyl acetate, dried over anhydrous sodium sulphate, and concentrated to afford 2-[3,4-bis(benzyloxy)-2-fluoro-5-methoxyphenyl]-1-(3-methyloxetan-3-yl)-1H-1,3-benzodiazole-6-carboxylic acid (150 mg, 82.83% Yield) as a pale brown solid. LCMS (ESI)m/z 569[M+1]+

### Step 1: 4-[6-(azetidine-1-carbonyl)-1-(3-methyloxetan-3-yl)-1H-1,3-benzodiazol-2-yl]-3-fluoro-6-methoxybenzene-1,2-diol

A suspension of 6-(azetidine-1-carbonyl)-2-[3,4-bis(benzyloxy)-2-fluoro-5-methoxyphenyl]-1-(3-methyloxetan-3-yl)-1H-1,3-benzodiazole (40.0 mg, 0.065 mmol) and 10% palladium hydroxide on carbon (100 mg, 0.702 mmol) in THF (20.0 mL) was stirred under hydrogen pressure (1 atm) for 2.5 hours at room temperature. The reaction mixture was filtered through celite, and the filtrate was concentrated. The crude was purified by preparative HPLC to afford 4-[6-(azetidine-1-carbonyl)-1-(3-methyloxetan-3-yl)-1H-1,3-benzodiazol-2-yl]-3-fluoro-6-methoxybenzene-1,2-diol (0.005 g, 18% Yield) as white solid. ¹H NMR (400 MHz, DMSO-d₆): δ ppm 2.02 (s, 3H), 2.24 (m, 2H), 3.77 (s, 3H), 4.05 (t, J=7.6 Hz, 2H), 4.31 (t, J=7.2 Hz, 2H), 4.41-4.40 (d, J=5.6 Hz, 2H), 4.73-4.71 (d, J=5.6 Hz, 2H), 6.59-6.57 (d, J=6 Hz, 1H), 7.37 (s, 1H), 7.50-7.52 (d, J=8 Hz, 1H), 7.71-7.73 (d, J=8.8 Hz, 1H), 9.5 (bs, 2H). LCMS (ESI)m/z 428.2 [M+H]+

### Example 178: Synthesis of 6-methoxy-4-[1-(2-methoxyethyl)-1H-1,3-benzodiazol-2-yl]-3-methylbenzene-1,2-diol; acetic acid

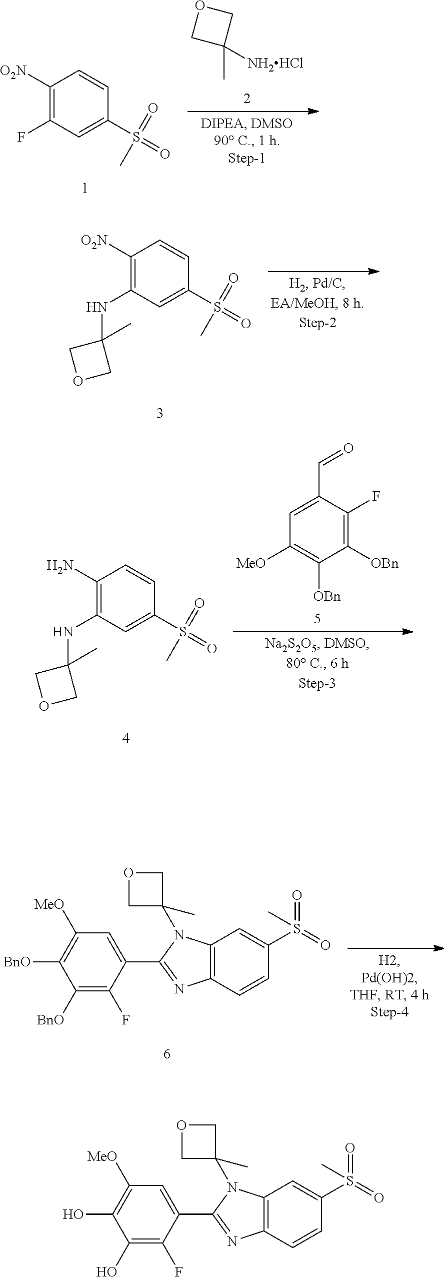

### Step 1: N-(5-methanesulfonyl-2-nitrophenyl)-3-methyloxetan-3-amine

To a stirred solution of 2-fluoro-4-methanesulfonyl-1-nitrobenzene (500 mg, 2.28 mmol) in DMSO (5 mL) was added ethyl bis(propan-2-yl) amine (1.20 mL, 3 eq., 6.84 mmol) and 3-methyloxetan-3-amine (401 μL, 2 eq., 4.56 mmol) at room temperature. The reaction mixture was heated at 90° C. for 1 hour. The reaction mixture was quenched with water and extracted in ethyl acetate, washed with brine, dried over sodium sulfate and concentrated to afford N-(5-methanesulfonyl-2-nitrophenyl)-3-methyloxetan-3-amine (420 mg, 64.31% Yield) as yellow solid. The crude product was taken as a next step without purification. LCMS (ESI)m/z 287.2[M+1]+

### Step 2: 5-methanesulfonyl-N1-(3-methyloxetan-3-yl) benzene-1,2-diamine

A mixture of N-(5-methanesulfonyl-2-nitrophenyl)-3-methyloxetan-3-amine (300 mg, 1.05 mmol) palladium (10% w/w, 112 mg, 0.105 mmol) in methanol (5.00 mL) and ethyl acetate (5.00 mL) was stirred under hydrogen pressure (1 atm) at room temperature for 8 hours. The progress of the reaction mixture was monitored by TLC. After completion of the starting material, reaction mixture was filtered and the filtrate was concentrated to afford 5-methanesulfonyl-N1-(3-methyloxetan-3-yl) benzene-1,2-diamine (270 mg, Crude) as a yellow liquid. LCMS (ESI)m/z 256.9[M+1]+

### Step 3: 2-[3,4-bis(benzyloxy)-2-fluoro-5-methoxyphenyl]-6-methanesulfonyl-1-(3-methyloxetan-3-yl)-1H-1,3-benzodiazole

A mixture of 5-methanesulfonyl-N1-(3-methyloxetan-3-yl) benzene-1,2-diamine (115 mg, 1.1 eq., 450 μmol), 3,4-bis(benzyloxy)-2-fluoro-5-methoxybenzaldehyde (150 mg, 409 μmol) and sodium metabisulphite(117 mg, 1.5 eq., 614 μmol) in DMSO (10 mL) was stirred at 80° C. for 5 hours. The reaction was monitored by TLC. After completion of the starting material, diluted with water and extracted in ethyl acetate, dried over sodium sulfate and concentrated. The crude was purified by flash chromatography to afford 2-[3,4-bis(benzyloxy)-2-fluoro-5-methoxyphenyl]-6-methanesulfonyl-1-(3-methyloxetan-3-yl)-1H-1,3-benzodiazole (70.0 mg, 28.37% Yield) as a black liquid. LCMS (ESI)m/z 603.3 [M+H]+

### Step 4: 3-fluoro-4-[6-methanesulfonyl-1-(3-methyloxetan-3-yl)-1H-1,3-benzodiazol-2-yl]-6-methoxybenzene-1,2-diol

A suspension of 2-[3,4-bis(benzyloxy)-2-fluoro-5-methoxyphenyl]-6-methanesulfonyl-1-(3-methyloxetan-3-yl)-1H-1,3-benzodiazole (70.0 mg, 116 μmol) and palladium(2+) hydroxide 20% w/w (711 μg, 0.01 eq., 1.16 μmol) in THF (10 mL) was stirred under hydrogen pressure (1 atm) for 8 hours. The progress of the reaction mixture monitored by TLC and LCMS. The reaction mixture was filtered, and the filtrate was concentrated. The crude compound was purified by preparative HPLC to afford 3-fluoro-4-[6-methanesulfonyl-1-(3-methyloxetan-3-yl)-1H-1,3-benzodiazol-2-yl]-6-methoxybenzene-1,2-diol (22.0 mg, 44.84% Yield) as a white solid. ¹H NMR (400 MHz, DMSO-d₆): δ ppm 2.08 (s, 3H), 3.30 (s, 3H), 3.80 (s, 3H), 4.43 (d, J=6.4 Hz, 2H), 4.79 (d, J=6 Hz, 2H), 6.65 (d, J=6 Hz, 1H), 7.73 (s, 1H), 7.85-7.82 (m, 1H), 7.48 (d, J=8.4 Hz, 1H), 9.52(bs, 2H). LCMS (ESI)m/z 423.1 [M+H]+ Melting point: 257.6° C.

### Example 179: Synthesis of 6-methoxy-4-[1-(2-methoxyethyl)-1H-1,3-benzodiazol-2-yl]-3-methylbenzene-1,2-diol; acetic acid

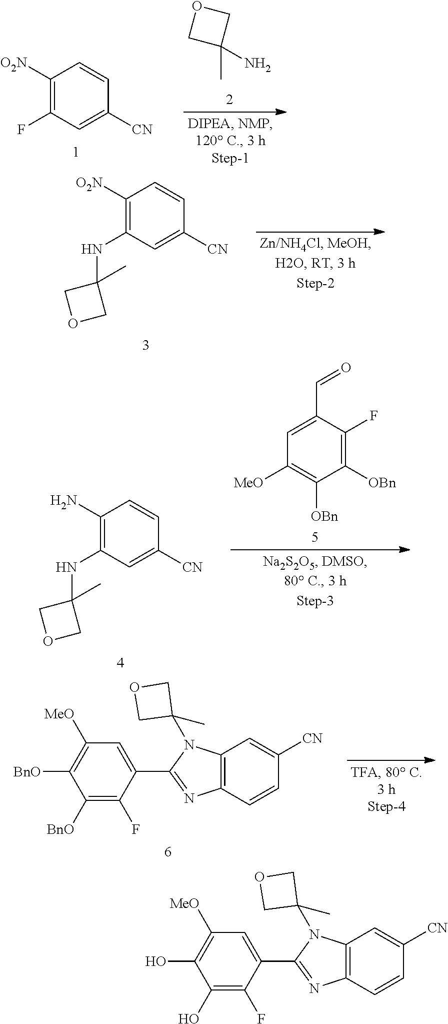

### Step 1: Methyl 3-[(3-methyloxetan-3-yl)amino]-4-nitrobenzoate

To a stirred solution of 3-fluoro-4-nitrobenzonitrile (500 mg, 3.01 mmol) in 1-methylpyrrolidin-2-one (2.00 mL) was added ethylbis(propan-2-yl) amine (1.57 mL, 3 eq., 9.03 mmol) and 3-methyloxetan-3-amine (265 μL, 3.01 mmol) at room temperature. The reaction mixture was heated at 120° C. for 16 hours. The reaction mixture was quenched with water and extracted in ethyl acetate, dried over sodium sulfate and concentrated. The crude product was purified by flash chromatography to afford 3-[(3-methyloxetan-3-yl) amino]-4-nitrobenzonitrile (290 mg, 41% Yield) as a yellow solid. ¹H NMR (400 MHz, DMSO-d₆): δ ppm 1.65 (s, 1H), 4.58-4.57 (d, J=6 Hz, 2H), 4.66-4.64 (d, J=6.4 Hz, 2 H), 6.76 (s, 1H), 7.08-7.06 (d, J=9.6 Hz, 1H), 8.21-8.19 (d, J=8.8 Hz, 1H), 8.4 (s, 1H),

### Step 2: 4-amino-3-[(3-methyloxetan-3-yl)amino]benzonitrile

To a stirred solution of 3-[(3-methyloxetan-3-yl)amino]-4-nitrobenzonitrile (200 mg, 858 μmol) in methanol (10 mL) was added ammonium chloride (229 mg, 5 eq., 4.29 mmol) followed by zinc (392 mg, 7 eq., 6.00 mmol) at 0° C. and stirred at room temperature for 16 hours. The progress of the reaction mixture was monitored by TLC. After completion of the starting material, the reaction mixture was dissolved in water and extracted in ethyl acetate, washed with brine, dried over sodium sulfate and concentrated to afford 4-amino-3-[(3-methyloxetan-3-yl)amino] benzonitrile (170 mg, Crude) as a purple solid. The crude was taken in next step without purification. LCMS (ESI)m/z 204.1 [M+H]+

### Step 3: 2-[3,4-bis(benzyloxy)-2-fluoro-5-methoxyphenyl]-1-(3-methyloxetan-3-yl)-1H-1,3-benzodiazole-6-carbonitrile

A mixture of 4-amino-3-[(3-methyloxetan-3-yl)amino]benzonitrile (110 mg, 1.2 eq., 541 μmol), 3,4-bis(benzyloxy)-2-fluoro-5-methoxybenzaldehyde (160 mg, 437 μmol) and sodium metabisulphite (125 mg, 1.5 eq., 655 μmol) in DMSO (5 mL) was stirred 80° C. for 2 hours. The progress of the reaction mixture was monitored by TLC. After completion of the starting material, the reaction mixture was dissolved in water and extracted in ethyl acetate, washed with brine, dried over sodium sulfate and concentrated. The crude was purified by flash chromatography to afford 2-[3,4-bis(benzyloxy)-2-fluoro-5-methoxyphenyl]-1-(3-methyloxetan-3-yl)-1H-1,3-benzodiazole-6-carbonitrile (80.0 mg, crude) as a white solid. LCMS (ESI)m/z 549.9[M+H]+

### Step 4: 2-(2-fluoro-3,4-dihydroxy-5-methoxyphenyl)-1-(3-methyloxetan-3-yl)-1H-1,3-benzodiazole-6-carbonitrile

To a stirred solution of 2-[3,4-bis(benzyloxy)-2-fluoro-5-methoxyphenyl]-1-(3-methyloxetan-3-yl)-1H-1,3-benzodiazole-6-carbonitrile (60.0 mg, 109 μmol) in TFA was stirred at 80° C. for 2 hours. The progress of the reaction mixture monitored by TLC and LCMS. After completion of the starting material, the mixture was concentrated. The crude was purified by following preparative HPLC to afford 2-(2-fluoro-3,4-dihydroxy-5-methoxyphenyl)-1-(3-methyloxetan-3-yl)-1H-1,3-benzodiazole-6-carbonitrile (15.0 mg, 37.2% Yield) as a white solid. ¹H NMR (400 MHz, DMSO-d₆): δ ppm 2.04 (s, 3H), 3.74 (s, 3H), 4.42 (d, J=6 Hz, 2H), 4.51 (d, J=6 Hz, 2H), 6.61 (d, J=6 Hz, 1H), 7.67-7.64(m, 1H), 7.85 (d, J=8.4 Hz, 1H), 9.34 (s, 1H), 9.51(sb, 2H). LCMS (ESI)m/z 370.1[M+H]+ Melting point: 209° C.

### Example 180: Synthesis of 6-methoxy-4-[1-(2-methoxyethyl)-1H-1,3-benzodiazol-2-yl]-3-methylbenzene-1,2-diol

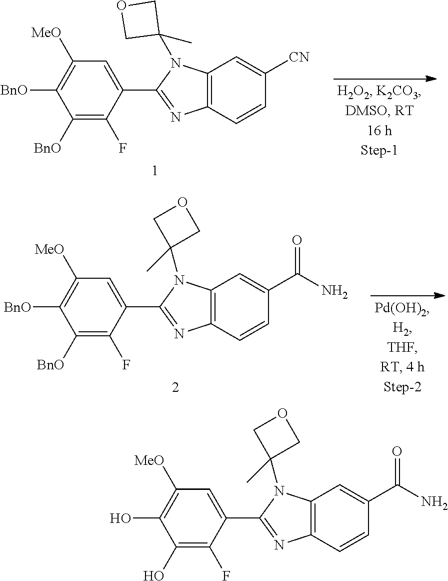 [SMILES: COc1cc(-c2nc3ccc(C#N)cc3n2C2(C)COC2)c(F)c(OCc2ccccc2)c1OCc1ccccc1 ; COc1cc(-c2nc3ccc(C(N)=O)cc3n2C2(C)COC2)c(F)c(OCc2ccccc2)c1OCc1ccccc1 ; COc1cc(-c2nc3ccc(C(N)=O)cc3n2C2(C)COC2)c(F)c(O)c1O]

### Step 1: 2-[3,4-bis(benzyloxy)-2-fluoro-5-methoxyphenyl]-1-(3-methyloxetan-3-yl)-1H-1,3-benzodiazole-6-carboxamide

To a stirred solution of 2-[3,4-bis(benzyloxy)-2-fluoro-5-methoxyphenyl]-1-(3-methyloxetan-3-yl)-1H-1,3-benzodiazole-6-carbonitrile (150 mg, 273 μmol) in DMSO (5.00 mL) was added hydrogen peroxide (8.19 μL, 1.5 eq., 409 μmol) followed by dipotassium carbonate at 0° C. Then the mixture was stirred at room temperature for 16 hours. The progress of the reaction mixture was monitored by TLC. After completion of the starting material, the reaction mixture was poured into chilled water; the precipitated solids were filtered and dried to afford 2-[3,4-bis(benzyloxy)-2-fluoro-5-methoxyphenyl]-1-(3-methyloxetan-3-yl)-1H-1,3-benzodiazole-6-carboxamide (120 mg, 77.465 Yield) as a white solid. LCMS (ESI)m/z 568.6[M+H]+.

### Step 2: 2-(2-fluoro-3,4-dihydroxy-5-methoxyphenyl)-1-(3-methyloxetan-3-yl)-1H-1,3-benzodiazole-6-carboxamide

A suspension of 2-[3,4-bis(benzyloxy)-2-fluoro-5-methoxyphenyl]-1-(3-methyloxetan-3-yl)-1H-1,3-benzodiazole-6-carboxamide (120 mg, 211 μmol) and palladium hydroxide (80.0 mg, 755 μmol) in THF (20.0 mL) was stirred under hydrogen pressure (1 atm) for 2.5 hours. Reaction mixture was filtered through celite. and the filtrate was concentrated. The crude was purified by preparative HPLC to afford 2-(2-fluoro-3,4-dihydroxy-5-methoxyphenyl)-1-(3-methyloxetan-3-yl)-1H-1,3-benzodiazole-6-carboxamide (35.0 mg, 43% Yield) as an off white solid. ¹H NMR (400 MHz, CD30D-d₆): 6 ppm 2.04 (s, 3H), 3.77 (s, 3H), 4.40 (s, 2H), 4.71 (s, 2H), 6.60 (d, J=8 Hz, 1H), 7.37 (s, 1H), 7.70 (d, J=8 Hz, 2H), 7.83 (d, J=8 Hz, 1H), 8.07 (s, 1H), 9.43 (s, 1H), 9.50 (s, 1H). LCMS (ESI)m/z 387.4[M+H]+ Melting Point: 170.4° C.

### Example 181: Synthesis of 6-methoxy-4-[1-(2-methoxyethyl)-1H-1,3-benzodiazol-2-yl]-3-methylbenzene-1,2-diol; acetic acid

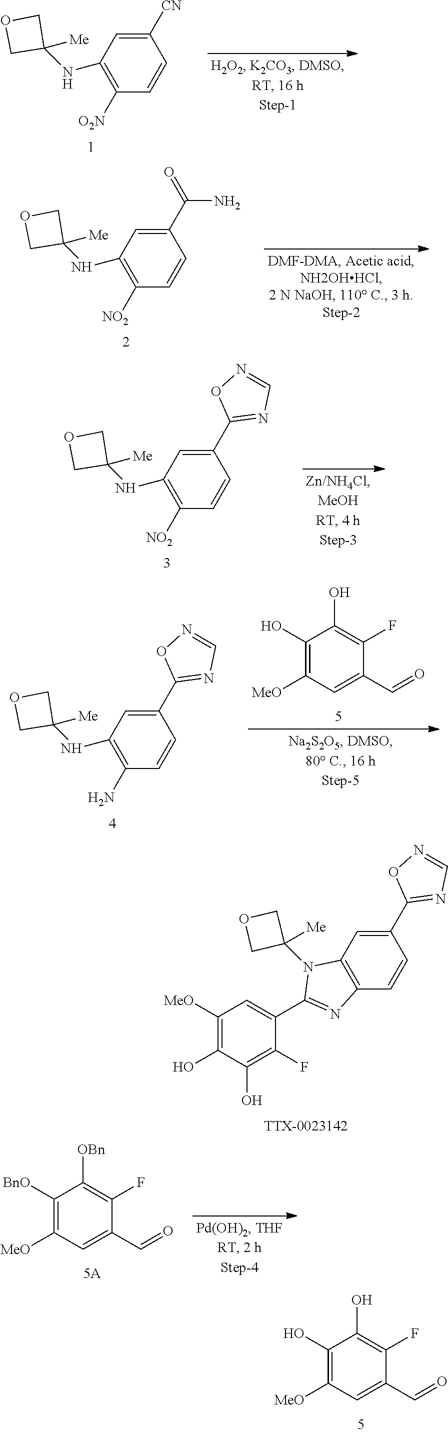

### Step 1: 3-[(3-methyloxetan-3-yl)amino]-4-nitrobenzamide

To a suspension of 3-[(3-methyloxetan-3-yl)amino]-4-nitrobenzonitrile (550 mg, 2.36 mmol) in DMSO (2.00 mL) was added dipotassium carbonate (978 mg, 3 eq., 7.07 mmol) and hydrogen peroxides (70.7 μL, 1.5 eq., 3.54 mmol) at 0° C. The reaction mixture was stirred at room temperature for 16 hours. Reaction progress was monitored by LC-MS and TLC. After completion of starting material, reaction mass was quenched with water and extracted with ethyl acetate (2×100 ml). Combined organic layer was dried over sodium sulfate and concentrated. The crude compound was purified by flash chromatography to afford 3-[(3-methyloxetan-3-yl)amino]-4-nitrobenzamide (450 mg, 76% Yield) as a yellow solid. LCMS (ESI)m/z 252.2 [M+H]+

### Step 2: 3-methyl-N-[2-nitro-5-(1,2,4-oxadiazol-5-yl) phenyl]oxetan-3-amine

A mixture of 3-[(3-methyloxetan-3-yl) amino]-4-nitrobenzamide (450 mg, 1.79 mmol) and dimethylformamide dimethylacetal (5.00 mL) was stirred at 110° C. for 1 hour. The reaction mixture was concentrated and the obtained residue was dissolved in 1,4-dioxane (5.00 mL). Then acetic acid (102 μL, 1.79 mmol), hydroxyazanium chloride (249 mg, 2 eq., 3.58 mmol) and 2 N NaOH Sol (3.00 mL) were added at room temperature. The mixture was stirred at 90° C. for 30 min. The reaction mixture was concentrated, diluted with water, and extracted with dichloromethane, dried over sodium sulfate and concentrated. The crude was purified by flash chromatography to afford 3-methyl-N-[2-nitro-5-(1,2,4-oxadiazol-5-yl) phenyl]oxetan-3-amine (219 mg, 52% Yield) as a pale-yellow solid. LCMS (ESI)m/z 277.3 [M+H]+

### Step 3: N1-(3-methyloxetan-3-yl)-5-(1,2,4-oxadiazol-5-yl) benzene-1,2-diamine

To a suspension of 3-methyl-N-[2-nitro-5-(1,2,4-oxadiazol-5-yl) phenyl]oxetan-3-amine (350 mg, 1.27 mmol) in THF (4.00 mL) and water (1.00 mL) was added ammonium chloride (339 mg, 5 eq., 6.33 mmol) followed by zinc (249 mg, 3 eq., 3.80 mmol) at 0° C. and reaction mixture was stirred for 4 hours at room temperature. Reaction progress monitored by TLC and LC-MS. Reaction mass was quenched with water and extracted with ethyl acetate (2×25 ml). dried over sodium sulfate and concentrated to afford N1-(3-methyloxetan-3-yl)-5-(1,2,4-oxadiazol-5-yl) benzene-1,2-diamine (350 mg, crude). The crude compound was taken for next step without purification. LCMS (ESI)m/z 247.1 [M+H]+

### Step 4: 2-fluoro-3,4-dihydroxy-5-methoxybenzaldehyde

A suspension of 3,4-bis(benzyloxy)-2-fluoro-5-methoxybenzaldehyde (100 mg, 273 μmol) and palladium hydroxide (57.9 mg, 2 eq., 546 μmol) in THF (5.00 mL) was stirred under hydrogen pressure (1 atm) for 2 hours. The reaction mixture was filtered, and the filtrate was concentrated to afford 2-fluoro-3,4-dihydroxy-5-methoxybenzaldehyde (45 mg, crude) LCMS (ESI)m/z 187.1 [M+H]+

### Step 5: 3-fluoro-6-methoxy-4-[1-(3-methyloxetan-3-yl)-6-(1,2,4-oxadiazol-5-yl)-1H-1,3-benzodiazol-2-yl] benzene-1,2-diol

A mixture of N1-(3-methyloxetan-3-yl)-5-(1,2,4-oxadiazol-5-yl) benzene-1,2-diamine (40.0 mg, 162 μmol), 2-fluoro-3,4-dihydroxy-5-methoxybenzaldehyde (45.3 mg, 1.5 eq., 244 μmol) and sodium metabisulphite (46.3 mg, 1.5 eq., 244 μmol) in DMSO (5.00 mL) was stirred 80° C. for 12 hours. The progress of the reaction mixture was monitored by TLC. After completion of the starting material, the reaction mixture was dissolved in water and extracted in ethyl acetate (2×20 ml), dried over sodium sulfate and concentrated. The crude was purified by preparative HPLC to afford 3-fluoro-6-methoxy-4-[1-(3-methyloxetan-3-yl)-6-(1,2,4-oxadiazol-5-yl)-1H-1,3-benzodiazol-2-yl] benzene-1,2-diol (22.0 mg, 32% Yield) as an off white solid. ¹H NMR (400 MHz, DMSO-d₆): δ ppm 2.08 (bs, 3H), 3.78 (bs, 3H), 4.37 (d, J=6 Hz, 2H), 4.78 (d, J=5.6 Hz, 2H), 6.64 (d, J=6.4 Hz, 1H), 7.92 (d, J=8.8 Hz, 2H), 8.04 (d, J=8.4 Hz, 1H), 9.09 (bs, 1H), 9.51 (bs, 1H). LCMS (ESI)m/z 412.4 [M+H]+.

### Example 182: Synthesis of 6-methoxy-4-[1-(2-methoxyethyl)-1H-1,3-benzodiazol-2-yl]-3-methylbenzene-1,2-diol

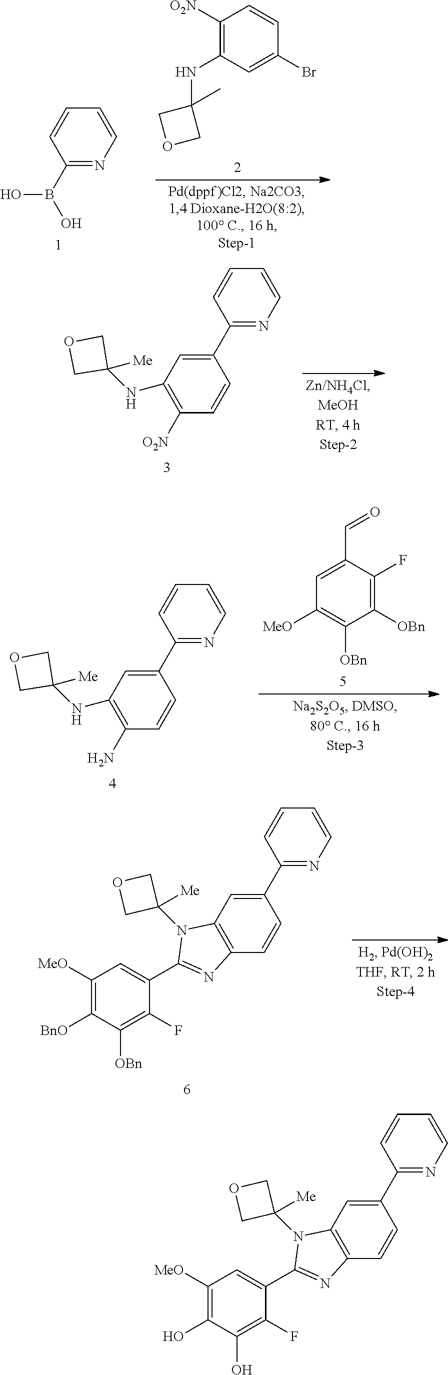

### Step 1: 3-methyl-N-[2-nitro-5-(pyridin-2-yl)phenyl]oxetan-3-amine

To a suspension of N-(5-bromo-2-nitrophenyl)-3-methyloxetan-3-amine (500 mg, 1.74 mmol) in 1,4-dioxane (8.00 mL) and water (2.00 mL) was added (pyridin-2-yl) boronic acid (321 mg, 1.5 eq., 2.61 mmol) followed by addition of disodium carbonate (369 mg, 2 eq., 3.48 mmol) was degassed with argon for 10 minutes at room temperature. Then (1,1′-Bis (diphenylphosphino) ferrocene) palladium (II) dichloride (255 mg, 0.2 eq., 348 μmol) was added and stirred for 16 hours at 100° C. The reaction mixture was monitored by TLC (50% ethyl acetate in hexane). After completion of the reaction, the reaction mixture was quenched with water and extracted with ethyl acetate, washed with water & brine dried over sodium sulfate and concentrated. The crude was purified by flash column chromatography with 30-35% ethyl acetate in hexane to afford 3-methyl-N-[2-nitro-5-(pyridin-2-yl) phenyl]oxetan-3-amine (290.0 mg, 58% Yield) LCMS (ESI)m/z 586 [M+H]+.

### Step 2: N1-(3-methyloxetan-3-yl)-5-(pyridin-2-yl) benzene-1,2-diamine

To a stirred solution of 3-methyl-N-[2-nitro-5-(pyridin-2-yl)phenyl]oxetan-3-amine (300 mg, 1.05 mmol) in THF (8.00 L) and water (2.00 L) was added ammonium chloride (56.2 mg, 1 eq., 1.05 mmol) and zinc Dust (68.8 mg, 1.05 mmol). The reaction mixture was stirred at room temperature for 4 hours. The reaction progress was monitored by TLC & LCMS. After completion of the reaction, the reaction mixture was quenched with water and extracted with ethyl acetate, washed with water & brine, dried over sodium sulfate and concentrated to afford N1-(3-methyloxetan-3-yl)-5-(pyridin-2-yl) benzene-1,2-diamine (250 mg, crude) LCMS (ESI)m/z256.2 [M+H]+

### Step 3: 2-[3,4-bis(benzyloxy)-2-fluoro-5-methoxyphenyl]-1-(3-methyloxetan-3-yl)-6-(pyridin-2-yl)-1H-1,3-benzodiazole

To a suspension of N1-(3-methyloxetan-3-yl)-5-(pyridin-2-yl) benzene-1,2-diamine (94.1 mg, 368 μmol), 3,4-bis(benzyloxy)-2-fluoro-5-methoxybenzaldehyde (135 mg, 1 eq., 368 μmol), sodium metabisulphite (70.0 mg, 368 μmol) in dimethylsulfoxide (5 mL) and stirred at 80° C. for 16 h. The progress of the reaction mixture was monitored by TLC. After completion of the starting material, the reaction mixture was dissolved in water and extracted in ethyl acetate (2×20 ml), dried over sodium sulfate and concentrated. The crude was purified by flash chromatography to afford 2-[3,4-bis(benzyloxy)-2-fluoro-5-methoxyphenyl]-1-(3-methyloxetan-3-yl)-6-(pyridin-2-yl)-1H-1,3-benzodiazole (95.0 mg, 42% Yield) as an off white solid. LCMS (ESI)m/z 602.3 [M+H]+

### Step 4: 3-fluoro-6-methoxy-4-[1-(3-methyloxetan-3-yl)-6-(pyridin-2-yl)-1H-1,3-benzodiazol-2-yl]benzene-1,2-diol

A suspension of 2-[3,4-bis(benzyloxy)-2-fluoro-5-methoxyphenyl]-1-(3-methyloxetan-3-yl)-6-(pyridin-2-yl)-1H-1,3-benzodiazole (95.0 mg, 158 μmol) and palladium(2+) hydroxide (38.7 mg, 2 eq., 316 μmol) in THF (20.0 mL) was stirred under hydrogen pressure (1 atm) for 3 hours. The reaction mixture was filtered, and the filtrate was concentrated. The crude compound was purified by preparative HPLC to afford 3-fluoro-6-methoxy-4-[1-(3-methyloxetan-3-yl)-6-(pyridin-2-yl)-1H-1,3-benzodiazol-2-yl]benzene-1,2-diol (3.00 mg, 4% Yield) as a grey solid. ¹H NMR (400 MHz, CD3OD-d₆): 6 ppm 2.04 (s, 3H), 3.77 (s, 3H), 4.40 (s, 2H), 4.71 (s, 2H), 6.60 (d, J=8 Hz, 1H), 7.37 (s, 1H), 7.70 (d, J=8 Hz, 2 H), 7.83 (d, J=8 Hz, 1H), 8.07 (s, 1H), 9.43 (s, 1H), 9.50 (s, 1H). LCMS (ESI)m/z 421.4 [M+H]⁺

### Example 183: Synthesis of 6-methoxy-4-[1-(2-methoxyethyl)-1H-1,3-benzodiazol-2-yl]-3-methylbenzene-1,2-diol

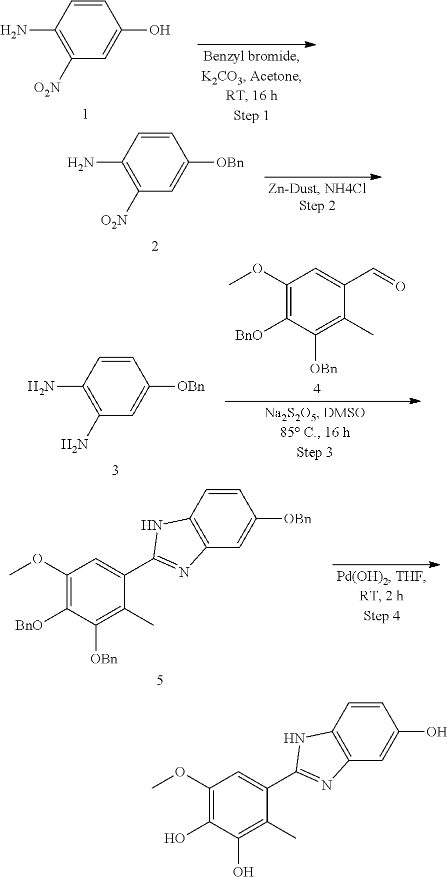 [SMILES: Nc1ccc(O)cc1[N+](=O)[O-] ; Nc1ccc(OCc2ccccc2)cc1[N+](=O)[O-] ; Nc1ccc(OCc2ccccc2)cc1N ; COc1cc(C=O)c(C)c(OCc2ccccc2)c1OCc1ccccc1 ; COc1cc(-c2nc3cc(OCc4ccccc4)ccc3[nH]2)c(C)c(OCc2ccccc2)c1OCc1ccccc1 ; COc1cc(-c2nc3cc(O)ccc3[nH]2)c(C)c(O)c1O]

### Step 1: 4-(benzyloxy)-2-nitroaniline

To a stirred solution of 4-amino-3-nitrophenol (750 mg, 4.87 mmol) in acetone (15.0 mL) was added dipotassium carbonate (560 μL, 1.2 eq., 5.84 mmol) followed by (bromomethyl)benzene (832 mg, 4.87 mmol) at 0° C. The mixture was stirred for 16 hours at room temperature. After completion of the reaction, the solvent was removed, and diluted with ethyl acetate, washed with water and brine, dried over sodium sulfate and concentrated. The crude was purified by flash column chromatography with 10-15% ethyl acetate in hexane to afford 4-(benzyloxy)-2-nitroaniline (700 mg, 58% Yield). LCMS (ESI)m/z 245 [M+H]+.

### Step 2: 4-(benzyloxy)benzene-1,2-diamine

To a stirred solution of 4-(benzyloxy)-2-nitroaniline (400 mg, 1.64 mmol) in acetic acid (5.00 mL) was added ammonium chloride (2.00 mL) and zinc (375 mg, 3.5 eq., 5.73 mmol) portion wise at 0° C. The reaction mixture was stirred for 16 hours. The solvent was evaporated. The residue was diluted with water and 5 M NaOH was added to basify up to pH˜10. The insoluble material was removed by filtration through a pad of celite. The filtrate was extracted with DCM. The combined organic layers were washed with water and brine, dried over sodium sulfate and concentrated. The crude was purified by flash column chromatography with at 20-25% ethyl acetate in hexane to afford 4-(benzyloxy)benzene-1,2-diamine (180 mg, 51% Yield) as a red solid. LCMS (ESI)m/z 215 [M+H]+.

### Step 3: 5-(benzyloxy)-2-[3,4-bis(benzyloxy)-5-methoxy-2-methylphenyl]-1H-1,3-benzodiazole

A mixture of 3,4-bis(benzyloxy)-5-methoxy-2-methylbenzaldehyde (200 mg, 552 μmol), 4-(benzyloxy)benzene-1,2-diamine (177 mg, 1.5 eq., 828 μmol) and sodium metabisulphite(157 mg, 1.5 eq., 828 μmol) in DMSO (3.00 mL) was stirred at 85° C. for 12 hours. The reaction mixture was diluted with water (15 mL), extracted with ethyl acetate (2×20 mL), dried over sodium sulphate and concentrated. The crude was purified by flash column chromatography with 20-25% ethyl acetate in hexane to afford 5-(benzyloxy)-2-[3,4-bis(benzyloxy)-5-methoxy-2-methylphenyl]-1H-1,3-benzodiazole (200 mg, 65% Yield) as a yellow solid. LCMS (ESI)m/z 557 [M+H]+.

### Step 4: 4-(5-hydroxy-1H-1,3-benzodiazol-2-yl)-6-methoxy-3-methylbenzene-1,2-diol; acetic acid

A suspension of 5-(benzyloxy)-2-[3,4-bis(benzyloxy)-5-methoxy-2-methylphenyl]-1H-1,3-benzodiazole (200 mg, 0.359 mmol) in THF (15.0 mL) and palladium hydroxide (100 mg, 702 μmol) was stirred under hydrogen pressure (1 atm) for 3 hours. The reaction mixture was filtered, and the filtrate was concentrated. The crude compound was purified by preparative HPLC to afford 4-(5-hydroxy-1H-1,3-benzodiazol-2-yl)-6-methoxy-3-methylbenzene-1,2-diol; acetic acid (52.0 mg, 41.7% Yield) as an off white solid. ¹H NMR (400 MHz, DMSO-d₆): δ ppm 1.93 (s, 3H), 2.34 (s, 3H), 3.82 (s, 3 H), 6.67 (m, J=3.6 Hz, 1H), 6.80-6.82 (d, J=5.6 Hz, 2H), 7.38-7.40 (d, J=8.4 Hz, 1H), 8.90 (s, 1H), 9.10 (bs, 2H), 11.97 (s, 1H). LCMS (ESI)m/z 287.2 [M+H]+.

### Example 184: Synthesis of 6-methoxy-4-[1-(2-methoxyethyl)-1H-1,3-benzodiazol-2-yl]-3-methylbenzene-1,2-diol

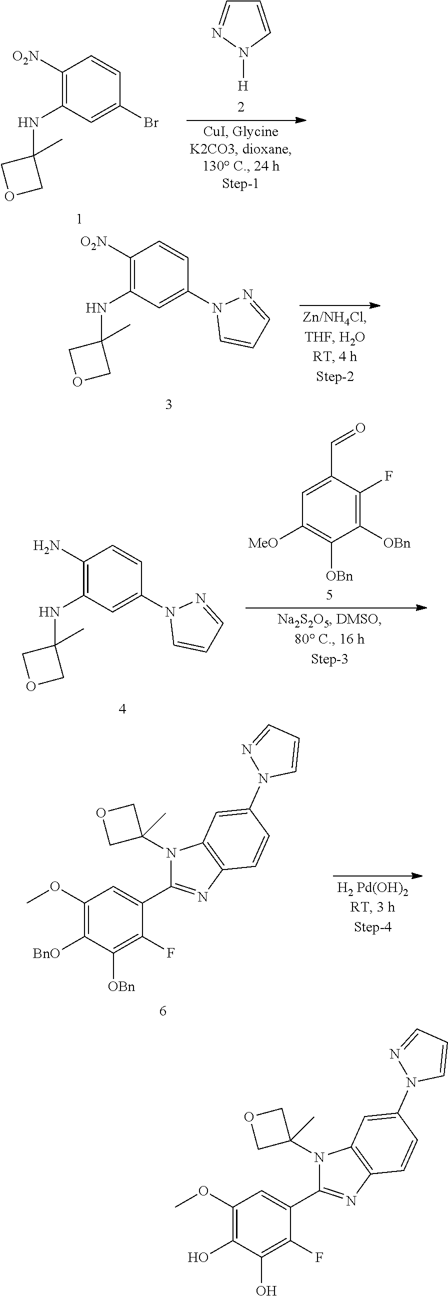

### Step 1: 3-methyl-N-[2-nitro-5-(1H-pyrazol-1-yl)phenyl]oxetan-3-amine

To a suspension of N-(5-bromo-2-nitrophenyl)-3-methyloxetan-3-amine (400 mg, 1.39 mmol), 1H-pyrazole (190 mg, 2 eq., 2.79 mmol) and diiodocopper (44.2 mg, 0.1 eq., 139 μmol) in 1,4-dioxane (4.00 mL) was degassed with nitrogen gas for 10 min. Then 2-aminoacetic acid (10.5 mg, 0.1 eq., 139 μmol), dipotassium carbonate (578 mg, 3 eq., 4.18 mmol) was added and reaction mixture was stirred for 48 hours at 140° C. Reaction mass was quenched with water and extracted with ethyl acetate (2×50 ml), dried over sodium sulfate and concentrated. The crude was purified by flash chromatography to afford, 3-methyl-N-[2-nitro-5-(1H-pyrazol-1-yl)phenyl]oxetan-3-amine (280 mg, 73% Yield) as yellow solid. LCMS (ESI)m/z 275.1 [M+H]+

### Step 2: N1-(3-methyloxetan-3-yl)-5-(1H-pyrazol-1-yl)benzene-1,2-diamine

To a suspension of 3-methyl-N-[2-nitro-5-(1H-pyrazol-1-yl)phenyl]oxetan-3-amine (230 mg, 839 μmol) in THF (8.00 mL) and water (2.00 mL) was added ammonium chloride (44.9 mg, 839 μmol) followed by zinc dust (54.9 mg, 839 μmol) at 0° C. The reaction mixture was stirred at room temperature for 4 hours. Reaction progress monitored by TLC and LC-MS. After completion of reaction mass was diluted with ethyl acetate and filtered on celite. The filtrate was extracted with ethyl acetate (2×100 ml), dried over sodium sulfate and concentrated to afford N1-(3-methyloxetan-3-yl)-5-(1H-pyrazol-1-yl)benzene-1,2-diamine (90.0 mg, Crude) as a brown solid. LCMS (ESI)m/z 244.9 [M+H]+.

### Step 3: 2-[3,4-bis(benzyloxy)-2-fluoro-5-methoxyphenyl]-1-(3-methyloxetan-3-yl)-6-(1H-pyrazol-1-yl)-1H-1,3-benzodiazole

To a suspension of N1-(3-methyloxetan-3-yl)-5-(1H-pyrazol-1-yl)benzene-1,2-diamine (90.0 mg, 368 μmol), 3,4-bis(benzyloxy)-2-fluoro-5-methoxybenzaldehyde (135 mg, 1 eq., 368 μmol), sodium metabisulphite (70.0 mg, 368 μmol) in dimethylsulfoxide (5 mL) was stirred at 80° C. for 16 hours. The reaction mass was quenched with water and extracted with ethyl acetate (2×100 ml), dried over sodium sulfate and concentrated. The crude was purified by column chromatography with 50% ethyl acetate in hexane to afford 2-[3,4-bis(benzyloxy)-2-fluoro-5-methoxyphenyl]-1-(3-methyloxetan-3-yl)-6-(1H-pyrazol-1-yl)-1H-1,3-benzodiazole (90.0 mg, 41% Yield) as an off white solid. LCMS (ESI)m/z 591.2 [M+H]+.

### Step 4: 3-fluoro-6-methoxy-4-[1-(3-methyloxetan-3-yl)-6-(1H-pyrazol-1-yl)-1H-1,3-benzodiazol-2-yl]benzene-1,2-diol

A suspension of 2-[3,4-bis(benzyloxy)-2-fluoro-5-methoxyphenyl]-1-(3-methyloxetan-3-yl)-6-(1H-pyrazol-1-yl)-1H-1,3-benzodiazole (90.0 mg, 152 μmol) and palladium hydroxide (70.0 mg, 660 μmol) in THF(15.0 mL) was stirred under hydrogen pressure (1 atm) for 3 hours. The reaction mixture was filtered, and the filtrate was concentrated. The crude compound was purified by preparative HPLC to afford 3-fluoro-6-methoxy-4-[1-(3-methyloxetan-3-yl)-6-(1H-pyrazol-1-yl)-1H-1,3-benzodiazol-2-yl]benzene-1,2-diol (17.0 mg, 27% Yield) as an off white solid. ¹H NMR (400 MHz, DMSO-d₆): δ ppm 2.06 (s, 3H), 3.81 (bs, 3H), 4.45 (d, J=4 Hz, 2H), 4.78 (d, J=4 Hz, 2H), 7.55 (s, 1H), 7.76 (s, 1H), 7.80 (s, 2H), 8.60 (s, 1H), 9.46-9.41 (m, 2H). LCMS (ESI)m/z 411.2 [M+H]+.

### Example 185: Synthesis of 6-methoxy-4-[1-(2-methoxyethyl)-1H-1,3-benzodiazol-2-yl]-3-methylbenzene-1,2-diol

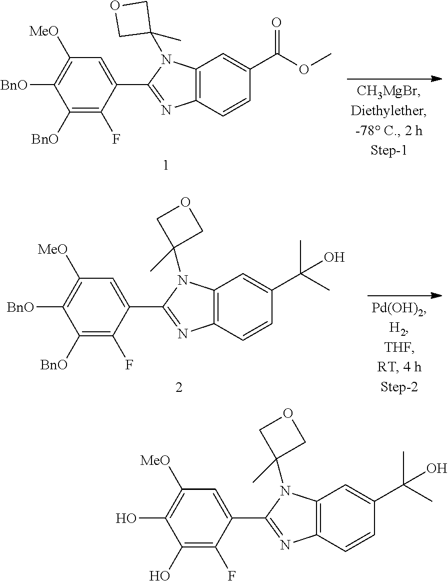 [SMILES: COC(=O)c1ccc2nc(-c3cc(OC)c(OCc4ccccc4)c(OCc4ccccc4)c3F)n(C3(C)COC3)c2c1 ; COc1cc(-c2nc3ccc(C(C)(C)O)cc3n2C2(C)COC2)c(F)c(OCc2ccccc2)c1OCc1ccccc1 ; COc1cc(-c2nc3ccc(C(C)(C)O)cc3n2C2(C)COC2)c(F)c(O)c1O]

### Step 1: 2-{2-[3,4-bis(benzyloxy)-2-fluoro-5-methoxyphenyl]-1-(3-methyloxetan-3-yl)-1H-1,3-benzodiazol-6-yl}propan-2-ol

To a stirred solution of methyl 2-[3,4-bis(benzyloxy)-2-fluoro-5-methoxyphenyl]-1-(3-methyloxetan-3-yl)-1H-1,3-benzodiazole-6-carboxylate (150 mg, 257 μmol) in diethyl ether (3.00 mL) was added CH3MgBr (257 μL, 2 eq., 515 μmol) at −78° C. under nitrogen atmosphere. The reaction mixture was stirred at room temperature for 12 hours. The reaction mixture was monitored by TLC (50% of Ethyl acetate in hexane). After completion of the reaction, reaction mixture was quenched with aqueous NH4C₁ solution dropwise and then extracted with ethyl acetate, washed with brine, dried with sodium sulphate and concentrated. to afford 2-{2-[3,4-bis(benzyloxy)-2-fluoro-5-methoxyphenyl]-1-(3-methyloxetan-3-yl)-1H-1,3-benzodiazol-6-yl}propan-2-ol (50.0 mg, Crude) as a color less oil. LCMS (ESI)m/z 583 [M+H]+

### Step 2: 3-fluoro-4-[6-(2-hydroxypropan-2-yl)-1-(3-methyloxetan-3-yl)-1H-1,3-benzodiazol-2-yl]-6-methoxybenzene-1,2-diol

A suspension of 2-{2-[3,4-bis(benzyloxy)-2-fluoro-5-methoxyphenyl]-1-(3-methyloxetan-3-yl)-1H-1,3-benzodiazol-6-yl}propan-2-ol (100 mg, 172 μmol) and palladium(2+) hydroxide (30.0 mg, 245 μmol) in THF (3.00 mL), was stirred under hydrogen pressure (1 atm) for 3 hours, After completion of the reaction, the reaction mixture was filtered through celite bed, and the filtrate was concentrated. The crude was purified by preparative HPLC to afford 3-fluoro-4-[6-(2-hydroxypropan-2-yl)-1-(3-methyloxetan-3-yl)-1H-1,3-benzodiazol-2-yl]-6-methoxy benzene-1,2-diol (0.015 g, 22% Yield) as white solid. ¹H NMR (400 MHz, DMSO-d₆): δ ppm 1.21 (s, 1H), 1.469 (s, 5H), 2.0 (s, 3H), 3.76 (s, 3H), 4.35 (d, J=5.6 Hz, 2H), 4.72 (d, J=5.6, 2H), 5.08 (s, 1H), 6.54 (d, J=6 Hz, 1H), 7.21 (s, 1H), 7.33 (d, J=8.8 Hz, 1H), 7.56 (d, J=8.8 Hz, 1H), 9.36-9.44 (m, 2H). LCMS (ESI)m/z 403.2 [M+H]+

### Example 186: Synthesis of 3-fluoro-6-methoxy-4-(6-((methylamino)methyl)-1-(3-methyloxetan-3-yl)-1H-benzo[d]imidazol-2-yl)benzene-1,2-diol

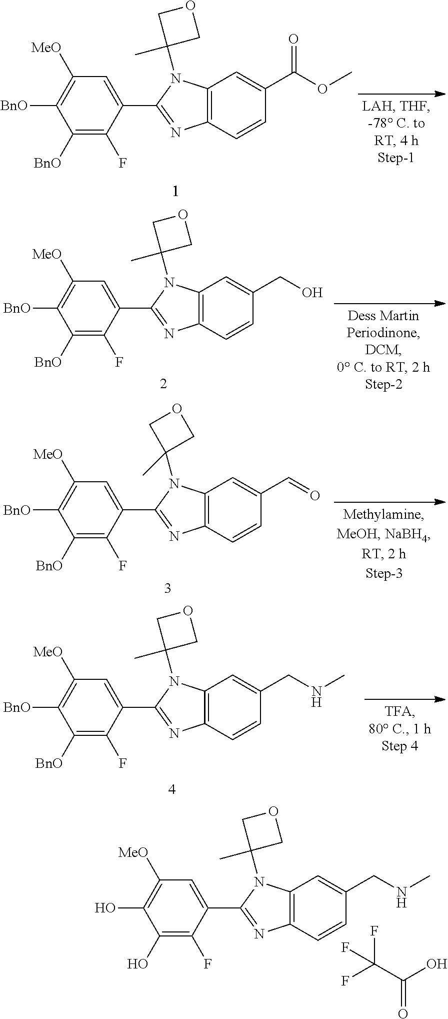

Step 1: {2-[3,4-bis(benzyloxy)-2-fluoro-5-methoxyphenyl]-1-(3-methyloxetan-3-yl)-1H-1,3-benzodiazol-6-yl}methanol

To a stirred solution of methyl 2-[3,4-bis(benzyloxy)-2-fluoro-5-methoxyphenyl]-1-(3-methyloxetan-3-yl)-1H-1,3-benzodiazole-6-carboxylate (450 mg, 772 μmol) in THF (20.0 mL) was added Lithium aluminium hydride in THF (2M Solution) (579 μL, 1.5 eq., 1.16 mmol) at −78° C. The reaction mixture was stirred for 4 hours at −78° C. The reaction mixture quenched with ammonium chloride solution and extracted with ethyl acetate. dried over sodium sulfate and concentrated to afford {2-[3,4-bis(benzyloxy)-2-fluoro-5-methoxy phenyl]-1-(3-methyloxetan-3-yl)-1H-1,3-benzodiazol-6-yl}methanol (350.0 mg, Crude) as color less oil. LCMS (ESI)m/z 555 [M+H]+

### Step 2: 2-[3,4-bis(benzyloxy)-2-fluoro-5-methoxyphenyl]-1-(3-methyloxetan-3-yl)-1H-1,3-benzodiazole-6-carbaldehyde

To a stirred solution of {2-[3,4-bis(benzyloxy)-2-fluoro-5-methoxyphenyl]-1-(3-methyloxetan-3-yl)-1H-1,3-benzodiazol-6-yl}methanol (350 mg, 631 μmol) in dichloromethane (5.00 mL) was added 1,1-bis(acetyloxy)-3-oxo-3H-1λ⁵,2-benziodaoxol-1-yl acetate (401 mg, 1.5 eq., 947 μmol) at 0° C. The reaction mixture was stirred at 0° C. for 2 hours. The reaction mixture quenched with NaHCO₃ and extracted into ethyl acetate, washed with brine, dried with sodium sulphate and concentrated. The crude was purified by flash column chromatography with 55-60% ethyl acetate in hexane to afford 2-[3,4-bis(benzyloxy)-2-fluoro-5-methoxyphenyl]-1-(3-methyloxetan-3-yl)-1H-1,3-benzodiazole-6-carbaldehyde (200 mg crude) as color less oil. LCMS (ESI)m/z 553 [M+H]+.

### Step 3: ({2-[3,4-bis(benzyloxy)-2-fluoro-5-methoxyphenyl]-1-(3-methyloxetan-3-yl)-1H-1,3-benzodiazol-6-yl} methyl)(methyl)amine

To a stirred solution of 2-[3,4-bis(benzyloxy)-2-fluoro-5-methoxyphenyl]-1-(3-methyloxetan-3-yl)-1H-1,3-benzodiazole-6-carbaldehyde (200 mg, 362 μmol) in methanol (15.0 mL) was added methanamine (1.81 mL, 10 eq., 3.62 mmol) at 0° C. The reaction mixture was stirred at room temperature for 16 hours, and then sodium borohydride (64.8 mg, 5 eq., 1.81 mmol) was added lot-wise and the mixture was further stirred for 16 hours at room temperature. The reaction was quenched with water and washed with diethyl ether. The organic layer was washed with water, dried over sodium sulfate and concentrated to afford ({2-[3,4-bis(benzyloxy)-2-fluoro-5-methoxyphenyl]-1-(3-methyloxetan-3-yl)-1H-1,3-benzodiazol-6-yl} methyl)(methyl)amine as a yellow oil. LCMS (ESI)m/z 568[M+H]+

### Step 4: Synthesis of 3-fluoro-6-methoxy-4-{6-[(methylamino)methyl]-1-(3-methyloxetan-3-yl)-1H-1,3-benzodiazol-2-yl}benzene-1,2-diol; trifluoroacetic acid

A mixture of ({2-[3,4-bis(benzyloxy)-2-fluoro-5-methoxyphenyl]-1-(3-methyloxetan-3-yl)-1H-1,3-benzodiazol-6-yl}methyl)(methyl)amine (80.0 mg, 141 μmol) and TFA (1.00 mL). was stirred at 60° C. for 3 hours. Then reaction mixture monitored by TLC and LCMS. The reaction mixture was concentrated. The crude was purified by preparative HPLC to afford 3-fluoro-6-methoxy-4-{6-[(methylamino)methyl]-1-(3-methyloxetan-3-yl)-1H-1,3-benzodiazol-2-yl}benzene-1,2-diol; trifluoroacetic acid (24.0 mg, 33.89% Yield) as an off white solid. ¹H NMR (400 MHz, DMSO-d₆): δ ppm 2.06 (s, 3H), 3.81 (bs, 3H), 4.45 (d, J=4 Hz, 2H), 4.78 (d, J=4 Hz, 2H), 7.55 (s, 1H), 7.76 (s, 1H), 7.80 (s, 2H), 8.60 (s, 1H), 9.46-9.41 (m, 2H).

LCMS (ESI)m/z 411.2 [M+H]+

### Example 187: Synthesis of 4-(5-(azetidin-1-yl)-1-(oxetan-3-yl)-1H-benzo[d]imidazol-2-yl)-6-methoxy-3-methylbenzene-1,2-diol

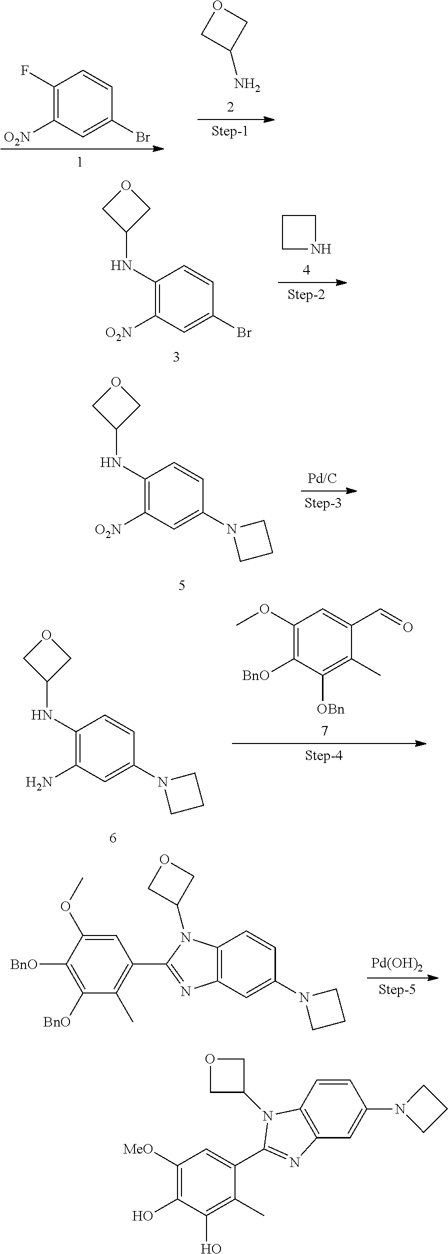

Step-1: To a solution of 4-bromo-1-fluoro-2-nitrobenzene (2.25 mL, 18.2 mmol) in Isopropyl Alcohol (30.0 mL), oxetan-3-amine (1.90 mL, 1.5 eq., 27.3 mmol) and N,N-Diisopropylethylamine (9.53 mL, 3 eq., 54.5 mmol) was added to the reaction mixture and stirred and heated at 90° C. for 16 h After completion of the reaction, the reaction mixture was concentrated under reduced pressure to get the crude and water was added to the reaction mixture was filtered and solid obtained. The solid afford N-(4-bromo-2-nitrophenyl)oxetan-3-amine (3.30 g, 12.0 mmol) as yellow solid. Yield: 3.30 g, (65.8%)

Step-2: To a stirred solution N-(4-bromo-2-nitrophenyl)oxetan-3-amine (2.70 g, 9.89 mmol) of in toluene (30.0 mL), azetidine (1.99 mL, 3 eq., 29.7 mmol) was added at room temperature. The reaction mixture was degassed with argon for 5 min. XPhos (471 mg, 0.1 eq., 989 μmol) and Tris(dibenzylideneacetone)dipalladium(0) (453 mg, 0.05 eq., 494 μmol) were added to the above suspension, degassed for 5 min. The reaction mixture stirred and heated at 110° C. for 16 h. After completion, the reaction mixture cooled to room temperature and passed through celite bed. The filtrate diluted with ethyl acetate and washed with water. The organic layer dried over anhydrous sodium sulphate, filtered and concentrated to give crude. The crude purified by flash chromatography. The desired fractions were concentrated to afford N-[4-(azetidin-1-yl)-2-nitrophenyl]oxetan-3-amine (2.50 g, 9.63 mmol) as off violet color solid. Yield: 2.50 g, (97.38%)

Step-3: To a solution of N-[4-(azetidin-1-yl)-2-nitrophenyl]oxetan-3-amine (700 mg, 2.81 mmol) in methanol (15.0 mL) was added 10% Palladium on carbon (50% wet) (500 mg) at room temperature and the reaction mixture stirred for 3 h under hydrogen atmosphere. After completion, the reaction mixture passed through celite bed. Filtrate was concentrated to get 4-(azetidin-1-yl)-N1-(oxetan-3-yl)benzene-1,2-diamine (600 mg, 2.74 mmol) as brown semisolid. Yield: 0.60 g, Crude

Step-4: To a stirred solution of 3,4-bis(benzyloxy)-5-methoxy-2-methylbenzaldehyde (397 mg, 0.8 eq., 1.09 mmol) and 4-(azetidin-1-yl)-N1-(oxetan-3-yl)benzene-1,2-diamine (600 mg, 2.74 mmol) in methanesulfinylmethane (5.00 mL) was added disodium sulfinatosulfonate (312 mg, 1.2 eq., 1.64 mmol) at room temperature. The resulting mixture stirred for 16 h at 85° C. After completion, reaction mixture diluted with water and extracted with ethyl acetate. The organic layer was dried over anhydrous sodium sulphate, filtered and concentrated to give crude. The crude purified by flash chromatography. The desired fractions concentrated to afford 5-(azetidin-1-yl)-2-[3,4-bis(benzyloxy)-5-methoxy-2-methylphenyl]-1-(oxetan-3-yl)-1H-1,3-benzodiazole (90.0 mg, 125 μmol) as light brown sticky. Yield: 0.09 g, 9.14%

Step-5: To a solution of 5-(azetidin-1-yl)-2-[3,4-bis(benzyloxy)-5-methoxy-2-methylphenyl]-1-(oxetan-3-yl)-1H-1,3-benzodiazole (40.0 mg, 62.0 μmol) in Tetrahydrofuran (10.0 mL) was added 20% Palladium hydroxide (200 mg) at room temperature and the reaction mixture stirred for 2 h under hydrogen atmosphere. After completion, the reaction mixture passed through celite bed. Filtrate was concentrated to get crude. The crude was purified by reverse phase HPLC and pure fractions were lyophilized to get 4-[5-(azetidin-1-yl)-1-(oxetan-3-yl)-1H-1,3-benzodiazol-2-yl]-6-methoxy-3-methylbenzene-1,2-diol (16.0 mg, 41.1 μmol) as off white semisolid. Yield: 0.016 g, 66.35%

ES MS M/Z=382.199 (M+1)+, ¹H NMR (400 MHz, DMSO-d₆) δ 8.96 (s, 1H), 8.65 (s, 1H), 7.82 (d, J=8.4 Hz, 1H), 6.60 (s, 1H), 6.56 (d, J=8.0 Hz, 1H), 6.43 (s, 1H), 5.25 (t, J=6.4 Hz, 1H), 4.98 (s, 4H), 3.83 (t, J=6.8 Hz, 4H), 3.74 (s, 4H), 2.32 (t, J=6.8 Hz, 2H), 1.80 (s, 3H).

### Example 188: Synthesis of 4-(5-(azetidin-1-yl)-1-(oxetan-3-yl)-1H-benzo[d]imidazol-2-yl)-3-fluoro-6-methoxybenzene-1,2-diol

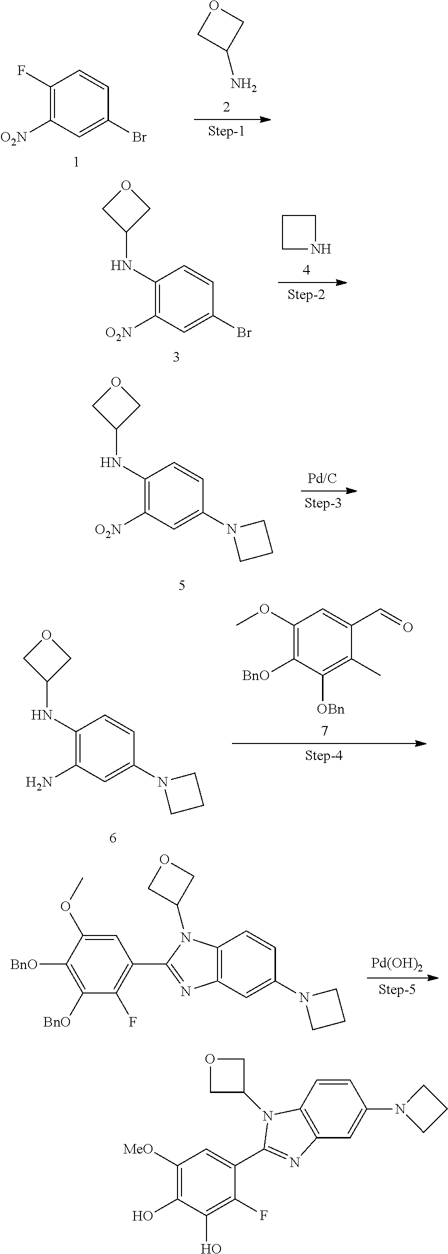

Step-1: To a solution of 4-bromo-1-fluoro-2-nitrobenzene (2.25 mL, 18.2 mmol) in Isopropyl Alcohol (30.0 mL), oxetan-3-amine (1.90 mL, 1.5 eq., 27.3 mmol) and N,N-Diisopropylethylamine (9.53 mL, 3 eq., 54.5 mmol) was added to the reaction mixture and stirred and heated at 90° C. for 16 h After completion of the reaction, the reaction mixture was concentrated under reduced pressure to get the crude and water was added to the reaction mixture was filtered and solid obtained. The solid afford N-(4-bromo-2-nitrophenyl)oxetan-3-amine (3.30 g, 12.0 mmol) as off yellow solid. Yield: 3.30 g, 65.8%

Step-2: To a stirred solution N-(4-bromo-2-nitrophenyl)oxetan-3-amine (2.70 g, 9.89 mmol) of in toluene (30.0 mL), azetidine (1.99 mL, 3 eq., 29.7 mmol) was added at room temperature. The reaction mixture was degassed with argon for 5 min. XPhos (471 mg, 0.1 eq., 989 μmol) and Tris(dibenzylideneacetone)dipalladium(0) (453 mg, 0.05 eq., 494 μmol) were added to the above suspension, degassed for 5 min. The reaction mixture stirred and heated at 110° C. for 16 h. After completion, the reaction mixture cooled to room temperature and passed through celite bed. The filtrate diluted with ethyl acetate and washed with water. The organic layer dried over anhydrous sodium sulphate, filtered and concentrated to give crude. The crude purified by flash chromatography. The desired fractions were concentrated to afford N-[4-(azetidin-1-yl)-2-nitrophenyl]oxetan-3-amine (2.50 g, 9.63 mmol) as off violet color solid. Yield: 2.50 g, 97.38%

Step-3: To a solution of N-[4-(azeidin-1-yl)-2-nitrophenyl]oxetan-3-amine (700 mg, 2.81 mmol) in methanol (15.0 mL) was added Palladium on carbon (500 mg) at room temperature and the reaction mixture stirred for 3 h under hydrogen atmosphere After completion, the reaction mixture passed through celite bed. Filtrate was concentrated to get 4-(azetidin-1-yl)-N1-(oxetan-3-yl)benzene-1,2-dianine (600 mg, 2.74 mmol) as brown semisolid, Yield: 600 mg, 97.43%

Step-4: To a stirred solution 4-(azetidin-1-yl)-N1-(oxetan-3-yl)benzene-1,2-diamine (224 mg, 1.02 mmol) and 3,4-bis(benzyloxy)-2-fluoro-5-methoxybenzaldehyde (300 mg, 0.8 eq., 819 μmol) in methanesulfinylmethane (5.00 mL) and disodium sulfinatosulfonate (233 mg, 1.2 eq., 1.23 mmol) was added at room temperature. The resulting mixture stirred and heated at 85° C. for 16 hr. After completion of reaction, the reaction mixture diluted with ice cold water and extracted with ethyl acetate. The organic layer dried over anhydrous sodium sulphate, filtered and concentrated to give crude. The crude purified by flash chromatography. The desired fractions were concentrated to afford 5-(azetidin-1-yl)-2-[3,4-bis(benzyloxy)-2-fluoro-5-methoxyphenyl]-1-(oxetan-3-yl)-1H-1,3-benzodiazole (100 mg, 150 μmol) as off violet color solid. Yield: 100 mg, 14.68%

Step-5: To a solution of 5-(azetidin-1-yl)-2-[3,4-bis(benzyloxy)-2-fluoro-5-methoxyphenyl]-1-(oxetan-3-yl)-1H-1,3-benzodiazole (80.0 mg, 141 μmol) in Tetrahydrofuran (10.0 mL) and Palladium hydroxide on carbon 20% w/w (150 mg, 1.7 eq., 245 μmol) was added at room temperature and the reaction mixture stirred for 2 h under hydrogen atmosphere. After completion, the reaction mixture passed through celite bed. Filtrate was concentrated to get 4-[5-(azetidin-1-yl)-1-(oxetan-3-yl)-1H-1,3-benzodiazol-2-yl]-3-fluoro-6-methoxybenzene-1,2-diol (5.00 mg, 12.9 μmol) as white solid. Yield: 0.005 g, (9.15%)

LCMS and NMR Data: ES MS M/Z=386.13 (M+1)⁺; UPLC: 99.69%; 1H NMR (400 MHz, DMSO-d6) δ 9.41 (d, J=7.6 Hz, 2H) 6.67 (s, 1H), 6.58 (t, J=7.1 Hz, 2H), 5.37 (s, 1H), 5.01 (d, J=6.0 Hz, 4H) 3.81 (t, J=6.8 Hz, 7H), 3.71 (s, 3H), 2.31 (t, J=6.8 Hz, 2H).

### Example 189: Synthesis of 4-(5-(azetidin-1-yl)-1H-benzo[d]imidazol-2-yl)-6-methoxy-3-methylbenzene-1,2-diol

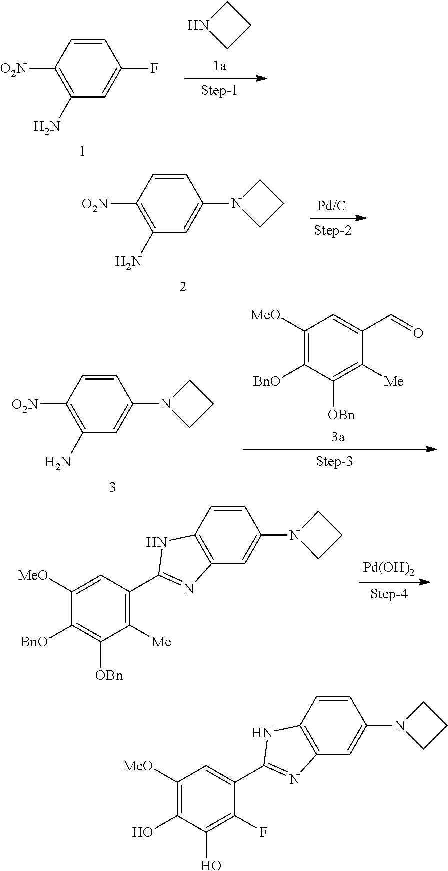

Step-1: To a solution of 5-fluoro-2-nitroaniline (5.00 g, 32.0 mmol) in N-Methyl-2-pyrrolidone (50.0 mL), N,N-Diisopropylethylamine (5.59 mL, 32.0 mmol) and azetidine (2.74 g, 1.5 eq., 48.0 mmol) was added to the reaction mixture and stirred and heated at 90° C. for 16 h After completion of the reaction, the water was added to the reaction mixture and compound was precipitated. Then reaction mixture was filtered and solid compound was dried under vacuum to afford 5-(azetidin-1-yl)-2-nitroaniline (5.40 g, 27.7 mmol) as off yellow solid. Yield: 5.40 g, (86.39%)

Step-2: To a solution of 5-(azetidin-1-yl)-2-nitroaniline (500 mg, 2.59 mmol) in methanol (20.0 mL) was added Palladium on carbon (700 mg) at room temperature and the reaction mixture stirred for 2 h under hydrogen atmosphere. After completion, the reaction mixture passed through celite bed. Filtrate was concentrated to get 4-(azetidin-1-yl)benzene-1,2-diamine (450 mg, 1.87 mmol) as brown sticky. Yield: 0.45 g, 72.44%

Step-3: To a stirred solution of 4-(azetidin-1-yl)benzene-1,2-diamine (150 mg, 919 μmol) and 3,4-bis(benzyloxy)-5-methoxy-2-methylbenzaldehyde (266 mg, 0.8 eq., 735 μmol) in methanesulfinylmethane (10.0 mL) was added disodium sulfinatosulfonate (210 mg, 1.2 eq., 1.10 mmol) at room temperature. The resulting mixture stirred for 16 h at 85° C. After completion, reaction mixture cooled to room temperature and diluted with water and extracted with ethyl acetate. The organic layer was dried over anhydrous sodium sulphate and concentrated under reduced pressure to get crude. The crude purified by flash chromatography. The desired fractions concentrated to afford 5-(azetidin-1-yl)-2-[3,4-bis(benzyloxy)-5-methoxy-2-methylphenyl]-1H-1,3-benzodiazole (150 mg, 282 μmol) as off white solid. Yield: 0.15 g, 30.67%

Step-4: To a solution of 5-(azetidin-1-yl)-2-[3,4-bis(benzyloxy)-5-methoxy-2-methylphenyl]-1H-1,3-benzodiazole (150 mg, 297 μmol) in Tetrahydrofuran (10.0 mL) was added 20% Palladium hydroxide (100 mg, 0.48 eq., 143 μmol) at room temperature and the reaction mixture stirred for 3 h under hydrogen atmosphere. After completion, the reaction mixture passed through celite bed. Filtrate was concentrated to get crude. Crude was purified by reverse phase HPLC and desired fractions were lyophilized to get 4-[5-(azetidin-1-yl)-1H-1,3-benzodiazol-2-yl]-6-methoxy-3-methylbenzene-1,2-diol (14.0 mg, 40.9 μmol) as white solid. Yield: 0.014 g, 13.78%

(ESI) m/z 326.06 [M+1]⁺; 1H NMR (400 MHz, DMSO-d6) δ 11.99 (m, 1H), 8.89 (brs, 1H), 8.48 (brs, 1H), 7.42-7.26 (m, 1H), 6.80 (d, J=8.48 Hz, 1H), 6.60-6.31 (m, 2H), 3.81-3.77 (m, 7H), 2.33-2.28 (m, 5H).

### Example 190: Synthesis of 1-[2-(2-fluoro-3,4-dihydroxy-5-methoxyphenyl)-1-(3-methyloxetan-3-yl)-1H-1,3-benzodiazol-5-yl]pyrrolidin-2-one

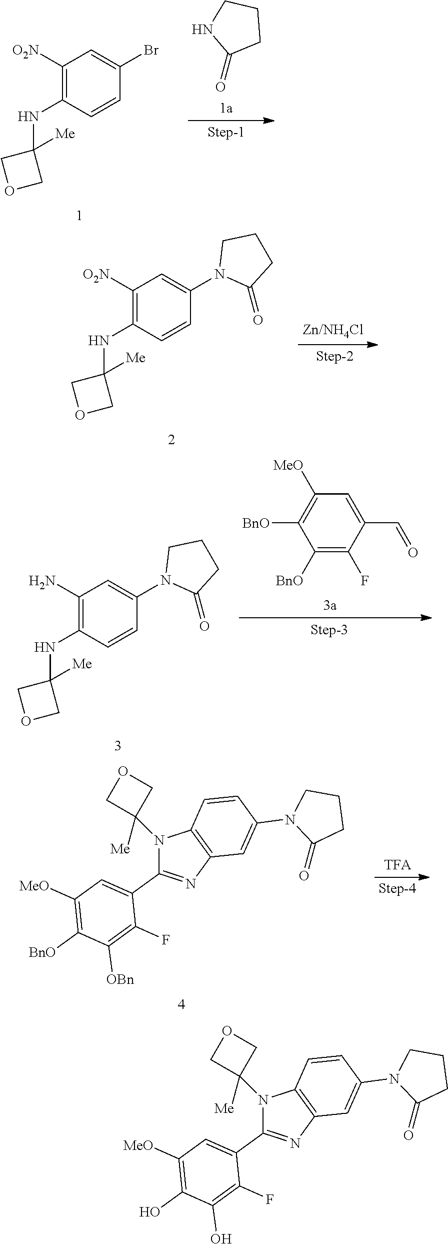

Step-1: To a stirred solution of N-(4-bromo-2-nitrophenyl)-3-methyloxetan-3-amine (500 mg, 1.74 mmol) in 1,4-dioxane (8.00 mL), and Potassium carbonate (722 mg, 3 eq., 5.22 mmol) were added and reaction mixture was purged with argon for 5 min then Copper (I)Iodide (166 mg, 0.3 eq., 522 μmol) and 2-aminoacetic acid (78.4 mg, 0.6 eq., 1.04 mmol) were added and again purged with argon. The reaction mixture was heated to 130° C. for 24 h. After completion of reaction, reaction mixture filtered through celite. The filtrate was distilled to obtain crude. The crude purified by flash chromatography. The desired fraction were concentrated to afford 1-{4-[(3-methyloxetan-3-yl)amino]-3-nitrophenyl}pyrrolidin-2-one (50.0 mg) as yellow solid. Yield: 50 mg, 9.86%

Step-2: To a solution of 1-{4-[(3-methyloxetan-3-yl)amino]-3-nitrophenyl}pyrrolidin-2-one (120 mg, 412 μmol) in methanol (20.0 mL) was added zinc (135 mg, 5 eq., 2.06 mmol) followed by addition of ammonium chloride (110 mg, 5 eq., 2.06 mmol) at room temperature and the reaction mixture stirred for 3 h under hydrogen atmosphere. After completion, the reaction mixture diluted with dichloromethane and passed through celite bed. Then water was added to filtrate and extracted with 10% methanol in dichloromethane solution. The organic fraction collected, dried over anhydrous sodium sulphate, concentrated to obtain 1-{3-amino-4-[(3-methyloxetan-3-yl)amino]phenyl}pyrrolidin-2-one as brown liquid. Yield: 106 mg, 98.47%

Step-3: To a stirred solution of 1-{3-amino-4-[(3-methyloxetan-3-yl)amino]phenyl}pyrrolidin-2-one (104 mg, 398 μmol) and 3,4-bis(benzyloxy)-2-fluoro-5-methoxybenzaldehyde (117 mg, 0.8 eq., 318 μmol) in methanesulfinylmethane (3.00 mL), disodium sulfinatosulfonate (113 mg, 1.5 eq., 597 μmol) was added at room temperature. The resulting mixture stirred for 16 h at 80° C. After completion, water and ethyl acetate was added to reaction mixture. The organic fractions were collected, dried over anhydrous sodium sulphate, filtered and concentrated to get crude. The crude purified by flash chromatography. The desired fractions were concentrated to afford 1-{2-[3,4-bis(benzyloxy)-2-fluoro-5-methoxyphenyl]-1-(3-methyloxetan-3-yl)-1H-1,3-benzodiazol-5-yl}pyrrolidin-2-one (145 mg, 239 μmol) as sticky solid. Yield: 145 mg, 59.96%

Step-4: To 1-{2-[3,4-bis(benzyloxy)-2-fluoro-5-methoxyphenyl]-1-(3-methyloxetan-3-yl)-1H-1,3-benzodiazol-5-yl}pyrrolidin-2-one (110 mg, 181 μmol) trifluoroacetic acid (1.00 mL) added at room temperature. The resulting reaction mixture stirred at 60° C. for 4 h. After the complete consumption of starting material, reaction mixture was concentrated to obtain the crude. The crude was purified by Reverse phase HPLC. The desired fraction were collected and lyophilized to obtain 1-[2-(2-fluoro-3,4-dihydroxy-5-methoxyphenyl)-1-(3-methyloxetan-3-yl)-1H-1,3-benzodiazol-5-yl]pyrrolidin-2-one (10.0 mg, 23.4 μmol) as off white solid. Yield: 10 mg, 12.92%

LCMS and NMR Data: ES MS M/Z=428.24 [M+1]⁺, UPLC: 98.81% ¹H NMR (400 MHz, DMSO-d6) δ 9.64 (bs, 2H), 7.95 (s, 1H), 7.66 (d, J=9.2 Hz, 1H), 7.37 (d, J=4.0 Hz, 1H), 6.64 (d, J=5.6 Hz, 1H), 4.73 (d, J=5.6 Hz, 1H), 4.42 (d, J=5.6 Hz, 1H), 3.92 (t, J=7.2 Hz, 2H), 3.80 (s, 3H), 2.54-2.52 (m, 2H), 2.12-2.06 (m, 5H).

### Example 191: Synthesis of 1-(2-(2-fluoro-3,4-dihydroxy-5-methoxyphenyl)-1-(oxetan-3-yl)-1H-benzo[d]imidazol-5-yl)pyrrolidin-2-one

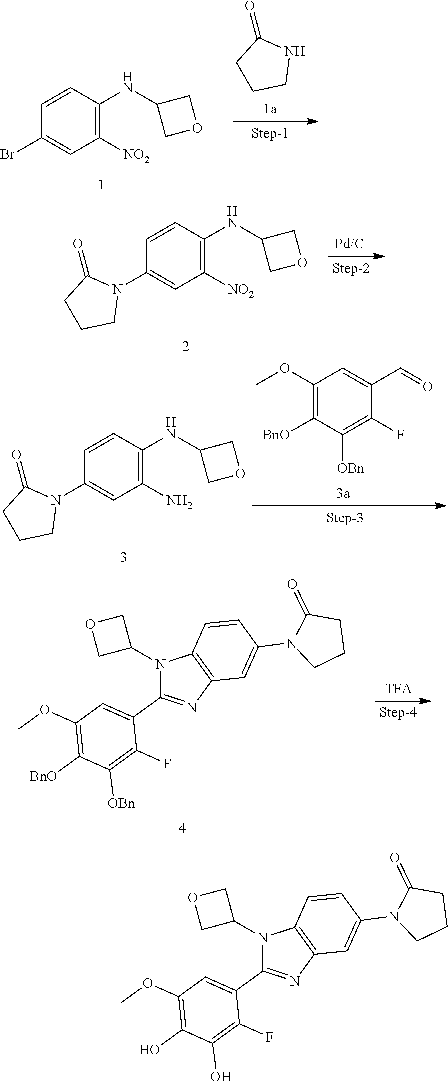

Step-1: To a stirred solution of N-(4-bromo-2-nitrophenyl)oxetan-3-amine (800 mg, 2.93 mmol) in 1,4-dioxane (8.00 mL), pyrrolidin-2-one (374 mg, 1.5 eq., 4.39 mmol) and Potassium carbonate (1.21 g, 3 eq., 8.79 mmol) were added and reaction mixture was purged with argon for 5 min then Copper (I)Iodide (93.0 mg, 0.1 eq., 293 μmol) and 2-aminoacetic acid (44.0 mg, 0.2 eq., 586 μmol) were added and again purged with argon. The reaction mixture was heated to 130° C. for 18 h. After completion of reaction, reaction mixture filtered through celite bed. The filtrate was distilled to obtain crude. The crude purified flash chromatography. The desired fraction were concentrated to afford 1-{3-nitro-4-[(oxetan-3-yl)amino]phenyl}pyrrolidin-2-one (500 mg, 1.68 mmol) as yellow solid. Yield: 500 mg, 57.25%

Step-2: To a stirred solution of 1-{3-nitro-4-[(oxetan-3-yl)amino]phenyl}pyrrolidin-2-one (150 mg, 541 μmol) in methanol (5.00 mL), 10% Palladium on carbon (50% wet) (123.0 mg, 0.4 eq., 216 μmol) was charged and stirred under hydrogen atmosphere at room temperature for 1 h. After completion of reaction, reaction mixture filtered through celite bed. The filtrate was distilled below 30° C. to obtain 1-{3-amino-4-[(oxetan-3-yl)amino]phenyl}pyrrolidin-2-one (130 mg, 499 μmol) as crude. Yield: 130 mg, 92.28%

Step-3: To a stirred solution 1-{3-amino-4-[(oxetan-3-yl)amino]phenyl}pyrrolidin-2-one (130 mg, 526 μmol) and 3,4-bis(benzyloxy)-2-fluoro-5-methoxybenzaldehyde (154 mg, 0.8 eq., 421 μmol) in methanol (4.00 mL) was added Acetic acid (500 μL) at room temperature. The resulting mixture stirred for 16 h at 90° C. After completion, reaction mixture concentrated under reduced pressure to get crude 1-{2-[3,4-bis(benzyloxy)-2-fluoro-5-methoxyphenyl]-1-(oxetan-3-yl)-1H-1,3-benzodiazol-5-yl}pyrrolidin-2-one (110 mg, 84.5 μmol). Yield: 110 mg, 16.06%

Step-4: To a stirred solution of 1-{2-[3,4-bis(benzyloxy)-2-fluoro-5-methoxyphenyl]-1-(oxetan-3-yl)-1H-1,3-benzodiazol-5-yl}pyrrolidin-2-one (130 mg, 219 μmol) in Tetrahydrofuran (5.00 mL), 20% Palladium hydroxide (26.8 mg, 219 μmol) was charged and stirred under hydrogen atmosphere at room temperature for 2 h. After completion of reaction, reaction mixture filtered through celite bed. The filtrate was distilled below 30° C. to obtain crude. The crude was purified by Reverse prep HPLC to get 1-[2-(2-fluoro-3,4-dihydroxy-5-methoxyphenyl)-1-(oxetan-3-yl)-1H-1,3-benzodiazol-5-yl]pyrrolidin-2-one (40.0 mg, 95.8 μmol) as light brown solid. Yield: 40 mg, 43.75%

ES MS M/Z=414.17 [M+1]⁺, UPLC: 99.01%. ¹HNMR (400 MHz, DMSO-d6): δ 9.46 (bs, 2H), 8.01 (d, J=8.8 Hz, 1H), 7.93 (d, J=2.0 Hz, 1H), 7.71 (dd, J=9.2 Hz, 2.0 Hz, 1H), 6.64 (d, J=6.0 Hz, 1H), 5.44 (bs, 1H), 5.07-5.00 (m, 4H), 3.94 (t, J=6.8 Hz, 2H), 3.79 (s, 3H), 2.54-2.49 (m, 3H), 2.14-2.07 (m, 2H).

### Example 192: Synthesis of 3-fluoro-6-methoxy-4-[1-(3-methyloxetan-3-yl)-5-(phenylamino)-1H-1,3-benzodiazol-2-yl]benzene-1,2-diol

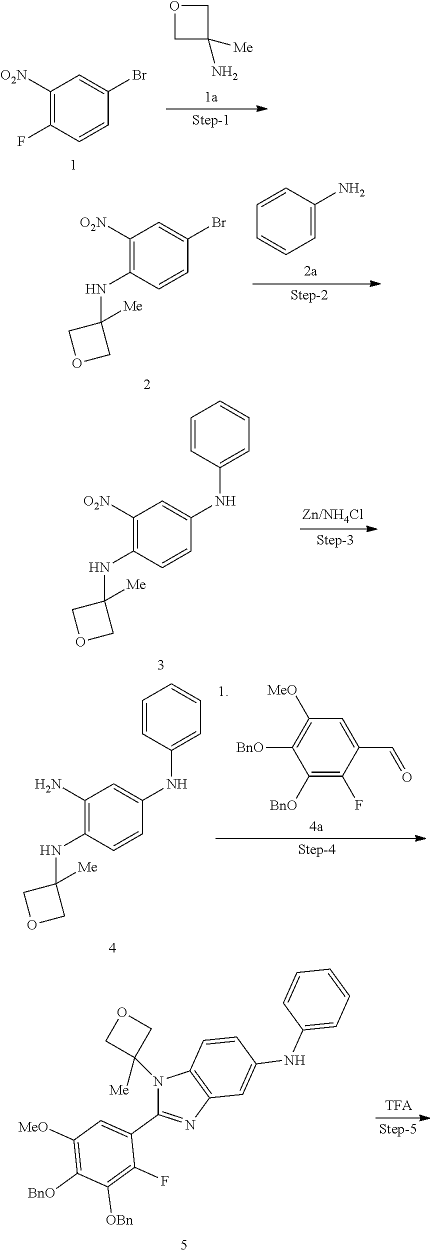

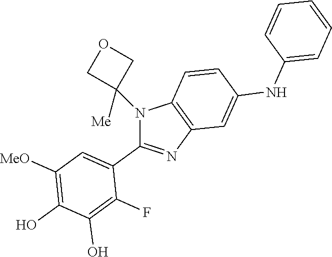 [SMILES: COc1cc(-c2nc3cc(Nc4ccccc4)ccc3n2C2(C)COC2)c(F)c(O)c1O]

Step-1: To a stirred solution of 4-bromo-1-fluoro-2-nitrobenzene (2.00 g, 9.09 mmol) in 1-methylpyrrolidin-2-one (10.0 mL) was added 3-methyloxetan-3-amine (1.14 mL, 1.5 eq., 13.6 mmol) and ethylbis(propan-2-yl)amine (4.76 mL, 3 eq., 27.3 mmol) and heated at 100° C. for 16 h. After completion of reaction, reaction mixture diluted with water and extracted with ethyl acetate. The organic layer was dried over anhydrous sodium sulphate, filtered and concentrated to afford N-(4-bromo-2-nitrophenyl)-3-methyloxetan-3-amine (2.48 g, 7.08 mmol) as orange solid. Yield: 2.48 g (77.91%)

Step-2: To a stirred solution of aniline (227 mg, 2.44 mmol) and N-(4-bromo-2-nitrophenyl)-3-methyloxetan-3-amine (700 mg, 2.44 mmol) in 1,4-dioxane (5.00 mL) was added sodium 2-methylpropan-2-olate (474 mg, 2 eq., 4.88 mmol) at room temperature. The reaction mixture was degassed with argon for 5 min. Then Tris(dibenzylideneacetone)dipalladium(0) (112 mg, 0.05 eq., 122 μmol) and dicyclohexyl[2′,4′,6′-tris(propan-2-yl)-[1,1′-biphenyl]-2-yl]phosphane (116 mg, 0.1 eq., 244 μmol) were added to the above suspension, degassed for 5 minutes and reaction mixture stirred was heated at 120° C. for 16 h. After completion, the reaction mixture cooled to room temperature, diluted with water and extracted ethyl acetate. The organic layer dried over anhydrous sodium sulphate, filtered and concentrated to give crude. The crude purified by flash chromatography. The desired fractions concentrated to afford N1-(3-methyloxetan-3-yl)-2-nitro-N4-phenylbenzene-1,4-diamine (300 mg, 601 μmol). Yield: 300 mg, 24.66%

Step-3: To a solution of N1-(3-methyloxetan-3-yl)-2-nitro-N4-phenylbenzene-1,4-diamine (300 mg, 601 μmol) in methanol (20.0 mL) was added zinc (197 mg, 5 eq., 3.01 mmol) followed by addition of ammonium chloride (161 mg, 5 eq., 3.01 mmol) at room temperature and the reaction mixture stirred for 3 h at 50° C. After completion, the reaction mixture diluted with dichloromethane and passed through celite bed. water was added to filtrate and extracted with 10% methanol in dichloromethane solution. The organic fraction collected, dried over anhydrous sodium sulphate, concentrated to obtain N1-(3-methyloxetan-3-yl)-N4-phenylbenzene-1,2,4-triamine (255 mg, 568 μmol) as brown liquid. Yield: 255 mg, 94.46%

Step-4: To a stirred solution of N1-(3-methyloxetan-3-yl)-N4-phenylbenzene-1,2,4-triamine (250 mg, 557 μmol) and 3,4-bis(benzyloxy)-2-fluoro-5-methoxybenzaldehyde (163 mg, 0.8 eq., 446 μmol) in methanesulfinylmethane (3.00 mL), disodium sulfinatosulfonate (159 mg, 1.5 eq., 835 μmol) was added at room temperature. The resulting mixture stirred for 16 h at 80° C. After completion, water and ethyl acetate was added to reaction mixture. The organic fractions were collected, dried over anhydrous sodium sulphate, filtered and concentrated to get crude. The crude purified by flash chromatography. The desired fractions were concentrated to afford 2-[3,4-bis(benzyloxy)-2-fluoro-5-methoxyphenyl]-1-(3-methyloxetan-3-yl)-N-phenyl-1H-1,3-benzodiazol-5-amine (270 mg, 414 μmol) as pink solid. Yield: 270 mg, 74.33%

Step-5: To 2-[3,4-bis(benzyloxy)-2-fluoro-5-methoxyphenyl]-1-(3-methyloxetan-3-yl)-N-phenyl-1H-1,3-benzodiazol-5-amine (250 mg, 406 μmol), trifluoroacetic acid (1.00 mL) added at room temperature. The resulting reaction mixture stirred at 60° C. for 4 h. After the complete consumption of starting material, reaction mixture was concentrated to obtain the crude. The crude was purified by reverse phase HPLC and desired fractions were collected and lyophilized to obtain 3-fluoro-6-methoxy-4-[1-(3-methyloxetan-3-yl)-5-(phenylamino)-1H-1,3-benzodiazol-2-yl]benzene-1,2-diol (63.0 mg, 141 μmol) as pink colored solid. Yield: 63 mg, 34.65%

LCMS and NMR Data: ES MS M/Z=436.31 (M+1)⁺, UPLC: 97.24%; ¹H NMR (400 MHz, DMSO-d6) δ 9.77 (bs, 2H), 8.41 (bs, 1H), 7.39-7.35 (m, 2H), 7.29-7.25 (m, 2H), 7.20-7.16 (m, 1H), 7.11 (d, J=8.0 Hz, 1H), 6.89-6.85 (m, 1H), 6.69 (d, J=5.6 Hz, 1H), 4.75 (d, J=6.0 Hz, 1H), 4.43 (d, J=6.0 Hz, 1H), 2.10 (s, 3H)

### Example 193: Synthesis of 5-[5-(azetidin-1-yl)-1-cyclobutyl-1H-1,3-benzodiazol-2-yl]-3-methoxy benzene-1,2-diol

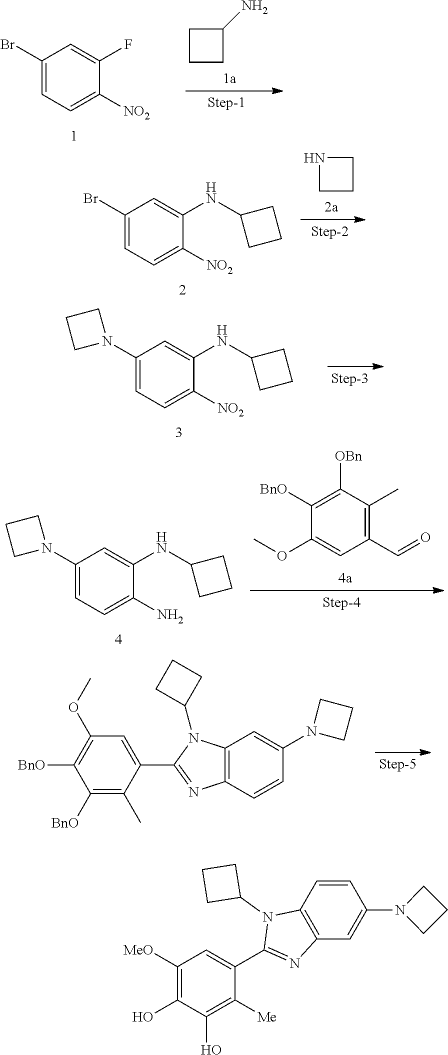

Step-1: To a solution of 4-bromo-1-fluoro-2-nitrobenzene (1.12 mL, 9.09 mmol) in propan-2-ol (30.0 mL), cyclobutanamine (1.17 mL, 1.5 eq., 13.6 mmol) and N,N-Diisopropylethylamine (4.76 mL, 3 eq., 27.3 mmol) was added to the reaction mixture and stirred and heated at 90° C. for 2-16 h After completion of the reaction, the reaction mixture was concentrated under reduced pressure to get the crude and water was added to the reaction mixture was filtered and solid obtained. The dried solid afforded 4-bromo-N-cyclobutyl-2-nitroaniline (2.00 g, 7.23 mmol) as off yellow solid. Yield: 2.000 g, (79.52%)

Step-2: To a stirred solution 4-bromo-N-cyclobutyl-2-nitroaniline (1.00 g, 3.69 mmol) of in toluene (30.0 mL), azetidine (496 μL, 2 eq., 7.38 mmol) was added at room temperature. The reaction mixture was degassed with argon for 5 min. dicyclohexyl[2′,4′,6′-tris(propan-2-yl)-[1,1′-biphenyl]-2-yl]phosphane (176 mg, 0.1 eq., 369 μmol) and Tris(dibenzylideneacetone)dipalladium(0) (169 mg, 0.05 eq., 184 μmol) were added to the above suspension, degassed for 5 min. The reaction mixture stirred and heated at 110° C. for 16 h. After completion, the reaction mixture cooled to room temperature and passed through celite bed. The filtrate diluted with ethyl acetate and washed with water. The organic layer dried over anhydrous sodium sulphate, filtered and concentrated to give crude. The crude purified by flash chromatography. The desired fractions were concentrated to afford 4-(azetidin-1-yl)-N-cyclobutyl-2-nitroaniline (700 mg, 2.12 mmol) as off violet color solid. Yield: 700 mg, 57.56%

Step-3: To a solution of 4-(azetidin-1-yl)-N-cyclobutyl-2-nitroaniline (700 mg, 2.83 mmol) in methanol (30.0 mL) was added 10% Palladium on Carbon (50% wet) (1.00 g, 9.40 mmol) at room temperature and the reaction mixture stirred for 2 h under hydrogen atmosphere. After completion, the reaction mixture passed through celite bed. Filtrate was concentrated to get as 4-(azetidin-1-yl)-N1-cyclobutylbenzene-1,2-diamine (1.00 g, 1.01 mmol) as violet color solid. Yield: 1.00 g, 35.76%

Step-4: To a stirred solution 4-(azetidin-1-yl)-N1-cyclobutylbenzene-1,2-diamine (180 mg, 1.2 eq., 828 μmol) and 3,4-bis(benzyloxy)-5-methoxy-2-methylbenzaldehyde (250 mg, 690 μmol) in methanesulfinylmethane (5.00 mL) and disodium sulfinatosulfonate (157 mg, 1.2 eq., 828 μmol) was added at room temperature. The resulting mixture stirred and heated at 85° C. for 16 hr. After completion of reaction, the reaction mixture diluted with ice cold water and extracted with ethyl acetate. The organic layer dried over anhydrous sodium sulphate, filtered and concentrated to give crude. The crude purified by flash chromatography. The desired fractions were concentrated to afford 5-(azetidin-1-yl)-2-[3,4-bis(benzyloxy)-5-methoxy-2-methylphenyl]-1-cyclobutyl-1H-1,3-benzodiazole (150 mg, 131 μmol) as off violet color solid. Yield: 0.15 g, 19.04%

Step-5: To a solution of 5-(azetidin-1-yl)-2-[3,4-bis(benzyloxy)-5-methoxyphenyl]-1-cyclobutyl-1H-1,3-benzodiazole (150 mg, 275 μmol) in Tetrahydrofuran (15.0 mL) and Palladium hydroxide on carbon 20% w/w (233 mg, 1.4 eq., 381 μmol) was added at room temperature and the reaction mixture stirred for 2 h under hydrogen atmosphere. After completion, the reaction mixture passed through celite bed. Filtrate was concentrated to get crude. The crude was purified using reverse phase prep HPLC and desired fraction were lyophilized to get 5-[5-(azetidin-1-yl)-1-cyclobutyl-1H-1,3-benzodiazol-2-yl]-3-methoxybenzene-1,2-diol (14.0 mg, 37.8 μmol) as white color solid. Yield: 0.014 g, 13.77%

LCMS and NMR Data: ES MS M/Z=380.20(M+1); UPLC: 98.77%; 1H NMR (400 MHz, DMSO-d6) δ 7.63 (d, J=8.8 Hz, 1H), 6.60 (d, J=2 Hz, 1H), 7.83 (dd, J=8.4, 2.0 Hz, 1H), 6.37 (s, 1H), 4.62-4.57 (m, 1H), 3.78 (t, J=7.2 Hz, 4H), 3.74 (s, 3H), 2.68 (t, J=10.0 Hz, 2H), 2.32-2.22 (m, 2H), 1.87 (t, J=7.2 Hz, 4H).

### Example 194: Synthesis of 3-fluoro-6-methoxy-4-(1-(3-methyloxetan-3-yl)-6-(1H-pyrazol-4-yl)-1H-benzo[d]imidazol-2-yl benzene-1,2-diol

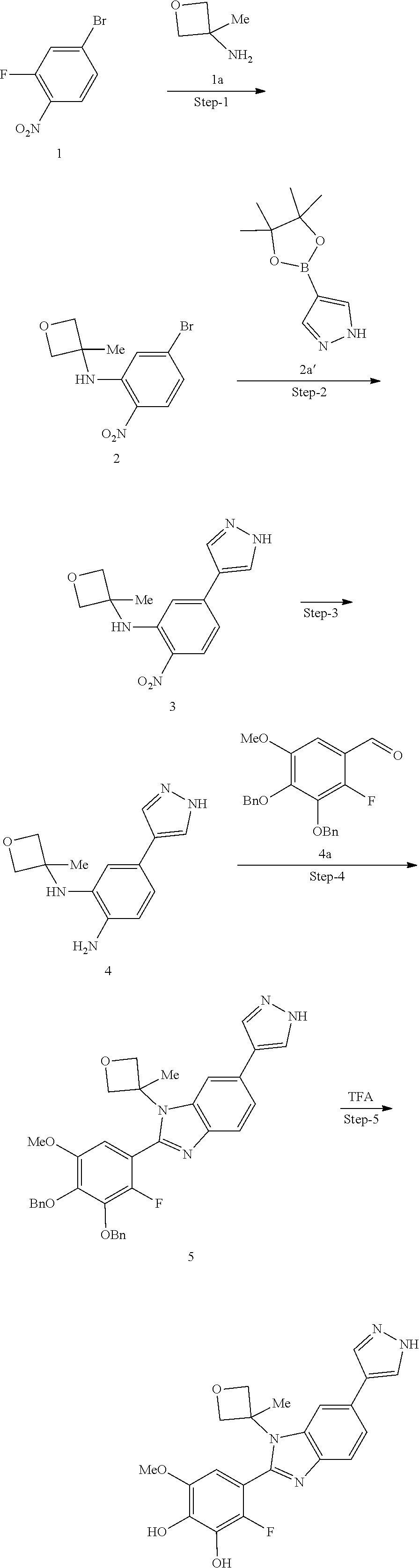

Step-1: To a solution of 4-bromo-2-fluoro-1-nitrobenzene (500.0 mg, 2.28 mmol) in 1-methylpyrrolidin-2-one (5.00 mL), 3-methyloxetan-3-amine (217.1 mg, 1.1 eq., 2.27 mmol) and ethylbis(propan-2-yl)amine (1.18 mL, 3 eq., 6.81 mmol) was added to the reaction mixture and heated at 100° C. for 16 h. After completion of the reaction, the reaction mixture was diluted with water solid obtained was filtered and dried to afford N-(5-bromo-2-nitrophenyl)-3-methyloxetan-3-amine (200.0 mg, 6.96 mmol) as yellow solid. Yield: 200 mg, 32.3%

Step-2: To a stirred solution of N-(5-bromo-2-nitrophenyl)-3-methyloxetan-3-amine (200 mg, 697 μmol) in ethanol (4.00 mL), toluene (4.00 mL) and water (1.00 mL), 4-(4,4,5,5-tetramethyl-1,3,2-dioxaborolan-2-yl)-1H-pyrazole (149 mg, 1.1 eq., 766 μmol) and disodium carbonate (221 mg, 3 eq., 2.09 mmol) were added and reaction mixture was purged with argon for 5 min, then tetrakis(triphenylphosphine)palladium(0) (161 mg, 0.2 eq., 139 μmol) was added and again purged with argon. The reaction mixture was heated to 90° C. for 2 h. After completion of reaction, reaction mixture filtered through celite. Filtrate was washed with water and extracted with ethyl acetate. The combined organic layers dried over anhydrous sodium sulphate, filtered and concentrated to obtain crude. The crude purified by flash chromatography. The desired fraction were concentrated to afford 3-methyl-N-[2-nitro-5-(1H-pyrazol-4-yl)phenyl]oxetan-3-amine (150 mg, 301 μmol) as yellow solid. Yield: 150 mg, 43.18% and 130 mg, 40% (From two batches)

Step-3: To a stirred solution 3-methyl-N-[2-nitro-5-(1H-pyrazol-4-yl)phenyl]oxetan-3-amine (280 mg, 510 μmol) in methanol (2.00 mL), zinc (167 mg, 5 eq., 2.55 mmol) and ammonium chloride (137 mg, 5 eq., 2.55 mmol) was added at 0° C. and reaction mixture was stirred at room temperature for 30 min. After completion, the reaction mixture passed through celite bed. The filtrate was concentrated under reduced pressure and washed with water. The organic layer dried over anhydrous sodium sulphate, filtered and concentrated to afford N1-(3-methyloxetan-3-yl)-5-(1H-pyrazol-4-yl)benzene-1,2-diamine (160 mg, 118 μmol) as brown solid. Yield: 160 mg, Crude

Step-4: To a stirred solution N1-(3-methyloxetan-3-yl)-5-(1H-pyrazol-4-yl)benzene-1,2-diamine (80.0 mg, 327 μmol) and 3,4-bis(benzyloxy)-2-fluoro-5-methoxybenzaldehyde (120 mg, 327 μmol) in methanesulfinylmethane (5.00 mL) was added disodium sulfinatosulfonate (93.4 mg, 1.5 eq., 491 μmol) at room temperature. The resulting mixture stirred for 16 h at 85° C. After completion, reaction mixture diluted with water solid obtained was filtered and dried to get 2-[3,4-bis(benzyloxy)-2-fluoro-5-methoxyphenyl]-1-(3-methyloxetan-3-yl)-6-(1H-pyrazol-4-yl)-1H-1,3-benzodiazole (130 mg, 220 μmol) as brown solid. Yield: 130 mg, 75.46%

Step 5: To a solution of 2-[3,4-bis(benzyloxy)-2-fluoro-5-methoxyphenyl]-1-(3-methyloxetan-3-yl)-6-(1H-pyrazol-4-yl)-1H-1,3-benzodiazole (130 mg, 220 μmol) in trifluroacetic acid (1.00 mL), was added at room temperature and the reaction mixture stirred at 0° C. for 2 h. After completion, the reaction mixture was concentrated to get crude as brown viscous liquid. Purification was done by Reverse Prep HPLC and desired fraction were lyophilized to get 2-(2-fluoro-3,4-dihydroxy-5-methoxyphenyl)-N-methyl-1-(3-methyloxetan-3-yl)-1H-1,3-benzodiazole-6-sulfonamide (11.0 mg, 25.1 μmol) as white solid. Yield: 11 mg, 12.08%

LCMS and NMR Data: ES MS M/Z=346.15 (M+1), 1HNMR (400 MHz, DMSO-d6): δ 9.7(bs, 1H), 8.22 (s, 2H), 7.72 (dd, J=17.0 Hz, 8.4 Hz, 1H), 7.45 (s, 1H), 6.68 (d, J=6.0 Hz, 1H), 4.75 (d, J=6.0 Hz, 2H), 4.52 (d, J=5.6 Hz, 2H), 3.80 (s, 3H), 2.13 (s, 3H).

### Example 195: Synthesis of 2-(2-fluoro-3,4-dihydroxy-5-methoxyphenyl)-N,N-dimethyl-1-(3-methyloxetan-3-yl)-1H-1,3-benzodiazole-5-carboxamide

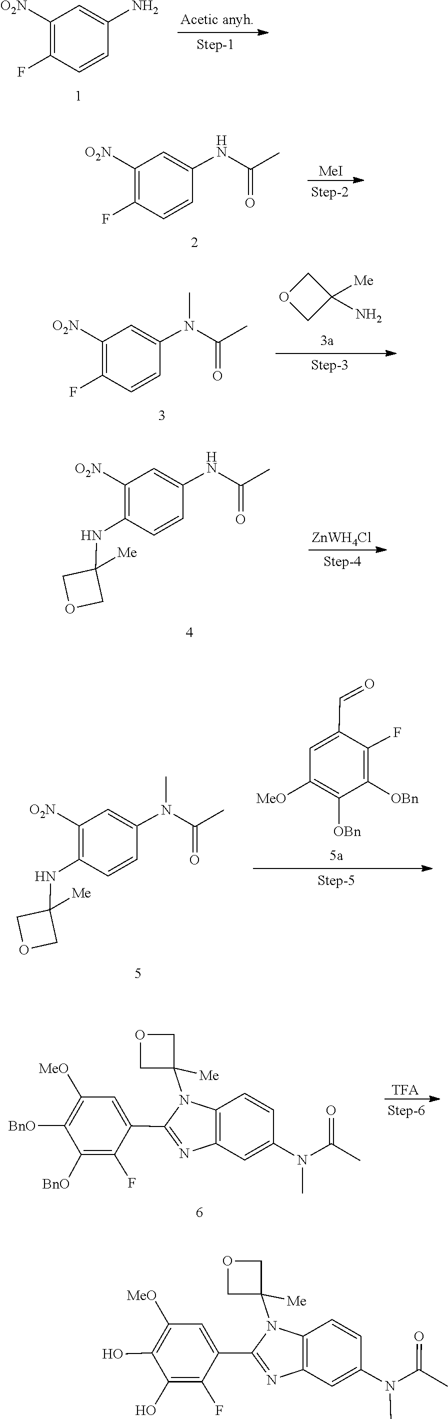

Step-1: A stirred solution of 4-fluoro-3-nitroaniline (1.00 g, 6.41 mmol) in dichloromethane (10.0 mL), acetyl acetate (666 μL, 1.1 eq., 7.05 mmol) was added and stirred at room temperature for 16 h. After completion of reaction, reaction mixture diluted with water and extracted with dichloromethane. The combined organic layers were dried over anhydrous sodium sulphate, filtered and concentrated to afford N-(4-fluoro-3-nitrophenyl)acetamide as off-white solid. Yield: 1.0 g, 57%

Step-2: To a stirred solution of N-(4-fluoro-3-nitrophenyl)acetamide (500 mg, 2.52 mmol) in N,N-dimethylformamide (5.00 mL), sodium hydride (101 mg, 2.52 mmol) was added at 0° C. and stirred for half an hour at room temperature. After half an hour, iodomethane (1.57 mL, 2.52 mmol) was added and reaction mixture stirred for 4 hours at room temperature. After completion, reaction mixture was quenched with water and extracted with ethyl acetate. The organic layer was dried over anhydrous sodium sulfate and concentrated under reduced pressure to get crude. The crude was purified by flash chromatography. The desired fractions concentrated to afford N-(4-fluoro-3-nitrophenyl)-N-methylacetamide (300 mg, 9.05 mmol) as yellow liquid. Yield: 0.30 g, 64%

Step-3: To a stirred solution of N-(4-fluoro-3-nitrophenyl)-N-methylacetamide (180 mg, 848 μmol) in N-Methyl-2-Pyrrolidone (NMP) (2.00 ML), 3-methyloxetan-3-amine (77.8 μL, 2 eq., 1.70 mmol) and ethylbis(propan-2-yl)amine (329 mg, 2.55 μmol) was added at room temperature and stirred at 130° C. for 1 h. After completion, reaction mixture quenched with ice cold water, precipitate was formed. The solid was filtered and dried to afford N-methyl-N-{4-[(3-methyloxetan-3-yl)amino]-3-nitrophenyl}acetamide (200 mg, 716 μmol) as a yellow solid. Yield: 0.20 g, 89%

Step-4: To a stirred solution of N-methyl-N-{4-[(3-methyloxetan-3-yl)amino]-3-nitrophenyl}acetamide (200 mg, 716 μmol) in methanol (10.0 mL), zinc (234 mg, 5 eq., 3.58 mmol) and ammonium chloride (192 mg, 5 eq., 3.58 mmol) were added at room temperature and the reaction mixture stirred for 2 h at 40° C. After completion, the reaction mixture passed through celite bed. Filtrate was extracted with dichloromethane, dried over anhydrous sodium sulfate and concentrated under reduced pressure to get as N-{3-amino-4-[(3-methyloxetan-3-yl)amino]phenyl}-N-methylacetamide (200 mg, 802 μmol) as violet color solid. Yield: 0.20 g (Crude)

Step-5: To a stirred solution of 3,4-bis(benzyloxy)-2-fluoro-5-methoxybenzaldehyde (188 mg, 0.8 eq., 513 μmol) and N-{3-amino-4-[(3-methyloxetan-3-yl)amino]phenyl}-N-methylacetamide (200 mg, 642 μmol) in methanesulfinylmethane (5.00 mL), disodium sulfinatosulfonate (183 mg, 1.5 eq., 963 μmol) was added at room temperature. The resulting mixture stirred for 12 h at 85° C. After completion, ice cold water was added in the reaction mixture and solid was filtered and dried to get crude. The crude purified by flash chromatography. The desired fractions were concentrated to afford N-{2-[3,4-bis(benzyloxy)-2-fluoro-5-methoxyphenyl]-1-(3-methyloxetan-3-yl)-1H-1,3-benzodiazol-5-yl}-N-methylacetamide (250 mg, 378 μmol) as sticky solid. Yield: 0.25 g, 90%

Step-6: To a stirred solution of N-{2-[3,4-bis(benzyloxy)-2-fluoro-5-methoxyphenyl]-1-(3-methyloxetan-3-yl)-1H-1,3-benzodiazol-5-yl}-N-methylacetamide (200 mg, 336 μmol) in trifluoroacetic acid (3.00 mL), reaction mixture stirred for 4 h at 65° C. After completion, reaction mixture was concentrated under reduced pressure to get the crude and further purified by reverse phase HPLC. The desired fractions were lyophilized to afford N-[2-(2-fluoro-3,4-dihydroxy-5-methoxyphenyl)-1-(3-methyloxetan-3-yl)-1H-1,3-benzodiazol-5-yl]-N-methylacetamide (49.0 mg, 117 μmol) as off white solid. Yield: 0.049 mg, 34%

LCMS and NMR data: ES MS M/Z=416 (M+1), UPLC: 99%; ¹H NMR (400 MHz, DMSO-d6) δ 9.59 (s, 1H), 7.72 (s, 1H), 7.39 (d, J=8.4 Hz, 1H), 7.29 (d, J=8.4 Hz, 1H), 6.62 (d, J=6.4 Hz, 1H), 4.72 (d, J=5.6 Hz, 2H), 4.39 (d, J=5.6 Hz, 2H), 3.79 (s, 3H), 3.19 (s, 3H), 2.05 (s, 3H), 1.79 (s, 3H).

### Example 196: Synthesis of methyl 5˜(5-(azetidin-1-yl)-1H-benzo[d]imidazol-2-yl)-2-hydroxy-3-methoxybenzoate

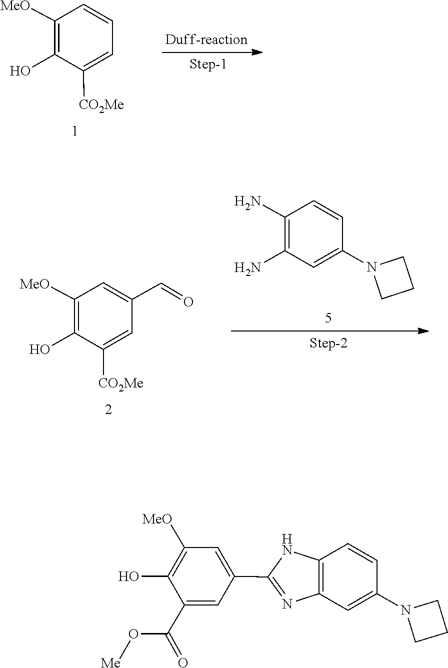 [SMILES: COC(=O)c1cccc(OC)c1O ; COC(=O)c1cc(C=O)cc(OC)c1O ; Nc1ccc(N2CCC2)cc1N ; COC(=O)c1cc(-c2nc3cc(N4CCC4)ccc3[nH]2)cc(OC)c1O]

Step-1: To a stirred solution of methyl 2-hydroxy-3-methoxybenzoate (5.00 g, 27.4 mmol) in trifluoroacetic acid (40.0 mL) hexamethylenetetramine (7.70 g, 2 eq., 54.9 mmol) and cuprous oxide (3.93 g, 27.4 mmol) were added at 0° C. The resulting reaction mixture stirred at 100° C. for 16 h. After the completion of reaction, the trifluoroacetic Acid-Complex was quenched with cold 6 N Hydrochloride solution and extracted with ethyl acetate. The combined organic layers dried over anhydrous sodium sulphate, filtered and concentrated to obtain crude. The crude purified by flash chromatography. The desired fractions were concentrated to afford methyl 5-formyl-2-hydroxy-3-methoxybenzoate (2.00 g, 9.52 mmol). Yield: 2.00 g, 34.67%

Step-2: To a stirred solution of 4-(azetidin-1-yl)benzene-1,2-diamine (155 mg, 952 μmol) and methyl 5-formyl-2-hydroxy-3-methoxybenzoate (200 mg, 952 μmol) in methanesulfinylmethane (5.00 mL), Sodium metabisulfite (217 mg, 1.2 eq., 1.14 mmol) was added at room temperature. The resulting reaction mixture stirred for 16 h at 80° C. After completion, reaction mixture quenched with ice cold water and extracted with ethyl acetate. The organic layer dried over anhydrous sodium sulphate, filtered and concentrated to give crude. The crude purified by Reverse-Phase HPLC chromatography. The desired fractions were collected and lyophilized to afford methyl 5-[5-(azetidin-1-yl)-1H-1,3-benzodiazol-2-yl]-2-hydroxy-3-methoxybenzoate (15.0 mg, 42.4 μmol) as off white solids. Yield: 15 mg, 4.46%

LCMS and NMR data: ES MS M/Z=354.12 (M+1), UPLC: 98.82% 1H NMR (400 MHz, DMSO-d6) δ 12.58-12.48 (m, 1H), 10.67 (s, 1H), 8.17-8.12 (m, 1H), 7.92-7.90 (m, 1H), 7.43-7.30 (m, 1H), 6.59-6.34 (m, 2H), 3.95 (s, 3H), 3.93 (s, 3H), 3.83-3.80 (t, 4H), 2.32-2.28 (t, 2H).

### Example 197: Synthesis of 1-(2-(2-fluoro-3,4-dihydroxy-5-methoxyphenyl)-1-(3-methyloxetan-3-yl)-1H-benzo[d]imidazol-5-yl)-3-methylimidazolidin-2-one

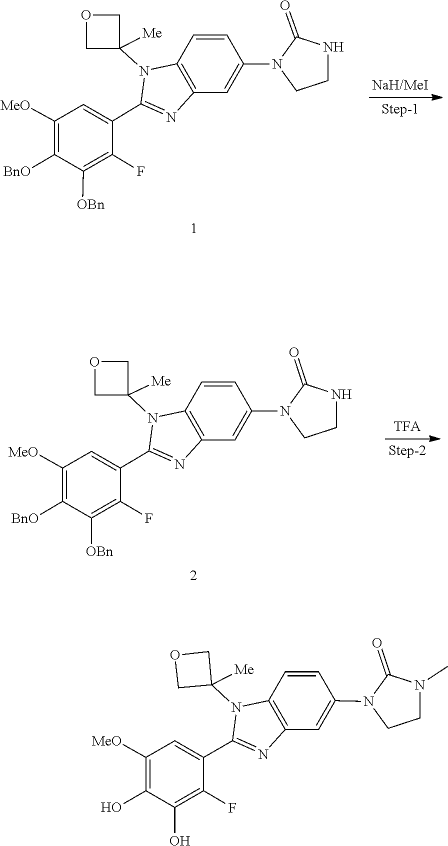 [SMILES: COc1cc(-c2nc3cc(N4CCNC4=O)ccc3n2C2(C)COC2)c(F)c(OCc2ccccc2)c1OCc1ccccc1 ; COc1cc(-c2nc3cc(N4CCNC4=O)ccc3n2C2(C)COC2)c(F)c(OCc2ccccc2)c1OCc1ccccc1 ; COc1cc(-c2nc3cc(N4CCN(C)C4=O)ccc3n2C2(C)COC2)c(F)c(O)c1O]

Step-1: To a stirred solution of 1-{2-[3,4-bis(benzyloxy)-2-fluoro-5-methoxyphenyl]-1-(3-methyloxetan-3-yl)-1H-1,3-benzodiazol-5-yl}imidazolidin-2-one (190 mg, 312 μmol) in N,N-dimethylformamide (2.00 mL) was added sodium hydride (12.5 mg, 312 μmol) at 0° C. and stirred for half an hour at room temperature. After half an hour, iodomethane (19.4 μL, 312 μmol) was added and the solution was stirred for 16 hours at room temperature. After completion, reaction mixture was quenched with water and extracted with ethyl acetate. The organic layer was dried over anhydrous sodium sulfate and concentrated under reduced pressure to get crude. The crude was purified by flash chromatography. The desired fractions concentrated to afford 1-{2-[3,4-bis(benzyloxy)-2-fluoro-5-methoxyphenyl]-1-(3-methyloxetan-3-yl)-1H-1,3-benzodiazol-5-yl}-3-methylimidazolidin-2-one (80.0 mg, 78.4 μmol). Yield: 0.080 g, (25%)

Step-2: To a stirred solution of 1-{2-[3,4-bis(benzyloxy)-2-fluoro-5-methoxyphenyl]-1-(3-methyloxetan-3-yl)-1H-1,3-benzodiazol-5-yl}-3-methylimidazolidin-2-one (70.0 mg, 112 μmol) was added trifluoroacetic acid (2.00 mL) and the resulting mixture was stirred for 2 h at 50° C. After completion, reaction mixture was concentrated under reduced pressure to get the crude and further purified by reverse phase HPLC. Desired fractions were lyophilized to afford 1-[2-(2-fluoro-3,4-dihydroxy-5-methoxyphenyl)-1-(3-methyloxetan-3-yl)-1H-1,3-benzodiazol-5-yl]-3-methylimidazolidin-2-one (8.00 mg, 17.8 μmol) as white solid.

1H NMR: ES MS M/Z=443 (M+1), NMR (400 MHz, DMSO-d6) δ 9.41 (br s, 1H), 7.70 (d, 1H), 7.64 (d, 1H), 7.21 (d, 1H), 6.57 (d, 1H), 4.70 (d, 2H), 4.37 (d, 2H), 3.84 (t, 2H), 3.78 (s, 3H), 3.45 (t, 2H), 2.78 (s, 3H), 2.00 (s, 3H), 1.23 (s, 1H). Yield: 0.008 g, 15.8%

### Example 198: Synthesis of 1-(2-(2-fluoro-3,4-dihydroxy-5-methoxyphenyl)-1-(3-methyloxetan-3-yl)-1H-benzo[d]imidazol-5-yl)imidazolidin-2-one

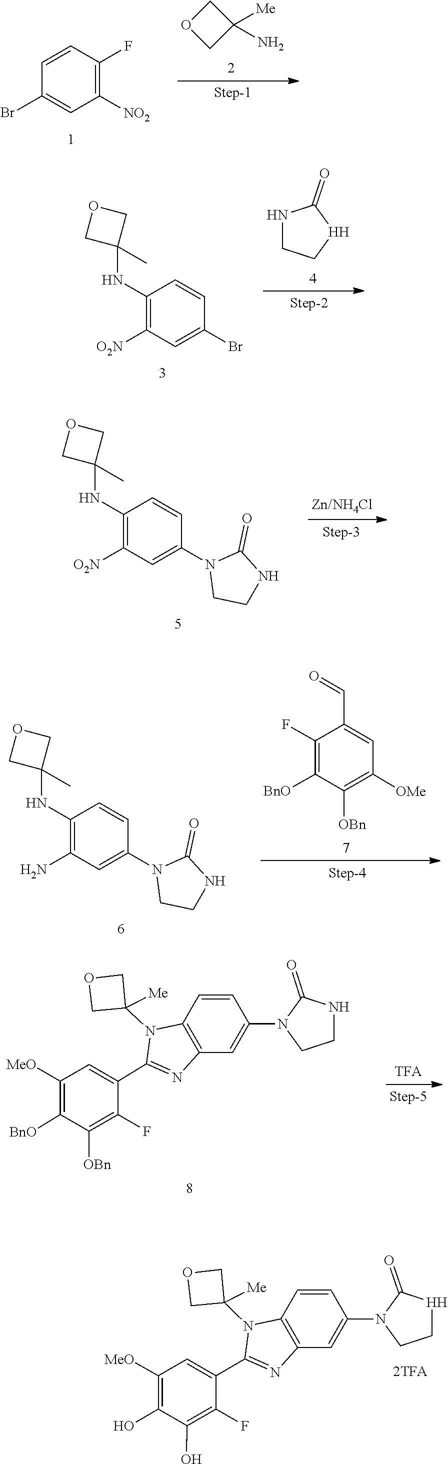

Step-1: To a stirred solution of 4-bromo-1-fluoro-2-nitrobenzene (2.00 g, 9.09 mmol) in 1-methylpyrrolidin-2-one (10.0 mL) was added 3-methyloxetan-3-amine (1.14 mL, 1.5 eq., 13.6 mmol) and ethylbis(propan-2-yl)amine (4.76 mL, 3 eq., 27.3 mmol) and heated at 100° C. for 16 h. After completion of reaction, reaction mixture diluted with water and extracted with ethyl acetate. The organic layer was dried over anhydrous sodium sulphate, filtered and concentrated to afford N-(4-bromo-2-nitrophenyl)-3-methyloxetan-3-amine (2.48 g, 7.08 mmol) as orange solid. Yield: 2.48 g, 77.91%

Step-2: To a stirred solution of N-(4-bromo-2-nitrophenyl)-3-methyloxetan-3-amine (500 mg, 1.74 mmol) in 1,4-dioxane (1.00 mL), imidazolidin-2-one (300 mg, 2 eq., 3.48 mmol) and Potassium carbonate (722 mg, 3 eq., 5.22 mmol) were added and reaction mixture was purged with argon for 5 min then Copper (I)Iodide (166 mg, 0.3 eq., 522 μmol) and 2-aminoacetic acid (78.4 mg, 0.6 eq., 1.04 mmol) were added and again purged with argon. The reaction mixture was heated to 130° C. for 16 h. After completion of reaction, reaction mixture diluted with water and extracted with ethyl acetate. The organic layer was dried over anhydrous sodium sulfate and concentrated under reduced pressure to get crude. The crude purified by flash chromatography. The desired fractions were concentrated to afford 1-{4-[(3-methyloxetan-3-yl)amino]-3-nitrophenyl}imidazolidin-2-one (90.0 mg, 274 μmol) as red solid. Yield: 90 mg, 15.7%

Step-3: To a stirred solution of 1-{4-[(3-methyloxetan-3-yl)amino]-3-nitrophenyl}imidazolidin-2-one (90.0 mg, 308 μmol) in methanol (10.0 mL), zinc (101 mg, 5 eq., 1.54 mmol) and ammonium chloride (82.4 mg, 5 eq., 1.54 mmol) were added at room temperature and stirred at 50° C. for 1 h. After completion of the reaction, reaction mixture was passed through celite and washed with 10% methanol in dichloromethane, filtrate was washed with water, dried over anhydrous sodium sulfate and concentrated under reduced pressure to get the desired product 1-{3-amino-4-[(3-methyloxetan-3-yl)amino]phenyl}imidazolidin-2-one (82.0 mg, 269 μmol) as pale green powdery solid. Yield: 82 mg, 87.31%

Step-4: To a stirred solution of 1-{3-amino-4-[(3-methyloxetan-3-yl)amino]phenyl}imidazolidin-2-one (82.0 mg, 313 μmol) and 3,4-bis(benzyloxy)-2-fluoro-5-methoxybenzaldehyde (91.6 mg, 0.8 eq., 250 μmol) in methanesulfinylmethane (3.00 mL) was added disodium sulfinatosulfonate (71.3 mg, 1.2 eq., 375 μmol) at room temperature. The resulting mixture was stirred for 16 h at 80° C. After completion, reaction mixture was diluted with water and extracted with ethyl acetate. The organic layer dried over anhydrous sodium sulfate, filtered and concentrated under reduced pressure to get crude. The crude purified by flash chromatography. The desired fractions concentrated to afford 1-{2-[3,4-bis(benzyloxy)-2-fluoro-5-methoxyphenyl]-1-(3-methyloxetan-3-yl)-1H-1,3-benzodiazol-5-yl}imidazolidin-2-one (65.0 mg, 69.0 μmol) as yellow solid. Yield: 0.065 g, 22%

Step-5: To 1-{2-[3,4-bis(benzyloxy)-2-fluoro-5-methoxyphenyl]-1-(3-methyloxetan-3-yl)-1H-1,3-benzodiazol-5-yl}imidazolidin-2-one (55.0 mg, 90.4 μmol) was added trifluoroacetic acid (2.00 mL) and the resulting solution was stirred for 2 h at 50° C. After completion, the reaction mixture was concentrated under reduced pressure to get the crude and further purified by reverse phase HPLC. Desired fractions were lyophilized to afford 1-[2-(2-fluoro-3,4-dihydroxy-5-methoxyphenyl)-1-(3-methyloxetan-3-yl)-1H-1,3-benzodiazol-5-yl]imidazolidin-2-one (8.00 mg, 18.3 μmol) (TFA Salt) as off white solid. Yield: 0.008 g, 20%

1H NMR and LCMS: ES MS M/Z=429 (M+1), NMR (400 MHz, DMSO-d6) δ 9.76 (br s, 2H), 7.87 (s, 1H), 7.70 (d, 1H), 7.40 (d, 1H), 7.04 (s, 1H), 6.67 (d, 1H), 4.73 (d, 2H), 4.44 (d, 2H), 3.95 (t, 2H), 3.81 (s, 3H), 3.44 (t, 2H), 2.09 (s, 3H).

### Example 199: Synthesis of 4-[5-(azetidin-1-yl)-1-(oxetan-3-yl)-1H-1,3-benzodiazol-2-yl]-3-fluoro-6-methoxy-5-methylbenzene-1,2-diol

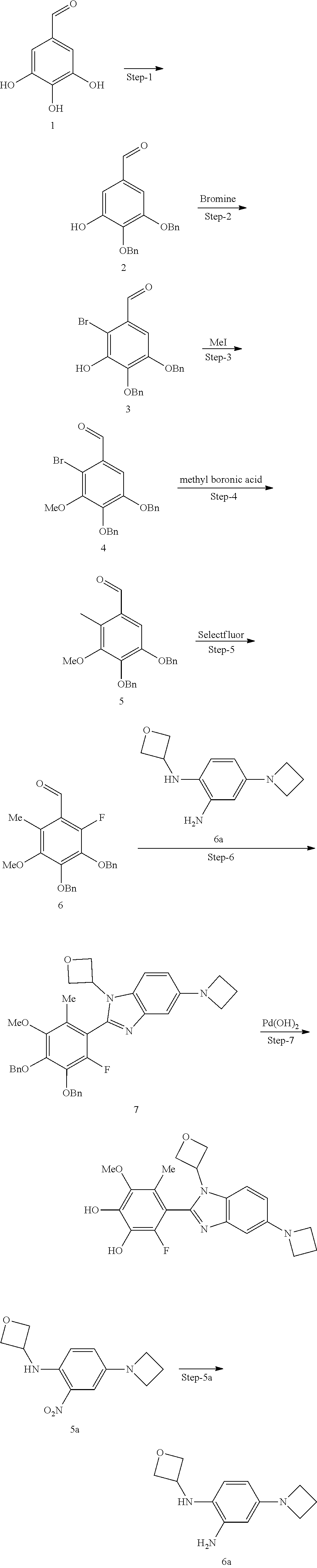

Step-1: To a solution of 3,4,5-trihydroxybenzaldehyde (20.0 g, 2.4 eq., 130 mmol) in Acetone (100 mL), (bromomethyl)benzene (9.75 mL, 1.5 eq., 79.8 mmol), dipotassium carbonate (13.2 g, 1.8 eq., 95.8 mmol) and potassium iodide (1.77 g, 0.2 eq., 10.6 mmol) were added to the reaction mixture and stirred and heated at 60° C. for 4 h. After completion of the reaction, the reaction mixture was filtered and the filtrate was concentrated under reduced pressure to get the crude. The crude purified by flash chromatography. The desired fractions were concentrated to afford methyl 3,4-bis(benzyloxy)-5-hydroxybenzoate as off white solid. Yield: 7.00 g, 24.39%

Step-2: To a solution of 3,4-bis(benzyloxy)-5-hydroxybenzaldehyde (2.56 g, 7.66 mmol) in Acetic acid (70.0 mL) and dibromine (393 μL, 7.66 mmol) diluted in acetic acid was added dropwise to the reaction mixture and stirred for 30 minute. After completion of the reaction, the reaction mixture was quenched with Sodium thiosulfate and stirred for 10 minute so that solid compound precipitate. After that reaction mixture filtered out and solid washed with ice cold water and dried under vacuum. The solid afford 4,5-bis(benzyloxy)-2-bromo-3-hydroxybenzaldehyde (2.00 g, 3.87 mmol) as off white solid. Yield: 2.00 g, 50.57%

Step-3: To a stirred solution 4,5-bis(benzyloxy)-2-bromo-3-hydroxybenzaldehyde (2.00 g, 3.87 mmol) in N,N-dimethylformamide (3.00 mL) and dipotassium carbonate (1.07 g, 2 eq., 7.74 mmol) was added to the reaction mixture at room temperature. The resulting mixture stirred for 1 h at room temperature. After 1 hr Methyl iodide (289 μL, 1.2 eq., 4.65 mmol) was added to the reaction mixture at room temperature. After completion, reaction mixture diluted with ice cold water and extracted with ethyl acetate. The organic layer dried over anhydrous sodium sulfate and concentrated under reduced pressured to get crude. The crude purified by flash chromatography. The desired fractions were concentrated to afford 4,5-bis(benzyloxy)-2-bromo-3-methoxybenzaldehyde (1.30 g, 2.74 mmol) as White solid. Yield: 1.30 g, 70.72%

Step-4: To a solution of 4,5-bis(benzyloxy)-2-bromo-3-methoxybenzaldehyde (600 mg, 1.40 mmol) and methylboronic acid (126 mg, 1.5 eq., 2.11 mmol) in 1,4-dioxane (2.50 mL) and water (500 μL). cesium carbonate (915 mg, 2 eq., 2.81 mmol) was added at room temperature and the reaction mixture was degassed with argon for 5 min. 1,1′-Bis(diphenylphosphino)ferrocene]dichloropalladium(II) (205 mg, 0.2 eq., 281 μmol) was added to the reaction, continued degassing for 5 min and heated the reaction mixture at 90° C. for 4 h. After completion, the reaction was cooled to room temperature and passed through celite bed. Filtrate was diluted with ethyl acetate and washed with water. The organic layer was dried over anhydrous sodium sulfate, filtered and concentrated to give crude. The crude purified by flash chromatography. The desired fractions were concentrated to afford 4,5-bis(benzyloxy)-2-bromo-3-methoxybenzaldehyde (1.30 g, 2.74 mmol) as White solid. Yield: 0.320 g, 41%

Step-5: To a stirred solution of 4,5-bis(benzyloxy)-3-methoxy-2-methylbenzaldehyde (320 mg, 883 μmol) in acetonitrile (10.0 mL) was added 4-fluoro-1-methyl-1,4-diazabicyclo[2.2.2]octane-1,4-diium; bis(tetrafluoroboranuide) (424 mg, 1.5 eq., 1.32 mmol) room temperature. The resulting mixture stirred for 2 h at 45° C. for 16 h. After completion, reaction mixture concentrated under reduced pressure to get the crude. Crude was purified by flash chromatography. Desired fractions were concentrated to get 3,4-bis(benzyloxy)-2-fluoro-5-methoxy-6-methylbenzaldehyde (60.0 mg, 142 μmol) as yellow color viscous liquid. Yield: 0.100 g, (16.61%)

Step-5a: To a solution of N-[4-(azetidin-1-yl)-2-nitrophenyl]oxetan-3-amine (700 mg, 2.81 mmol) in methanol (15.0 mL) was added Palladium on carbon (500 mg) at room temperature and the reaction mixture stirred for 3 h under hydrogen atmosphere. After completion, the reaction mixture passed through celite bed. The filtrate was concentrated to get 4-(azetidin-1-yl)-N1-(oxetan-3-yl)benzene-1,2-diamine (600 mg, 2.74 mmol) as brown semisolid. Yield: 0.60 g, (70%)

Step-6: To a stirred solution N-[4-(azetidin-1-yl)-2-nitrophenyl]oxetan-3-amine (100 mg, 401 μmol) and 3,4-bis(benzyloxy)-2-fluoro-5-methoxy-6-methylbenzaldehyde (76.3 mg, 0.5 eq., 201 μmol) in methanesulfinylmethane (5.00 mL) and disodium sulfinatosulfonate (91.5 mg, 1.2 eq., 481 μmol) was added at room temperature. The resulting mixture stirred and heated at 85° C. for 16 hr. After completion of reaction, the reaction mixture diluted with ice cold water and extracted with ethyl acetate. The organic layer dried over anhydrous sodium sulphate, filtered and concentrated to give 5-(azetidin-1-yl)-2-[3,4-bis(benzyloxy)-2-fluoro-5-methoxy-6-methylphenyl]-1-(oxetan-3-yl)-1H-1,3-benzodiazole (70.0 mg, 99.3 μmol) as brown color solid. Yield: 0.07 g, (24.76%)

Step-7: To a solution of 5-(azetidin-1-yl)-2-[3,4-bis(benzyloxy)-2-fluoro-5-methoxy-6-methylphenyl]-1-(oxetan-3-yl)-1H-1,3-benzodiazole (75.0 mg, 106 μmol) in Tetrahydrofuran (10.0 mL) and Palladium hydroxide on carbon (150 mg, 12 eq., 1.23 mmol) was added at room temperature and the reaction mixture stirred for 2 h under hydrogen atmosphere. After completion, the reaction mixture passed through celite bed. Filtrate was concentrated to get crude. The crude was purified using reverse phase prep HPLC to get 4-[5-(azetidin-1-yl)-1-(oxetan-3-yl)-1H-1,3-benzodiazol-2-yl]-3-fluoro-6-methoxy-5-methylbenzene-1,2-diol (4.00 mg, 9.52 μmol) as white solid. Yield: 0.004 g, 8.98%

LCMS and NMR Data: ES MS M/Z=400.101 (M+1), UPLC: 95.11%. 1HNMR (400 MHz, DMSO-d6): δ 9.45 (s, 1H), 9.35 (s, 1H), 7.86 (d, J=8.6 Hz, 1H), 6.68 s, 1H), 6.57 (d, J=8.6 Hz, 1H), 5.20-5.03 (m, 1H), 5.01-4.95 (m, 4H), 3.81 (t, J=6.8 Hz, 4H), 3.80 (s, 3H), 2.49 (t, J=1.68 Hz, 2H), 2.31 (s, 3H).

### Example 200: Synthesis of 4-[5-(azetidin-1-yl)-1-cyclobutyl-1H-1,3-benzodiazol-2-yl]-3-fluoro-6-methoxy-5-methylbenzene-1,2-diol

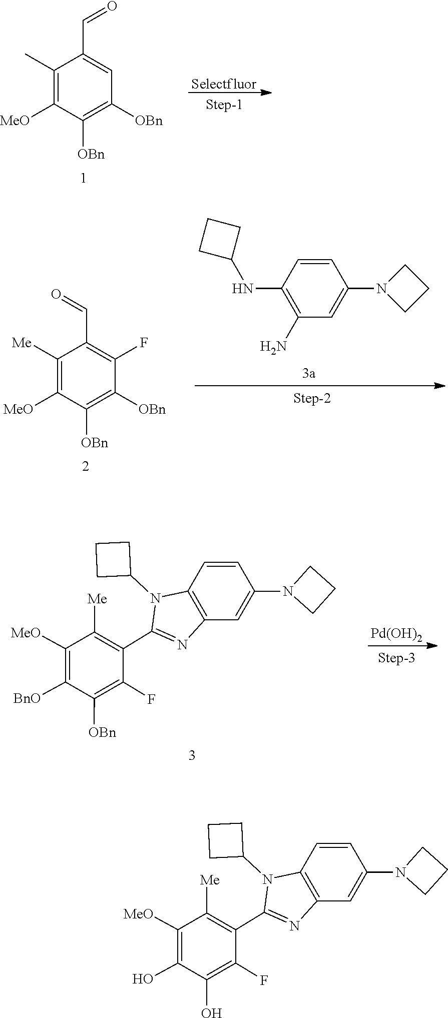

Step-1: To a stirred solution of 4,5-bis(benzyloxy)-3-methoxy-2-methylbenzaldehyde (520 mg, 1.43 mmol) in acetonitrile (10.0 mL) was added 4-fluoro-1-methyl-1,4-diazabicyclo[2.2.2]octane-1,4-diium; bis(tetrafluoroboranuide) (688 mg, 1.5 eq., 2.15 mmol) room temperature. The resulting mixture stirred for 2 h at 45° C. for 16 h. After completion, reaction mixture concentrated under reduced pressure to get the crude. Crude was purified by flash chromatography. Desired fractions were concentrated to get 3,4-bis(benzyloxy)-2-fluoro-5-methoxy-6-methylbenzaldehyde (120 mg, 284 μmol) as yellow color viscous liquid. Yield: 0.120 g, 19.79%

Step-2: To a stirred solution 4-(azetidin-1-yl)-N1-cyclobutylbenzene-1,2-diamine (68.6 mg, 1.2 eq., 315 μmol) and 3,4-bis(benzyloxy)-2-fluoro-5-methoxy-6-methylbenzaldehyde (100 mg, 263 μmol) in Dimethyl sulfoxide (3.00 mL) and disodium sulfinatosulfonate (60.0 mg, 1.2 eq., 315 μmol) was added at room temperature. The resulting mixture stirred and heated at 85° C. for 16 hr. After completion of reaction, the reaction mixture diluted with ice cold water and extracted with ethyl acetate. The organic layer dried over anhydrous sodium sulphate, filtered and concentrated to give crude. The crude purified by flash chromatography. The desired fractions were concentrated to afford 5-(azetidin-1-yl)-2-[3,4-bis(benzyloxy)-2-fluoro-5-methoxy-6-methylphenyl]-1-cyclobutyl-1H-1,3-benzodiazole (150 mg, 77.9 μmol) as white color viscous liquid. Yield: 0.15 g, 29.63%

Step-3: To a solution of 5-(azetidin-1-yl)-2-[3,4-bis(benzyloxy)-2-fluoro-5-methoxy-6-methylphenyl]-1-cyclobutyl-1H-1,3-benzodiazole (75.0 mg, 130 μmol) in methanol (30.0 mL) and Palladium hydroxide was added at room temperature and the reaction mixture stirred for 2 h under hydrogen atmosphere. After completion, the reaction mixture passed through celite bed. Filtrate was concentrated to get Crude. The crude was purified using reverse phase prep HPLC to get 4-[5-(azetidin-1-yl)-1-cyclobutyl-1H-1,3-benzodiazol-2-yl]-3-fluoro-6-methoxy-5-methylbenzene-1,2-diol (4.00 mg, 9.64 μmol) as a white solid. Yield: 0.004 g, 7.42%

LCMS and NMR Data: ES MS M/Z=398.06 (M+1)⁺, UPLC: 95.78%. 1HNMR (400 MHz, DMSO-d6): δ 9.43 (s, 1H), 9.3 (s, 1H), 7.65 (d, J=8.4 Hz, 1H), 6.61 (s, 1H), 6.49 (d, J=8.4 Hz, 1H), 4.53 (t, J=8.4 Hz, 1H), 3.76 (t, 4H), 3.65 (s, 3H), 2.67-2.50 (m, 2H), 2.25 (t, J=6.8 Hz, 2H), 2.15 (m, 2H), 1.89 (s, 3H).

### Example 201: Synthesis of 5-(1-cyclobutyl-1H-1,3-benzodiazol-2-yl)-3-ethoxybenzene-1,2-diol

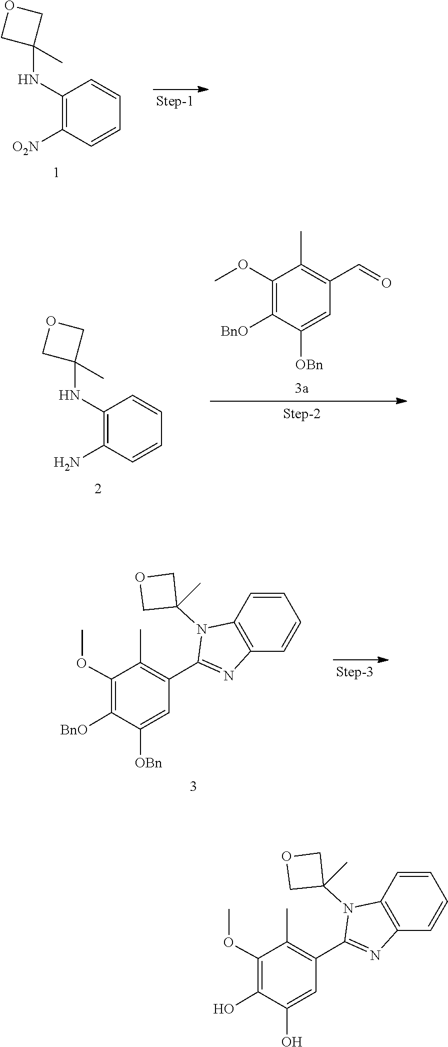 [SMILES: CC1(Nc2ccccc2[N+](=O)[O-])COC1 ; CC1(Nc2ccccc2N)COC1 ; COc1c(C)c(C=O)cc(OCc2ccccc2)c1OCc1ccccc1 ; COc1c(C)c(-c2nc3ccccc3n2C2(C)COC2)cc(OCc2ccccc2)c1OCc1ccccc1 ; COc1c(C)c(-c2nc3ccccc3n2C2(C)COC2)cc(O)c1O]

Step-1: To a solution of 3-methyl-N-(2-nitrophenyl)oxetan-3-amine (200 mg, 961 μmol) in methanol (30.0 mL) was added Palladium on Carbon (400 mg, 3.76 mmol) at room temperature and the reaction mixture stirred for 2 h under hydrogen atmosphere. After completion, the reaction mixture passed through celite bed. Filtrate was concentrated to get as N-(3-methyloxetan-3-yl)benzene-1,2-diamine (150 mg, 808 μmol) red color semi solid. Yield: 0.15 g, 84.11%

Step-2: To a stirred solution N1-(3-methyloxetan-3-yl)benzene-1,2-dianine (61.5 mg, 345 μmol) and 4,5-bis(benzyloxy)-3-methoxy-2-methylbenzaldehyde (100 mg, 0.8 eq., 276 μmol) in Dimethyl sulfoxide (3.00 mL) and disodium sulfinatosulfonate (78.7 mg, 1.2 eq., 414 μmol) was added at room temperature. The resulting mixture stirred and heated at 85° C. for 16 hr. After completion of reaction, the reaction mixture diluted with ice cold water and extracted with ethyl acetate. The organic layer dried over anhydrous sodium sulphate, filtered and concentrated to give crude. The crude purified by flash chromatography. The desired fractions were concentrated to afford 2-[4,5-bis(benzyloxy)-3-methoxy-2-methylphenyl]-1-(3-mnethyloxetan-3-yl)-1H-1,3-benzodiazole (150 mg, 254 μmol) as white color liquid Yield: 0.150 g, 73.51%

Step-3: To a solution of 2-[4,5-bis(benzyloxy)-3-methoxy-2-methylphenyl]-1-(3-methyloxetan-3-yl)-1H-L3-benzodiazole (110 mg, 211 μmol) in Tetrahydrofuran (15.0 mL) and Palladium hydroxide on carbon 20% w/w (300 mg, 2.3 eq., 490 μmol) was added at room temperature and the reaction mixture stirred for 2 h under hydrogen atmosphere. After completion, the reaction mixture passed through celite bed. Filtrate was concentrated low temperature to get crude. The crude was purified using reverse phase prep HPLC and desired fractions were lyophilized to get 3-methoxy-4-methyl-5-[1-(3-methyloxetan-3-yl)-1H-1,3-benzodiazol-2-yl]benzene-1,2-diol (39.0 mg, 114 μmol) as white color solid. Yield: 0.039 g, (53.73%)

LCMS and NMR Data: ES MS M/Z=341.12 (M+1); UPLC: 99.08%; 1H NMR (400 MHz, DMSO-d6) δ 9.01 (bs, 1H), 7.67-7.65 (m, 1H), 7.25-7.22 (m, 3H), 6.51 (s, 1H), 4.60 (s, 4H), 3.72(s, 3H), 2.01 (d, J=12.4 Hz 6H)

### Example 202: Synthesis of 4-(6,7-dichloro-1H/benzo[d]imidazol-2-yl)-3-fluoro-6-methoxybenzene-1,2-diol

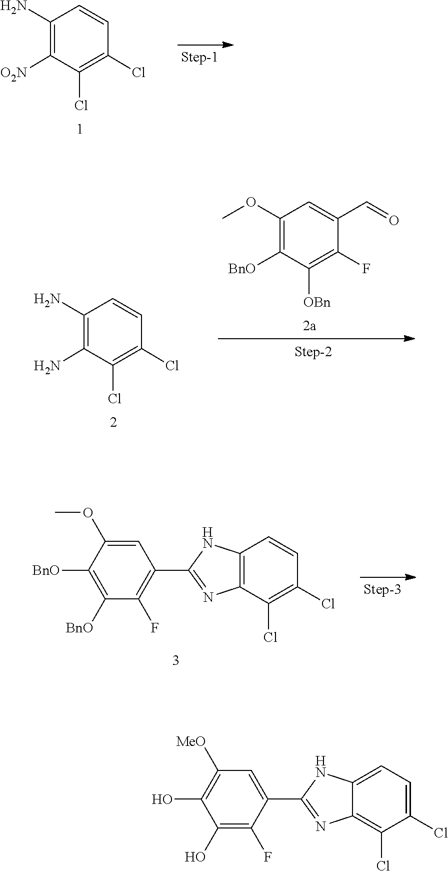 [SMILES: Nc1ccc(Cl)c(Cl)c1[N+](=O)[O-] ; Nc1ccc(Cl)c(Cl)c1N ; COc1cc(C=O)c(F)c(OCc2ccccc2)c1OCc1ccccc1 ; COc1cc(-c2nc3c(Cl)c(Cl)ccc3[nH]2)c(F)c(OCc2ccccc2)c1OCc1ccccc1 ; COc1cc(-c2nc3c(Cl)c(Cl)ccc3[nH]2)c(F)c(O)c1O]

Step-1: To a stirred solution of 2,3-dichloro-6-nitroaniline (500 mg, 2.42 mmol) and iron (405 mg, 3 eq., 7.25 mmol) and acetic acid (2.00 mL) was added at room temperature. The resulting mixture stirred and heated for 16 h at 65° C. After completion, reaction mixture diluted with water and extracted wit ethyl acetate. The organic layer dried over anhydrous sodium sulphate, filtered and concentrated to give crude. The crude purified by flash chromatography. The desired fractions were concentrated to afford 3,4-dichlorobenzene-1,2-diamine (300 mg, 1.69 mmol) as off yellow color solid. Yield: 0.30 g, 70.16%

Step-2: To a stirred solution 3,4-dichlorobenzene-1,2-diamine (96.6 mg, 546 μmol) and 3,4-bis(benzyloxy)-2-fluoro-5-methoxybenzaldehyde (200 mg, 546 μmol) in methanol (3.00 mL) and catalytic amount of acetic acid was added to the reaction mixture at room temperature. The resulting mixture stirred for 16 h at 80° C. After completion, reaction mixture concentrated under reduced pressure to get 2-[3,4-bis(benzyloxy)-2-fluoro-5-methoxyphenyl]-6,7-dichloro-1H-1,3-benzodiazole white solid. Yield: 210 mg. 40.43%

Step-3: To a stirred solution 2-[3,4-bis(benzyloxy)-2-fluoro-5-methoxyphenyl]-6,7-dichloro-1H-1,3-benzodiazole (200 mg, 382 μmol) in trifluoroacetic acid (3.00 mL) and the resulting mixture stirred and heated for 16 h at 65° C. After completion, reaction mixture concentrated under reduced pressure to get crude. The crude was purified using prep HPLC to get 2-[3,4-bis(benzyloxy)-2-fluoro-5-methoxyphenyl]-6,7-dichloro-1H-1,3-benzodiazole white solid. Yield: 29 mg, 21.32%

LCMS and NMR Data: ES MS M/Z=343.01 (M+1); UPLC: 96.41%; 1H NMR (400 MHz, DMSO-d6) δ 7.52 (d, J=8.4 Hz, 1H), 7.38 (d, J=8.8 Hz, 1H), 7.18 (d, J=4.3 Hz, 1H), 3.86 (s, J=7.0 Hz, 3H).

### Example 203: Synthesis of N-[2-(2-fluoro-3,4-dihydroxy-5-methoxyphenyl)-1-(3-methyloxetan-3-yl)-1H-1,3-benzodiazol-5-yl]pyridine-2-carboxamide

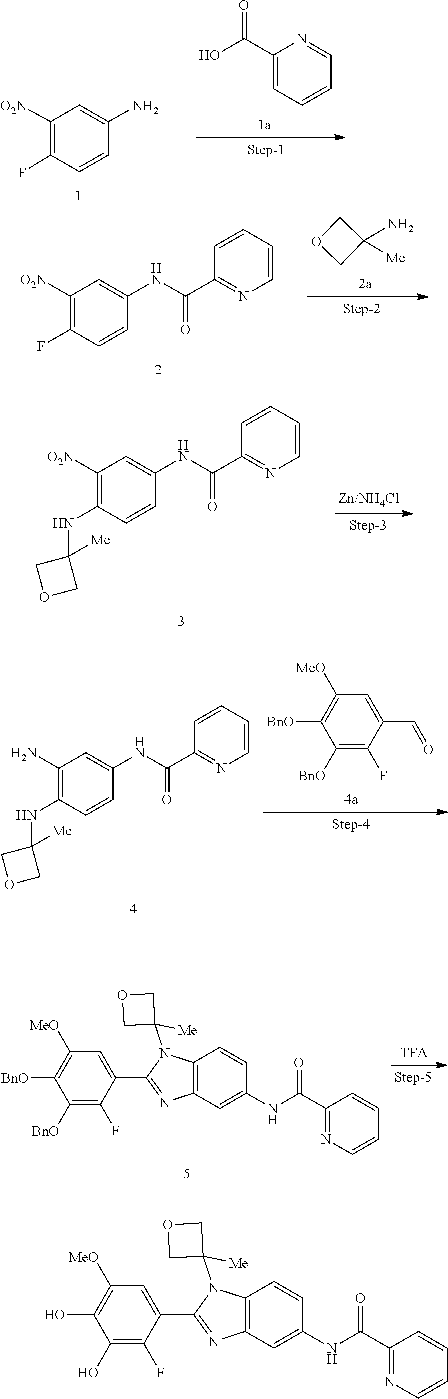

Step-1: To a stirred solution of 4-fluoro-3-nitroaniline (500 mg, 3.20 mmol) in N,N-dimethylformamide (2.00 mL) was added pyridine-2-carboxylic acid (394 mg, 3.20 mmol), hexafluoro-λ⁵-phosphanuide 1-[bis(dimethylamino)methylidene]-1H-1λs-[1,2,3]triazolo[4,5-b]pyridin-3-ium-1-ylium-3-olate (1.83 g, 1.5 eq., 4.80 mmol) and ethylbis(propan-2-yl)amine (1.67 mL, 3 eq., 9.61 mmol) and the solution was stirred for 16 hours. After completion of reaction, the reaction mixture diluted with water and extracted with ethyl acetate. The organic layer dried over anhydrous sodium sulphate, filtered and concentrated to obtain crude. The crude was purified by flash chromatography. The desired fractions concentrated to afford N-(4-fluoro-3-nitrophenyl)pyridine-2-carboxamide (650 mg, 2.49 mmol) as yellow solid. Yield: 650 mg, 77.7%

Step-2: To a stirred solution of N-(4-fluoro-3-nitrophenyl)pyridine-2-carboxamide (400 mg, 1.53 mmol) and N, N-Diisopropylethylamine (802 μL, 3 eq., 4.59 mmol) in N-Methyl-2-Pyrrolidone (NMP) (5.00 mL), 3-methyloxetan-3-amine (138 μL, 2 eq., 3.06 mmol) was added at room temperature and stirred at 100° C. for 16 h. After completion, reaction mixture quenched with ice cold water, precipitate was formed. The solid was filtered and dried to afford N-{4-[(3-methyloxetan-3-yl)amino]-3-nitrophenyl}pyridine-2-carboxamide (500 mg, 1.52 mmol) as a yellow solid. Yield: 500 mg, 99%

Step-3: To a solution of N-{4-[(3-methyloxetan-3-yl)amino]-3-nitrophenyl}pyridine-2-carboxamide (300 mg, 914 μmol) in methanol (20.0 mL) was added zinc (299 mg, 5 eq., 4.57 mmol) at room temperature and the reaction mixture stirred for 3 h under hydrogen atmosphere. After completion, the reaction mixture passed through celite bed. The filtrate was concentrated at room temperature to get as N-{3-amino-4-[(3-methyloxetan-3-yl)amino]phenyl}pyridine-2-carboxamide (270 mg, 905 μmol) brown color solid. Yield: 270 mg, 99%

Step-4: To a stirred solution of N-{3-amino-4-[(3-methyloxetan-3-yl)amino]phenyl}pyridine-2-carboxamide (203 mg, 681 μmol) and 3,4-bis(benzyloxy)-2-fluoro-5-methoxybenzaldehyde (200 mg, 0.8 eq., 545 μmol) in methanesulfinylmethane (3.00 mL), disodium sulfinatosulfonate (194 mg, 1.5 eq., 1.02 mmol) was added at room temperature. The resulting mixture stirred for 16 h at 80° C. After completion, water and ethyl acetate was added to reaction mixture. The organic fractions were collected, dried over anhydrous sodium sulfate, filtered and concentrated to get crude. The crude purified by flash chromatography. The desired fractions were concentrated to afford N-{2-[3,4-bis(benzyloxy)-2-fluoro-5-methoxyphenyl]-1-(3-methyloxetan-3-yl)-1H-1,3-benzodiazol-5-yl}pyridine-2-carboxamide (240 mg, 372 μmol) as sticky solid. Yield: 240 mg, 54.65%

Step-5: To N-{2-[3,4-bis(benzyloxy)-2-fluoro-5-methoxyphenyl]-1-(3-methyloxetan-3-yl)-1H-1,3-benzodiazol-5-yl}pyridine-2-carboxamide (150 mg, 233 μmol) trifluoroacetic acid (2.00 mL) added at room temperature. The resulting reaction mixture stirred at 60° C. for 4 h. After the complete consumption of starting material, reaction mixture was concentrated to obtain the crude. The crude purified in reverse-phase HPLC and desired fraction were collected, lyophilized to obtain N-[2-(2-fluoro-3,4-dihydroxy-5-methoxyphenyl)-1-(3-methyloxetan-3-yl)-1H-1,3-benzodiazol-5-yl]pyridine-2-carboxamide (15.0 mg, 32.3 μmol) as off white solid. Yield: 15 mg, 13.88%

LCMS and NMR Data: ES MS M/Z=464.96 (M+1); UPLC: 97.40%; ¹H NMR (400 MHz, DMSO-d6) δ 10.71 (s, 1H), 9.49 (s, 1H), 9.39 (s, 1H), 8.76 (d, J=4.8 Hz, 1H), 8.32 (d, J=1.6 Hz, 1H), 8.19 (d, J=8.0 Hz, 1H), 8.17-8.06 (m, 1H), 7.77 (dd, J=6.8, 2.0, 1H), 7.70-7.67 (m, 1H), 7.26 (d, J=8.8 Hz, 1H), 6.61 (d, J=6.0 Hz, 1H), 4.73 (d, J=6.0 Hz, 2H) 4.39 (d, J=6.0 Hz, 2H), 3.80 (s, 3H), 2.02 (s, 3H)

### Example 204: Synthesis of N-[2-(2-fluoro-3,4-dihydroxy-5-methoxyphenyl)-1-(3-methyloxetan-3-yl)-1H-1,3-benzodiazol-5-yl]cyclopropanecarboxamide

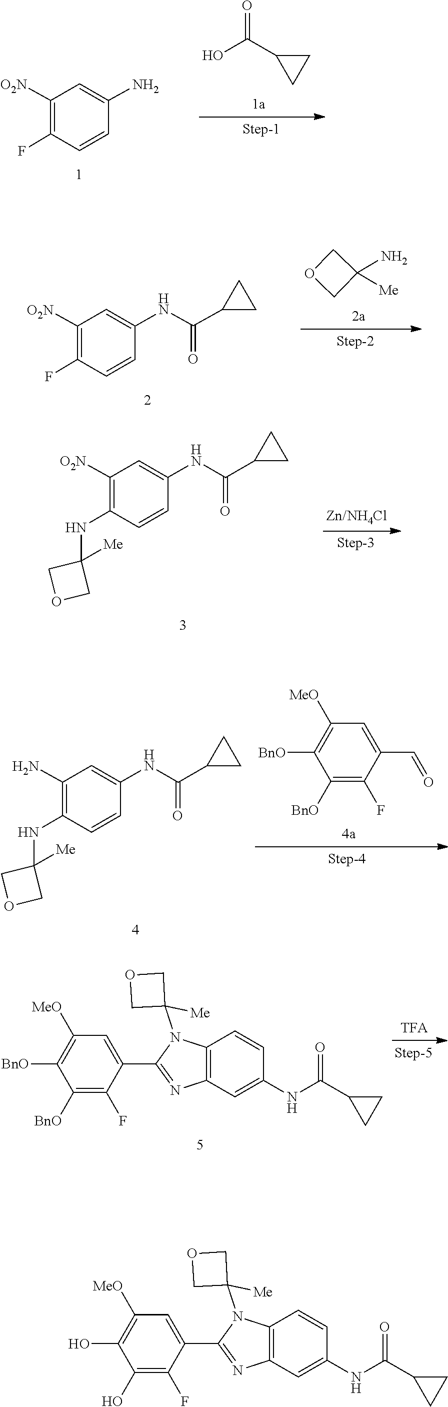

Step-1: To a stirred solution of 4-fluoro-3-nitroaniline (800 mg, 5.12 mmol) in N,N-dimethylformamide (5.00 mL) was added cyclopropanecarboxylic acid (441 mg, 5.12 mmol), hexafluoro-λ⁵-phosphanuide 1-[bis(dimethylamino)methylidene]-1H-1λ⁵-[1,2,3]triazolo[4,5-b]pyridin-3-ium-1-ylium-3-olate (2.92 g, 1.5 eq., 7.69 mmol) and ethylbis(propan-2-yl)amine (2.68 mL, 3 eq., 15.4 mmol) and the solution was stirred for 16 hours. After completion of reaction, the reaction mixture diluted with water and extracted with ethyl acetate. The organic layer dried over anhydrous sodium sulphate, filtered and concentrated to obtain crude. The crude was purified by flash chromatography. The desired fractions concentrated to afford N-(4-fluoro-3-nitrophenyl)cyclopropanecarboxamide (700 mg, 3.12 mmol) as yellow solid. Yield: 700 mg, 60.93%

Step-2: To a stirred solution of N-(4-fluoro-3-nitrophenyl)cyclopropanecarboxamide (500 mg, 2.23 mmol) in N, N-Diisopropylethylamine (1.17 mL, 3 eq., 6.69 mmol) in N-Methyl-2-Pyrrolidone (NMP) (5.00 mL), 3-methyloxetan-3-amine (201 μL, 2 eq., 4.46 mmol) was added at room temperature and stirred at 80° C. for 16 h. After completion, reaction mixture quenched with ice cold water, precipitate was formed. solid was filtered and dried to afford N-{4-[(3-methyloxetan-3-yl)amino]-3-nitrophenyl}cyclopropanecarboxamide (575 mg, 1.97 mmol) as a yellow solid. Yield: 510 mg, 69%

Step-3: To a solution of N-{4-[(3-methyloxetan-3-yl)amino]-3-nitrophenyl}cyclopropanecarboxamide (350 mg, 1.20 mmol) in methanol (20.0 mL) was added zinc (393 mg, 5 eq., 6.01 mmol) and ammonium chloride (321 mg, 5 eq., 6.01 mmol) at room temperature and the reaction mixture stirred for 3 h under hydrogen atmosphere. After completion, the reaction mixture passed through celite bed. Filtrate was concentrated at room temperature to get as N-{3-amino-4-[(3-methyloxetan-3-yl)amino]phenyl}cyclopropanecarboxamide (250 mg, 957 μmol) yellow color solid. Yield: 250 mg, 79.62%

Step-4: To a stirred solution of N-{3-amino-4-[(3-methyloxetan-3 yl)amino]phenyl}cyclopropanecarboxamide (178 mg, 681 μmol) and 3,4-bis(benzyloxy)-2-fluoro-5-methoxybenzaldehyde (200 mg, 0.8 eq., 545 μmol) in methanesulfinylmethane (3.00 mL), disodium sulfinatosulfonate (194 mg, 1.5 eq., 1.02 mmol) was added at room temperature. The resulting mixture stirred for 16 h at 80° C. After completion, water and ethyl acetate was added to reaction mixture. The organic fractions were collected, dried over anhydrous sodium sulfate, filtered and concentrated to get crude. The crude purified by flash chromatography. The desired fractions were concentrated to afford N-{2-[3,4-bis(benzyloxy)-2-fluoro-5-methoxyphenyl]-1-(3-methyloxetan-3-yl)-1H-1,3-benzodiazol-5-yl}cyclopropanecarboxamide (180 mg, 296 μmol) as yellow solid. Yield: 180 mg, 43.49%

Step-5: To N-{2-[3,4-bis(benzyloxy)-2-fluoro-5-methoxyphenyl]-1-(3-methyloxetan-3-yl)-1H-1,3-benzodiazol-5-yl}cyclopropanecarboxamide (170 mg, 280 μmol), trifluoroacetic acid (2.00 mL) added at room temperature. The resulting reaction mixture stirred at 60° C. for 4 h. After the complete consumption of starting material, reaction mixture was concentrated to obtain the crude. The crude purified in reverse-phase HPLC and desired fraction were collected, lyophilized to obtain N-[2-(2-fluoro-3,4-dihydroxy-5-methoxyphenyl)-1-(3-methyloxetan-3-yl)-1H-1,3-benzodiazol-5-yl]cyclopropanecarboxamide (19.0 mg, 44.5 μmol) as off white solid. Yield: 19 mg, 15.89%

LCMS and NMR Data: ES MS M/Z=428.21 (M+1); UPLC: 99.77%; 1H NMR (400 MHz, DMSO-d6) δ 10.20 (s, 1H), 9.43 (bs, 2H), 8.01 (s, 1H), 7.41 (d, J=9.2 Hz, 1H), 7.19 (d, J=8.8 Hz, 1H), 6.58 (d, J=6.4 Hz, 1H), 4.70 (d, J=5.6 Hz, 2H), 4.35 (d, J=5.2 Hz, 2H), 3.78 (s, 3H), 1.99 (s, 3H), 1.78-1.75 (m, 1H), 0.81-0.78 (m, 4H)

### Example 205: Synthesis of N-(3,3-difluorocyclobutyl)-2-(2-fluoro-3,4-dihydroxy-5-methoxyphenyl)-1-(3-methyloxetan-3-yl)-1H-benzo[d]imidazole-6-carboxamide

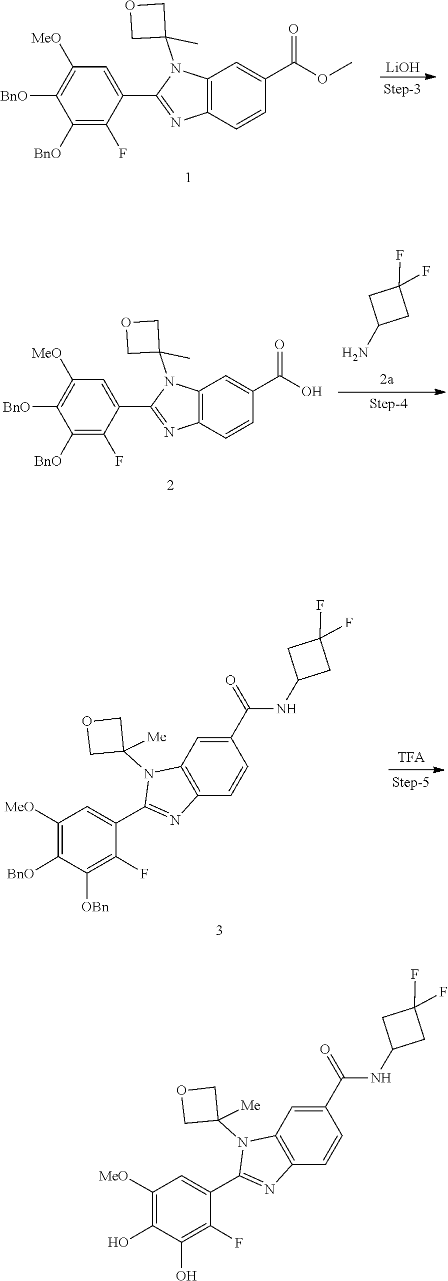

Step: 1 To a stirred solution of methyl 2-[3,4-bis(benzyloxy)-2-fluoro-5-methoxyphenyl]-1-(3-methyloxetan-3-yl)-1H-1,3-benzodiazole-6-carboxylate (150 mg, 257 μmol) in oxolane (1.20 mL) and water (400 μL) was added lithium hydroxide (30.8 mg, 5 eq., 1.29 mmol). The reaction mixture was heated at 40° C. for 16 h. After completion of reaction, reaction mixture was acidified with 2N HCl and extracted with ethyl acetate. The organic layer was separated, dried over sodium sulphate, filtered and concentrated under reduced pressure to get the crude. The crude was purified by flash chromatography. The desired fractions concentrated to get 2-[3,4-bis(benzyloxy)-2-fluoro-5-methoxyphenyl]-1-(3-methyloxetan-3-yl)-1H-1,3-benzodiazole-6-carboxylic acid (135 mg, 230 μmol) as pale yellow crystalline solid. Yield: 0.135 g, 89%

Step: 2 To a stirred solution of 3,3-difluorocyclobutan-1-amine hydrochloride (32.8 mg, 229 μmol) and 2-[3,4-bis(benzyloxy)-2-fluoro-5-methoxyphenyl]-1-(3-methyloxetan-3-yl)-1H-1,3-benzodiazole-6-carboxylic acid (130 mg, 229 μmol) in N,N-dimethylformamide (2.00 mL) was added hexafluoro-λ⁵-phosphanuide 1-[bis(dimethylamino)methylidene]-1H-1λ⁵-[1,2,3]triazolo[4,5-b]pyridin-3-ium-1-ylium-3-olate (130 mg, 1.5 eq., 343 μmol) and ethylbis(propan-2-yl)amine (119 μL, 3 eq., 686 μmol) and the solution was stirred at room temperature for 16 hours. After completion of reaction, the reaction mixture was diluted with water and extracted with ethyl acetate. The organic layer dried over anhydrous sodium sulphate, filtered and concentrated to obtain crude. The crude was purified by flash chromatography. The desired fractions concentrated to afford 2-[3,4-bis(benzyloxy)-2-fluoro-5-methoxyphenyl]-N-(3,3-difluorocyclobutyl)-1-(3-methyloxetan-3-yl)-1H-1,3-benzodiazole-6-carboxamide (75.0 mg, 106 μmol) as yellow sticky solid. Yield: 0.075 g, (46%)

Step:3 To 2-[3,4-bis(benzyloxy)-2-fluoro-5-methoxyphenyl]-N-(3,3-difluorocyclobutyl)-1-(3-methyloxetan-3-yl)-1H-1,3-benzodiazole-6-carboxamide (75.0 mg, 114 μmol) was added trifluoroacetic acid (1.00 mL) and the resulting solution was stirred for 3 h at 50° C. After completion, reaction mixture was concentrated under reduced pressure to get the crude and further purified by reverse phase HPLC. Desired fractions were lyophilized to afford N-(3,3-difluorocyclobutyl)-2-(2-fluoro-3,4-dihydroxy-5-methoxyphenyl)-1-(3-methyloxetan-3-yl)-1H-1,3-benzodiazole-6-carboxamide as white solid. Yield: 0.02 g, 36%

1H NMR: ES MS M/Z=443 (M+1), NMR (400 MHz, DMSO-d6) δ 9.41 (br s, 1H), 7.70 (d, 1H), 7.64 (d, 1H), 7.21 (d, 1H), 6.57 (d, 1H), 4.70 (d, 2H), 4.37 (d, 2H), 3.84 (t, 2H), 3.78 (s, 3H), 3.45 (t, 2H), 2.78 (s, 3H), 2.00 (s, 3H), 1.23 (s, 1H).

### Example 206: Synthesis of N-(2-(2-fluoro-3,4-dihydroxy-5-methoxyphenyl)-1-(3-methyloxetan-3-yl)-1H-benzo[d]imidazol-5-yl)isoxazole-4-carboxamide

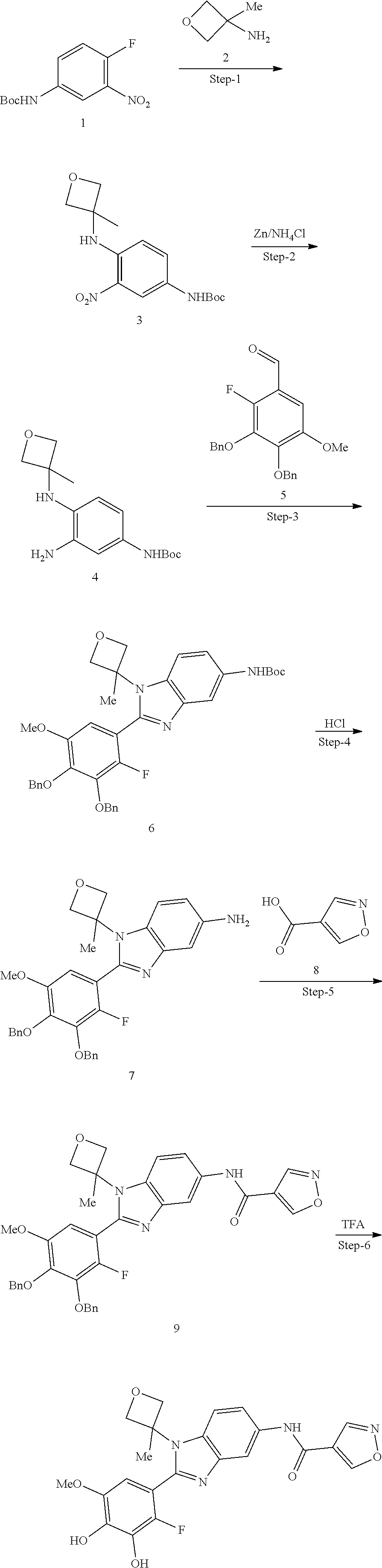

Step-1: To a stirred solution of tert-butyl N-(4-fluoro-3-nitrophenyl)carbamate (500 mg, 1.95 mmol) and 3-methyloxetan-3-amine (131 μL, 1.5 eq., 2.93 mmol) in 1-methylpyrrolidin-2-one (5.00 mL) was added ethylbis(propan-2-yl)amine (1.02 mL, 3 eq., 5.85 mmol) and the solution was stirred for 16 hours at 110° C. After completion of reaction, the reaction mixture diluted with water and extracted with ethyl acetate. The organic layer dried over anhydrous sodium sulphate, filtered and concentrated to obtain tert-butyl N-{4-[(3-methyloxetan-3-yl)amino]-3-nitrophenyl}carbamate (600 mg, 816 μmol) (crude) as yellow liquid. Yield: 0.60 g, Crude

Step-2: To a stirred solution of tert-butyl N-{4-[(3-methyloxetan-3-yl)amino]-3-nitrophenyl}carbamate (300 mg, 928 μmol) in methanol (5.00 mL) was added zinc (303 mg, 5 eq., 4.64 mmol) and ammonium chloride (248 mg, 5 eq., 4.64 mmol) were added at room temperature and stirred at 50° C. for 1 h. After completion of the reaction, reaction mixture was passed through celite and washed with 10% methanol in dichloromethane, filtrate was washed with water, dried over anhydrous sodium sulfate and concentrated under reduced pressure to get tert-butyl N-{3-amino-4-[(3-methyloxetan-3-yl)amino]phenyl}carbamate (250 mg, 648 μmol) (crude) as brown solid. Yield: 0.25 g, Crude

Step-3: To a stirred solution of disodium sulfinatosulfonate (144 mg, 1.2 eq., 757 μmol) and 3,4-bis(benzyloxy)-2-fluoro-5-methoxybenzaldehyde (185 mg, 0.8 eq., 504 μmol) in methanesulfinylmethane (5.00 mL) was added disodium sulfinatosulfonate (144 mg, 1.2 eq., 757 μmol) at room temperature. The resulting mixture stirred for 16 h at 85° C. After completion, reaction mixture cooled to room temperature and diluted with water and extracted with ethyl acetate. The organic layer was dried over anhydrous sodium sulfate and concentrated under reduced pressure to get crude. The crude purified by flash chromatography. The desired fractions concentrated to afford tert-butyl N-{2-[3,4-bis(benzyloxy)-2-fluoro-5-methoxyphenyl]-1-(3-methyloxetan-3-yl)-1H-1,3-benzodiazol-5-yl}carbamate (240 mg, 315 μmol) as off white solid. Yield: 0.24 g, 49.97%

Step-4: To a stirred solution of tert-butyl N-{2-[3,4-bis(benzyloxy)-2-fluoro-5-methoxyphenyl]-1-(3-methyloxetan-3-yl)-1H-1,3-benzodiazol-5-yl}carbamate (240 mg, 375 μmol) in dichloromethane (5.00 mL), 4 M hydrochloride in 1,4-Dioxane (5.00 mL) was added and stirred at room temperature for 3 h. After completion of reaction, reaction mixture was concentrated to get 2-[3,4-bis(benzyloxy)-2-fluoro-5-methoxyphenyl]-1-(3-methyloxetan-3-yl)-1H-1,3-benzodiazol-5-amine hydrochloride (200 mg, 316 μmol) as off-white solid. Yield: 0.20 g, 84.22%

Step-5: To a stirred solution of 2-[3,4-bis(benzyloxy)-2-fluoro-5-methoxyphenyl]-1-(3-methyloxetan-3-yl)-1H-1,3-benzodiazol-5-amine (200 mg, 347 μmol) and 1,2-oxazole-4-carboxylic acid (47.1 mg, 1.2 eq., 417 μmol) in dichloromethane (5.00 mL) was added triethylamine (109 mg, 3.1 eq., 1.08 mmol) and tripropyl-1,3,5,2λ⁵,4λ⁵,6μ⁵-trioxatriphosphinane-2,4,6-trione (707 μL, 3.2 eq., 1.11 mmol) and the solution was stirred for 16 hours at room temperature. After completion of reaction, the reaction mixture diluted with water and extracted with dichloromethane. The organic layer dried over anhydrous sodium sulphate, filtered and concentrated to obtain crude. Crude was purified by flash chromatography. Desired fractions were concentrated to get N-{2-[3,4-bis(benzyloxy)-2-fluoro-5-methoxyphenyl]-1-(3-methyloxetan-3-yl)-1H-1,3-benzodiazol-5-yl}-1,2-oxazole-4-carboxamide (140 mg, 207 μmol) as light brown oil. Yield: 0.14 g, 59.72%

Step-6: To a solution of N-{2-[3,4-bis(benzyloxy)-2-fluoro-5-methoxyphenyl]-1-(3-methyloxetan-3-yl)-1H-1,3-benzodiazol-5-yl}-1,2-oxazole-4-carboxamide (80.0 mg, 126 μmol) in trifluoroacetic acid (1.50 mL) was stirred for 3 h at 55° C. After completion, the reaction mixture was concentrated to get crude. Crude was purified by reverse phase HPLC and pure fractions were lyophilized to get N-[2-(2-fluoro-3,4-dihydroxy-5-methoxyphenyl)-1-(3-methyloxetan-3-yl)-1H-1,3-benzodiazol-5-yl]-1,2-oxazole-4-carboxamide (26.0 mg, 53.8 μmol) as off white solid. Yield: 0.026 g, 42.67%

ES MS M/Z=455.28 (M+1), 1H NMR (400 MHz, DMSO-d6) δ 10.33 (s, 1H), 9.60 (s, 1H), 9.54-9.47 (m, 2H), 8.13 (d, 1H), 7.54 (m, 1H), 7324 (m, 1H), 6.62 (d, 1H), 4.74 (d, 2H), 4.39 (d, 2H), 3.79 (s, 3H), 2.03 (s, 3H).

### Example 207: Synthesis of N-[2-(2-fluoro-3,4-dihydroxy-5-methoxyphenyl)-1-(3-methyloxetan-3-yl)-1H-1,3-benzodiazol-6-yl]acetamide

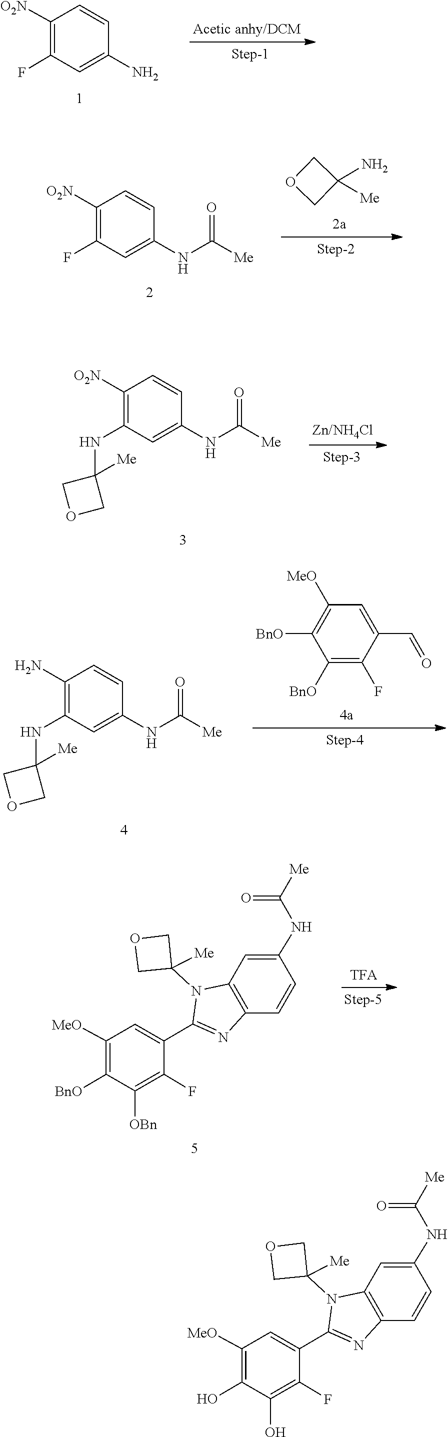

Step-1: To a stirred solution of 3-fluoro-4-nitroaniline (1.00 g, 6.41 mmol) in dichloromethane (10.0 mL), acetyl acetate (666 μL, 1.1 eq., 7.05 mmol) was added and stirred at room temperature for 16 h. After completion of reaction, reaction mixture diluted with water and extracted with dichloromethane. The combined organic layers were dried over anhydrous sodium sulphate, filtered and concentrated to afford N-(3-fluoro-4-nitrophenyl)acetamide (840 mg, 4.24 mmol) as off-white solid. Yield: 840 mg, 66.18%

Step-2: To a stirred solution of N-(3-fluoro-4-nitrophenyl)acetamide (345 mg, 1.74 mmol) and N, N-Diisopropylethylamine (912 μL, 3 eq., 5.22 mmol) in N-Methyl-2-Pyrrolidone (NMP) (5.00 mL), 3-methyloxetan-3-amine (157 μL, 2 eq., 3.48 mmol) was added at room temperature and stirred at 100° C. for 16 h. After completion, reaction mixture quenched with ice cold water, precipitate was formed. The solid was filtered and dried to afford N-{3-[(3-methyloxetan-3-yl)amino]-4-nitrophenyl}acetamide (395 mg, 1.49 mmol) as a yellow solid. Yield: 395 mg, 85.52%

Step-3: To a solution of N-{3-[(3-methyloxetan-3-yl)amino]-4-nitrophenyl}acetamide (250 mg, 942 μmol) in methanol (20.0 mL) was added zinc (308 mg, 5 eq., 4.71 mmol) followed by addition of ammonium chloride (252 mg, 5 eq., 4.71 mmol) at room temperature and the reaction mixture stirred for 3 h under hydrogen atmosphere. After completion, the reaction mixture diluted with dichloromethane and passed through celite bed. water was added to filtrate and extracted with 10% methanol in dichloromethane solution. The organic fraction collected, dried over anhydrous sodium sulphate, concentrated to obtain N-{4-amino-3-[(3-methyloxetan-3-yl)amino]phenyl}acetamide (130 mg, 553 μmol) as brown liquid. Yield: 130 mg, 58.63%

Step-4: To a stirred solution of N-{4-amino-3-[(3-methyloxetan-3-yl)amino]phenyl}acetamide (125 mg, 531 μmol) and 3,4-bis(benzyloxy)-2-fluoro-5-methoxybenzaldehyde (156 mg, 0.8 eq., 425 μmol) in methanesulfinylmethane (3.00 mL), disodium sulfinatosulfonate (151 mg, 1.5 eq., 797 μmol) was added at room temperature. The resulting mixture stirred for 16 h at 80° C. After completion, water and ethyl acetate was added to reaction mixture. The organic fractions were collected, dried over anhydrous sodium sulfate, filtered and concentrated to get crude. The crude purified by flash chromatography. The desired fractions were concentrated to afford N-{2-[3,4-bis(benzyloxy)-2-fluoro-5-methoxyphenyl]-1-(3-methyloxetan-3-yl)-1H-1,3-benzodiazol-6-yl}acetamide (150 mg, 224 μmol) as yellow solid. Yield: 150 mg, 42.23%

Step-5: A dried round bottom flask was charged with N-{2-[3,4-bis(benzyloxy)-2-fluoro-5-methoxyphenyl]-1-(3-methyloxetan-3-yl)-1H-1,3-benzodiazol-6-yl}acetamide (150 mg, 258 μmol) and trifluoroacetic acid (1.00 mL) at room temperature. The resulting reaction mixture stirred at 60° C. for 4 h. After the complete consumption of starting material, reaction mixture was concentrated to obtain the crude. The crude purified by reverse-phase High performance liquid chromatography. The desired fractions were collected and lyophilized to obtain N-[2-(2-fluoro-3,4-dihydroxy-5-methoxyphenyl)-1-(3-methyloxetan-3-yl)-1H-1,3-benzodiazol-6-yl]acetamide (71.0 mg, 177 μmol) as off white solid. Yield: 71 mg, 68.59%

1H NMR and LCMS data: ES MS M/Z=402.07 [M+H]⁺; UPLC: 99.19%; 1H NMR (400 MHz, DMSO-d6) δ 10.05 (s, 1H), 9.43 (Bs, 2H), 7.71 (s, 1H), 7.59 (d, J=8.8 Hz, 1H), 7.31-7.29 (m, 1H), 6.58 (d, J=6.0 Hz, 1H), 4.73 (d, J 6.0 Hz, 2H), 4.29 (d, J 6.0 Hz, 2H), 3.78 (s, 3H), 2.06 (s, 1H), 2.00 (s, 3H).

### Example 208: Synthesis of N-[2-(2-fluoro-3,4-dihydroxy-5-methoxyphenyl)-1-(3-methyloxetan-3-yl)-1H-1,3-benzodiazol-6-yl]acetamide

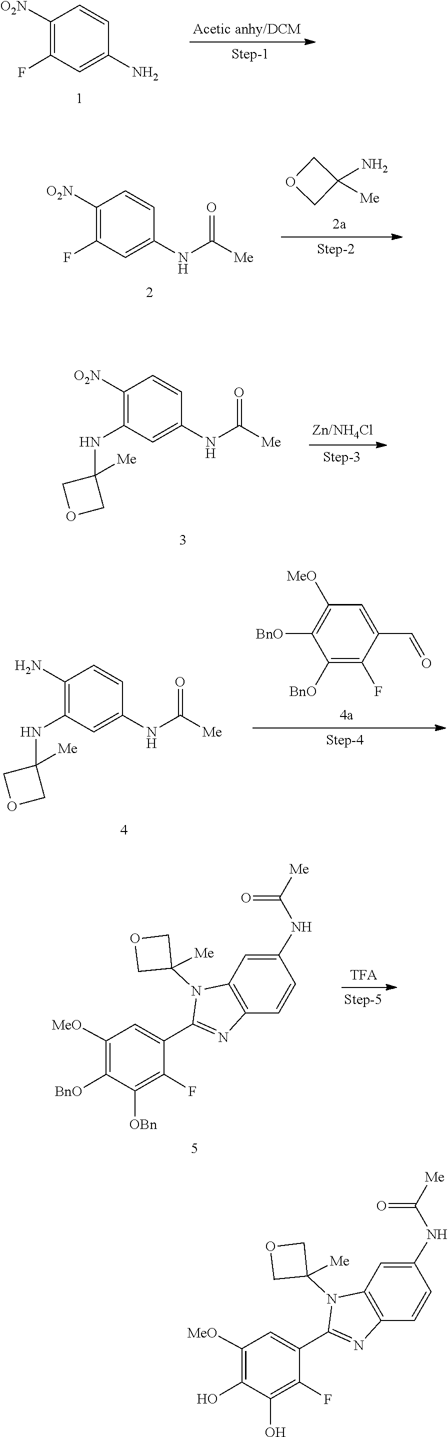

Step-1: To a stirred solution of 3-fluoro-4-nitroaniline (1.00 g, 6.41 mmol) in dichloromethane (10.0 mL), acetyl acetate (666 μL, 1.1 eq., 7.05 mmol) was added and stirred at room temperature for 16 h. After completion of reaction, reaction mixture diluted with water and extracted with dichloromethane. The combined organic layers were dried over anhydrous sodium sulphate, filtered and concentrated to afford N-(3-fluoro-4-nitrophenyl)acetamide (840 mg, 4.24 mmol) as off-white solid. Yield: 840 mg, 66.18%

Step-2: To a stirred solution of N-(3-fluoro-4-nitrophenyl)acetamide (345 mg, 1.74 mmol) and N, N-Diisopropylethylamine (912 μL, 3 eq., 5.22 mmol) in N-Methyl-2-Pyrrolidone (NMP) (5.00 mL), 3-methyloxetan-3-amine (157 μL, 2 eq., 3.48 mmol) was added at room temperature and stirred at 100° C. for 16 h. After completion, reaction mixture quenched with ice cold water, precipitate was formed. The solid was filtered and dried to afford N-{3-[(3-methyloxetan-3-yl)amino]-4-nitrophenyl}acetamide (395 mg, 1.49 mmol) as a yellow solid. Yield: 395 mg, 85.52%

Step-3: To a solution of N-{3-[(3-methyloxetan-3-yl)amino]-4-nitrophenyl}acetamide (250 mg, 942 μmol) in methanol (20.0 mL) was added zinc (308 mg, 5 eq., 4.71 mmol) followed by addition of ammonium chloride (252 mg, 5 eq., 4.71 mmol) at room temperature and the reaction mixture stirred for 3 h under hydrogen atmosphere. After completion, the reaction mixture diluted with dichloromethane and passed through celite bed. water was added to filtrate and extracted with 10% methanol in dichloromethane solution. The organic fraction collected, dried over anhydrous sodium sulphate, concentrated to obtain N-{4-amino-3-[(3-methyloxetan-3-yl)amino]phenyl}acetamide (130 mg, 553 μmol) as brown liquid. Yield: 130 mg, 58.63%

Step-4: To a stirred solution of N-{4-amino-3-[(3-methyloxetan-3-yl)amino]phenyl}acetamide (125 mg, 531 μmol) and 3,4-bis(benzyloxy)-2-fluoro-5-methoxybenzaldehyde (156 mg, 0.8 eq., 425 μmol) in methanesulfinylmethane (3.00 mL), disodium sulfinatosulfonate (151 mg, 1.5 eq., 797 μmol) was added at room temperature. The resulting mixture stirred for 16 h at 80° C. After completion, water and ethyl acetate was added to reaction mixture. The organic fractions were collected, dried over anhydrous sodium sulfate, filtered and concentrated to get crude. The crude purified by flash chromatography. The desired fractions were concentrated to afford N-{2-[3,4-bis(benzyloxy)-2-fluoro-5-methoxyphenyl]-1-(3-methyloxetan-3-yl)-1H-1,3-benzodiazol-6-yl}acetamide (150 mg, 224 μmol) as yellow solid. Yield: 150 mg, 42.23%

Step-5: A dried round bottom flask was charged with N-{2-[3,4-bis(benzyloxy)-2-fluoro-5-methoxyphenyl]-1-(3-methyloxetan-3-yl)-1H-1,3-benzodiazol-6-yl}acetamide (150 mg, 258 μmol) and trifluoroacetic acid (1.00 mL) at room temperature. The resulting reaction mixture stirred at 60° C. for 4 h. After the complete consumption of starting material, reaction mixture was concentrated to obtain the crude. The crude purified by reverse-phase High performance liquid chromatography. The desired fractions were collected and lyophilized to obtain N-[2-(2-fluoro-3,4-dihydroxy-5-methoxyphenyl)-1-(3-methyloxetan-3-yl)-1H-1,3-benzodiazol-6-yl]acetamide (71.0 mg, 177 μmol) as off white solid. Yield: 71 mg, 68.59%

1H NMR and LCMS data: ES MS M/Z=402.07 [M+H]⁺; UPLC: 99.19%; 1H NMR (400 MHz, DMSO-d6) δ 10.05 (s, 1H), 9.43 (Bs, 2H), 7.71 (s, 1H), 7.59 (d, J=8.8 Hz, 1H), 7.31-7.29 (m, 1H), 6.58 (d, J=6.0 Hz, 1H), 4.73 (d, J 6 Hz, 2H), 4.29 (d, J 6 Hz, 2H), 3.78 (s, 3H), 2.06 (s, 1H), 2.00 (s, 3H).

### Example 209: Synthesis of N-[2-(2-fluoro-3,4-dihydroxy-5-methoxyphenyl)-1-(3-methyloxetan-3-yl)-1H-1,3-benzodiazol-4-yl]acetamide

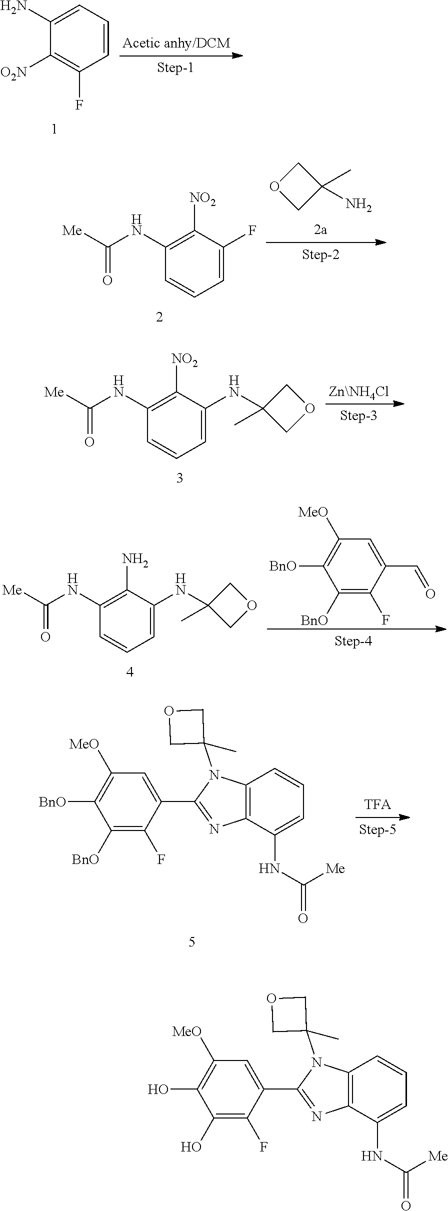

Step-1: To a stirred solution of 3-fluoro-2-nitroaniline (1.00 g, 6.41 mmol) in dichloromethane (10.0 mL), acetyl acetate (666 μL, 1.1 eq., 7.05 mmol) was added and stirred at room temperature for 16 h. After completion of reaction, reaction mixture diluted with water and extracted with dichloromethane. The combined organic layers were dried over anhydrous sodium sulphate, filtered and concentrated to afford N-(3-fluoro-2-nitrophenyl)acetamide (888 mg, 4.48 mmol) as off-white solid. Yield: 0.888 g, 69.96%

Step-2: To a stirred solution of N-(3-fluoro-2-nitrophenyl)acetamide (345 mg, 1.74 mmol) and N, N-Diisopropylethylamine (912 μL, 3 eq., 5.22 mmol) in N-Methyl-2-Pyrrolidone (NMP) (5.00 mL), 3-methyloxetan-3-amine (157 μL, 2 eq., 3.48 mmol) was added at room temperature and stirred at 100° C. for 16 h. After completion, reaction mixture quenched with ice cold water, precipitate was formed. solid was filtered and dried to afford N-{3-[(3-methyloxetan-3-yl)amino]-2-nitrophenyl}acetamide (370 mg, 1.39 mmol) as a yellow solid. Yield: 370 mg, 80%

Step-3: To a solution of N-{3-[(3-methyloxetan-3-yl)amino]-2-nitrophenyl}acetamide (300 mg, 1.13 mmol) in methanol (20.0 mL) was added zinc (370 mg, 5 eq., 5.65 mmol) followed by addition of ammonium chloride (302 mg, 5 eq., 5.65 mmol) at room temperature and the reaction mixture stirred for 3 h under hydrogen atmosphere. After completion, the reaction mixture diluted with dichloromethane and passed through celite bed. Then water was added to filtrate and extracted with 10% methanol in dichloromethane solution. The organic fraction collected, dried over anhydrous sodium sulfate, concentrated to obtain N-{2-amino-3-[(3-methyloxetan-3-yl)amino]phenyl}acetamide (230 mg, 978 μmol) as brown liquid. Yield: 230 mg, 86.44%

Step-4: To a stirred solution of N-{2-amino-3-[(3-methyloxetan-3-yl)amino]phenyl}acetamide (120 mg, 510 μmol) and 3,4-bis(benzyloxy)-2-fluoro-5-methoxybenzaldehyde (149 mg, 0.8 eq., 408 μmol) in methanesulfinylmethane (3.00 mL), disodium sulfinatosulfonate (145 mg, 1.5 eq., 765 μmol) was added at room temperature. The resulting mixture stirred for 16 h at 80° C. After completion, water and ethyl acetate was added to reaction mixture. The organic fractions were collected, dried over anhydrous sodium sulfate, filtered and concentrated to get crude. The crude purified by flash chromatography. The desired fractions were concentrated to afford N-{2-[3,4-bis(benzyloxy)-2-fluoro-5-methoxyphenyl]-1-(3-methyloxetan-3-yl)-1H-1,3-benzodiazol-4-yl}acetamide (100 mg, 167 μmol) as yellow solid. Yield: 100 mg, 32.70%

Step-5: To N-{2-[3,4-bis(benzyloxy)-2-fluoro-5-methoxyphenyl]-1-(3-methyloxetan-3-yl)-1H-1,3-benzodiazol-4-yl}acetamide (90.0 mg, 155 μmol), trifluoroacetic acid (2.00 mL) was added at room temperature. The resulting reaction mixture stirred at 60° C. for 4 h. After the complete consumption of starting material, reaction mixture was concentrated to obtain the crude. The crude purified by reverse-phase High performance liquid chromatography. The desired fractions were collected and lyophilized to obtain N-[2-(2-fluoro-3,4-dihydroxy-5-methoxyphenyl)-1-(3-methyloxetan-3-yl)-1H-1,3-benzodiazol-4-yl]acetamide (25.0 mg, 62.3 μmol) as off white solid. Yield: 25 mg, 40.25%

1H NMR and LCMS data: ES MS M/Z=402.07 [M+H]⁺; UPLC: 98.09%; ¹H NMR (400 MHz, DMSO-d6) δ 9.91 (s, 1H), 9.41 (bs, 2H), 8.00 (d, J=7.6 Hz, 1H), 7.18 (t, J=9.04 Hz, 1H), 6.94 (d, J=8.0 Hz, 1H), 6.62 (d, J=6.0 Hz, 1H), 4.77 (d, J 6.0 Hz, 2H), 4.35 (d, J 6.0 Hz, 2H), 3.77 (s, 3H), 2.16 (s, 1H), 1.98 (s, 3H)

### Example 210: Synthesis of N-[2-(2-fluoro-3,4-dihydroxy-5-methoxyphenyl)-1-(3-methyloxetan-3-yl)-1H-1,3-benzodiazol-7-yl]acetamide

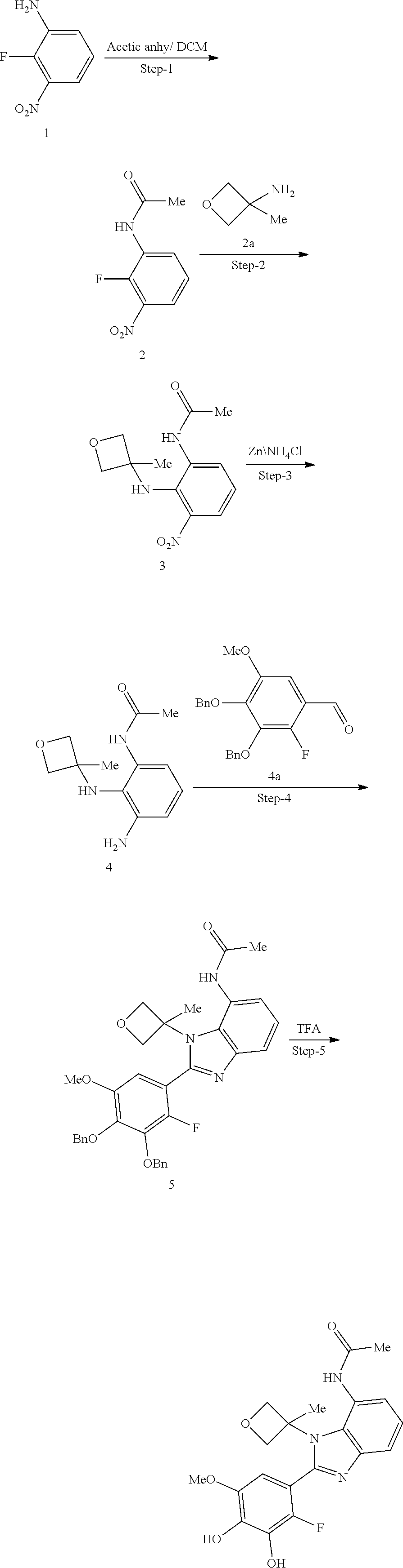

Step-1: To a stirred solution of 2-fluoro-3-nitroaniline (1.00 g, 6.41 mmol), acetyl acetate (666 μL, 1.1 eq., 7.05 mmol) was added and stirred at room temperature for 16 h. After completion of reaction, reaction mixture diluted with water and extracted with dichloromethane. The combined organic layers were dried over anhydrous sodium sulphate, filtered and concentrated to afford N-(2-fluoro-3-nitrophenyl)acetamide (890 mg, 4.49 mmol) as off-white solid. Yield: 890 mg g, 70.12%

Step-2: To a stirred solution of N-(2-fluoro-3-nitrophenyl)acetamide (345 mg, 1.74 mmol) and N, N-Diisopropylethylamine (912 μL, 3 eq., 5.22 mmol) in N-Methyl-2-Pyrrolidone (NMP) (5.00 mL), 3-methyloxetan-3-amine (157 μL, 2 eq., 3.48 mmol) was added at room temperature and stirred at 100° C. for 16 h. After completion, reaction mixture quenched with ice cold water, precipitate was formed. The solid was filtered and dried to afford N-{2-[(3-methyloxetan-3-yl)amino]-3-nitrophenyl}acetamide (170 mg, 641 μmol) as orange solid. Yield: 170 mg, 36.81%

Step-3: To a solution of N-{2-[(3-methyloxetan-3-yl)amino]-3-nitrophenyl}acetamide (170 mg, 641 μmol) in methanol (20.0 mL) was added zinc (209 mg, 5 eq., 3.20 mmol) followed by addition of ammonium chloride (171 mg, 5 eq., 3.20 mmol) at room temperature and the reaction mixture stirred for 3 h under hydrogen atmosphere. After completion, the reaction mixture diluted with dichloromethane and passed through celite bed. Then water was added to filtrate and extracted with 10% methanol in dichloromethane solution. The organic fraction collected, dried over anhydrous sodium sulphate, concentrated to obtain N-{3-amino-2-[(3-methyloxetan-3-yl)amino]phenyl}acetamide (140 mg, 518 μmol) as brown liquid. Yield: 140 mg, 80.78%

Step-4: To a stirred solution of N-{3-amino-2-[(3-methyloxetan-3-yl)amino]phenyl}acetamide (140 mg, 595 μmol) and 3,4-bis(benzyloxy)-2-fluoro-5-methoxybenzaldehyde (174 mg, 0.8 eq., 476 μmol) in methanesulfinylmethane (3.00 mL), disodium sulfinatosulfonate (170 mg, 1.5 eq., 893 μmol) was added at room temperature. The resulting mixture stirred for 16 h at 80° C. After completion, water and ethyl acetate was added to reaction mixture. The organic fractions were collected, dried over anhydrous sodium sulfate, filtered and concentrated to get crude. The crude purified by flash chromatography. The desired fractions were concentrated to afford N-{2-[3,4-bis(benzyloxy)-2-fluoro-5-methoxyphenyl]-1-(3-methyloxetan-3-yl)-1H-1,3-benzodiazol-7-yl}acetamide (155 mg, 245 μmol) as off white solid. Yield: 180 mg, 43.49%

Step-5: To N-{2-[3,4-bis(benzyloxy)-2-fluoro-5-methoxyphenyl]-1-(3-methyloxetan-3-yl)-1H-1,3-benzodiazol-7-yl}acetamide (150 mg, 258 μmol) trifluoroacetic acid (1.00 mL) added at room temperature. The resulting reaction mixture stirred at 60° C. for 4 h. After the complete consumption of starting material, reaction mixture was concentrated to obtain the crude. The crude was purified by reverse phase HPLC and desired fractions were collected and lyophilized to obtain N-[2-(2-fluoro-3,4-dihydroxy-5-methoxyphenyl)-1-(3-methyloxetan-3-yl)-1H-1,3-benzodiazol-7-yl]acetamide (13.0 mg, 32.4 μmol) as off white solid. Yield: 13 mg, 12.56%

LCMS and NMR Data: ES MS M/Z=402.28 (M+1)⁺, UPLC: 99.49% 1H NMR (400 MHz, DMSO-d6) δ 9.24 (bs, 1H), 7.19-7.15 (m, 1H), 6.97 (d, J=8.4 Hz, 1H), 6.76 (d, J=6.0 Hz, 1H), 6.61 (d, J=7.6 Hz, 1H), 4.18 (d, J=11.6 Hz, 1H), 4.09 (d, J=11.6 Hz, 1H), 3.79 (s, 3H), 3.52 (d, J=12.4 Hz, 1H), 3.32 (d, J=12.8 Hz, 1H), 1.91 (s, 3H), 1.31 (s, 3H)

### Example 211: Synthesis of methyl 2-(2-fluoro-3,4-dihydroxy-5-methoxyphenyl)-1-(3-methyloxetan-3-yl)-1H-benzo[d]imidazole-6-carboxylate

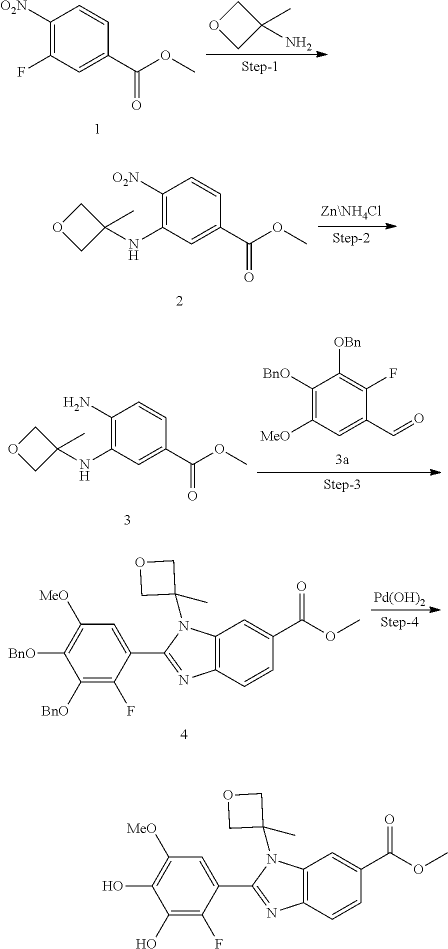

Step-1: To a stirred solution of 3-methyloxetan-3-amine (691 μL, 1.5 eq., 15.1 mmol) in N-Methyl-2-Pyrrolidone (NMP) (5.00 mL), 3-methyloxetan-3-amine (691 μL, 1.5 eq., 15.1 mmol) and N, N-Diisopropylethylamine (5.26 mL, 3 eq., 30.1 mmol) was added at room temperature and stirred at 130° C. for 1 h. After completion, reaction mixture quenched with ice cold water, precipitate was formed. solid was filtered and dried to afford methyl 4-[(3-methyloxetan-3-yl)amino]-3-nitrobenzoate (600 mg, 2.25 mmol) as a yellow solid. Yield: 0.60 g, 89%

Step-2: To a stirred solution of methyl 3-[(3-methyloxetan-3-yl)amino]-4-nitrobenzoate (700 mg, 2.63 mmol) in methanol (10.0 mL), zinc (859 mg, 5 eq., 13.1 mmol) and ammonium chloride (703 mg, 5 eq., 13.1 mmol) were added at room temperature and stirred at 50° C. for 1 h. After completion of the reaction, reaction mixture was passed through celite and washed with dichloromethane, filtrate was washed with water, dried over anhydrous sodium sulfate and concentrated under reduced pressure to get crude. Yield: 0.600 g, 59%

Step-3: To a stirred solution of 3,4-bis(benzyloxy)-2-fluoro-5-methoxybenzaldehyde (595 mg, 0.8 eq., 1.63 mmol) and methyl 4-amino-3-[(3-methyloxetan-3-yl)amino]benzoate (600 mg, 2.03 mmol) in methanesulfinylmethane (6.00 mL), disodium sulfinatosulfonate (579 mg, 1.5 eq., 3.05 mmol) was added at room temperature. The resulting mixture stirred for 16 h at 80° C. After completion, ice cold water was added in the reaction mixture and solid was filtered and dried to get crude. The crude purified by flash chromatography. The desired fractions were concentrated to afford methyl 2-[3,4-bis(benzyloxy)-2-fluoro-5-methoxyphenyl]-1-(3-methyloxetan-3-yl)-1H-1,3-benzodiazole-6-carboxylate (700 mg, 1.14 mmol) as sticky solid. Yield: 0.700 g, 56%

Step-4: To a solution of methyl 2-[3,4-bis(benzyloxy)-2-fluoro-5-methoxyphenyl]-1-(3-methyloxetan-3-yl)-1H-1,3-benzodiazole-6-carboxylate (100 mg, 172 μmol) in trifluoroacetic acid (2.00 mL) was stirred for 3 h under. After completion, the reaction mixture was concentrated to get crude. Crude was purified by reverse phase HPLC and desired fractions were lyophilized to get methyl 2-(2-fluoro-3,4-dihydroxy-5-methoxyphenyl)-1-(3-methyloxetan-3-yl)-1H-1,3-benzodiazole-6-carboxylate (45.0 mg, 111 μmol) as white solid. Yield: 0.045 g, 64.51%

ES MS M/Z=403.29 (M+1), 1H NMR (400 MHz, DMSO-d6) δ 9.52 (brs, 2H), 7.92 (dd, J=8.4, 1.2 Hz, 1H), 7.81-7.79 (m, 1H), 7.75 (s, 1H), 6.642 (d, J=6.4 Hz, 1H), 4.77 (d, J=5.6 Hz, 2H), 4.42 (d, J=6 Hz, 2H), 3.89 (s, 3H), 3.79 (s, 3H), 2.05 (s, 3H).

### Example 212: Synthesis of 2-(2-fluoro-3,4-dihydroxy-5-methoxyphenyl)-N-methyl-1-(3-methyloxetan-3-yl)-1H-benzo[d]imidazole-6-carboxamide

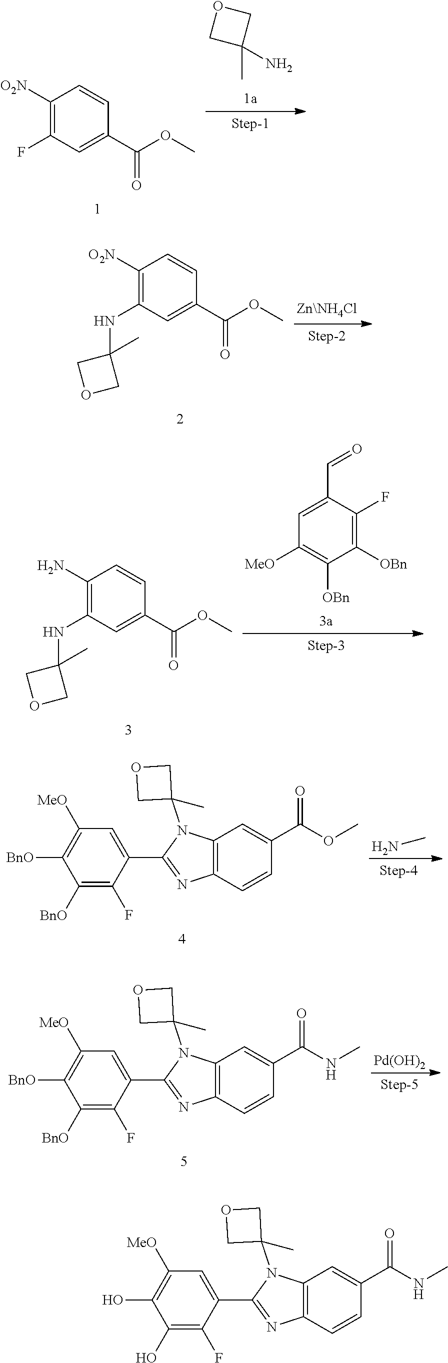

Step-1: To a stirred solution of 3-methyloxetan-3-amine (437 mg, 2 eq., 5.02 mmol) in N-Methyl-2-Pyrrolidone (NMP) (5.00 mL), methyl 4-[(3-methyloxetan-3-yl)amino]-3-nitrobenzoate (600 mg, 2.25 mmol) and N, N-Diisopropylethylamine (1.32 mL, 3 eq., 7.53 mmol) was added at room temperature and stirred at 130° C. for 1 h. After completion, reaction mixture quenched with ice cold water, precipitate was formed. solid was filtered and dried to afford methyl 4-[(3-methyloxetan-3-yl)amino]-3-nitrobenzoate (600 mg, 2.25 mmol) as a yellow solid. Yield: 0.60 g, 89%

Step-2: To a stirred solution of methyl 4-[(3-methyloxetan-3-yl)amino]-3-nitrobenzoate (500 mg, 1.88 mmol) in methanol (10.0 mL), zinc (614 mg, 5 eq., 9.39 mmol) and ammonium chloride (502 mg, 5 eq., 9.39 mmol) were added at room temperature and stirred at 40° C. for 1 h. After completion of the reaction, reaction mixture was passed through celite, filtrate was extracted with dichloromethane and concentrated under reduced pressure to get the crude methyl 4-amino-3-((3-methyloxetan-3-yl)amino)benzoate (400 mg, 1.13 mmol). Yield: 0.40 g, Crude

Step-3: To a stirred solution of methyl 4-amino-3-[(3-methyloxetan-3-yl)amino]benzoate (220 mg, 931 μmol) and 3,4-bis(benzyloxy)-2-fluoro-5-methoxybenzaldehyde (273 mg, 0.8 eq., 745 μmol) in methanesulfinylmethane (5.00 mL), disodium sulfinatosulfonate (266 mg, 1.5 eq., 1.40 mmol) was added at room temperature. The resulting mixture stirred for 16 h at room temperature. After completion, reaction mixture was concentrated to get crude. The crude purified by flash chromatography. The desired fractions were concentrated to afford methyl 2-[3,4-bis(benzyloxy)-2-fluoro-5-methoxyphenyl]-1-(3-methyloxetan-3-yl)-1H-1,3-benzodiazole-6-carboxylate (350 mg, 601 μmol) as sticky solid. Yield: 0.35 g, 64%

Step-4: To a stirred solution of methyl 2-[3,4-bis(benzyloxy)-2-fluoro-5-methoxyphenyl]-1-(3-methyloxetan-3-yl)-1H-1,3-benzodiazole-6-carboxylate (300 mg, 515 μmol) in Methyl amine (40% in ethanol) (30.0 mL, 515 μmol), reaction mixture was stirred at 60° C. for 16 h. After completion, reaction mixture was concentrated to afford the Crude 2-(3,4-bis(benzyloxy)-2-fluoro-5-methoxyphenyl)-N-methyl-1-(3-methyloxetan-3-yl)-1H-benzo[d]imidazole-6-carboxamide (300 mg, 0.418 mmol). Yield: 0.30 g, 81%

Step-5: To a stirred solution of 2-[3,4-bis(benzyloxy)-2-fluoro-5-methoxyphenyl]-N-methyl-1-(3-methyloxetan-3-yl)-1H-1,3-benzodiazole-6-carboxamide (200 mg, 344 μmol) in trifluoroacetic acid (5.00 mL), reaction mixture stirred for 4 h at 65° C. After completion, reaction mixture was concentrated under reduced pressure to get the crude and further purified by reverse phase HPLC. Desired fractions were lyophilized to afford 2-(2-fluoro-3,4-dihydroxy-5-methoxyphenyl)-N-methyl-1-(3-methyloxetan-3-yl)-1H-1,3-benzodiazole-6-carboxamide (66.0 mg, 163 μmol) (TFA Salt) as an off white solid. Yield: 0.066 g, 47%

LCMS and NMR Data: ES MS M/Z=402 (M+1), NMR (400 MHz, DMSO-d6) δ 9.60 (s, 1H), 8.54 (d, J=4.56 Hz, 1H), 7.86 (d, J=7.44 Hz, 1H), 7.77 (d, J=8.48 Hz, 1H), 7.68 (s, 1H), 6.65 (d, J=4.56 Hz, 1H), 4.76 (d, J=5.84 Hz, 2H), 4.43 (d, J=6.08 Hz, 2H), 3.80 (s, 3H), 2.84 (s, 3H), 2.09 (s, 3H).

### Example 213: Synthesis of 2-(3,4-dihydroxy-5-methoxy-2-methylphenyl)-N-methyl-1-(3-methyloxetan-3-yl)-1H-1,3-benzodiazole-6-carboxamide

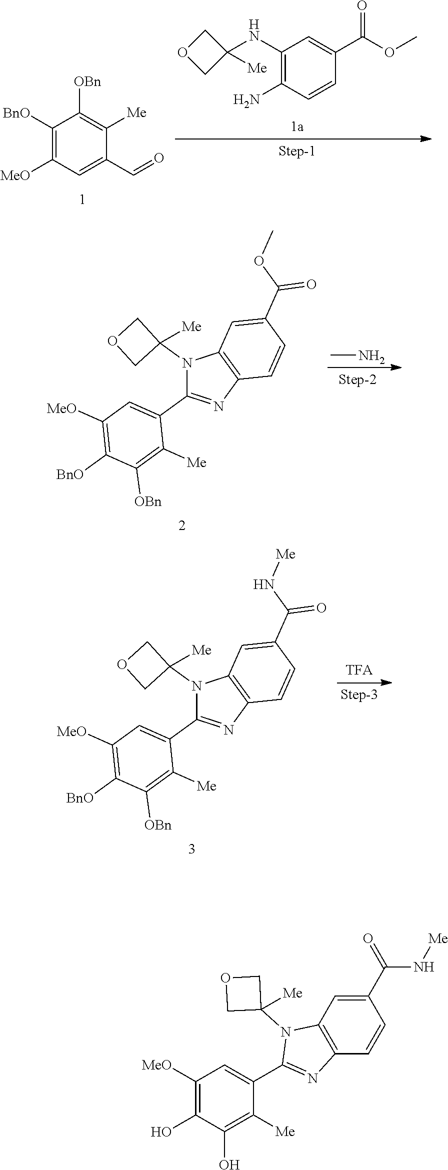

Step-1: To a stirred solution of methyl 4-amino-3-[(3-methyloxetan-3-yl)amino]benzoate (400 mg, 1.3 eq., 914 μmol) and 3,4-bis(benzyloxy)-5-methoxy-2-methylbenzaldehyde (250 mg, 690 μmol) in Dimethyl sulfoxide (3.00 mL) and disodium sulfinatosulfonate (157 mg, 1.2 eq., 828 μmol) was added at room temperature. The resulting mixture was stirred in microwave at 140° C. for 1 hr. After completion of reaction, the reaction mixture diluted with water and extracted with ethyl acetate. The organic layer dried over anhydrous sodium sulphate, filtered and concentrated to give crude. The crude purified by flash chromatography. The desired fractions were concentrated to afford methyl 2-[3,4-bis(benzyloxy)-5-methoxy-2-methylphenyl]-1-(3-methyloxetan-3-yl)-1H-1,3-benzodiazole-6-carboxylate (150 L, 238 μmol). Yield: 0.150 g, (34.57%)

Step-2: A stirred solution of methyl 2-[3,4-bis(benzyloxy)-5-methoxy-2-methylphenyl]-1-(3-methyloxetan-3-yl)-1H-1,3-benzodiazole-6-carboxylate (150 mg, 259 μmol) and methanamine (12.1 mg, 1.5 eq., 389 μmol) in ethanol at 0° C. and stirred at room temperature for 16 h. After completion of the reaction, the reaction mixture was concentrated under reduced pressure to get the crude. The crude purified by flash chromatography. The desired fractions were concentrated to afford 2-[3,4-bis(benzyloxy)-5-methoxy-2-methylphenyl]-N-methyl-1-(3-methyloxetan-3-yl)-1H-1,3-benzodiazole-6-carboxamide (160 mg, 193 μmol) as off white color solid. Yield: 0.160 g, 74.53%

Step-3: To 2-[3,4-bis(benzyloxy)-5-methoxy-2-methylphenyl]-N-methyl-1-(3-methyloxetan-3-yl)-1H-1,3-benzodiazole-6-carboxamide (160 mg, 277 μmol), trifluoroacetic acid (3.00 mL) added at room temperature. The resulting reaction mixture stirred at 60° C. for 4 h. After the complete consumption of starting material, reaction mixture was concentrated to obtain the crude. The crude was purified by reverse phase HPLC, the desired fractions were collected and lyophilized to obtain 2-(3,4-dihydroxy-5-methoxy-2-methylphenyl)-N-methyl-1-(3-methyloxetan-3-yl)-1H-1,3-benzodiazole-6-carboxamide (36.0 mg, 90.6 μmol) as off white solid. Yield: 36 mg, 32.7%

LCMS and NMR Data: ES MS M/Z=398.34(M+1)⁺, ¹H NMR (400 MHz, DMSO-d6) δ 9.08 (s, 1H), 8.75 (s, 1H), 7.81 (d, 1H), 7.73 (d, 1H), 7.65 (s, 1H), 6.50 (s, 1H), 4.92 (s, 1H), 4.67 (s, 1H), 4.43 (s, 1H), 3.77 (s, 3H), 2.83 (d, 3H), 2.08 (s, 3H), 3.01 (s, 3H).

### Example 214: Synthesis of 2-(3,4-bis(benzyloxy)-2-fluoro-5-methoxyphenyl)-N-methyl-1-(3-methyloxetan-3-yl)-1H-benzo[d]imidazole-4-carboxamide

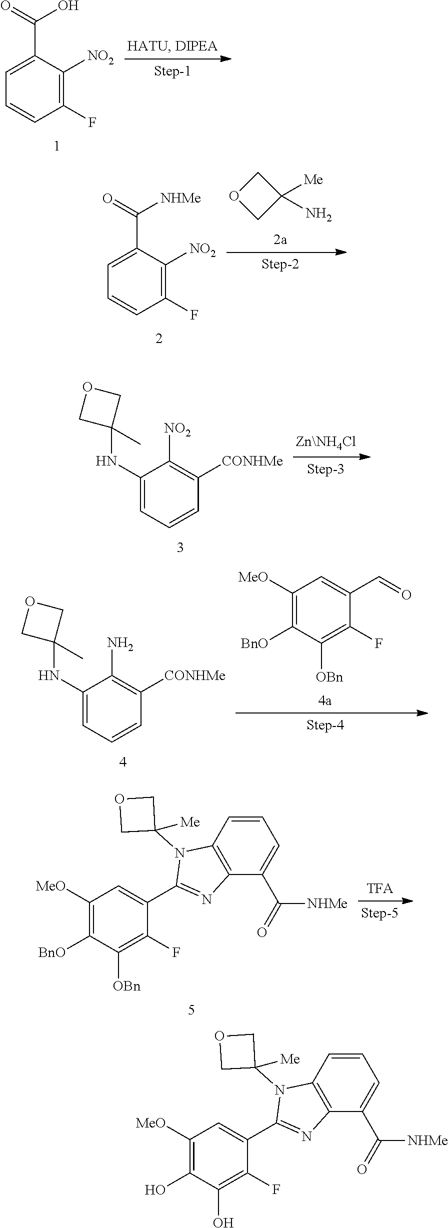

Step-1: To a stirred solution of Methanamine hydrochloride (320 mg, 4.74 mmol) in N,N-dimethylformamide (2.00 mL) was added 3-fluoro-2-nitrobenzoic acid (1.05 g, 1.2 eq., 5.69 mmol), hexafluoro-λ⁵-phosphanuide 1-[bis(dimethylamino)methylidene]-1H-1λ⁵-[1,2,3]triazolo[4,5-b]pyridin-3-ium-1-ylium-3-olate (2.70 g, 1.5 eq., 7.11 mmol) and ethylbis(propan-2-yl)amine (2.48 mL, 3 eq., 14.2 mmol) and the solution was stirred for 16 hours at room temperature. After completion of reaction, the reaction mixture was diluted with water and extracted with ethyl acetate. The organic layer dried over anhydrous sodium sulphate, filtered and concentrated to obtain crude. The crude was purified by flash chromatography. The desired fractions concentrated to afford 3-fluoro-N-methyl-2-nitrobenzamide (900 mg, 3.27 mmol) as off-white solid. Yield: 0.900 g, (69%)

Step-2: To a stirred solution of 3-fluoro-N-methyl-2-nitrobenzamide (300 mg, 1.51 mmol) in 1-methylpyrrolidin-2-one (2.50 mL) was added 3-methyloxetan-3-amine (198 mg, 1.5 eq., 2.27 mmol) and ethylbis(propan-2-yl)amine (529 μL, 2 eq., 3.03 mmol) and heated at 120° C. for 16 h. After completion of reaction, reaction mixture diluted with water and extracted with ethyl acetate. The organic layer was dried over anhydrous sodium sulphate, filtered and concentrated to get crude. Yield: 0.360 g, crude.

Step-3: To a stirred solution of N-methyl-3-[(3-methyloxetan-3-yl)amino]-2-nitrobenzamide (360 mg, 1.36 mmol) in methanol (5.00 mL), zinc (444 mg, 5 eq., 6.79 mmol) and ammonium chloride (363 mg, 5 eq., 6.79 mmol) were added at room temperature and stirred at 50° C. for 2 h. After completion of the reaction, reaction mixture was passed through celite and washed with dichloromethane, filtrate was washed with water, dried over anhydrous sodium sulfate and concentrated under reduced pressure to get 2-amino-N-methyl-3-[(3-methyloxetan-3-yl)amino]benzamide (270 mg, 367 μmol). Yield: 0.270 g, crude

Step-4: To a stirred solution of 2-amino-N-methyl-3-[(3-methyloxetan-3-yl)amino]benzamide (270 mg, 803 μmol) and 3,4-bis(benzyloxy)-2-fluoro-5-methoxybenzaldehyde (235 mg, 0.8 eq., 643 μmol) was added disodium sulfinatosulfonate (183 mg, 1.2 eq., 964 μmol) at room temperature. The resulting mixture stirred for 16 h at 85° C. After completion, ice cold water was added in the reaction mixture and solid was filtered and dried to get crude. The crude was purified by flash chromatography. The desired fractions were concentrated to afford 2-[3,4-bis(benzyloxy)-2-fluoro-5-methoxyphenyl]-N-methyl-1-(3-methyloxetan-3-yl)-1H-1,3-benzodiazole-4-carboxamide (90.0 mg, 144 μmol) as pale yellow liquid. Yield: 0.090 g, 17.9%

Step:5 To 2-[3,4-bis(benzyloxy)-2-fluoro-5-methoxyphenyl]-N-methyl-1-(3-methyloxetan-3-yl)-1H-1,3-benzodiazole-4-carboxamide (90.0 mg, 155 μmol) was added trifluoroacetic acid (1.50 mL) and the resulting solution was stirred for 3 h at 50° C. After completion, the reaction mixture was concentrated under reduced pressure to get the crude and further purified by reverse phase HPLC. Desired fractions were lyophilized to afford 2-(2-fluoro-3,4-dihydroxy-5-methoxyphenyl)-N-methyl-1-(3-methyloxetan-3-yl)-1H-1,3-benzodiazole-4-carboxamide (30.0 mg, 74.7 μmol) as white solid. Yield: 0.030 g, 48%

1H NMR: ES MS M/Z=443 (M+1), NMR (400 MHz, DMSO-d6) δ 9.41 (br s, 1H), 7.70 (d, 1H), 7.64 (d, 1H), 7.21 (d, 1H), 6.57 (d, 1H), 4.70 (d, 2H), 4.37 (d, 2H), 3.84 (t, 2H), 3.78 (s, 3H), 3.45 (t, 2H), 2.78 (s, 3H), 2.00 (s, 3H), 1.23 (s, 1H).

### Example 215: Synthesis of N-(2-(2-fluoro-3,4-dihydroxy-5-methoxyphenyl)-1-(3-methyloxetan-3-yl)-1H-benzo[d]imidazol-5-yl)butyramide

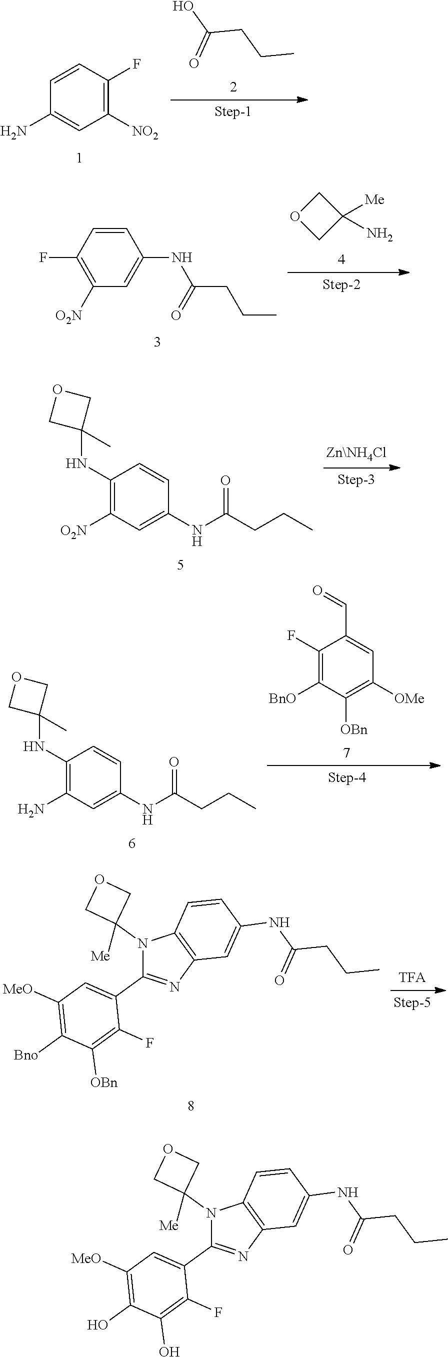

Step-1: To a stirred solution of 4-fluoro-3-nitroaniline hydrochloride (500 mg, 2.60 mmol) in N,N-dimethylformamide (5.00 mL) was added butanoic acid (287 μL, 1.2 eq., 3.12 mmol), hexafluoro-λ⁵-phosphanuide 1-[bis(dimethylamino)methylidene]-1H-1λ⁵-[1,2,3]triazolo[4,5-b]pyridin-3-ium-1-ylium-3-olate (1.48 g, 1.5 eq., 3.90 mmol) and ethylbis(propan-2-yl)amine (1.36 mL, 3 eq., 7.81 mmol) and the solution was stirred for 16 hours. After completion, reaction mixture was quenched with water and extracted with ethyl acetate. The organic layer was dried over anhydrous sodium sulfate and concentrated under reduced pressure to get crude. The crude was purified by flash chromatography. The desired fractions concentrated to afford N-(4-fluoro-3-nitrophenyl)butanamide (510 mg, 2.23 mmol) as off white solid. Yield: 0.510 g, (85.59%)

Step-2: To a stirred solution of N-(4-fluoro-3-nitrophenyl)butanamide (300 mg, 1.33 mmol) in 1-methylpyrrolidin-2-one (2.00 mL) was added 3-methyloxetan-3-amine (167 μL, 1.5 eq., 1.99 mmol) and ethylbis(propan-2-yl)amine (463 μL, 2 eq., 2.65 mmol) and heated at 100° C. for 16 h. After completion of reaction, reaction mixture was diluted with water and extracted with ethyl acetate. The organic layer was dried over anhydrous sodium sulphate, filtered and concentrated to afford N-{4-[(3-methyloxetan-3-yl)amino]-3-nitrophenyl}butanamide (364 mg, 980 μmol) as orange solid. Yield: 364 mg (73.92%)

Step-3: To a stirred solution of N-{4-[(3-methyloxetan-3-yl)amino]-3-nitrophenyl}butanamide (364 mg, 1.24 mmol) in methanol (10.0 mL), zinc (406 mg, 5 eq., 6.20 mmol) and ammonium chloride (332 mg, 5 eq., 6.20 mmol) were added at room temperature and stirred at 50° C. for 1 h. After completion of the reaction, reaction mixture was passed through celite and washed with 10% methanol in dichloromethane, filtrate was washed with water, dried over anhydrous sodium sulfate and concentrated under reduced pressure to get the desired product N-{3-amino-4-[(3-methyloxetan-3-yl)amino]phenyl}butanamide (263 mg, 829 μmol). Yield: 0.263 g, (66.8%)

Step-4: To a stirred solution of N-{3-amino-4-[(3-methyloxetan-3-yl)amino]phenyl}butanamide (263 mg, 999 μmol) and 3,4-bis(benzyloxy)-2-fluoro-5-methoxybenzaldehyde (293 mg, 0.8 eq., 799 μmol) in methanesulfinylmethane (3.00 mL) was added disodium sulfinatosulfonate (228 mg, 1.2 eq., 1.20 mmol) at room temperature. The resulting mixture was stirred for 16 h at 80° C. After completion, reaction mixture was diluted with ice cold water and filtered. The filtered solid was dissolved in dichloromethane, dried over anhydrous sodium sulfate, filtered and concentrated under reduced pressure to get crude. The crude purified by flash chromatography. The desired fractions concentrated to afford N-{2-[3,4-bis(benzyloxy)-2-fluoro-5-methoxyphenyl]-1-(3-methyloxetan-3-yl)-1H-1,3-benzodiazol-5-yl}butanamide (288 mg, 449 μmol) as yellow solid. Yield: 0.288 g, (44.93%)

Step-5: To a stirred solution of N-{2-[3,4-bis(benzyloxy)-2-fluoro-5-methoxyphenyl]-1-(3-methyloxetan-3-yl)-1H-1,3-benzodiazol-5-yl}butanamide (288 mg, 472 μmol) was added trifluoroacetic acid (2.00 mL). The resulting mixture was stirred for 2 h at 50° C. After completion, the reaction mixture was concentrated under reduced pressure to get the crude and further purified by reverse phase HPLC. Desired fractions were lyophilized to afford N-[2-(2-fluoro-3,4-dihydroxy-5-methoxyphenyl)-1-(3-methyloxetan-3-yl)-1H-1,3-benzodiazol-5-yl]butanamide (120 mg, 274 μmol) as white solid. Yield: 0.120 g, 58%

1H NMR: ES MS M/Z=430 (M+1), NMR (400 MHz, DMSO-d6) δ 10.13 (s, 1H), 9.77 (br s, 2H), 8.24 (s, 1H), 7.51-7.48 (m, 1H), 7.42 (d, J=8.80 Hz, 1H), 6.68 (d, J=6.00 Hz, 1H), 4.74 (d, J=5.60 Hz, 2H), 4.43 (d, J=6.00 Hz, 2H), 3.81 (s, 3H), 2.33 (t, J=7.20 Hz, 2H), 2.09 (s, 3H), 1.68-1.59 (m, 2H), 0.93 (t, J=7.20 Hz, 3H).

### Example 216: Synthesis of 2,3-dihydroxy-4-methoxy-6-[1-(3-methyloxetan-3-yl)-1H-1,3-benzodiazol-2-yl]benzonitrile

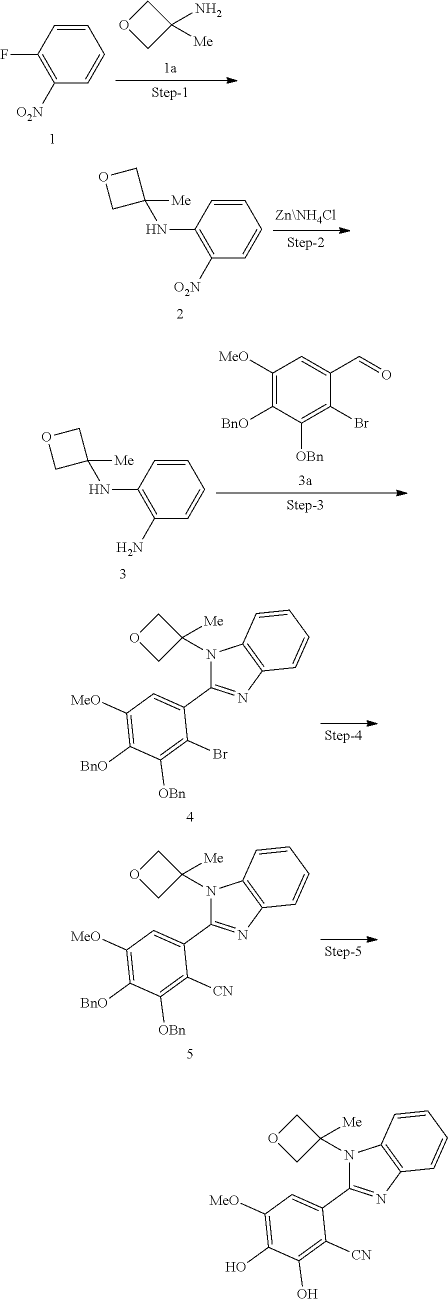

Step-1: To a stirred solution of 1-fluoro-2-nitrobenzene (1.49 mL, 14.2 mmol) in 1-methylpyrrolidin-2-one (10.0 mL) was added 3-methyloxetan-3-amine (1.28 mL, 2 eq., 28.3 mmol) and ethylbis(propan-2-yl)amine (4.95 mL, 2 eq., 28.3 mmol) and heated at 100° C. for 16 h. After completion of reaction, reaction mixture diluted with water and extracted with ethyl acetate. The organic layer was dried over anhydrous sodium sulphate, filtered and concentrated to afford 3-methyl-N-(2-nitrophenyl)oxetan-3-amine (2.30 g, 10.8 mmol) as orange solid. Yield: 2.30 g (76.54%)

Step-2: To a stirred solution of 3-methyl-N-(2-nitrophenyl)oxetan-3-amine (300 mg, 1.44 mmol) in methanol (5.00 mL) was added zinc (471 mg, 5 eq., 7.20 mmol) and ammonium chloride (385 mg, 5 eq., 7.20 mmol) were added at room temperature and stirred at 50° C. for 1 h. After completion of the reaction, reaction mixture was passed through celite and washed with 10% methanol in dichloromethane, filtrate was washed with water, dried over anhydrous sodium sulphate and concentrated under reduced pressure to get N1-(3-methyloxetan-3-yl)benzene-1,2-diamine (350 mg, 1.04 mmol) (crude) as brown solid. Yield: 0.350 g, Crude

Step-3: To a stirred solution of N1-(3-methyloxetan-3-yl)benzene-1,2-diamine (250 mg, 1.40 mmol) and 3,4-bis(benzyloxy)-2-bromo-5-methoxybenzaldehyde (479 mg, 0.8 eq., 1.12 mmol) in methanesulfinylmethane (3.00 mL), disodium sulfinatosulfonate (400 mg, 1.5 eq., 2.10 mmol) was added at room temperature. The resulting mixture stirred for 16 h at 80° C. After completion, water and ethyl acetate was added to reaction mixture. The organic fractions were collected, dried over anhydrous sodium sulphate, filtered and concentrated to get crude. The crude purified by flash chromatography. The desired fractions were concentrated to afford 2-[3,4-bis(benzyloxy)-2-bromo-5-methoxyphenyl]-1-(3-methyloxetan-3-yl)-1H-1,3-benzodiazole (230 mg, 393 μmol) as off white solid. Yield: 230 mg, 26.28%

Step-4: To a stirred solution of 2-[3,4-bis(benzyloxy)-2-bromo-5-methoxyphenyl]-1-(3-methyloxetan-3-yl)-1H-1,3-benzodiazole (240 mg, 410 μmol) in N,N-dimethylformamide (5.00 mL), λ¹-copper iminomethanide (CuCN) (55.1 mg, 1.5 eq., 615 μmol) added at room temperature. The reaction mixture was degassed with argon for 5 min and reaction mixture stirred was heated at 150° C. for 16 h. After completion, the reaction mixture cooled to room temperature, filtered through celite, the filtrate was diluted with water and extracted ethyl acetate. The organic layer dried over anhydrous sodium sulphate, filtered and concentrated to give crude. The crude purified by flash chromatography. The desired fractions concentrated to afford 2,3-bis(benzyloxy)-4-methoxy-6-[1-(3-methyloxetan-3-yl)-1H-1,3-benzodiazol-2-yl]benzonitrile (190 mg, 357 μmol) as off white solid. Yield: 190 mg, 87.19%

Step-5: To 2,3-bis(benzyloxy)-4-methoxy-6-[1-(3-methyloxetan-3-yl)-1H-1,3-benzodiazol-2-yl]benzonitrile (180 mg, 339 μmol) in oxolane (5.00 mL) added palladium(2+) hydroxide (209 mg, 5 eq., 1.69 mmol) at room temperature. The resulting reaction mixture stirred under hydrogen atmosphere for 4 h at room temperature. After the complete consumption of starting material, reaction mixture was concentrated to obtain the crude. The crude purified by reverse-phase High performance liquid chromatography. The desired fractions were collected and lyophilized to obtain 2,3-dihydroxy-4-methoxy-6-[1-(3-methyloxetan-3-yl)-1H-1,3-benzodiazol-2-yl]benzonitrile (46.0 mg, 131 μmol) as off white solids. Yield: 46 mg, 38.67%

1H NMR and LCMS data: ES MS M/Z=352.25 [M+1]+; UPLC: 99.28%; ¹H NMR (400 MHz, DMSO-d6) 10.53 (s, 1H), δ 9.68 (s, 1H), 7.75-7.72 (m, 1H), 7.30-7.28 (m, 1H), 7.69 (S, 1H), 4.67 (d, J=6.0 Hz, 2H), 4.42 (d, J=6.0 Hz, 2H), 3.90 (s, 3H), 2.08 (s, 3H).

### Example 217: Synthesis of 3-(difluoromethoxy)-6-methoxy-4-(1-(3-methyloxetan-3-yl)-1H-benzo[d]imidazol-2-yl)benzene-1,2-diol

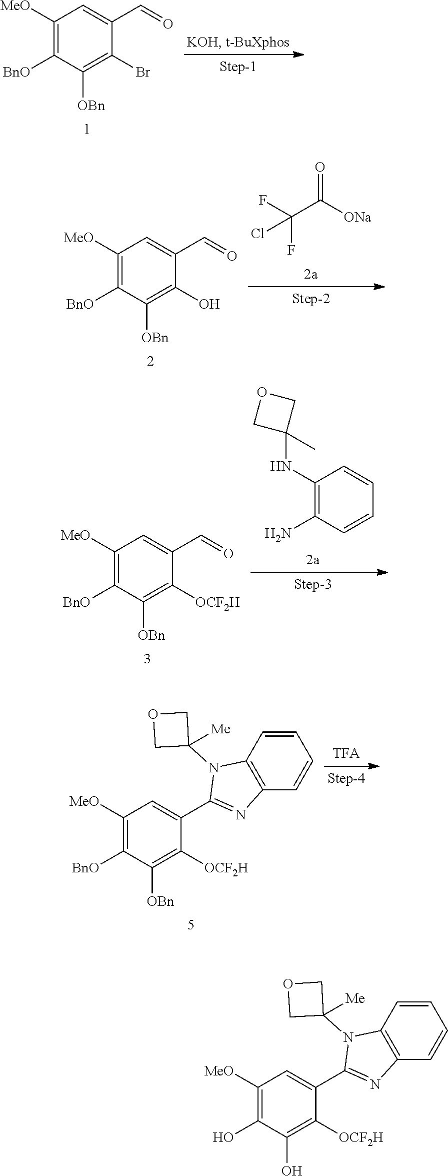

Step:1 To a stirred solution of 3,4-bis(benzyloxy)-2-bromo-5-methoxybenzaldehyde (1.00 g, 2.34 mmol) in 1,4-dioxane (7.50 mL) and water (2.50 mL) was added potassium hydroxide (394 mg, 3 eq., 7.02 mmol) at room temperature and the reaction mixture was degassed with argon for 5 min. tris(1,5-diphenylpenta-1,4-dien-3-one) dipalladium (107 mg, 0.05 eq., 117 μmol) and di-tert-butyl[2′,4′,6′-tris(propan-2-yl)-[1,1′-biphenyl]-2-yl]phosphane (99.4 mg, 0.1 eq., 234 μmol) were added to the reaction, continued degassing for 5 min and heated the reaction mixture at 100° C. for 16 h. After completion, the reaction was cooled to room temperature and passed through celite. The filtrate was diluted with 1N hydrochloric acid and extracted with ethyl acetate. The organic layer was dried over anhydrous sodium sulfate, filtered and concentrated to give crude. The crude was purified by flash chromatography. Desired fractions were concentrated to get 3,4-bis(benzyloxy)-2-hydroxy-5-methoxybenzaldehyde (900 mg, 1.72 mmol) as thick yellow liquid. Yield: 0.900 g, 73%

Step:2 To a stirred solution of 3,4-bis(benzyloxy)-2-hydroxy-5-methoxybenzaldehyde (800 mg, 2.20 mmol) in acetonitrile (8.00 mL), sodium 2,2-difluoroethanecarboperoxoyl chloride (1.00 g, 3 eq., 6.59 mmol) and cesium carbonate (1.79 g, 2.5 eq., 5.49 mmol) were added and stirred at 80° C. for 16 h. After the completion of reaction, water was added and extracted with ethyl acetate, dried over anhydrous sodium sulfate, filtered and concentrated to obtain crude. The crude purified by flash chromatography. The desired fractions were concentrated to afford 3,4-bis(benzyloxy)-2-(difluoromethoxy)-5-methoxybenzaldehyde (120 mg, 246 μmol) as thick greenish liquid. Yield: 0.120 g, 11%

Step:3 To a stirred solution of N1-(3-methyloxetan-3-yl)benzene-1,2-diamine (110 mg, 617 μmol) and 3,4-bis(benzyloxy)-2-(difluoromethoxy)-5-methoxybenzaldehyde (205 mg, 0.8 eq., 494 μmol) in methanesulfinylmethane (1.20 mL), disodium sulfinatosulfonate (176 mg, 1.5 eq., 926 μmol) was added at room temperature. The resulting mixture stirred for 16 h at 80° C. After completion, chilled water was added to reaction mixture and filtered. The solid filtrate was dissolved in dichloromethane, dried over anhydrous sodium sulfate, filtered and concentrated to get crude. The crude purified by flash chromatography. The desired fractions were concentrated to afford 2-[3,4-bis(benzyloxy)-2-(difluoromethoxy)-5-methoxyphenyl]-1-(3-methyloxetan-3-yl)-1H-1,3-benzodiazole (108 mg, 166 μmol). Yield: 108 mg, 27%

Step-4: To 2-[3,4-bis(benzyloxy)-2-(difluoromethoxy)-5-methoxyphenyl]-1-(3-methyloxetan-3-yl)-1H-1,3-benzodiazole (100 mg, 175 μmol) was added trifluoroacetic acid (2.00 mL) and the resulting solution was stirred for 2 h at 60° C. After completion, the reaction mixture was concentrated under reduced pressure to get crude and further purified by reverse phase HPLC. Desired fractions were lyophilized to afford 3-(difluoromethoxy)-6-methoxy-4-[1-(3-methyloxetan-3-yl)-1H-1,3-benzodiazol-2-yl]benzene-1,2-diol (27.0 mg, 66.4 μmol) as the product. Yield: 0.027 g, 38%

1H NMR: ES MS M/Z=374 (M+1), NMR (400 MHz, DMSO-d6) δ 9.43 (s, 1H), 9.33 (s, 1H), 8.13 (s, 1H), 6.96 (d, 1H), 6.68 (s, 1H), 6.62 (d, 1H), 6.53 (d, 1H), 4.67 (d, 2H), 4.33 (d, 2H), 3.78 (s, 3H), 2.69 (s, 3H), 1.97 (s, 3H).

### Example 218: Synthesis of 4-[6-ethyl-3-(3-methyloxetan-3-yl)-3H-imidazo[4,5-c]pyridin-2-yl]-3-fluoro-6-methoxybenzene-1,2-diol

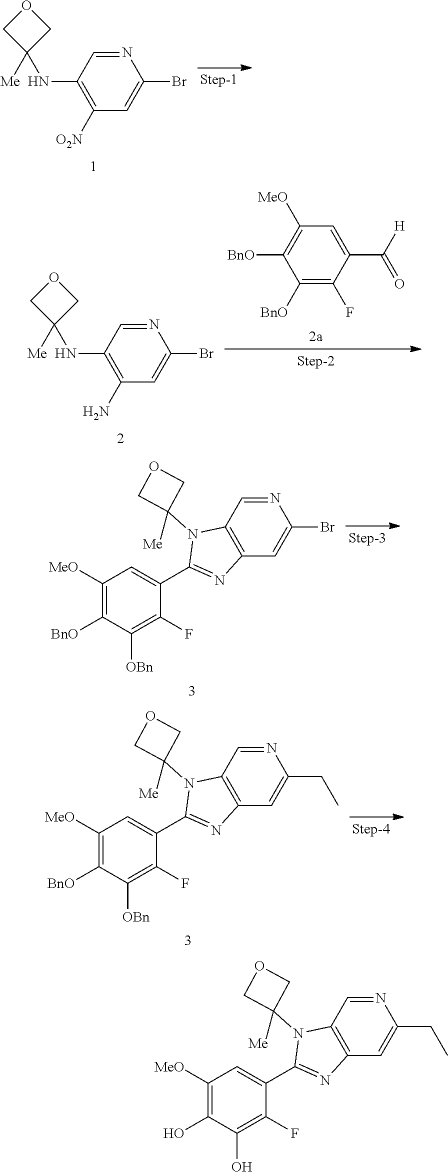 [SMILES: CC1(Nc2cnc(Br)cc2[N+](=O)[O-])COC1 ; CC1(Nc2cnc(Br)cc2N)COC1 ; COc1cc(C=O)c(F)c(OCc2ccccc2)c1OCc1ccccc1 ; COc1cc(-c2nc3cc(Br)ncc3n2C2(C)COC2)c(F)c(OCc2ccccc2)c1OCc1ccccc1 ; CCc1cc2nc(-c3cc(OC)c(OCc4ccccc4)c(OCc4ccccc4)c3F)n(C3(C)COC3)c2cn1 ; CCc1cc2nc(-c3cc(OC)c(O)c(O)c3F)n(C3(C)COC3)c2cn1]

Step-1: To a stirred solution of 6-bromo-N-(3-methyloxetan-3-yl)-4-nitropyridin-3-amine (800 mg, 2.78 mmol) in methanol (5.00 mL), Zinc dust (902 mg, 5 eq., 13.9 mmol) and ammonium chloride (743 mg, 5 eq., 13.9 mmol) were added at room temperature and the reaction mixture stirred for 2 h at 30° C. After completion, the reaction mixture passed through celite bed. Filtrate was extracted with ethyl acetate, organic layer dried over anhydrous sodium sulfate, filtered and concentrated to get as 6-bromo-N3-(3-methyloxetan-3-yl)pyridine-3,4-diamine (450 mg, 1.74 mmol) as a sticky liquid. Yield: 450 mg, 62.78%

Step-2: To a stirred solution of 6-bromo-N3-(3-methyloxetan-3-yl)pyridine-3,4-diamine (450 mg, 1.55 mmol) and 3,4-bis(benzyloxy)-2-fluoro-5-methoxybenzaldehyde (455 mg, 0.8 eq., 1.24 mmol) in methanesulfinylmethane (3.00 mL), disodium sulfinatosulfonate (442 mg, 1.5 eq., 2.33 mmol) was added at room temperature. The resulting mixture stirred for 16 h at 80° C. After completion, water and ethyl acetate was added to reaction mixture. The organic fractions were collected, dried over anhydrous sodium sulphate, filtered and concentrated to get crude. The crude purified by flash chromatography. The desired fractions were concentrated to afford 2-[3,4-bis(benzyloxy)-2-fluoro-5-methoxyphenyl]-6-bromo-3-(3-methyloxetan-3-yl)-3H-imidazo[4,5-c]pyridine (410 mg, 631 μmol) as pink solid. Yield: 410 mg, 40.65%

Step-3: To a stirred solution of 2-[3,4-bis(benzyloxy)-2-fluoro-5-methoxyphenyl]-6-bromo-3-(3-methyloxetan-3-yl)-3H-imidazo[4,5-c]pyridine (200 mg, 331 μmol) and ethylboronic acid (196 mg, 8 eq., 2.65 mmol) in toluene (1.50 mL) and water (500 μL) was added dipotassium carbonate (137 mg, 3 eq., 993 μmol) at room temperature. The reaction mixture was degassed with argon for 5 min. Then [1,1′-Bis(diphenylphosphino)ferrocene]dichloropalladium(II), complex with dichloromethane (24.3 mg, 0.09 eq., 29.8 μmol) added to the above suspension, degassed for 5 minutes and reaction mixture stirred was heated at 120° C. for 16 h. After completion, the reaction mixture cooled to room temperature, diluted with water and extracted ethyl acetate. The organic layer dried over anhydrous sodium sulphate, filtered and concentrated to give crude. The crude purified by flash chromatography. The desired fractions concentrated to afford 2-[3,4-bis(benzyloxy)-2-fluoro-5-methoxyphenyl]-6-ethyl-3-(3-methyloxetan-3-yl)-3H-imidazo[4,5-c]pyridine (50.0 mg, 90.3 μmol). Yield: 50 mg, 27.3%

Step-4: To 2-[3,4-bis(benzyloxy)-2-fluoro-5-methoxyphenyl]-6-ethyl-3-(3-methyloxetan-3-yl)-3H-imidazo[4,5-c]pyridine (85.0 mg, 154 μmol) trifluoroacetic acid (1.00 mL) added at room temperature. The resulting reaction mixture stirred at 60° C. for 4 h. After the complete consumption of starting material, reaction mixture was concentrated to obtain the crude. The crude was purified by reverse phase HPLC, the desired fractions were collected and lyophilized to obtain 4-[6-ethyl-3-(3-methyloxetan-3-yl)-3H-imidazo[4,5-c]pyridin-2-yl]-3-fluoro-6-methoxybenzene-1,2-diol (16.0 mg, 42.4 μmol) as off-white solid. Yield: 16 mg, 27.63%

LCMS and NMR Data: ES MS M/Z=374.32 (M+1)⁺, UPLC: 99.68%; 1H NMR (400 MHz, DMSO-d6) δ 9.69 (s, 2H), 8.93 (s, 1H), 8.01 (s, 1H), 6.66 (d, J=6.0 Hz, 1H), 4.75 (d, J=6.0 Hz, 2H), 4.46 (d, J=6.0 Hz, 1H), 3.81 (s, 3H), 2.02 (m, 2H), 2.11 (s, 3H), 1.33 (t, J=7.6 Hz, 3H).

### Example 219: Synthesis of 2-(2-fluoro-3,4-dihydroxy-5-methoxyphenyl)-3-(3-methyloxetan-3-yl)-3H-imidazo[4,5-c]pyridine-6-carboxamide

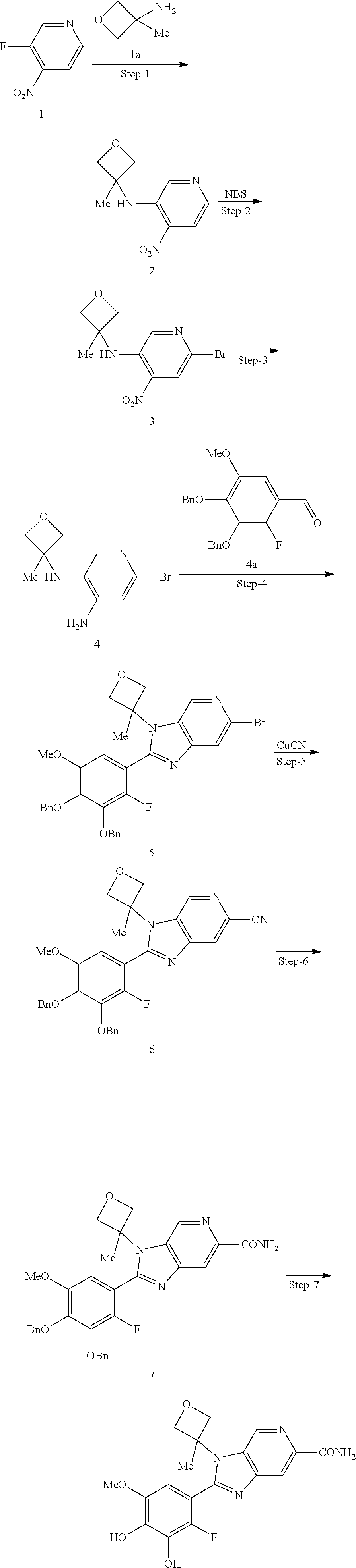

Step-1: A stirred solution of 3-fluoro-4-nitropyridine (7.00 g, 49.3 mmol) and 3-methyloxetan-3-amine (3.34 mL, 1.5 eq., 73.9 mmol) in 1-methylpyrrolidin-2-one (5.00 mL) and N,N-Diispropylethylamine (25.8 mL, 3 eq., 148 mmol) was added and stirred and heated at 110° C. for 16 h. After completion of reaction, reaction mixture diluted with water and extracted with ethyl acetate. The combined organic layers were dried over anhydrous sodium sulphate, filtered and concentrated to get crude. The crude purified by flash chromatography. The desired fractions were concentrated to afford N-(3-methyloxetan-3-yl)-4-nitropyridin-3-amine (5.40 g, 25.6 mmol) as orange color solid. Yield: 5.400 g, 51.87%

Step-2: A stirred solution of N-(3-methyloxetan-3-yl)-4-nitropyridin-3-amine (2.00 g, 9.56 mmol) in N,N-dimethylformamide (5.00 mL) and N-Bromosuccinamide (2.24 g, 1.2 eq., 11.5 mmol) was added at 0° C. and stirred for 16 h. After completion of the reaction, the reaction mixture was diluted with water and extracted ethyl acetate and dried and concentrated under reduced pressure to get the crude. The crude purified by flash chromatography. The desired fractions were concentrated to afford 6-bromo-N-(3-methyloxetan-3-yl)-4-nitropyridin-3-amine (2.50 g, 7.46 mmol) as orange color solid. Yield: 2.500 g, 78.06%

Step-3: A stirred solution of 6-bromo-N-(3-methyloxetan-3-yl)-4-nitropyridin-3-amine (1.00 g, 3.47 mmol) in methanol (10.0 mL), zinc (1.13 g, 5 eq., 17.4 mmol) and ammonium chloride (928 mg, 5 eq., 17.4 mmol) were added and stirred at room temperature for 3 h. After completion of reaction, reaction mixture filtered through celite and filtrate was diluted with water and extracted with dichloromethane. The combined organic layers were dried over anhydrous sodium sulphate, filtered and concentrated to afford 6-bromo-N3-(3-methyloxetan-3-yl)pyridine-3,4-diamine (800 mg, 2.76 mmol) as brown sticky solid. Yield: 0.800 g, 79.47%

Step-4: To a stirred solution 3,4-bis(benzyloxy)-2-fluoro-5-methoxybenzaldehyde (650 mg, 1.77 mmol) and 6-bromo-N3-(3-methyloxetan-3-yl)pyridine-3,4-diamine (458 mg, 1.77 mmol) in Dimethyl sulfoxide (5.00 mL) and 2-[3,4-bis(benzyloxy)-2-fluoro-5-methoxyphenyl]-6-bromo-1-(3-methyloxetan-3-yl)-1H-imidazo[4,5-c]pyridine (500 mg, 678 μmol) was added at room temperature. The resulting mixture stirred and heated at 85° C. for 16 hr. After completion of reaction, the reaction mixture diluted with ice cold water and extracted with ethyl acetate. The organic layer dried over anhydrous sodium sulphate, filtered and concentrated to give crude. The crude purified by flash chromatography. The desired fractions were concentrated to afford 2-[3,4-bis(benzyloxy)-2-fluoro-5-methoxyphenyl]-6-bromo-1-(3-methyloxetan-3-yl)-1H-imidazo[4,5-c]pyridine (500 mg, 678 μmol) as brown color solid. Yield: 0.50 g, 38.23%

Step-5: To a stirred solution of 2-[3,4-bis(benzyloxy)-2-fluoro-5-methoxyphenyl]-3-(3-methyloxetan-3-yl)-3H-imidazo[4,5-c]pyridine-6-carbonitrile (150 mg, 272 μmol) in N,N-dimethylformamide (4.00 mL) and peroxol (92.7 mg, 3 eq., 817 μmol) and dipotassium carbonate (113 mg, 3 eq., 817 μmol) were added and heated at 140° C. for 16 hours. Reaction monitored by LCMS Reaction mixture concentrated under reduced pressure to get crude. The crude purified by flash chromatography. The desired fractions were concentrated to afford 2-[3,4-bis(benzyloxy)-2-fluoro-5-methoxyphenyl]-3-(3-methyloxetan-3-yl)-3H-imidazo[4,5-c]pyridine-6-carboxamide (90.0 mg, 95.0 μmol) as brown color solid. Yield: 0.090 g, 34.86%

Step-6: To a solution of 2-[3,4-bis(benzyloxy)-2-fluoro-5-methoxyphenyl]-3-(3-methyloxetan-3-yl)-3H-imidazo[4,5-c]pyridine-6-carboxamide (90.0 mg, 158 μmol) in trifluoroacetic acid was at 60° C. temperature and the reaction mixture stirred for 2 h. After completion, the reaction mixture concentrated under reduced pressure to get crude. The crude was purified on reverse phase prep HPLC to get 2-(2-fluoro-3,4-dihydroxy-5-methoxyphenyl)-3-(3-methyloxetan-3-yl)-3H-imidazo[4,5-c]pyridine-6-carboxamide (5.00 mg, 12.9 μmol) as white solid. Yield: 0.005 g (8.13%)

LCMS and NMR Data: ES MS M/Z=389.32 (M+1), 1H NMR (400 MHz, DMSO-d6) δ 9.56 (d, J=17.2 Hz, 2H), 8.70 (s, 1H), 8.31 (s, 1H), 8.05 (s, 1H), 7.63 (s, 1H), 6.67 (d, J=8.4 Hz, 1H), 4.78 (d, J=6.0 Hz, 2H), 4.44 (d, J=6.0 Hz, 2H), 3.80 (s, 3H), 2.09 (s, 3H)

### Example 220: Synthesis of 6-methoxy-3-methyl-4-(1-(1-methylcyclobutyl)-1H-benzo[d]imidazol-2-yl)benzene-1,2-diol

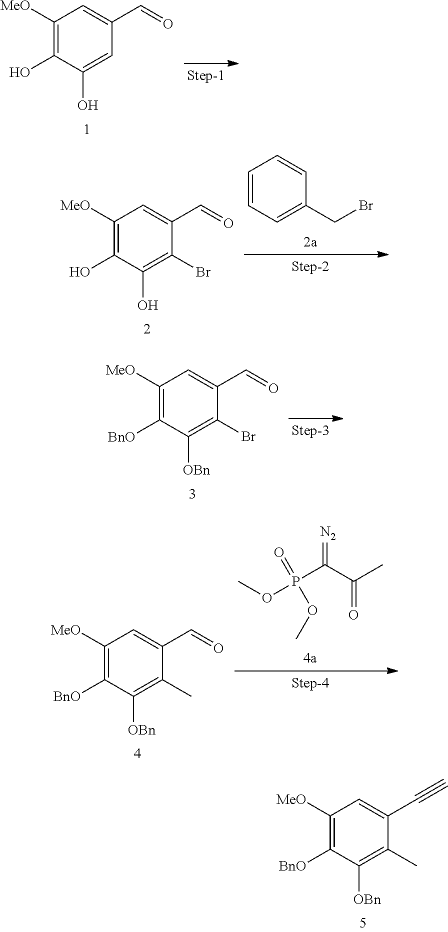

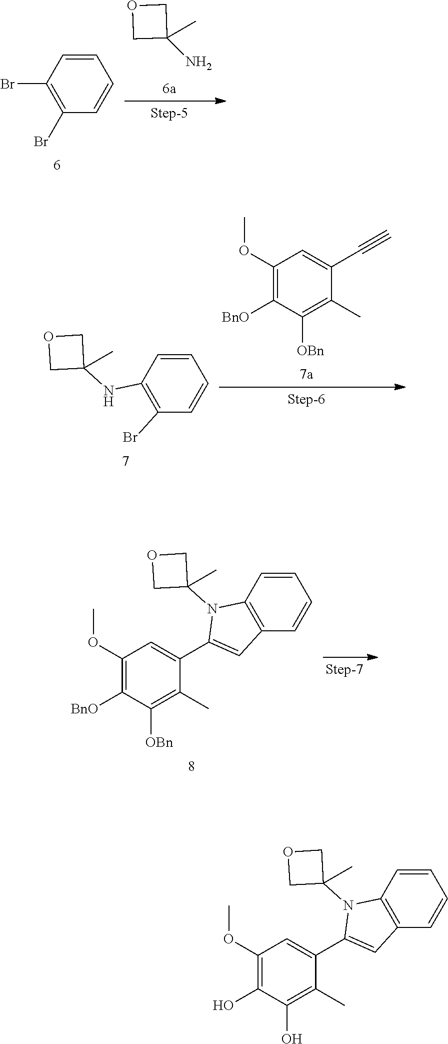

Step-1: To a solution of 3,4-dihydroxy-5-methoxybenzaldehyde (15.0 g, 89.2 mmol) in acetic acid (100 mL) and bromine (1.08 mL, 20.9 mmol) diluted in acetic acid was added dropwise to the reaction mixture and stirred for 16 hr. After completion of the reaction, the reaction mixture was quenched with Sodium thiosulfate and stirred for 10 minute so that solid compound precipitate. After that reaction mixture filtered out and solid washed with ice cold water and dried under vacuum. The solid afford 2-bromo-3,4-dihydroxy-5-methoxybenzaldehyde (22.0 g, 80.1 mmol) as off white solid. Yield: 22.00 g, 89.83%

Step-2: To a solution of 2-bromo-3,4-dihydroxy-5-methoxybenzaldehyde (22.0 g, 89.1 mmol) in N,N-dimethylformamide (200 mL), (bromomethyl)benzene (31.7 mL, 3 eq., 267 mmol), dipotassium carbonate (36.9 g, 3 eq., 267 mmol) and potassium iodide (2.96 g, 0.2 eq., 17.8 mmol) were added to the reaction mixture and stirred and heated at 60° C. for 4 h. After completion of the reaction, the reaction mixture was filtered and the filtrate was concentrated under reduced pressure to get the crude. The crude purified by flash chromatography. The desired fractions were concentrated to afford 3,4-bis(benzyloxy)-2-bromo-5-methoxybenzaldehyde (23.0 g, 45.8 mmol) as off white solid. Yield: 23.0 g, 74.46%

Step-3: To a solution of 3,4-bis(benzyloxy)-2-bromo-5-methoxybenzaldehyde (4.00 g, 9.36 mmol) and methylboronic acid (672 mg, 1.2 eq., 11.2 mmol) in 1,4-dioxane (30.0 mL) and water (2.00 mL). Cesium carbonate (6.10 g, 2 eq., 18.7 mmol) added at room temperature and the reaction mixture was degassed with argon for 5 min. was added to the reaction, continued degassing for 5 min and heated the reaction mixture at 90° C. for 4 h. After completion, the reaction was cooled to room temperature and passed through celite bed. Filtrate was diluted with ethyl acetate and washed with water. The organic layer was dried over anhydrous sodium sulfate, filtered and concentrated to give crude. The crude purified in flash chromatography. The desired fractions were concentrated to afford 3,4-bis(benzyloxy)-5-methoxy-2-methylbenzaldehyde (2.50 g, 6.48 mmol) as pale yellow oil. Yield: 2.5 g, 69.21%

Step-4: A solution of N-(2-bromophenyl)-3-methyloxetan-3-amine (500 mg, 2.07 mmol) and 2,3-bis(benzyloxy)-5-ethynyl-1-methoxy-4-methylbenzene (740 mg, 2.07 mmol) in N,N-dimethylformamide (5.00 mL), triethylamine (746 μL, 2.6 eq., 5.37 mmol) was added, and reaction mixture was purged with argon for 5 min, then Bis(triphenylphosphine)palladium(II) dichloride (58.0 mg, 0.04 eq., 82.6 μmol) and diiodocopper (45.9 mg, 0.07 eq., 145 μmol) and again purged with argon for 5 min and heated to 100° C. for 24 h. After completion of reaction, the reaction mixture diluted with water and extracted with dichloromethane. The combined organic layers dried over anhydrous sodium sulphate, filtered and concentrated to obtain crude. The crude purified by flash chromatography. The desired fractions were concentrated to afford 2-[3,4-bis(benzyloxy)-5-methoxy-2-methylphenyl]-1-(3-methyloxetan-3-yl)-1H-indole (180 mg, 149 μmol) as brown mass. Yield: 180.0 mg, 43%

Step-5: To a solution of 1,2-dibromobenzene (1.00 g, 4.24 mmol) and N-(2-bromophenyl)-3-methyloxetan-3-amine (500 mg, 2.03 mmol) in toluene (5.00 mL) sodium 2-methylpropan-2-olate (823 mg, 2 eq., 8.48 mmol) added at room temperature and the reaction mixture was degassed with argon for 5 min. tris((1E,4E)-1,5-diphenylpenta-1,4-dien-3-one) dipalladium (77.6 mg, 0.02 eq., 84.8 μmol) and [2′-(diphenylphosphanyl)-[1,1′-binaphthalen]-2-yl]diphenylphosphane (52.8 mg, 0.02 eq., 84.8 μmol) was added to the reaction, continued degassing for 5 min and heated the reaction mixture at 90° C. for 6 h. After completion, the reaction was cooled to room temperature and passed through celite bed. Filtrate was diluted with ethyl acetate and washed with water. The organic layer was dried over anhydrous sodium sulfate, filtered and concentrated to give crude. The crude purified by flash chromatography. The desired fractions were concentrated to afford N-(2-bromophenyl)-3-methyloxetan-3-amine as pale yellow oil. Yield: 0.45 g, 45.96%

Step-6: A solution of N-(2-bromophenyl)-3-methyloxetan-3-amine (500 mg, 2.07 mmol) and 2,3-bis(benzyloxy)-5-ethynyl-1-methoxy-4-methylbenzene (740 mg, 2.07 mmol) in N,N-dimethylformamide (5.00 mL), triethylamine (746 μL, 2.6 eq., 5.37 mmol) was added, and reaction mixture was purged with argon for 5 min, then Bis(triphenylphosphine)palladium(II) dichloride (58.0 mg, 0.04 eq., 82.6 μmol) and diiodocopper (45.9 mg, 0.07 eq., 145 μmol) and again purged with argon for 5 min and heated to 100° C. for 24 h. After completion of reaction, the reaction mixture diluted with water and extracted with dichloromethane. The combined organic layers dried over anhydrous sodium sulphate, filtered and concentrated to obtain crude. Purification was done by Reverse Prep HPLC to afford 2-[3,4-bis(benzyloxy)-5-methoxy-2-methylphenyl]-1-(3-methyloxetan-3-yl)-1H-indole (180 mg, 149 μmol) as brown mass. Yield: 72.0 mg, 18%

Step-7: To a stirred solution of 2-[3,4-bis(benzyloxy)-5-methoxy-2-methylphenyl]-1-(3-methyloxetan-3-yl)-1H-indole (70.0 mg, 135 μmol) in Tetrahydrofuran (5.00 mL), Palladium hydroxide (16.5 mg, 135 μmol) was charged and stirred under hydrogen atmosphere at room temperature for 2 h. After completion of reaction, reaction mixture filtered through celite. Filtrate was distilled below 30° C. to obtain crude. Purification was done by Reverse Prep HPLC to get 6-methoxy-3-methyl-4-[1-(3-methyloxetan-3-yl)-1H-indol-2-yl]benzene-1,2-diol (5.40 mg, 15.9 μmol) as off white solid. Yield: 5.40 mg, 11.78%

ES MS M/Z=338.03 (M−1), UPLC: 99.74%. ¹HNMR (400 MHz, DMSO-d6): δ 7.55 (J=7.52 Hz, 1H), 7.12-6.99 (m, 3H), 6.37 (s, 1H), 6.27 (s, 1H), 4.89 (J=5.4 Hz, 1H), 4.59 (J=4.5 Hz, 1H), 4.89 (J=5.8 Hz, 1H)), 3.82 (4.89 (J=5.4 Hz, 1H)), 3.74 (s, 3H), 2.00 (s, 3H), 1.97 (s, 3H), 1.77 (s, 3H), 1.70-1.63 (m, 1H), 1.56 (bs, 1H).

### Example 221: Synthesis of 4-(5-(azetidin-1-yl)-1-methyl-1H-benzo[d]imidazol-2-yl)-6-methoxy-3-(trifluoromethyl)benzene-1,2-diol

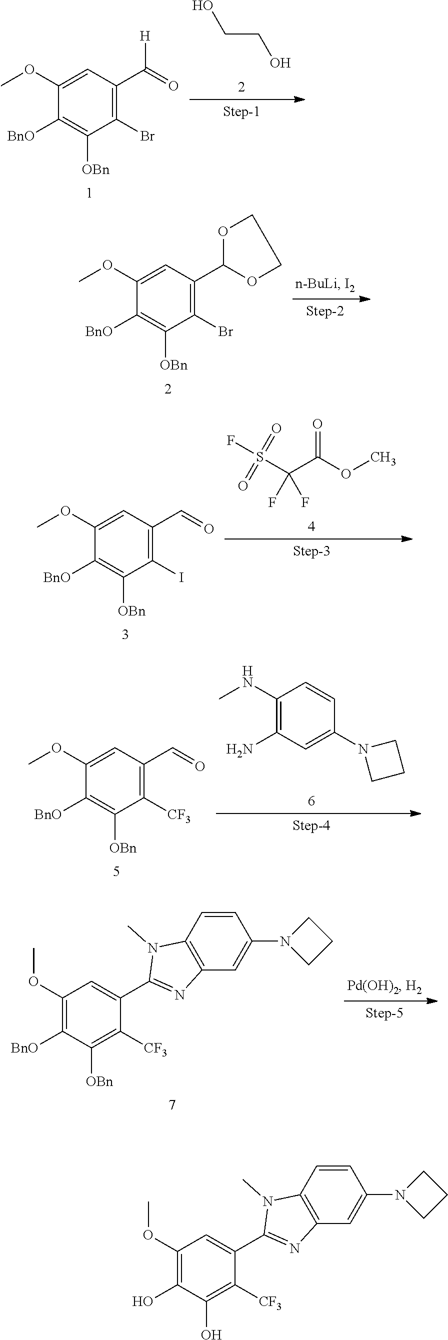

Step-1: To a solution of 3,4-bis(benzyloxy)-2-bromo-5-methoxybenzaldehyde (2.00 g, 4.68 mmol) in dry toluene (20.0 mL), ethane-1,2-diol (785 μL, 3 eq., 14.0 mmol) and dry 4-methylbenzene-1-sulfonic acid (80.6 mg, 0.1 eq., 468 μmol) were added to the reaction mixture and stirred and heated at 130° C. for 8 h. After completion of the reaction, the reaction mixture was concentrated under reduced pressure to get the crude. The crude purified by flash chromatography. The desired fractions were concentrated to afford 2-[3,4-bis(benzyloxy)-2-bromo-5-methoxyphenyl]-1,3-dioxolane (1.00 g, 2.10 mmol) as off white solid. Yield: 1.0 g, 44.87%

Step-2: To a stirred solution of 2-[3,4-bis(benzyloxy)-2-bromo-5-methoxyphenyl]-1,3-dioxolane (1.00 g, 2.12 mmol) in Tetrahydrofuran (10.0 mL) under inert atmosphere. The solution was cooled at −78° C. and lithium butyl (1.27 mL, 1.5 eq., 3.18 mmol) was added dropwise. The reaction mixture stirred for 20 minutes at −78° C. Iodine (535 mg, 2 eq., 4.24 mmol) in tetrahydrofuran (5 ml) was added dropwise at −78° C. and stirred for 1 h and allowed to come at room temperature. After completion, the reaction mixture quenched with 6N hydrochloric acid solution at 0° C. and extracted with ethyl acetate. The combined organic layers dried over anhydrous sodium sulphate, filtered and concentrated to obtain crude. The crude was purified by flash chromatography. Pure fractions were concentrated to get 3,4-bis(benzyloxy)-2-iodo-5-methoxybenzaldehyde (480 mg, 972 μmol) as off white solid. Yield: 0.48 g, 45.79%

Step-3: To a stirred solution of 3,4-bis(benzyloxy)-2-iodo-5-methoxybenzaldehyde (575 mg, 1.21 mmol) in N,N-dimethylformamide (5.00 mL), [bis(dimethylamino)phosphoryl]dimethylamine (844 μL, 4 eq., 4.85 mmol) and Copper(I) iodide (277 mg, 1.2 eq., 1.45 mmol) and methyl 2,2-difluoro-2-(fluorosulfonyl)acetate (1.23 mL, 8 eq., 9.70 mmol) were added at room temperature in presence of nitrogen atmosphere. The resulting mixture stirred for 16 h at 85° C. After completion, reaction mixture was extracted with ethyl acetate, dried over anhydrous sodium sulfate and concentrated under reduced pressure to get crude. The crude purified by flash chromatography. The desired fractions were concentrated to afford 3,4-bis(benzyloxy)-5-methoxy-2-(trifluoromethyl)benzaldehyde (300 mg, 576 μmol) as sticky solid. Yield: 0.30 g, 53%

Step-4: To a stirred solution of 4-(azetidin-1-yl)-N1-methylbenzene-1,2-diamine (240 mg, 1.08 mmol) and 3,4-bis(benzyloxy)-5-methoxy-2-(trifluoromethyl)benzaldehyde (361 mg, 0.8 eq., 867 μmol) in methanesulfinylmethane (10.0 mL) was added disodium sulfinatosulfonate (247 mg, 1.2 eq., 1.30 mmol) at room temperature. The resulting mixture stirred for 16 h at 85° C. After completion, reaction mixture cooled to room temperature and diluted with water and extracted with ethyl acetate. The organic layer was dried over anhydrous sodium sulfate and concentrated under reduced pressure to get crude. The crude purified by flash chromatography. The desired fractions concentrated to afford 5-(azetidin-1-yl)-2-[3,4-bis(benzyloxy)-5-methoxy-2-(trifluoromethyl)phenyl]-1-methyl-1H-1,3-benzodiazole (230 mg, 317 μmol) as off white solid. Yield: 0.23 g, 29.24%

Step-5: To a solution of 5-(azetidin-1-yl)-2-[3,4-bis(benzyloxy)-5-methoxy-2-(trifluoromethyl)phenyl]-1-methyl-1H-1,3-benzodiazole (200 mg, 275 μmol) in Tetrahydrofuran (10.0 mL) was added 20% Palladium hydroxide (250 mg, 1.3 eq., 357 μmol) at room temperature and the reaction mixture stirred for 3 h under hydrogen atmosphere. After completion, the reaction mixture passed through celite bed. Filtrate was concentrated to get crude. Crude was purified by reverse phase HPLC and desired fractions were lyophilized to get 4-[5-(azetidin-1-yl)-1-methyl-1H-1,3-benzodiazol-2-yl]-6-methoxy-3-(trifluoromethyl)benzene-1,2-diol (29.0 mg, 70.0 μmol) as off white solid. Yield: 0.029 g, 25.43%

ES MS M/Z=394.00 (M+1), 1H NMR (400 MHz, DMSO-d6) δ 9.71 (brs, 1H), 7.36 (d, J=8.8 Hz, 1H), 6.57-6.56 (m, 2H), 6.48 (dd, J=2, 8.4 Hz, 1H), 3.8 (s, 3H), 3.80 (t, J=7.2 Hz, 4H), 3.45 (s, 3H), 2.32-2.25 (m, 2H).

### Example 222: Synthesis of 4-(5-(benzylamino)-1-(3-methyloxetan-3-yl)-1H-benzo[d]imidazol-2-yl)-3-fluoro-6-methoxybenzene-1,2-diol

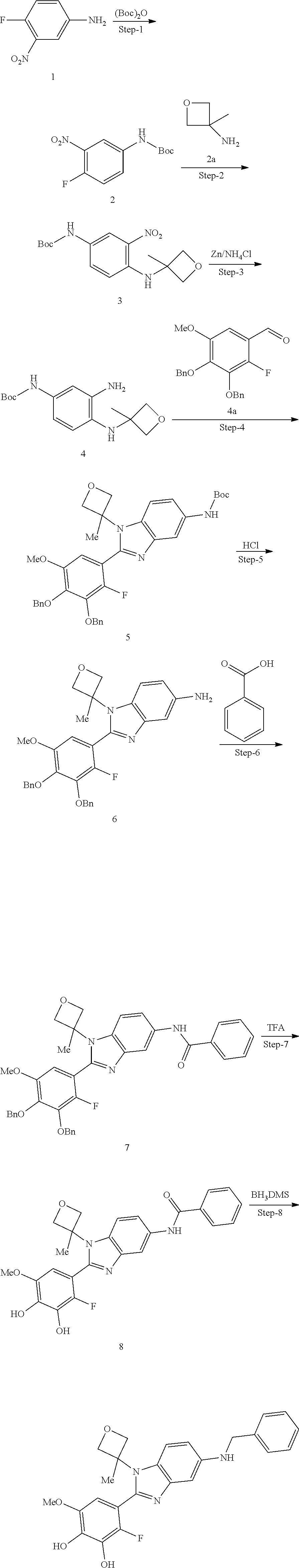

Step:1 To a stirred solution of 4-fluoro-3-nitroaniline (3.00 g, 19.2 mmol) in Tetrahydrofuran (30.0 mL) was added di-tert-butyl dicarbonate (5.56 mL, 1.3 eq., 25.0 mmol). The reaction mixture was heated at 70° C. for 16 h. After completion of reaction, reaction mixture was diluted with water and extracted with ethyl acetate. The organic layer was separated, dried over sodium sulphate, filtered and concentrated under reduced pressure to get the crude. The crude purified by flash chromatography. The desired fractions concentrated to get tert-butyl N-(4-fluoro-3-nitrophenyl)carbamate (2.00 g, 7.79 mmol) as pale yellow solid. Yield: 2.0 g, 40.5%

Step:2 To a stirred solution of tert-butyl N-(4-fluoro-3-nitrophenyl)carbamate (350 mg, 1.37 mmol) in 1-methylpyrrolidin-2-one (3.00 mL) was added 3-methyloxetan-3-amine (179 mg, 1.5 eq., 2.05 mmol) and ethylbis(propan-2-yl)amine (477 μL, 2 eq., 2.73 mmol) and heated at 120° C. for 16 h. After completion of reaction, reaction mixture diluted with water and extracted with ethyl acetate. The organic layer was dried over anhydrous sodium sulphate, filtered and concentrated to get crude. Crude was purified by flash chromatography. Desired fractions were collected and concentrated to get tert-butyl N-{4-[(3-methyloxetan-3-yl)amino]-3-nitrophenyl}carbamate (200 mg, 579 μmol) as orange solid. Yield: 0.200 g (42.4%)

Step-3: To a stirred solution of tert-butyl N-{4-[(3-methyloxetan-3-yl)amino]-3-nitrophenyl}carbamate (200 mg, 619 μmol) in methanol (5.00 mL), zinc (202 mg, 5 eq., 3.09 mmol) and ammonium chloride (165 mg, 5 eq., 3.09 mmol) were added at room temperature and stirred at 50° C. for 1 h. After completion of the reaction, reaction mixture was passed through celite and washed with dichloromethane, filtrate was washed with water, dried over anhydrous sodium sulfate and concentrated under reduced pressure to get the desired product tert-butyl N-{3-amino-4-[(3-methyloxetan-3-yl)amino]phenyl}carbamate (170 mg, 539 μmol). Yield: 0.170 g, 87%

Step-4: To a stirred solution of tert-butyl N-{3-amino-4-[(3-methyloxetan-3-yl)amino]phenyl}carbamate (170 mg, 579 μmol) and 3,4-bis(benzyloxy)-2-fluoro-5-methoxybenzaldehyde (170 mg, 0.8 eq., 464 μmol) in methanesulfinylmethane (2.00 mL) was added disodium sulfinatosulfonate (132 mg, 1.2 eq., 695 μmol) at room temperature. The resulting mixture was stirred for 16 h at 80° C. After completion, ice cold water was added in the reaction mixture and solid was dissolved in dichloromethane and concentrated under reduced pressure to get crude. The crude was purified by flash chromatography. The desired fractions were concentrated to afford tert-butyl N-{2-[3,4-bis(benzyloxy)-2-fluoro-5-methoxyphenyl]-1-(3-methyloxetan-3-yl)-1H-1,3-benzodiazol-5-yl}carbamate (190 mg, 275 μmol) as shiny yellow crystalline solid. Yield: 0.190 g, 47%

Step-5: To a stirred solution of tert-butyl N-{2-[3,4-bis(benzyloxy)-2-fluoro-5-methoxyphenyl]-1-(3-methyloxetan-3-yl)-1H-1,3-benzodiazol-5-yl}carbamate (50.0 mg, 78.2 μmol) in dichloromethane (50.0 μL) was added 4M HCl in 1,4-Dioxane (2.00 mL) and the reaction mixture was stirred at room temperature for 3 h. After completion of the reaction, the reaction mixture was concentrated under reduced pressure to get the desired product 2-[3,4-bis(benzyloxy)-2-fluoro-5-methoxyphenyl]-1-(3-methyloxetan-3-yl)-1H-1,3-benzodiazol-5-amine (60.0 mg, 74.1 μmol) as hydrochloride salt. Yield: 0.060 g, 94.7%

Step-6: To a stirred solution of 2-[3,4-bis(benzyloxy)-2-fluoro-5-methoxyphenyl]-1-(3-methyloxetan-3-yl)-1H-1,3-benzodiazol-5-amine hydrochloride (60.0 mg, 104 μmol) in N,N-dimethylformamide (600 μL) was added benzoic acid (14.0 mg, 1.1 eq., 114 μmol), hexafluoro-λ⁵-phosphanuide 1-[bis(dimethylamino)methylidene]-1H-1λ⁵-[1,2,3]triazolo[4,5-b]pyridin-3-ium-1-ylium-3-olate (59.3 mg, 1.5 eq., 156 μmol) and ethylbis(propan-2-yl)amine (54.4 μL, 3 eq., 312 μmol) and the solution was stirred at room temperature for 16 hours. After completion of reaction, the reaction mixture was diluted with water and extracted with ethyl acetate. The organic layer dried over anhydrous sodium sulphate, filtered and concentrated to obtain crude. The crude was purified by flash chromatography. Desired fractions were concentrated to get N-{2-[3,4-bis(benzyloxy)-2-fluoro-5-methoxyphenyl]-1-(3-methyloxetan-3-yl)-1H-1,3-benzodiazol-5-yl}benzamide (28.0 mg, 41.3 μmol) as pale yellow solid. Yield: 0.028 g, 39.7%

Step-7: To N-{2-[3,4-bis(benzyloxy)-2-fluoro-5-methoxyphenyl]-1-(3-methyloxetan-3-yl)-1H-1,3-benzodiazol-5-yl}benzamide (25.0 mg, 38.8 μmol) was added trifluoroacetic acid (1.00 mL) and the resulting solution was stirred for 2 h at 60° C. After completion, the reaction mixture was concentrated under reduced pressure to get N-[2-(2-fluoro-3,4-dihydroxy-5-methoxyphenyl)-1-(3-methyloxetan-3-yl)-1H-1,3-benzodiazol-5-yl]benzamide (22.0 mg, 33.2 μmol) as the desired product. Yield: 0.022 g, 85%

Step-8: To a stirred solution of N-[2-(2-fluoro-3,4-dihydroxy-5-methoxyphenyl)-1-(3-methyloxetan-3-yl)-1H-1,3-benzodiazol-5-yl]benzamide (75.0 mg, 162 μmol) in Tetrahydrofuran (1.00 mL), Borane dimethylsulfide (46.1 μL, 3 eq., 485 μmol) was added at 0° C. and stirred for 3 hr at 60° C. After 3 hr methanol was added to reaction mixture and stirred for 1 hr at RT. After completion, reaction mixture was evaporated under reduced pressured to give crude and further purified by reverse phase HPLC. Desired fractions were lyophilized to afford 4-[5-(benzylamino)-1-(3-methyloxetan-3-yl)-1H-1,3-benzodiazol-2-yl]-3-fluoro-6-methoxybenzene-1,2-diol (8.00 mg, 17.8 μmol) as white solid. Yield: 8 mg, 77.76%

1H NMR and LCMS: ES MS M/Z=443 (M+1), NMR (400 MHz, DMSO-d6) δ 9.41 (br s, 1H), 7.70 (d, 1H), 7.64 (d, 1H), 7.21 (d, 1H), 6.57 (d, 1H), 4.70 (d, 2H), 4.37 (d, 2H), 3.84 (t, 2H), 3.78 (s, 3H), 3.45 (t, 2H), 2.78 (s, 3H), 2.00 (s, 3H), 1.23 (s, 1H).

### Example 223: Synthesis of 4-(5-(ethylamino)-1-(3-methyloxetan-3-yl)-1H-benzo[d]imidazol-2-yl)-3-fluoro-6-methoxybenzene-1,2-diol

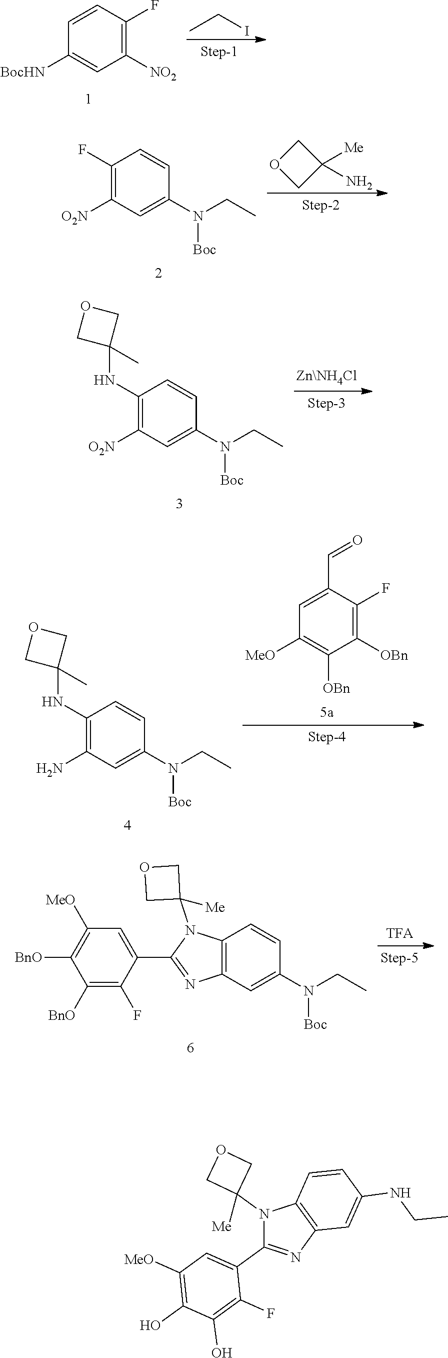

Step-1: To a stirred solution of tert-butyl N-(4-fluoro-3-nitrophenyl)carbamate (500 mg, 1.95 mmol) in N,N-dimethylformamide (7.00 mL) was added sodium hydride (93.7 mg, 1.2 eq., 2.34 mmol) at 0° C. and stirred for half an hour at room temperature. After half an hour, iodoethane (240 μL, 1.5 eq., 2.93 mmol) was added and the solution was stirred for 2 hours at room temperature. After completion, reaction mixture was quenched with water and extracted with ethyl acetate. The organic layer was dried over anhydrous sodium sulfate and concentrated under reduced pressure to get crude. The crude was purified by flash chromatography. The desired fractions concentrated to afford tert-butyl N-ethyl-N-(4-fluoro-3-nitrophenyl)carbamate (418 mg, 1.40 mmol) as yellow liquid. Yield: 0.418 g, 71.58%

Step-2: To a stirred solution of tert-butyl N-ethyl-N-(4-fluoro-3-nitrophenyl)carbamate (418 mg, 1.47 mmol) in 1-methylpyrrolidin-2-one (2.00 mL) was added 3-methyloxetan-3-amine (185 μL, 1.5 eq., 2.21 mmol) and ethylbis(propan-2-yl)amine (514 μL, 2 eq., 2.94 mmol) and heated at 100° C. for 16 h. After completion of reaction, reaction mixture diluted with water and extracted with ethyl acetate. The organic layer was dried over anhydrous sodium sulphate, filtered and concentrated to afford tert-butyl N-ethyl-N-{4-[(3-methyloxetan-3-yl)amino]-3-nitrophenyl}carbamate (483 mg, 1.22 mmol) as yellow solid. Yield: 0.483 g (83.2%)

Step-3: To a stirred solution of tert-butyl N-ethyl-N-{4-[(3-methyloxetan-3-yl)amino]-3-nitrophenyl}carbamate (300 mg, 854 μmol) in methanol (10.0 mL), zinc (279 mg, 5 eq., 4.27 mmol) and ammonium chloride (228 mg, 5 eq., 4.27 mmol) were added at room temperature and stirred at 50° C. for 1 h. After completion of the reaction, reaction mixture was passed through celite and washed with 10% methanol in dichloromethane, filtrate was washed with water, dried over anhydrous sodium sulfate and concentrated under reduced pressure to get the desired product. Yield: 0.256 g, (78.37%)

Step-4: To a stirred solution of tert-butyl N-{3-amino-4-[(3-methyloxetan-3-yl)amino]phenyl}-N-ethylcarbamate (250 mg, 778 μmol) and 3,4-bis(benzyloxy)-2-fluoro-5-methoxybenzaldehyde (285 mg, 778 μmol) in methanesulfinylmethane (5.00 mL) was added disodium sulfinatosulfonate (177 mg, 1.2 eq., 933 μmol) at room temperature. The resulting mixture stirred for 2 h at 85° C. After completion, reaction mixture cooled to room temperature and diluted with water and extracted with ethyl acetate. The organic layer was dried over anhydrous sodium sulfate and concentrated under reduced pressure to get crude. The crude purified by flash chromatography. The desired fractions concentrated to afford tert-butyl N-{2-[3,4-bis(benzyloxy)-2-fluoro-5-methoxyphenyl]-1-(3-methyloxetan-3-yl)-1H-1,3-benzodiazol-5-yl}-N-ethylcarbamate (240 mg, 349 μmol) as off white solid. Yield: 0.24 g, 44.82%

Step-5: To a stirred solution of tert-butyl N-{2-[3,4-bis(benzyloxy)-2-fluoro-5-methoxyphenyl]-1-(3-methyloxetan-3-yl)-1H-1,3-benzodiazol-5-yl}-N-ethylcarbamate (140 mg, 210 μmol) was added trifluoroacetic acid (2.00 mL) and the resulting mixture was stirred for 2 h at 50° C. After completion, the reaction mixture was concentrated under reduced pressure to get the crude and further purified by reverse phase HPLC. Desired fractions were lyophilized to afford 4-[5-(ethylamino)-1-(3-methyloxetan-3-yl)-1H-1,3-benzodiazol-2-yl]-3-fluoro-6-methoxybenzene-1,2-diol (53.0 mg, 136 μmol) as white solid. Yield: 0.053 g, 65%

1H NMR and LCMS: ES MS M/Z=388 (M+1), 1H NMR (400 MHz, DMSO-d6) δ 14.65 (s, 1H), 9.90 (br s, 2H), 7.37 (d, 1H), 7.02 (s, 1H), 6.84 (s, 1H), 6.73 (d, 1H), 4.73 (d, 2H), 4.44 (d, 2H), 3.81 (s, 3H), 3.16 (d, 2H), 2.11 (s, 3H), 1.21 (t, 3H).

### Example 224: Synthesis of N-(2-(2-fluoro-3,4-dihydroxy-5-methoxyphenyl)-1-(3-methyloxetan-3-yl)-1H-benzo[d]imidazol-5-yl)-3-hydroxypropanamide

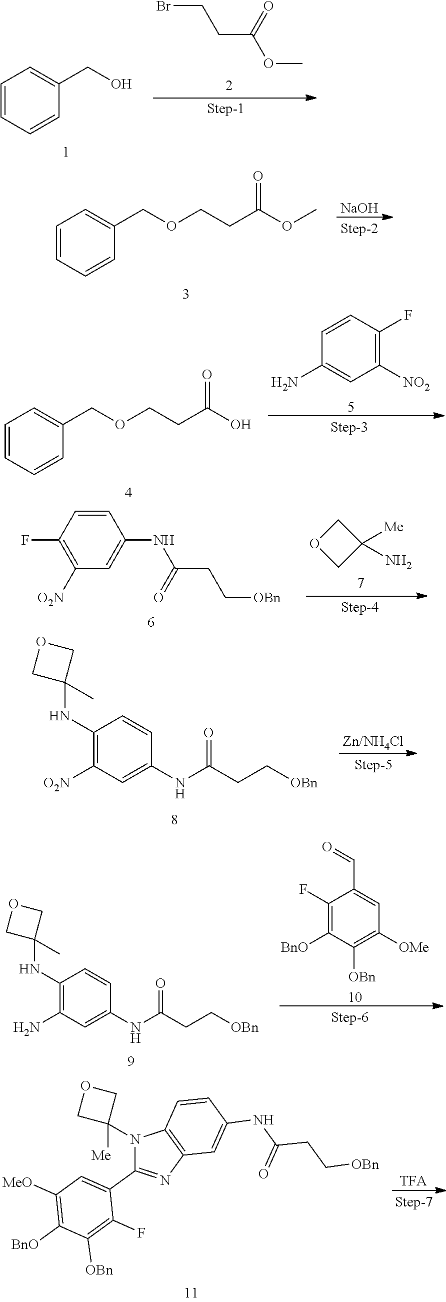

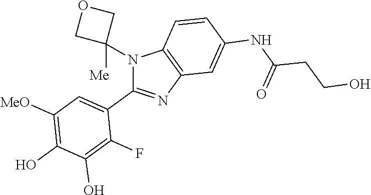 [SMILES: COc1cc(-c2nc3cc(NC(=O)CCO)ccc3n2C2(C)COC2)c(F)c(O)c1O]

Step-1: To a stirred solution of phenylmethanol (962 μL, 9.25 mmol) in oxolane (20.0 mL) was added sodium hydride (444 mg, 1.2 eq., 11.1 mmol) at 0° C. and stirred for 20 minutes at room temperature. methyl 3-bromopropanoate (1.11 mL, 1.1 eq., 10.2 mmol) was added at 0° C. and stirred for 16 h at room temperature. After completion, reaction mixture quenched with water and extracted with ethyl acetate. The organic layer was dried over anhydrous sodium sulfate, filtered and concentrated to get crude. as off white solid. Crude was purified by flash chromatography. Desired fractions were concentrated to get methyl 3-(benzyloxy)propanoate (500 mg, 1.18 mmol) as transparent oil. Yield: 0.50 g, 12.81%

Step-2: To a stirred solution of methyl 3-(benzyloxy)propanoate (500 mg, 2.57 mmol) in methanol (5.00 mL) and water (5.00 mL) was added sodium hydroxide (515 mg, 5 eq., 12.9 mmol) at 0° C. and stirred for 2 h at 50° C. After completion, reaction mixture organic layer was concentrated and crude was acidified with 6N hydrochloric acid and extracted with ethyl acetate. The organic layer was dried over anhydrous sodium sulfate, filtered and concentrated to get 3-(benzyloxy)propanoic acid (270 mg, 1.12 mmol) as transparent oil. Yield: 0.27 g, 43.65%

Step-3: To a stirred solution of 4-fluoro-3-nitroaniline (200 mg, 1.04 mmol) and 3-(benzyloxy)propanoic acid (238 mg, 1.04 mmol) in N,N-dimethylformamide (10.0 mL) was added hexafluoro-λ⁵-phosphanuide 1-[bis(dimethylamino)methylidene]-1H-1λ⁵-[1,2,3]triazolo[4,5-b]pyridin-3-ium-1-ylium-3-olate (594 mg, 1.5 eq., 1.56 mmol) and ethylbis(propan-2-yl)amine (544 μL, 3 eq., 3.13 mmol) and the solution was stirred for 6 hours at room temperature. After completion of reaction, the reaction mixture diluted with water and extracted with ethyl acetate. The organic layer dried over anhydrous sodium sulphate, filtered and concentrated to obtain crude. The crude was purified by flash chromatography. The desired fractions concentrated to afford 3-(benzyloxy)-N-(4-fluoro-3-nitrophenyl)propanamide (230 mg, 715 μmol) as yellow liquid. Yield: 0.23 g, 68.67%

Step-4: To a stirred solution of 3-(benzyloxy)-N-(4-fluoro-3-nitrophenyl)propanamide (230 mg, 723 μmol) and 3-methyloxetan-3-amine (64.9 μL, 2 eq., 1.45 mmol) in 1-methylpyrrolidin-2-one (5.00 mL) was added ethylbis(propan-2-yl)amine (378 μL, 3 eq., 2.17 mmol) and the solution was stirred for 6 hours at 110° C. After completion of reaction, the reaction mixture diluted with water and extracted with ethyl acetate. The organic layer dried over anhydrous sodium sulphate, filtered and concentrated to obtain 3-(benzyloxy)-N-{4-[(3-methyloxetan-3-yl)amino]-3-nitrophenyl}propanamide (240 mg, 455 μmol) crude. Yield: 0.24 g, 62.91%

Step-5: To a stirred solution of 3-(benzyloxy)-N-{4-[(3-methyloxetan-3-yl)amino]-3-nitrophenyl}propanamide (230 mg, 597 μmol) in methanol (5.00 mL) was added zinc (195 mg, 5 eq., 2.98 mmol) and ammonium chloride (160 mg, 5 eq., 2.98 mmol) were added at room temperature and stirred at 50° C. for 1 h. After completion of the reaction, reaction mixture was passed through celite and washed with dichloromethane, filtrate was washed with water, dried over anhydrous sodium sulfate and concentrated under reduced pressure to get N-{3-amino-4-[(3-methyloxetan-3-yl)amino]phenyl}-3-(benzyloxy)propanamide (200 mg, 501 μmol) (crude) as brown solid. Yield: 0.20 g, 83.92%

Step-6: To a stirred solution of N-{3-amino-4-[(3-methyloxetan-3-yl)amino]phenyl}-3-(benzyloxy)propanamide (190 mg, 476 μmol) and 3,4-bis(benzyloxy)-2-fluoro-5-methoxybenzaldehyde (139 mg, 0.8 eq., 381 μmol) in methanesulfinylmethane (5.00 mL) was added disodium sulfinatosulfonate (109 mg, 1.2 eq., 571 μmol) at room temperature. The resulting mixture stirred for 16 h at 85° C. After completion, reaction mixture cooled to room temperature and diluted with water and extracted with ethyl acetate. The organic layer was dried over anhydrous sodium sulfate and concentrated under reduced pressure to get crude. The crude purified by flash chromatography. The desired fractions concentrated to afford 3-(benzyloxy)-N-{2-[3,4-bis(benzyloxy)-2-fluoro-5-methoxyphenyl]-1-(3-methyloxetan-3-yl)-1H-1,3-benzodiazol-5-yl}propanamide (150 mg, 203 μmol) as off white solid. Yield: 0.15 g, 42.68%

Step-7: To a stirred solution of 3-(benzyloxy)-N-{2-[3,4-bis(benzyloxy)-2-fluoro-5-methoxyphenyl]-1-(3-methyloxetan-3-yl)-1H-1,3-benzodiazol-5-yl}propanamide (120 mg, 171 μmol) in Tetrahydrofuran (10.0 mL) was added 20% Palladium hydroxide (155 mg, 1.3 eq., 222 μmol) at room temperature and the reaction mixture stirred for 3 h under hydrogen atmosphere. After completion, the reaction mixture passed through celite bed. Filtrate was concentrated to get crude. Crude was purified by reverse phase HPLC and desired fractions were lyophilized to get N-[2-(2-fluoro-3,4-dihydroxy-5-methoxyphenyl)-1-(3-methyloxetan-3-yl)-1H-1,3-benzodiazol-5-yl]-3-hydroxypropanamide (30.0 mg, 68.8 μmol) as off white solid. Yield: 0.030 g, 40.26%

ES MS M/Z=432.08 (M+1), 1H NMR (400 MHz, DMSO-d6) δ 9.92 (s, 1H), 9.36 (brs, 1H), 8.05 (d, 1H), 7.42-7.40 (dd, 1H), 7.19 (d, 1H), 6.58 (d, 1H), 4.71 (d, 2H), 4.37 (d, 2H), 3.78 (s, 3H), 3.74 (t, 2H), 2.49 (s, 2H), 1.99 (s, 3H).

### Example 225: Synthesis of 6-methoxy-3-methyl-4-(1-(1-methylcyclobutyl)-1H-benzo[d]imidazol-2-yl)benzene-1,2-diol

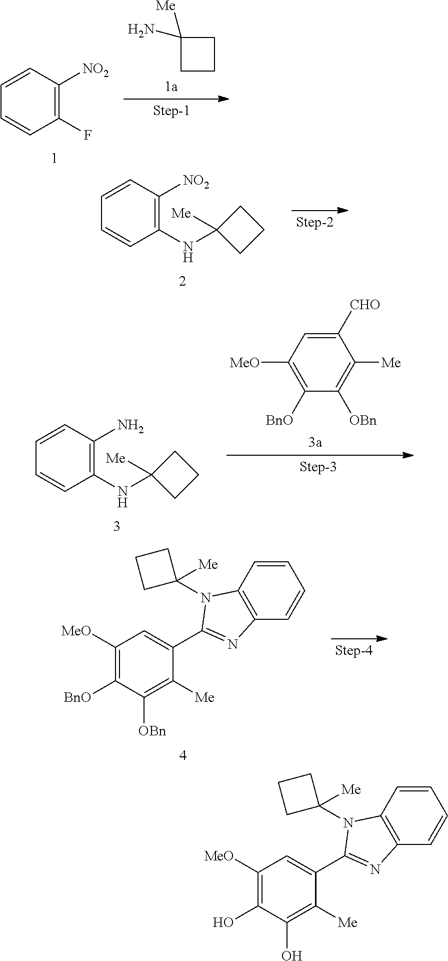

Step-1: To a stirred solution of 1-fluoro-2-nitrobenzene (800 mg, 5.67 mmol) in 1-methylpyrrolidin-2-one (4.00 mL) cyclobutanamine (484 mg, 1.2 eq., 6.80 mmol) and N-cyclobutyl-2-nitroaniline (800 mg, 3.07 mmol) were added and reaction mixture was heated to 90° C. for 16 h. After completion, the reaction mixture diluted with water and extracted with dichloromethane. The combined organic layers dried over anhydrous sodium sulphate, filtered and concentrated to obtain crude. The crude purified by flash chromatography. The desired fractions were concentrated to afford N-cyclobutyl-2-nitroaniline (800 mg, 3.07 mmol) as yellow oil. Yield: 800 mg, 54.12%

Step-2: To a stirred solution of N-cyclobutyl-2-nitroaniline (800 mg, 4.16 mmol) in methanol zinc (1.63 g, 6 eq., 25.0 mmol) and Ammonium chloride (1.34 g, 6 eq., 25.0 mmol) were added and reaction mixture was stirred at room temperature for 2 h. After completion, the reaction mixture was filtered through celite bed and solvent was removed under reduced pressure. The crude diluted with water and extracted with dichloromethane. The combined organic layers dried over anhydrous sodium sulphate, filtered and concentrated to obtain N1-cyclobutylbenzene-1,2-diamine (700 mg, 2.09 mmol) as yellow oil. Yield: 700 mg, 50.31%

Step-3: To a stirred solution N1-(1-methylcyclobutyl)benzene-1,2-diamine (200 mg, 1.13 mmol) and 3,4-bis(benzyloxy)-5-methoxy-2-methylbenzaldehyde (288 mg, 0.7 eq., 794 μmol) in Methanol (2.00 mL) was added Acetic acid (200 mL) at 80° C. for 6 h. After completion, reaction mixture concentrated under reduced pressure to get crude 2-[3,4-bis(benzyloxy)-5-methoxy-2-methylphenyl]-1-(1-methylcyclobutyl)-1H-1,3-benzodiazole (150 mg, 217 μmol) as brown mass. Yield: 150 mg, 19.12%

Step-4: To a stirred solution of 2-[3,4-bis(benzyloxy)-5-methoxy-2-methylphenyl]-1-(1-methylcyclobutyl)-1H-1,3-benzodiazole (150 mg, 289 μmol) in Tetrahydrofuran (5.00 mL), Palladium hydroxide (35.4 mg, 289 μmol) was charged and stirred under hydrogen atmosphere at room temperature for 2 h. After completion of reaction, reaction mixture filtered through celite. Filtrate was distilled below 30° C. to obtain 6-methoxy-3-methyl-4-[1-(1-methylcyclobutyl)-1H-1,3-benzodiazol-2-yl]benzene-1,2-diol (34.0 mg, 100 μmol) as pale green solid. Crude was submitted for Prep purification which afforded 6-methoxy-3-methyl-4-[1-(1-methylcyclobutyl)-1H-1,3-benzodiazol-2-yl]benzene-1,2-diol (34.0 mg, 100 μmol). Yield: 34 mg, 34.66%

ES MS M/Z=339.13 (M+1), UPLC: 99.78%. ¹HNMR (400 MHz, DMSO-d6): δ 8.86 (bs, 1H), 8.57 (bs, 1H), 7.62 (dd, J=5.2 Hz, 2 Hz, 1H), 7.44 (dd, J=7.2 Hz, 4.0 Hz, 1H), 7.20-7.18 (m, 2H), 6.42 (s, 1H), 3.74 (s, 3H), 2.44-2.41 (m, 2H), 2.11 (d, J=10.0 Hz, 1H), 1.93 (s, 3H), 1.89-1.82 (m, 2H), 1.77 (s, 3H), 1.70-1.63 (m, 1H), 1.56 (bs, 1H).

### Example 226: Synthesis of 4-(1-isopropyl-1H-benzo[d]imidazol-2-yl)-6-methoxy-3-methylbenzene-1,2-diol

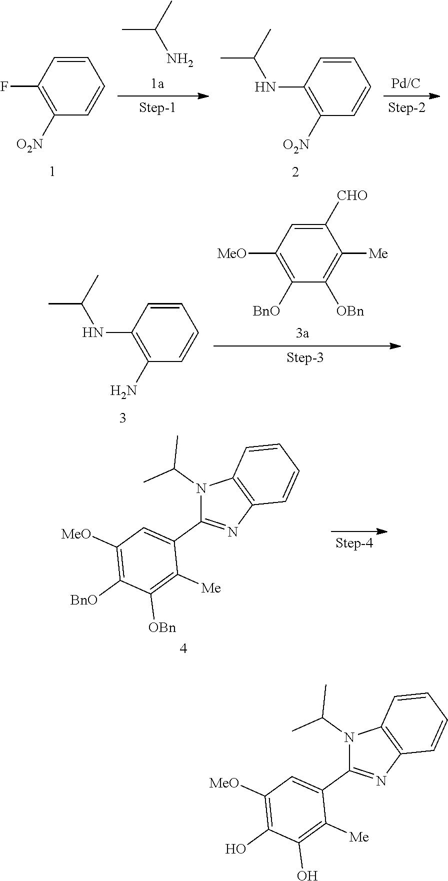

Step-1: To a stirred solution of 1-fluoro-2-nitrobenzene (500 mg, 3.54 mmol) in Isopropyl alcohol (5.00 mL) propan-2-amine (290 μL, 3.54 mmol) was added followed by addition of ethylbis(propan-2-yl)amine (1.86 mL, 3 eq., 10.6 mmol) and reaction mixture was heated to 90° C. for 16 h. After completion solvent was removed under reduced pressure to get the crude. The crude purified by flash chromatography. The desired fraction were concentrated to afford 2-nitro-N-(propan-2-yl)aniline (450 mg, 2.42 mmol) as yellow oil. Yield: 450 mg, 68.36%

Step-2: To a stirred solution of 2-nitro-N-(propan-2-yl)aniline (450 mg, 2.50 mmol) in methanol (10.0 mL), Palladium on carbon (133 mg, 0.5 eq., 1.25 mmol) was charged and stirred under hydrogen atmosphere at room temperature for 1 h. After completion of reaction, reaction mixture filtered through celite. Filtrate was distilled below 30° C. to obtain N1-(propan-2-yl)benzene-1,2-diamine (350 mg, 2.33 mmol) as black sticky mass. Yield: 200 mg, 93.3%

Step-3: To a stirred solution N1-(propan-2-yl)benzene-1,2-diamine (200 mg, 1.50 mmol) and 3,4-bis(benzyloxy)-5-methoxy-2-methylbenzaldehyde (434 mg, 0.8 eq., 1.20 mmol) in Methanol (5.00 mL) was added Acetic acid (200 μL) at room temperature. The resulting mixture was stirred at 85° C. for 6 h. After completion, reaction mixture concentrated under reduced pressure to get crude. The crude purified by flash chromatography. The desired fractions were concentrated to afford 2-[3,4-bis(benzyloxy)-5-methoxy-2-methylphenyl]-1-(propan-2-yl)-1H-1,3-benzodiazole (180 mg, 226 μmol) as yellow sticky mass. Yield: 180.0 mg, 15.1%

Step-4: To a stirred solution of 2-[3,4-bis(benzyloxy)-5-methoxy-2-methylphenyl]-1-(propan-2-yl)-1H-1,3-benzodiazole (150 mg, 304 μmol) in Tetrahydrofuran (5.00 mL), Palladium hydroxide (37.3 mg, 304 μmol) was charged and stirred under hydrogen atmosphere at room temperature for 2 h. After completion of reaction, reaction mixture filtered through celite. Filtrate was distilled below 30° C. to obtain crude and purification of compound was purified by Reverse Prep HPLC to get 6-methoxy-3-methyl-4-[1-(propan-2-yl)-1H-1,3-benzodiazol-2-yl]benzene-1,2-diol (34.0 mg, 109 μmol) as light pink solid. Yield: 34.0 mg, 35.71%

1 H NMR and LCMS: ES MS M/Z=313.14 (M+1), UPLC: 99.89%. ¹HNMR (400 MHz, DMSO-d6): δ 8.91(bs, 1H), 8.65 (bs, 1H), 7.76 (dd, J=6.0 Hz, 2.0 Hz, 1H), 7.63 (dd, J=6.4 Hz, 3.2 Hz, 1H), 7.23-7.18 (m, 2H), 6.45 (s, 1H), 4.35-4.28 (m, 1H), 3.75 (s, 3H), 1.86 (s, 3H), 1.50 (s, 3H), 1.49 (s, 3H).

### Example 227: Synthesis of 4-(1-(tert-butyl)-1H-benzo[d]imidazol-2-yl)-6-methoxy-3-methylbenzene-1,2-diol

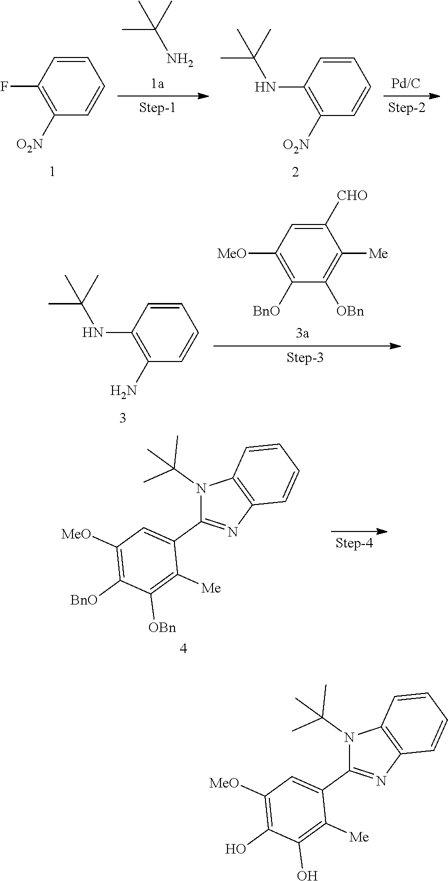

Step-1: To a stirred solution of 1-fluoro-2-nitrobenzene (500 mg, 3.54 mmol) in propan-2-ol, ethylbis(propan-2-yl)amine (458 mg, 3.54 mmol) was added followed by 2-methylpropan-2-amine (259 mg, 3.54 mmol) and reaction mixture was heated to 90° C. for 48 h. After completion, isopropyl alcohol was removed under reduced pressured and diluted with water and extracted with ethyl acetate. The combined organic layers dried over anhydrous sodium sulphate, filtered and concentrated to obtain crude. The crude purified by flash chromatography. The desired fractions were concentrated to afford N-tert-butyl-2-nitroaniline (500 mg, 2.57 mmol) as yellow oil. Yield: 500 mg, 72.64%

Step-2: To a stirred solution of N-tert-butyl-2-nitroaniline (500 mg, 2.57 mmol) in methanol (5.00 mL) Palladium on carbon (54.8 mg, 0.2 eq., 515 μmol) was charged and stirred under hydrogen atmosphere at room temperature for 2 h. After completion of reaction, reaction mixture filtered through celite. Filtrate was distilled below 30° C. to obtain N1-tert-butylbenzene-1,2-diamine (400 mg, 2.33 mmol) as brown mass. Yield: 450 mg, 90.35%

Step-3: To a stirred solution N1-tert-butylbenzene-1,2-diamine (250 mg, 1.52 mmol) and 3,4-bis(benzyloxy)-5-methoxy-2-methylbenzaldehyde (386 mg, 0.7 eq., 1.07 mmol) in Methanol (5.00 mL) was added Acetic acid (100 μL) at room temperature. The resulting mixture stirred for 6 h at 85° C. After completion, reaction mixture concentrated under reduced pressure to get crude. The crude purified by flash chromatography. The desired fractions were concentrated to afford 2-[3,4-bis(benzyloxy)-5-methoxy-2-methylphenyl]-1-tert-butyl-1H-1,3-benzodiazole (150 mg, 224 μmol) as yellow wax. Yield: 150 mg, 14.73%

Step-4: To a stirred solution of 2-[3,4-bis(benzyloxy)-5-methoxy-2-methylphenyl]-1-tert-butyl-1H-1,3-benzodiazole (160 mg, 316 μmol) in Tetrahydrofuran (5.00 mL), Palladium hydroxide (38.7 mg, 316 μmol) was charged and stirred under hydrogen atmosphere at room temperature for 2 h. After completion of reaction, reaction mixture filtered through celite. Filtrate was distilled below 30° C. to obtain 4-(1-tert-butyl-1H-1,3-benzodiazol-2-yl)-6-methoxy-3-methylbenzene-1,2-diol (32.0 mg, 97.4 μmol) as pale green solid. Yield: 32 mg, 30.85%

ES MS M/Z=327.11 (M+1), UPLC: 99.38%. ¹HNMR (400 MHz, DMSO-d6): δ 8.74(bs, 1H), 8.56 (bs, 1H), 7.85-7.82 (m, 1H), 7.59-7.57 (m, 1H), 7.23-7.17 (m, 2H), 6.44 (s, 1H), 3.73 (s, 3H), 1.78 (s, 3H), 1.53 (s, 9H).

### Example 228: Synthesis of 2-(2-fluoro-3,4-dihydroxy-5-methoxyphenyl)-N,N-dimethyl-1-(3-methyloxetan-3-yl)-1H-benzo[d]imidazole-6-sulfonamide

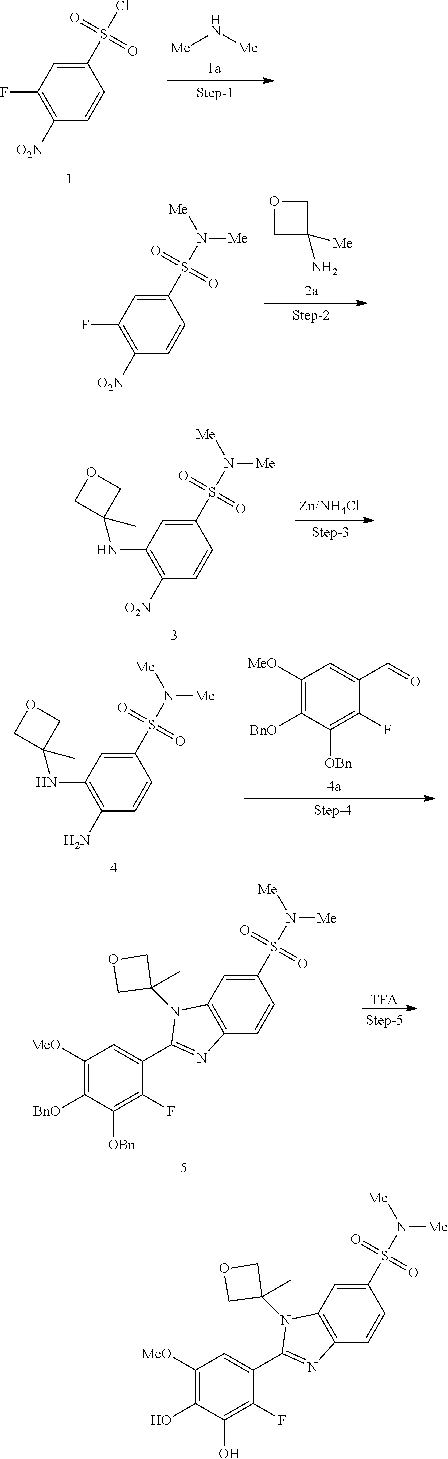

Step-1: To a stirred solution of Dimethylamine hydrochloride (67.6 mg, 835 μmol) in dichloromethane (10.0 mL) was added triethylamine (233 μL, 2 eq., 1.67 mmol) and stirred for 5 minutes. 3-fluoro-4-nitrobenzene-1-sulfonyl chloride (200 mg, 835 μmol) was added to the solution and was stirred for 1 hour at room temperature. After completion of reaction, the reaction mixture diluted with water and extracted with dichloromethane. The organic layer dried over anhydrous sodium sulphate, filtered and concentrated to obtain crude. The crude was purified by flash chromatography. The desired fractions concentrated to afford 3-fluoro-N,N-dimethyl-4-nitrobenzene-1-sulfonamide (160 mg, 625 μmol) as yellow solid. Yield: 0.16 g, 74.9%

Step-2: To a stirred solution of 3-fluoro-N,N-dimethyl-4-nitrobenzene-1-sulfonamide (160 mg, 645 μmol) and 3-methyloxetan-3-amine (112 mg, 2 eq., 1.29 mmol) in 1-methylpyrrolidin-2-one (5.00 mL) was added ethylbis(propan-2-yl)amine (337 μL, 3 eq., 1.93 mmol) and the solution was stirred for 16 hours at 110° C. After completion of reaction, the reaction mixture diluted with water and extracted with ethyl acetate. The organic layer dried over anhydrous sodium sulphate, filtered and concentrated to obtained N,N-dimethyl-3-[(3-methyloxetan-3-yl)amino]-4-nitrobenzene-1-sulfonamide (180 mg, 537 μmol) (crude) as brown solid. Yield: 0.180 g, 83.24%

Step-3: To a stirred solution of N,N-dimethyl-3-[(3-methyloxetan-3-yl)amino]-4-nitrobenzene-1-sulfonamide (180 mg, 571 μmol) in methanol (5.00 mL) was added zinc (187 mg, 5 eq., 2.85 mmol) and ammonium chloride (153 mg, 5 eq., 2.85 mmol) were added at room temperature and stirred at 50° C. for 1 h. After completion of the reaction, reaction mixture was passed through celite and washed with dichloromethane, filtrate was washed with water, dried over anhydrous sodium sulfate and concentrated under reduced pressure to get 4-amino-N,N-dimethyl-3-[(3-methyloxetan-3-yl)amino]benzene-1-sulfonamide (130 mg, 424 μmol) (crude) as brown solid. Yield: 0.13 g, 74.22%

Step-4: To a stirred solution of disodium sulfinatosulfonate (104 mg, 1.2 eq., 547 μmol) and 3,4-bis(benzyloxy)-2-fluoro-5-methoxybenzaldehyde (134 mg, 0.8 eq., 364 μmol) in methanesulfinylmethane (5.00 mL) was added disodium sulfinatosulfonate (104 mg, 1.2 eq., 547 μmol) at room temperature. The resulting mixture stirred for 16 h at 85° C. After completion, reaction mixture cooled to room temperature and diluted with water and extracted with ethyl acetate. The organic layer was dried over anhydrous sodium sulfate and concentrated under reduced pressure to get crude. The crude purified by flash chromatography. The desired fractions concentrated to afford 2-[3,4-bis(benzyloxy)-2-fluoro-5-methoxyphenyl]-N,N-dimethyl-1-(3-methyloxetan-3-yl)-1H-1,3-benzodiazole-6-sulfonamide (165 mg, 248 μmol) as brown oil. Yield: 0.165 g, 54.47%

Step-5: To a solution of 2-[3,4-bis(benzyloxy)-2-fluoro-5-methoxyphenyl]-N,N-dimethyl-1-(3-methyloxetan-3-yl)-1H-1,3-benzodiazole-6-sulfonamide (100 mg, 158 μmol) in trifluoroacetic acid (1.00 mL) was stirred for 3 h at 60° C. After completion, the reaction mixture was concentrated to get crude. Crude was purified by reverse phase HPLC and pure fractions were lyophilized to get 2-(2-fluoro-3,4-dihydroxy-5-methoxyphenyl)-N,N-dimethyl-1-(3-methyloxetan-3-yl)-1H-1,3-benzodiazole-6-sulfonamide (28.0 mg, 61.4 μmol) as off white solid. Yield: 0.028 g, 38.79%

ES MS M/Z=452.10 (M+1), 1H NMR (400 MHz, DMSO-d6) δ 9.58 (brs, 2H), 7.96 (d, 1H), 7.66-7.64 (dd, 1H), 7.49 (s, 1H), 6.65 (d, 1H), 4.7 (d, 2H), 4.45 (d, 2H), 3.79 (s, 3H), 2.61 (s, 6H), 2.07 (s, 3H).

### Example 229: Synthesis of 3-fluoro-6-methoxy-4-(1-(3-methyloxetan-3-yl)-1H-imidazo[4,5-c]pyridin-2-yl)benzene-1,2-diol

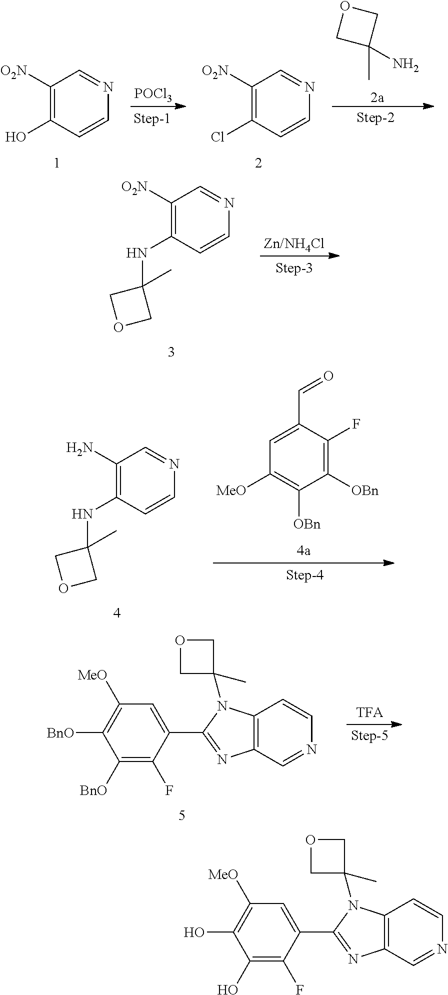

Step-1: To a stirred solution of 3-nitropyridin-4-ol (2.00 g, 14.3 mmol) in phosphoroyl trichloride (10.0 mL) was heated to 100° C. for 16 h. After completion of reaction concentrated completely and basified with 10% sodium hydroxide solution and extracted with ethyl acetate. The organic layer was dried over anhydrous sodium sulfate and filtered and concentrated to get 4-chloro-3-nitropyridine (1.50 g, 8.99 mmol) as brown solid. Yield: 1.50 g, 62.96%

Step-2: To a stirred solution of 4-chloro-3-nitropyridine (500 mg, 3.15 mmol) in N-Methyl-2-Pyrrolidone (NMP) (5.00 mL) were added 3-methyloxetan-3-amine (285 μL, 2 eq., 6.31 mmol) and N, N-Diisopropylethylamine (1.65 mL, 3 eq., 9.46 mmol) at room temperature and stirred for 16 h at 80° C. After completion, reaction mixture quenched with ice cold water and extracted with ethyl acetate and organic layer was dried over anhydrous sodium sulfate, filtered and concentrated to get N-(3-methyloxetan-3-yl)-3-nitropyridin-4-amine (200 mg, 841 μmol) crude. Yield: 0.20 g, 26.68%

Step-3: To a stirred solution of N-(3-methyloxetan-3-yl)-3-nitropyridin-4-amine (200 mg, 841 μmol) in methanol (5.00 mL), zinc (275 mg, 5 eq., 4.21 mmol) and ammonium chloride (225 mg, 5 eq., 4.21 mmol) were added at room temperature and stirred at 50° C. for 1 h. After completion of the reaction, reaction mixture was passed through celite and washed with 10% methanol in dichloromethane, filtrate was washed with water, dried over anhydrous sodium sulfate and concentrated under reduced pressure to get N4-(3-methyloxetan-3-yl)pyridine-3,4-diamine (120 mg, 402 μmol) (crude). Yield: 0.12 g, Crude

Step-4: To a stirred solution of N4-(3-methyloxetan-3-yl)pyridine-3,4-diamine (100 mg, 279 μmol) and 3,4-bis(benzyloxy)-2-fluoro-5-methoxybenzaldehyde (81.8 mg, 0.8 eq., 223 μmol) in toluene (1.00 mL) and N,N-dimethylformamide (1.00 mL) was added 4-methylbenzene-1-sulfonic acid (9.61 mg, 0.2 eq., 55.8 μmol) at room temperature. The resulting mixture stirred for 16 h at 100° C. After completion, reaction mixture cooled to room temperature and diluted with water and extracted with ethyl acetate. The organic layer was dried over anhydrous sodium sulfate and concentrated under reduced pressure to get crude. The crude purified by flash chromatography. The desired fractions concentrated to afford 2-[3,4-bis(benzyloxy)-2-fluoro-5-methoxyphenyl]-1-(3-methyloxetan-3-yl)-1H-imidazo[4,5-c]pyridine (40.0 mg, 57.1 μmol) as off white solid. Yield: 0.04 g, 20.46%

Step 5: To a solution of 2-[3,4-bis(benzyloxy)-2-fluoro-5-methoxyphenyl]-1-(3-methyloxetan-3-yl)-1H-imidazo[4,5-c]pyridine (40.0 mg, 76.1 μmol) in trifluoroacetic acid (1.00 mL) was stirred for 3 h at 60° C. After completion, the reaction mixture was concentrated to get crude. Crude was purified by reverse phase HPLC and pure fractions were lyophilized to get 3-fluoro-6-methoxy-4-[1-(3-methyloxetan-3-yl)-1H-imidazo[4,5-c]pyridin-2-yl]benzene-1,2-diol (6.00 mg, 17.2 μmol) as off white solid. Yield: 0.006 g, 22.6% ES MS M/Z=346.15 (M+1), 1H NMR (400 MHz, DMSO-d6) δ 9.56 (brs, 1H), 9.00 (s, 1H), 8.36 (d, 1H), 7.38 (d, 1H), 6.65 (d, 1H), 4.74 (d, 2H), 4.39 (d, 1H), 3.79 (s, 3H), 2.00 (s, 3H).

### Example 230: Synthesis of 3-fluoro-6-methoxy-4-[3(3-methyloxetan-3-yl)-3H-imidazo[4,5-c]pyridin-2-yl]benzene-1,2-diol

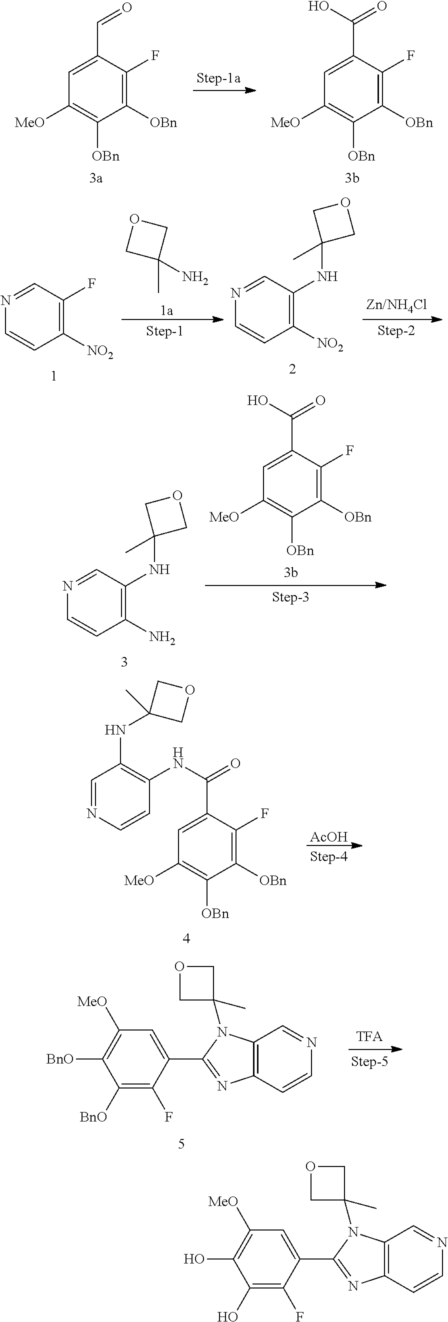

Step-1a: To a stirred solution of 3,4-bis(benzyloxy)-2-fluoro-5-methoxybenzaldehyde (200 mg, 546 μmol) in oxolane (4.00 mL) and water (1.00 mL) sulfamic acid (106 mg, 2 eq., 1.09 mmol) followed by addition of sodium chlorite (74.1 mg, 1.5 eq., 819 μmol) at 0° C. The resulting reaction mixture stirred at room temperature for 12 h. After the completion of reaction, water was added and extracted with ethyl acetate. The combined organic fractions were collected, dried over anhydrous sodium sulfate, filtered and concentrated to obtain crude. The crude purified by flash chromatography. The desired fractions were collected and concentrated to obtain 3,4-bis(benzyloxy)-2-fluoro-5-methoxybenzoic acid (110 mg, 288 μmol) as cream colored solid. Yield: 110 mg, 52.7%

Step-1: To a stirred solution of N, N-Diisopropylethylamine (369 μL, 3 eq., 2.11 mmol) in N-Methyl-2-Pyrrolidone (NMP) (5.00 mL), 3-methyloxetan-3-amine (63.3 μL, 2 eq., 1.41 mmol) was added at room temperature and stirred at 130° C. for 1 h. After completion, reaction mixture quenched with ice cold water, precipitate was formed, solid was filtered and dried to afford N-(3-methyloxetan-3-yl)-4-nitropyridin-3-amine (200 mg, 287 μmol) as a yellow oil. Yield: 0.20 g, 40.75%

Step-2: To a stirred solution of N-(3-methyloxetan-3-yl)-4-nitropyridin-3-amine (100 mg, 478 μmol) in methanol (5.00 mL), Zinc dust (155 mg, 5 eq., 2.39 mmol) and ammonium chloride (128 mg, 5 eq., 2.39 mmol) were added at room temperature and the reaction mixture stirred for 2 h at 30° C. After completion, the reaction mixture passed through celite bed. The filtrate was extracted with ethyl acetate, organic layer dried over anhydrous sodium sulphate, filtered and concentrated to get as N3-(3-methyloxetan-3-yl)pyridine-3,4-diamine (75.0 mg, 418 μmol) as a sticky liquid. Yield: 75 mg, 87.55%

Step-3: To a stirred solution of N3-(3-methyloxetan-3-yl)pyridine-3,4-diamine (46.9 mg, 262 μmol) in N,N-dimethylformamide (2.00 mL) was added 3,4-bis(benzyloxy)-2-fluoro-5-methoxybenzoic acid (100 mg, 262 μmol), hexafluoro-λ⁵-phosphanuide 1-[bis(dimethylamino)methylidene]-1H-1λ⁵-[1,2,3]triazolo[4,5-b]pyridin-3-ium-1-ylium-3-olate (149 mg, 1.5 eq., 392 μmol) and ethylbis(propan-2-yl)amine (137 μL, 3 eq., 785 μmol) and the solution was stirred for 16 hours. After completion of reaction, the reaction mixture diluted with water and extracted with ethyl acetate. The organic layer dried over anhydrous sodium sulphate, filtered and concentrated to obtain crude. The crude was purified by flash chromatography. The desired fractions concentrated to afford 3,4-bis(benzyloxy)-2-fluoro-5-methoxy-N-{3-[(3-methyloxetan-3-yl)amino]pyridin-4-yl}benzamide (80.0 mg, 147 μmol) as yellow liquid. Yield: 80 mg, 56.28%

Step-4: 3,4-bis(benzyloxy)-2-fluoro-5-methoxy-N-{3-[(3-methyloxetan-3-yl)amino]pyridin-4-yl}benzamide (70.0 mg, 129 μmol) taken in acetic acid (1.00 mL) at room temperature. The resulting mixture was stirred at 80° C. for 16 h. The progress of reaction monitored by LCMS and TLC analysis. After the completion of starting material, acetic acid was concentrated to obtain the crude. The crude washed with n-pentane and diethylether to obtain 2-[3,4-bis(benzyloxy)-2-fluoro-5-methoxyphenyl]-3-(3-methyloxetan-3-yl)-3H-imidazo[4,5-c]pyridine (65.0 mg, 124 μmol) as yellow liquid. Yield: 65 mg, 96.04%

Step-5: To 2-[3,4-bis(benzyloxy)-2-fluoro-5-methoxyphenyl]-3-(3-methyloxetan-3-yl)-3H-imidazo[4,5-c]pyridine (70.0 mg, 133 μmol), trifluoroacetic acid (2.00 mL) added at room temperature. The resulting reaction mixture stirred at 60° C. for 4 h. After the complete consumption of starting material, reaction mixture was concentrated to obtain the crude. The crude purified in reverse-phase HPLC and desired fraction were collected, lyophilized to obtain 3-fluoro-6-methoxy-4-[3-(3-methyloxetan-3-yl)-3H-imidazo[4,5-c]pyridin-2-yl]benzene-1,2-diol (17.0 mg, 49.2 μmol) as beige solid. Yield: 17 mg, 36.96%

LCMS and NMR Data: ES MS M/Z=345.92 (M+1); UPLC: 99.22% ¹H NMR (400 MHz, DMSO-d6) δ 9.52 (Bs, —OH peaks), 8.71 (s, 1H), 8.41 (d, J=5.2 Hz, 1H), 7.71 (d, J=5.2 Hz, 1H), 6.64 (d, J=6.0 Hz, 1H), 4.75 (d, J=6.0 Hz, 2H), 4.43 (d, J=6.0 Hz, 2H), 3.79 (s, 3H), 2.06 (s, 3H),

### Example 231: Synthesis of 3-fluoro-4-(5-fluoro-1H-benzo[d]imidazol-2-yl)-6-methoxybenzene-1,2-diol

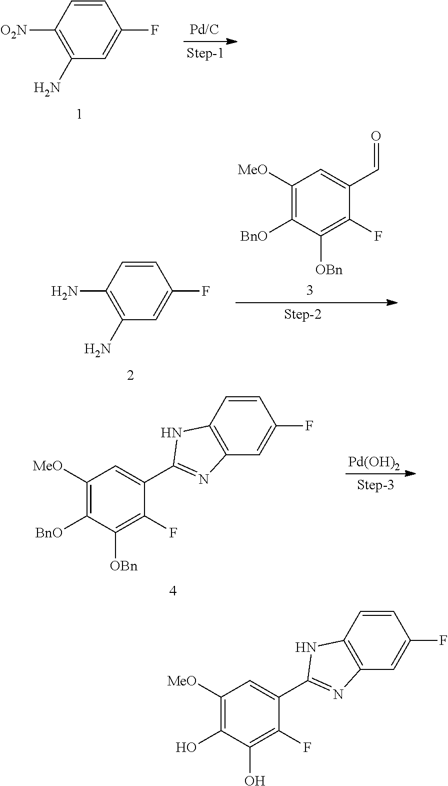

Step-1: To a solution of 5-fluoro-2-nitroaniline (300 mg, 1.92 mmol) in methanol (10.0 mL) was added 10% Palladium on Carbon (50% wet) (300 mg, 2.82 mmol) at room temperature and the reaction mixture stirred for 2 h under hydrogen atmosphere. After completion, the reaction mixture passed through celite bed. The filtrate was concentrated to get 4-fluorobenzene-1,2-diamine (200 mg, 1.11 mmol) as green solid. Yield: 0.20 g, 58%

Step-2: To a stirred solution 4-fluorobenzene-1,2-diamine (130 mg, 1.03 mmol) and 3,4-bis(benzyloxy)-2-fluoro-5-methoxybenzaldehyde (302 mg, 0.8 eq., 825 μmol) in methanol (10.0 mL) was added acetic acid (5.89 μL, 0.1 eq., 103 μmol) at room temperature. The resulting mixture stirred for 16 h at room temperature. After completion, reaction mixture concentrated under reduced pressure to get the crude. Crude was purified by flash chromatography. Desired fractions were concentrated to get 2-[3,4-bis(benzyloxy)-2-fluoro-5-methoxyphenyl]-5-fluoro-1H-1,3-benzodiazole (145 mg, 301 μmol) as off white semi solid. Yield: 0.145 g, 29.18%

Step-3: To a solution of 2-[3,4-bis(benzyloxy)-2-fluoro-5-methoxyphenyl]-5-fluoro-1H-1,3-benzodiazole (120 mg, 254 μmol) in tetrahydrofuran (5.00 mL) was added 20% Palladium hydroxide (600 mg, 3.4 eq., 857 μmol) at room temperature and the reaction mixture stirred for 3 h under hydrogen atmosphere. After completion, the reaction mixture passed through celite bed. Filtrate was concentrated to get crude. Crude was purified by reverse phase HPLC and desired fractions were lyophilized to get 3-fluoro-4-(5-fluoro-1H-1,3-benzodiazol-2-yl)-6-methoxybenzene-1,2-diol (38.0 mg, 129 μmol) as white solid. Yield: 0.038 g, 50.69%

ES MS M/Z=293.04 (M+1)+, ¹H NMR (400 MHz, DMSO-d₆) δ 9.83-9.71 (m, 2H), 7.70-7.67 (m, 1H), 7.50 (dd, J=2.4, 9.2 Hz, 1H), 7.23-7.21 (m, 2H), 3.87 (s, 3H).

### Example 232: Synthesis of 3-fluoro-6-methoxy-4-(1-(3-methyloxetan-3-yl)-1H-benzo[d]imidazol-2-yl)benzene-1,2-diol

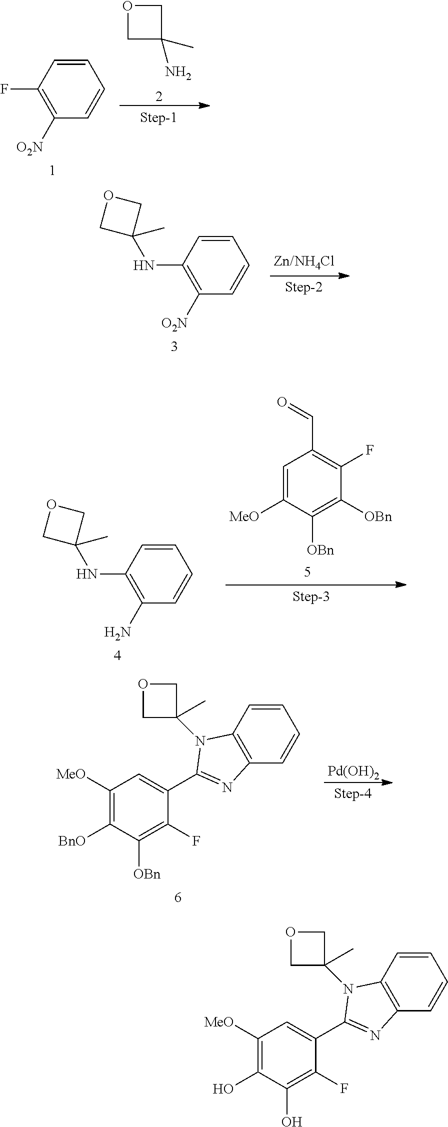

Step-1: To a stirred solution of 1-fluoro-2-nitrobenzene (4.00 g, 28.3 mmol) in 1-methylpyrrolidin-2-one (20.0 mL), 3-methyloxetan-3-amine (3.70 g, 1.5 eq., 42.5 mmol) and ethylbis(propan-2-yl)amine (13.9 mL, 3 eq., 85.0 mmol) were added at room temperature and stirred at 100° C. for 16 h. After completion, reaction mixture quenched with ice cold water and obtained precipitate was filtered and dried to afford 3-methyl-N-(2-nitrophenyl)oxetan-3-amine (4.50 g, 20.3 mmol) as a yellow solid. Yield: 4.50 g, 71.66%

Step-2: To a stirred solution of 3-methyl-N-(2-nitrophenyl)oxetan-3-amine (6.50 g, 29.3 mmol) in methanol (50.0 mL) was added zinc (9.59 g, 5 eq., 147 mmol) and ammonium chloride (7.85 g, 5 eq., 147 mmol) were added at room temperature and stirred at 50° C. for 1 h. After completion of the reaction, reaction mixture was passed through celite and washed with 10% methanol in dichloromethane, filtrate was washed with water, dried over anhydrous sodium sulfate and concentrated under reduced pressure to get N1-(3-methyloxetan-3-yl)benzene-1,2-diamine (4.00 g, 20.9 mmol) (crude) as brown solid. Yield: 4.00 g, 92.4%

Step-3: To a stirred solution of N1-(3-methyloxetan-3-yl)benzene-1,2-diamine (150 mg, 842 μmol) and 3,4-bis(benzyloxy)-2-fluoro-5-methoxybenzaldehyde (308 mg, 842 μmol) in methanesulfinylmethane (5.00 mL), disodium sulfinatosulfonate (208 mg, 1.3 eq., 1.09 mmol) was added at room temperature. The resulting mixture stirred for 12 h at 85° C. After completion, ice cold water was added in the reaction mixture and solid was filtered and dried to get crude. The crude purified by flash chromatography. The desired fractions were concentrated to afford 2-[3,4-bis(benzyloxy)-2-fluoro-5-methoxyphenyl]-1-(3-methyloxetan-3-yl)-1H-1,3-benzodiazole (190 mg, 359 μmol) as sticky solid. Yield: 0.19 g, 42%

Step-4: To a solution of 2-[3,4-bis(benzyloxy)-2-fluoro-5-methoxyphenyl]-1-(3-methyloxetan-3-yl)-1H-1,3-benzodiazole (180 mg, 343 μmol) in oxolane (10.0 mL) was added 20% Palladium hydroxide (300 mg) at room temperature and the reaction mixture stirred for 3 h under hydrogen atmosphere. After completion, the reaction mixture passed through celite bed. Filtrate was concentrated to get crude. Crude was purified by reverse phase HPLC and desired fractions were lyophilized to get 3-fluoro-6-methoxy-4-[1-(3-methyloxetan-3-yl)-1H-1,3-benzodiazol-2-yl]benzene-1,2-diol (54.0 mg, 154 μmol) as white solid.

ES MS M/Z=345.1 (M+1), 1H NMR (400 MHz, DMSO-d6) δ 9.47 (brs, 2H), 7.70-7.68 (m, 1H), 7.28-7.26 (m, 3H), 6.60 (d, J=6.4 Hz, 1H), 4.73 (d, J=6.0 Hz, 2H), 4.39 (d, J=6.0 Hz, 2H), 3.78 (s, 3H), 2.01 (s, 3H).

### Example 233: Synthesis of 5-(1-cyclobutyl-1H-benzo[d]imidazol-2-yl)-3-(trifluoromethoxy)benzene-1,2-diol

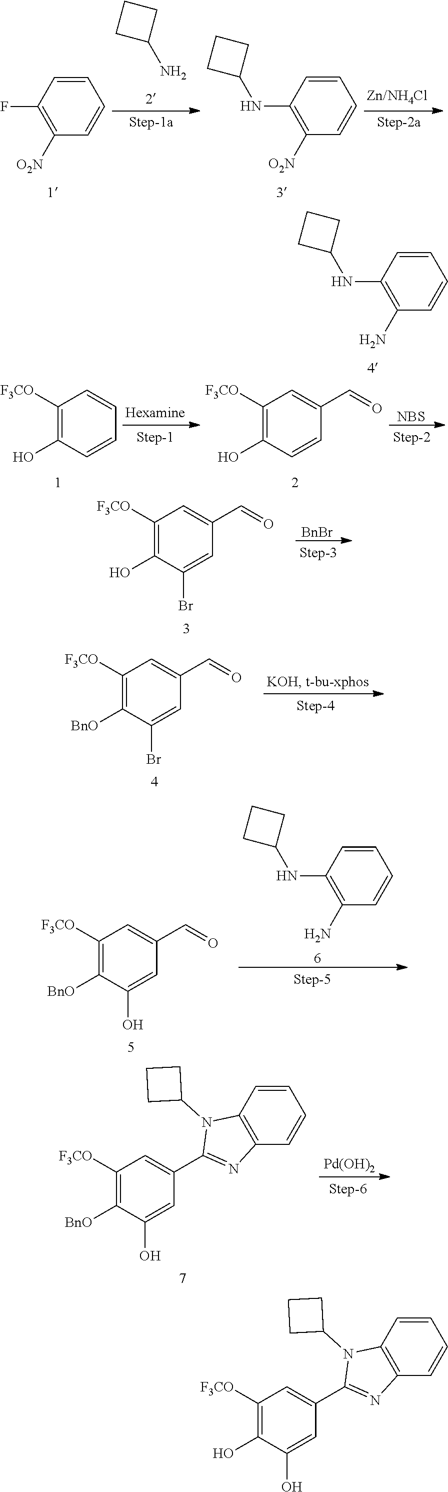

Step-1a: To a stirred solution of 1-fluoro-2-nitrobenzene (5.00 g, 35.4 mmol) in 1-methylpyrrolidin-2-one (30.0 mL) cyclobutanamine (3.02 g, 1.2 eq., 42.5 mmol) was added and reaction mixture was heated to 90° C. for 16 h. After completion, the reaction mixture diluted with water and extracted with dichloromethane. The combined organic layers dried over anhydrous sodium sulphate, filtered and concentrated to obtain crude. The crude purified by flash chromatography. The desired fractions were concentrated to afford N-cyclobutyl-2-nitroaniline (2.00 g, 10.4 mmol) as yellow oil. Yield: 2.00 g, 29.36%

Step-2a: To a stirred solution of N-cyclobutyl-2-nitroaniline (500 mg, 2.60 mmol) in methanol (10.0 mL), zinc (1.02 g, 6 eq., 15.6 mmol) and Ammonium chloride (835 mg, 6 eq., 15.6 mmol) were added and reaction mixture was stirred at room temperature for 4 h. After completion, the reaction mixture was filtered through celite bed and solvent was removed under reduced pressure. The crude diluted with water and extracted with dichloromethane. The combined organic layers dried over anhydrous sodium sulphate, filtered and concentrated to obtain N1-cyclobutylbenzene-1,2-diamine (400 mg, 2.47 mmol) as yellow oil. Yield: 400 mg, Crude

Step-1: To a stirred solution 2-(trifluoromethoxy)phenol (8.00 g, 44.9 mmol) in trifluoroacetic acid (60.0 mL) was added 1,3,5,7-tetraazatricyclo[3.3.1.1³,⁷]decane (12.6 g, 2 eq., 89.8 mmol) and stirred for 16 h at 70° C. After completion, reaction mixture diluted with water and extracted with ethyl acetate. Organic layer dried over anhydrous sodium sulfate, filtered and concentrated under reduced pressure to get the crude. The crude purified by flash chromatography. The desired fractions concentrated to afford 4-hydroxy-3-(trifluoromethoxy) benzaldehyde (2.30 g, 10.6 mmol) as off white solid. Yield: 2.30 g, 23.6%

Step-2: To a stirred solution 4-hydroxy-3-(trifluoromethoxy)benzaldehyde (700 mg, 3.40 mmol) in dichloromethane (10.0 mL) was added 1-bromopyrrolidine-2,5-dione (635 mg, 1.1 eq., 3.57 mmol) at 0° C. and stirred for 2 h at room temperature. After completion, reaction mixture diluted with water and extracted with dichloromethane. Organic layer dried over anhydrous sodium sulphate, filtered and concentrated under reduced pressure to get the crude. The crude was purified by flash chromatography and pure fractions were concentrated to get 3-bromo-4-hydroxy-5-(trifluoromethoxy)benzaldehyde (800 mg, 1.68 mmol) as off white solid. Yield: 0.80 g, 49.59%

Step-3: To a stirred solution 3-bromo-4-hydroxy-5-(trifluoromethoxy)benzaldehyde (800 mg, 2.81 mmol) in N,N-dimethylformamide (10.0 mL) was added dipotassium carbonate (970 mg, 2.5 eq., 7.02 mmol) followed by (bromomethyl)benzene (500 μL, 1.5 eq., 4.21 mmol) and stirred for 16 h at room temperature. After completion, reaction mixture diluted with water and extracted with ethyl acetate. The organic layer dried over anhydrous sodium sulphate, filtered and concentrated under reduced pressure to get the crude. Crude was purified by flash chromatography and pure fractions were concentrated to get 4-(benzyloxy)-3-bromo-5-(trifluoromethoxy)benzaldehyde (800 mg, 1.86 mmol) as light brown oil. Yield: 0.80 g, 66.1%

Step-4: To a solution of 4-(benzyloxy)-3-bromo-5-(trifluoromethoxy)benzaldehyde (750 mg, 2.00 mmol) in 1,4-dioxane (10.0 mL) and water (2.50 mL) was added potassium hydroxide (337 mg, 3 eq., 6.00 mmol) was added at room temperature and the reaction mixture was degassed with argon for 5 min. tris(1,5-diphenylpenta-1,4-dien-3-one) dipalladium (91.5 mg, 0.05 eq., 100 μmol) and di-tert-butyl[2′,4′,6′-tris(propan-2-yl)-[1,1′-biphenyl]-2-yl]phosphane (84.9 mg, 0.1 eq., 200 μmol) was added to the reaction, continued degassing for 5 min and heated the reaction mixture at 100° C. for 16 h. After completion, the reaction was cooled to room temperature and diluted with 6 N hydrochloric acid and extracted with ethyl acetate. The organic layer was dried over anhydrous sodium sulphate, filtered and concentrated to give crude. The crude was purified by flash chromatography. The desired fractions were concentrated to get 4-(benzyloxy)-3-hydroxy-5-(trifluoromethoxy)benzaldehyde (300 mg, 845 μmol) as off white solid. Yield: 0.30 g, 42%

Step-5: To a stirred solution of 4-(benzyloxy)-3-hydroxy-5-(trifluoromethoxy)benzaldehyde (154 mg, 0.8 eq., 493 μmol) and 2-(benzyloxy)-5-(1-cyclobutyl-1H-1,3-benzodiazol-2-yl)-3-(trifluoromethoxy)phenol (150 mg, 297 μmol) in Dimethyl sulfoxide (10.0 mL) was added disodium sulfinatosulfonate (141 mg, 1.2 eq., 740 μmol) at room temperature. The resulting mixture stirred for 2 h at 85° C. After completion, reaction mixture cooled to room temperature and diluted with water and extracted with ethyl acetate. The organic layer was dried over anhydrous sodium sulphate and concentrated under reduced pressure to get crude. The crude purified by flash chromatography. The desired fractions concentrated to afford 2-(benzyloxy)-5-(1-cyclobutyl-1H-1,3-benzodiazol-2-yl)-3-(trifluoromethoxy)phenol (150 mg, 297 μmol) as off white solid. Yield: 0.150 g, 48.19%

Step-6: To a solution of 2-(benzyloxy)-5-(1-cyclobutyl-1H-1,3-benzodiazol-2-yl)-3-(trifluoromethoxy)phenol (150 mg, 330 μmol) in Tetrahydrofuran (10.0 mL) was added 20% Palladium hydroxide (200 mg, 0.87 eq., 286 μmol) at room temperature and the reaction mixture stirred for 3 h under hydrogen atmosphere. After completion, the reaction mixture passed through celite bed. The filtrate was concentrated to get crude. The crude was purified by reverse phase HPLC and desired fractions were lyophilized to get 5-(1-cyclobutyl-1H-1,3-benzodiazol-2-yl)-3-(trifluoromethoxy)benzene-1,2-diol (51.0 mg, 139 μmol) as white solid. Yield: 0.051 g, 41.99%

ES MS M/Z=365.05 (M+1), 1H NMR (400 MHz, DMSO-d6) δ 7.83-7.81 (m, 1H), 7.66-7.64 (m, 1H), 7.28-7.21 (m, 2H), 7.08 (d, 2H), 7.02 (s, 1H), 5.14-5.05 (m, 1H), 2.75-2.65 (m, 1H), 2.42-2.32 (m, 2H), 1.92-1.87 (m, 1H), 1.84-1.77 (m, 1H).

### Example 234: Synthesis of 5-(1-cyclobutyl-1H-1,3-benzodiazol-2-yl)-3-ethoxybenzene-1,2-diol

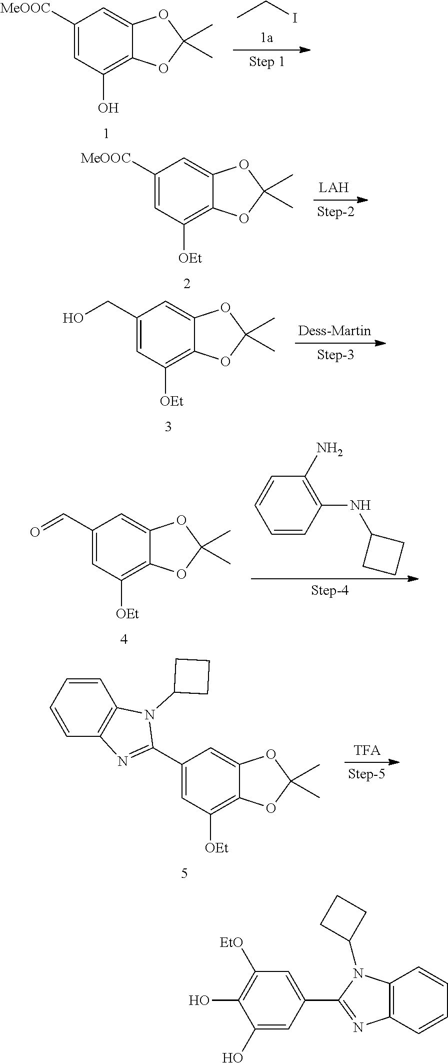

Step-1: To a solution of methyl 7-hydroxy-2,2-dimethyl-2H-1,3-benzodioxole-5-carboxylate (100 mg, 446 μmol) in N,N-Dimethylformamide (10.0 mL), methyl 7-ethoxy-2,2-dimethyl-2H-1,3-benzodioxole-5-carboxylate (100 mg, 309 μmol), dipotassium carbonate (123 mg, 2 eq., 892 μmol) were added to the reaction mixture and stirred at room temperature for 12. After completion of the reaction, the reaction mixture was quenched with ice cold water to get the precipitate which were filtered to get the crude. The crude purified by flash chromatography. The desired fractions were concentrated to afford methyl 7-ethoxy-2,2-dimethyl-2H-1,3-benzodioxole-5-carboxylate as off white solid. Yield: 100 mg. 69.33%

Step-2: To a solution of methyl 7-hydroxy-2,2-dimethyl-2H-1,3-benzodioxole-5-carboxylate (1.00 g, 4.46 mmol) in dry oxolane (7.00 mL), lithium aluminum hydride (4.16 mL, 1.5 eq., 4.16 mmol) added at 0° C. under inert atmosphere. The resulting reaction mixture stirred at room temperate. After completion of the reaction, the reaction mixture was quenched with aqueous ammonium chloride to get the precipitate which were filtered, and washed with ethyl acetate. The organic fractions were collected, dried over anhydrous sodium sulphate, filtered and concentrated to obtain (7-ethoxy-2,2-dimethyl-2H-1,3-benzodioxol-5-yl)methanol (460 mg, 2.05 mmol) as sticky brown liquid. Yield: 460 mg, 73.92%

Step-3: To a solution of (7-ethoxy-2,2-dimethyl-2H-1,3-benzodioxol-5-yl)methanol (460 mg, 2.05 mmol) in 1,2-dichloromethane (10.0 mL), Dess Martin (1.28 g, 1.5 eq., 3.01 mmol) added at 0° C. The resulting reaction mixture stirred at room temperature. After completion of the reaction, the reaction mixture was quenched with saturated solution of sodium thiosulfate and sodium bicarbonate, extracted with ethyl acetate. The combined organic fractions collected, dried over anhydrous sodium sulphate, filtered and concentrated to get the crude. The crude purified by flash chromatography. The desired fractions were concentrated to afford 7-ethoxy-2,2-dimethyl-2H-1,3-benzodioxole-5-carbaldehyde (550 mg, 1.09 mmol) as off white solid. Yield: 550 mg, 54.27%

Step-4: To a stirred solution of N1-cyclobutylbenzene-1,2-diamine (219 mg, 1.35 mmol) and 7-ethoxy-2,2-dimethyl-2H-1,3-benzodioxole-5-carbaldehyde (300 mg, 1.35 mmol) in methanesulfinylmethane (10.0 mL), disodium sulfinatosulfonate (385 mg, 1.5 eq., 2.02 mmol) was added at room temperature. The resulting mixture stirred for 16 h at 85° C. After completion, ice cold water was added in the reaction mixture and solid was filtered and dried to get crude. The crude purified by flash chromatography. The desired fractions were concentrated to afford 1-cyclobutyl-2-(7-ethoxy-2,2-dimethyl-2H-1,3-benzodioxol-5-yl)-1H-1,3-benzodiazole (185 mg, 340 μmol) as sticky solid. Yield: 185 mg, 25.2%

Step-5: A mixture of 1-cyclobutyl-2-(7-ethoxy-2,2-dimethyl-2H-1,3-benzodioxol-5-yl)-1H-1,3-benzodiazole (180 mg, 494 μmol) in trifluoroacetic acid (2.00 mL) heated at 100° C. for 4 h. After the completion of reaction, reaction mixture was concentrated to obtain crude. The crude purified in reverse phase HPLC chromatography. The desired fractions were collected and lyophilized to obtain 5-(1-cyclobutyl-1H-1,3-benzodiazol-2-yl)-3-ethoxybenzene-1,2-diol (45.0 mg, 139 μmol) as off white solid. Yield: 45 mg, 28.09%

LCMS and NMR Data: ES MS M/Z=325.08 [M+1]+: UPLC: 99.47%; 1H NMR (400 MHz, DMSO-d6) δ 9.32 (d, J=7.6 Hz, 1H), 8.61 (d, J=3.6 Hz, 1H), 7.83 (d, J=7.1 Hz, 1H), 7.62 (t, J=7.0 Hz, 1H), 7.26-7.19 (m, 2H), 6.69 (s, 2H), 5.13 (quin, J=8.8 Hz, 1H), 4.11 (q, J=13.6 Hz, 2H), 2.77-2.71 (m, 2H), 2.44-2.37 (m, 2H), 1.96-1.76 (m, 2H), 1.35 (t, J=13.8 Hz, 3H).

### Example 235: Synthesis of 4-(5-amino-1-cyclobutyl-1H-benzo[d]imidazol-2-yl)-6-methoxy-3-methylbenzene-1,2-diol

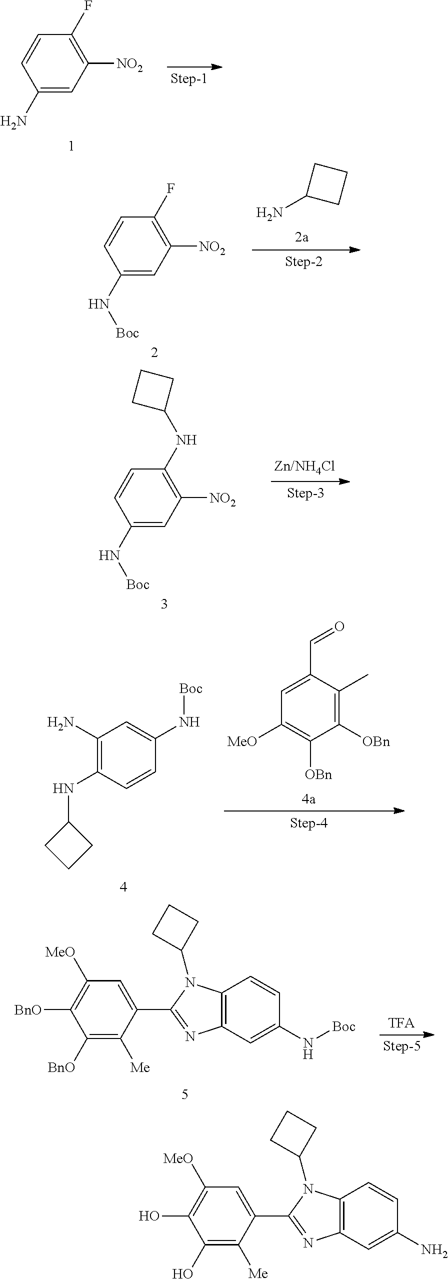

Step-1: A stirred solution of 4-fluoro-3-nitroaniline (1.00 g, 6.41 mmol) in dichloromethane (10.0 mL), di-tert-butyl dicarbonate (1.68 g, 1.2 eq., 7.69 mmol) and diethyl(propan-2-yl)amine (1.11 g, 1.5 eq., 9.61 mmol) were added and stirred at room temperature for 3 h. After completion of reaction, reaction mixture diluted with water and extracted with dichloromethane. The combined organic layers were dried over anhydrous sodium sulphate, filtered and concentrated to obtained crude. The crude purified in flash chromatography. The desired fractions were concentrated to afford tert-butyl (4-fluoro-3-nitrophenyl)carbamate as off-white solid. Yield: 1.50 g, 91%

Step-2: A stirred solution of tert-butyl N-(4-fluoro-3-nitrophenyl)carbamate (1.50 g, 5.85 mmol) and cyclobutanamine (625 mg, 1.5 eq., 8.78 mmol) in 1-methylpyrrolidin-2-one (3.00 mL), diethyl(propan-2-yl)amine (2.73 mL, 3 eq., 17.6 mmol) was added and heated at 100° C. for 8 h. After completion of reaction, reaction mixture poured on crushed ice and obtained solid was filtered and dried to afford tert-butyl (4-(cyclobutylamino)-3-nitrophenyl)carbamate as orange solid. Yield: 1.50 g, 72%

Step-3: To a stirred solution of tert-butyl N-[4-(cyclobutylamino)-3-nitrophenyl]carbamate (500 mg, 1.63 mmol) in methanol (5.00 mL), zinc (532 mg, 5 eq., 8.13 mmol) and ammonium chloride (435 mg, 5 eq., 8.13 mmol) were added and reaction mixture was heated to 90° C. for 16 h. After completion, the reaction mixture filtered through celite bed was concentrated. Then diluted with water and extracted with dichloromethane. The combined organic layers dried over anhydrous sodium sulphate, filtered and concentrated to obtain tert-butyl N-[3-amino-4-(cyclobutylamino)phenyl]carbamate (300 mg, 898 μmol) as yellow oil. Yield: 300 mg, 55.18%

Step-4: To a stirred solution tert-butyl N-[3-amino-4-(cyclobutylamino)phenyl]carbamate (276 mg, 1.2 eq., 993 μmol) and 3,4-bis(benzyloxy)-5-methoxy-2-methylbenzaldehyde (300 mg, 828 μmol) in Methanol (3.00 mL) was added Acetic acid (100 μL) (catalytic amount) at room temperature for 16 h. After completion, reaction mixture concentrated under reduced pressure to get crude. The crude was purified in flash chromatography to afford tert-butyl N-{2-[3,4-bis(benzyloxy)-5-methoxy-2-methylphenyl]-1-cyclobutyl-1H-1,3-benzodiazol-5-yl}carbamate (200 mg, 242 μmol) as light yellow solid. Yield:200 mg, 29%.

Step-5: To a solution of tert-butyl N-{2-[3,4-bis(benzyloxy)-5-methoxy-2-methylphenyl]-1-cyclobutyl-1H-1,3-benzodiazol-5-yl}carbamate (180 mg, 290 μmol) in trifluoroacetic acid (2.00 mL) was reflux at 60° C. for 3 h After completion of reaction, the reaction mixture concentrated to obtained crude. The crude was purified by prep HPLC to afford 4-(5-amino-1-cyclobutyl-1H-1,3-benzodiazol-2-yl)-6-methoxy-3-methylbenzene-1,2-diol (19.0 mg, 52.2 μmol) as pink solid.

Yield: 19 mg, 17.97%

LCMS and NMR Data: LCMS and NMR Data: ES MS M/Z 340.14 (M+1), 1H NMR (400 MHz, DMSO-d6) δ 7.49 (d, J=8.8 Hz, 1H), 7.78 (d, J=1.6 Hz, 1H), 6.60 (dd, J=2, 8.8 Hz, 1H), 6.38 (s, 1H), 4.75 (bs, 1H), 4.57 (t, J=8.8 Hz, 1H), 3.76 (d, J=14 Hz, 3H), 2.68 (t, J=9.6 Hz, 2H), 2.19 (bs, 2H), 1.83 (s, 4H), 1.80-1.66 (m, 1H).

### Example 236: Synthesis of 3-fluoro-6-methoxy-4-(1-(3-methyloxetan-3-yl)-6-((phenylamino)methyl)-1H-benzo[d]imidazol-2-yl)benzene-1,2-diol

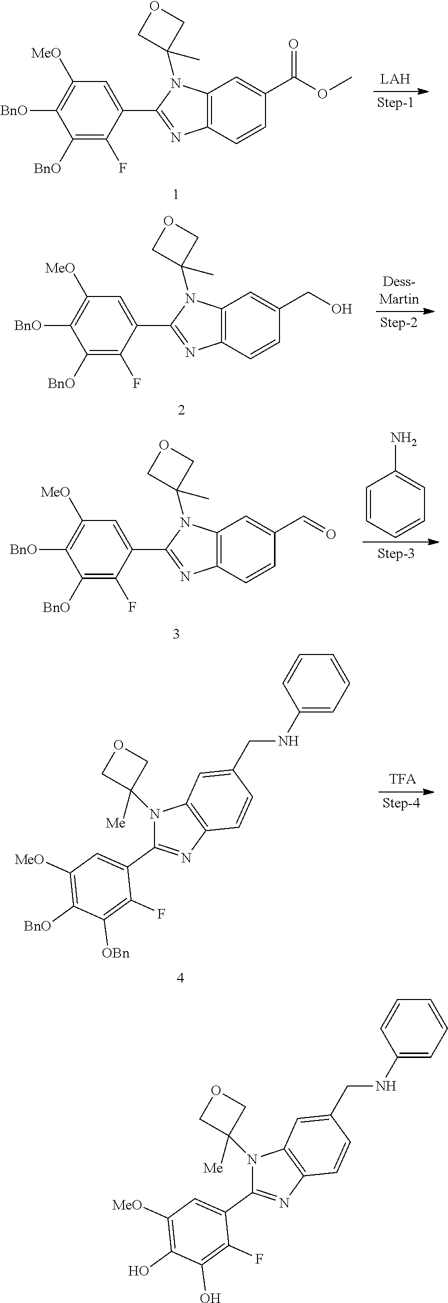

Step:1 To a solution of methyl 2-[3,4-bis(benzyloxy)-2-fluoro-5-methoxyphenyl]-1-(3-methyloxetan-3-yl)-1H-1,3-benzodiazole-6-carboxylate (430 mg, 738 μmol) in dry oxolane (5.00 mL), lithium alumanuide (1.11 mL, 1.5 eq., 1.11 mmol) added at 0° C. under inert atmosphere. The resulting reaction mixture stirred at room temperature till the complete consumption of starting material. After completion of the reaction, the reaction mixture was quenched with aqueous ammonium chloride to get the precipitate which were filtered, and ethyl acetate was added to filtrate. The organic fractions were collected, dried over anhydrous sodium sulphate, filtered and concentered to obtain {2-[3,4-bis(benzyloxy)-2-fluoro-5-methoxyphenyl]-1-(3-methyloxetan-3-yl)-1H-1,3-benzodiazol-6-yl}methanol (380 mg, 651 μmol) as sticky brown liquid. Yield: 380 mg, 88.19%

Step:2 To a solution of {2-[3,4-bis(benzyloxy)-2-fluoro-5-methoxyphenyl]-1-(3-methyloxetan-3-yl)-1H-1,3-benzodiazol-6-yl}methanol (460 mg, 829 μmol) in 1,2-dichloromethane (10.0 mL), 1,1-bis(acetyloxy)-3-oxo-3H-1λ⁵,2-benziodaoxol-1-yl acetate (528 mg, 1.5 eq., 1.24 mmol) added at 0° C. The resulting reaction mixture stirred at room temperature for 2 h. After completion of the reaction, the reaction mixture was quenched with saturated solution of sodium thiosulfate and sodium bicarbonate, extracted with ethyl acetate. The combined organic fractions collected, dried over anhydrous sodium sulphate, filtered and concentrated to get the crude. The crude purified by flash chromatography. The desired fractions were concentrated to afford 2-[3,4-bis(benzyloxy)-2-fluoro-5-methoxyphenyl]-1-(3-methyloxetan-3-yl)-1H-1,3-benzodiazole-6-carbaldehyde (350 mg, 602 μmol) as yellow liquid. Yield: 0.320 g, 72%

Step:3 To a stirred solution of 2-[3,4-bis(benzyloxy)-2-fluoro-5-methoxyphenyl]-1-(3-methyloxetan-3-yl)-1H-1,3-benzodiazole-6-carbaldehyde (120 mg, 217 μmol) in methanol (10.0 mL), aniline (27.8 mg, 1.1 eq., 299 μmol) was added at room temperature. The reaction mixture was stirred at room temperature for 2 h, and then sodium boranuide (38.9 mg, 5 eq., 1.09 mmol) was added in batches and the mixture was further stirred for another period of 16 h. The reaction was concentrated and extracted with Dichloromethane. The combined organic phases were dried over sodium sulphate and reduced under pressure to get N-({2-[3,4-bis(benzyloxy)-2-fluoro-5-methoxyphenyl]-1-(3-methyloxetan-3-yl)-1H-1,3-benzodiazol-6-yl}methyl)aniline (140 mg, 160 μmol) as white solid. Yield: 0.14 g, 58%

Step:4 To a stirred solution of N-({2-[3,4-bis(benzyloxy)-2-fluoro-5-methoxyphenyl]-1-(3-methyloxetan-3-yl)-1H-1,3-benzodiazol-6-yl}methyl)aniline (150 mg, 238 μmol) in trifluoroacetic acid (1.00 mL). The resulting mixture stirred for 2 h at 60° C. After completion, reaction mixture was concentrated under reduced pressure to get the crude and further purified by reverse phase HPLC. The desired fractions were lyophilized to afford 3-fluoro-6-methoxy-4-(1-(3-methyloxetan-3-yl)-6-((phenylamino)methyl)-1H-benzo[d]imidazol-2-yl)benzene-1,2-diol (0.024 g, 52.9 μmol) as off white solid. Yield: 0.024 g, 22.19%

1H NMR: ES MS M/Z=456.46 (M+1), ¹HNMR (400 MHz, DMSO-d6) δ 9.41 (brs, 2H), 7.62 (d, J=8.4 Hz, 1H), 7.28 (d, J=8.4 Hz, 1H), 7.20 (s, 1H), 7.04 (t, J=8.0 Hz, 1H), 6.62 (d, J=7.6 Hz, 2H), 6.57 (d, J=6.4 Hz, 1H), 6.51 (t, J=7.2 Hz, 1H), 6.25 (t, J=6.0 Hz, 1H), 4.66 (d, J=6.0 Hz, 2H), 4.39 (d, J=6.0 Hz, 2H), 4.32 (d, J=6.0 Hz, 2H), 3.77 (s, 3H), 1.97 (s, 3H).

### Example 237: Synthesis of N-(2-(2-fluoro-3,4-dihydroxy-5-methoxyphenyl)-3-(3-methyloxetan-3-yl)-3H-imidazo[4,5-c]pyridin-6-yl)acetamide

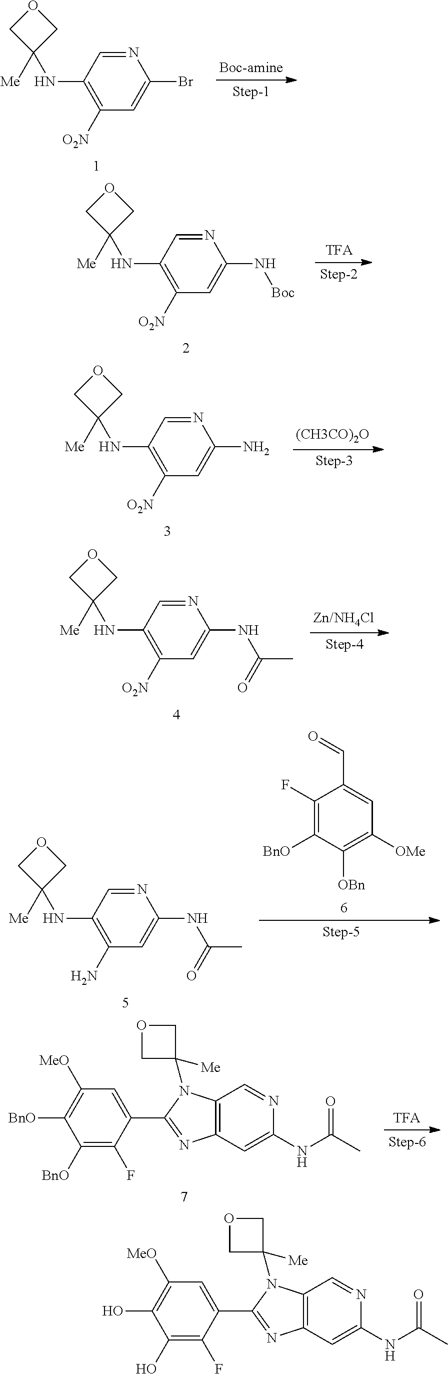

Step-1: To a stirred solution of 6-bromo-N-(3-methyloxetan-3-yl)-4-nitropyridin-3-amine (300 mg, 1.04 mmol) in 1,4-dioxane (5.00 mL), tert-butyl N-{5-[(3-methyloxetan-3-yl)amino]-4-nitropyridin-2-yl}carbamate (220 mg, 651 μmol) and cesium carbonate (679 mg, 2 eq., 2.08 mmol) were added and reaction mixture was purged with argon and then tris((1E,4E)-1,5-diphenylpenta-1,4-dien-3-one) dipalladium (47.7 mg, 0.05 eq., 52.1 μmol) and [5-(diphenylphosphanyl)-9,9-dimethyl-9H-xanthen-4-yl]diphenylphosphane (30.1 mg, 0.05 eq., 52.1 μmol) was added and again purged with argon. The reaction mixture was heated to 80° C. for 6 h. After completion, the reaction cooled to room temperature and passed through celite bed. The filtrate diluted with ethyl acetate and washed with water. The organic layer dried over anhydrous sodium sulphate, filtered and concentrated to give crude. The crude purified by flash chromatography. The desired fractions were concentrated to afford tert-butyl N-{5-[(3-methyloxetan-3-yl)amino]-4-nitropyridin-2-yl}carbamate (220 mg, 651 μmol) as off white solid. Yield: 220 mg, 62.5%

Step-2: To a stirred solution of tert-butyl N-{5-[(3-methyloxetan-3-yl)amino]-4-nitropyridin-2-yl}carbamate (220 mg, 678 μmol) in dichloromethane (2.00 mL), trifluoroacetic acid (1.00 mL) was added and reaction mixture was stirred at room temperature for 2 h. After completion, solvent was removed under reduced pressured to get the N5-(3-methyloxetan-3-yl)-4-nitropyridine-2,5-diamine (170 mg, 732 μmol) as brown solid. Yield: 170 mg, Crude

Step-3: To a stirred solution N5-(3-methyloxetan-3-yl)-4-nitropyridine-2,5-diamine TFA salt (170 mg, 758 μmol) in acetic acid (5.00 mL), acetyl acetate (232 mg, 3 eq., 2.27 mmol) was added and reaction mixture was heated to 60° C. for 16 h. After completion, reaction mixture concentrated under reduced pressure to get crude. The crude purified in flash chromatography. Combined fractions were concentrated under reduced pressure to get N-{5-[(3-methyloxetan-3-yl)amino]-4-nitropyridin-2-yl}acetamide (160 mg, 577 μmol) as white solid. Yield: 160 mg, 76.09%

Step-4: To a stirred solution N-{5-[(3-methyloxetan-3-yl)amino]-4-nitropyridin-2-yl}acetamide (170 mg, 638 μmol) in methanol (2.00 mL), zinc (209 mg, 5 eq., 3.19 mmol) and ammonium chloride (171 mg, 5 eq., 3.19 mmol) was added at 0° C. and reaction mixture was stirred at room temperature for 30 min. After completion, the reaction mixture passed through celite bed. The filtrate was concentrated under reduced pressure and washed with water. The organic layer dried over anhydrous sodium sulphate, filtered and concentrated to afford N-{4-amino-5-[(3-methyloxetan-3-yl)amino]pyridin-2-yl}acetamide (150 mg, 216 μmol) as brown solid. Yield: 150 mg, Crude

Step-5: To a stirred solution N-{4-amino-5-[(3-methyloxetan-3-yl)amino]pyridin-2-yl}acetamide (140 mg, 593 μmol) and 3,4-bis(benzyloxy)-2-fluoro-5-methoxybenzaldehyde (174 mg, 0.8 eq., 474 μmol) in methanesulfinylmethane (5.00 mL) was added disodium sulfinatosulfonate (169 mg, 1.5 eq., 889 μmol) at room temperature. The resulting mixture stirred for 1 h at 85° C. After completion, reaction mixture diluted with water solid obtained was filtered and dried to get N-{2-[3,4-bis(benzyloxy)-2-fluoro-5-methoxyphenyl]-3-(3-methyloxetan-3-yl)-3H-imidazo[4,5-c]pyridin-6-yl}acetamide (140 mg, 42.1 μmol) as brown solid. Yield: 140 mg, Crude

Step-6: A stirred solution of N-{2-[3,4-bis(benzyloxy)-2-fluoro-5-methoxyphenyl]-3-(3-methyloxetan-3-yl)-3H-imidazo[4,5-c]pyridin-6-yl}acetamide (100 mg, 172 μmol) in trifluoroacetic acid (1.00 mL) was heated to 60° C. for 2 h. After completion of reaction, reaction mixture was distilled below 30° C. to obtain crude as pale green mass. Crude was submitted for prep purification to get pure compound N-[2-(2-fluoro-3,4-dihydroxy-5-methoxyphenyl)-3-(3-methyloxetan-3-yl)-3H-imidazo[4,5-c]pyridin-6-yl]acetamide (6.00 mg, 14.9 μmol) as off white solid. Yield: 6 mg, 6.77%

LCMS and NMR Data: ES MS M/Z=403.35 (M+1), 1HNMR (400 MHz, DMSO-d6): δ 10.45 (s, 1H), 9.5 (bs, 2H), 8.43 (s, 1H), 8.32 (s, 1H), 6.64 (d, J=6.4 Hz, 1H), 4.74 (d, J=6.4 Hz, 2H), 4.41 (d, J=6.4 Hz, 2H), 3.79 (s, 3H), 2.11 (s, 3H), 2.02 (s, 3H).

### Example 238: Synthesis of 3-fluoro-6-methoxy-4-[1-(3-methyloxetan-3-yl)-6-(1,3,4-oxadiazol-2-yl)-1H-1,3-benzodiazol-2-yl] benzene-1,2-diol

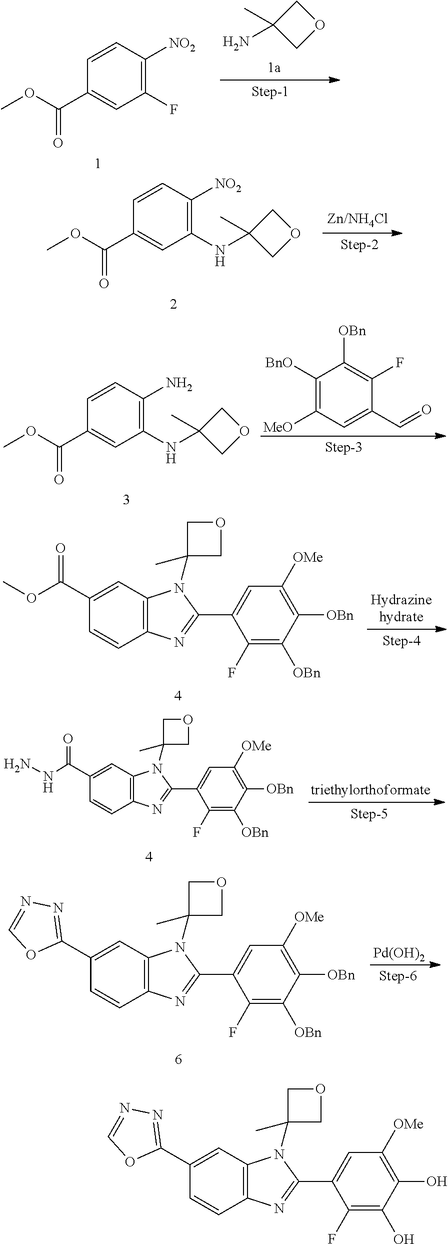

Step-2: To a stirred solution of methyl 3-fluoro-4-nitrobenzoate (500 mg, 2.51 mmol) in N-Methyl-2-Pyrrolidone (NMP) (5 ml), 3-methyloxetan-3-amine (230 μL, 2 eq., 5.02 mmol) and ethylbis(propan-2-yl)amine (1.32 mL, 7.53 mmol, 3 eq) were added at room temperature and stirred at 130° C. for h. After completion, reaction mixture quenched with ice cold water, solid was filtered and dried to afford methyl 3-[(3-methyloxetan-3-yl) amino]-4-nitrobenzoate (300 mg, 1.13 mmol) as a yellow solid. Yield: 0.30 g, 44%

Step-2: To a stirred solution of methyl 3-[(3-methyloxetan-3-yl)amino]-4-nitrobenzoate (200 mg, 0.66 eq., 751 μmol) in methanol (10.0 mL), zinc (370 mg, 1 eq., 5.65 mmol) and ammonium chloride (302 mg, 5 eq., 5.65 mmol) were added at room temperature and the reaction mixture stirred for 2 h at 40° C. After completion, the reaction mixture passed through celite bed. Filtrate was extracted with dichloromethane, dried over anhydrous sodium sulfate and concentrated under reduced pressure to get as methyl 4-amino-3-[(3-methyloxetan-3-yl)amino]benzoate (150 mg, 635 μmol) Violet color solid. Yield: 0.15 g (Crude)

Step-3: To a stirred solution of 3,4-bis(benzyloxy)-2-fluoro-5-methoxybenzaldehyde (161 mg, 0.8 eq., 440 μmol) and methyl 4-amino-3-[(3-methyloxetan-3-yl)amino]benzoate (130 mg, 550 mol) in methanesulfinylmethane (5.00 mL), disodium sulfatosulfonate (157 mg, 1.5 eq., 825 mol) was added at room temperature. The resulting mixture stirred for 12 h at 85° C. After completion, ice cold water was added in the reaction mixture and solid was filtered and dried to get crude. The crude purified by flash chromatography. The desired fractions were concentrated to afford methyl 2-[3,4-bis(benzyloxy)-2-fluoro-5-methoxyphenyl]-1-(3-methyloxetan-3-yl)-1H-1,3-benzodiazole-6-carboxylate (180 mg, 300 μmol) as sticky solid. Yield: 0.18 g, 54%

Step-4: To a solution of methyl 2-[3,4-bis(benzyloxy)-2-fluoro-5-methoxyphenyl]-1-(3-methyloxetan-3-yl)-1H-1,3-benzodiazole-6-carboxylate (50.0 mg, 85.8 μmol) in ethanol (2.00 mL), hydrazine hydrate monohydrate (333 μL, 4 eq., 343 μmol) was added to the reaction mixture and stirred at 80° C. for 16 h. After completion of reaction, reaction mixture concentrated and extracted with ethyl acetate. The combined organic layers were dried over anhydrous sodium sulphate, filtered and concentrated to obtained crude. Yield: 0.08 g, 62%

Step-5: To a solution of 2-[3,4-bis(benzyloxy)-2-fluoro-5-methoxyphenyl]-1-(3-methyloxetan-3-yl)-1H-1,3-benzodiazole-6-carbohydrazide (110 mg, 189 μmol) in Triethylorthoformate (1 ml) was added to the reaction mixture and stirred at 80° C. for 16 h. After completion of reaction, reaction mixture concentrated and extracted with ethyl acetate. The combined organic layers were dried over anhydrous sodium sulphate, filtered and concentrated to obtained crude. Yield: 0.10 g, 75%

Step-6: To a stirred solution of 2-[3,4-bis(benzyloxy)-2-fluoro-5-methoxyphenyl]-1-(3-methyloxetan-3-yl)-5-(1,3,4-oxadiazol-2-yl)-1H-1,3-benzodiazole (60.0 mg, 101 μmol) in Tetrahydrofuran (5.00 mL), palladium hydroxide (50.0 mg, 408 μmol) was added and reaction mixture stirred for 4 h at room temperature under H2 atmosphere. After completion, reaction mixture was passed through celite bed and filtrate was concentrated under reduced pressure to get the crude, further purified by reverse phase HPLC. The desired fractions were lyophilized to afford as an off white solid. Yield: 0.012 g, 28%

LCMS and NMR Data: ES MS M/Z=413 (M+1), NMR (400 MHz, DMSO-d6) δ 9.58 (s, 1H), 9.51 (s, 1H), 9.37 (s, 1H), 7.96 (d, J=8.4 Hz, 1H), 7.91 (d, J=8.4 Hz, 1H), 7.80 (s, 1H), 6.65 (d, J=6.0 Hz, 1H), 4.78 (d, J=5.6 Hz, 2H), 4.44 (d, J=5.6 Hz, 2H), 3.80 (s, 3H), 2.08 (s, 3H).

### Examples 239-335: Examples 239-335 were Synthesized as Described in Examples 1-6 and 272-238. LC-MS Data is Found in Table 1

### Example A. Human TREX1 Enzymatic Assay with dsDNA Native Oligonucleotide

Human TREX1 enzyme (amino acids 1-242) was diluted in assay buffer (20 mM Tris pH 7.7, 5 mM MgCl₂, 0.01% human serum albumin, 0.01% Brij™-35, 2 mM dithiothreitol) to a final concentration of 0.8 nM to 0.16 nM and added to a 96-well low-binding polypropylene plate. Test compounds were diluted in DMSO to 50× concentration (final DMSO concentration in reaction mixture of 2%) and added to the wells with a concentration ranging from 300 μM to 13 nM or 30 μM to 1.5 nM. The reaction mixtures were incubated for 30 minutes at 25° C. and a solution of annealed dsDNA oligonucleotide (5′-ACATTTCCCCGAAAAGTGCCACCCTTGGCG-3′ and Comp: 5′-CAAGGGTGGCACTTTTCGGGGAAATGT-3′) was added to a final concentration of 50 nM. The reaction mixtures were incubated at 25° C. for 5-15 minutes and subsequently quenched by transferring part of the assay reaction to a solution of 100 mM ethylenediaminetetraacetic acid and 1:100 Picogreen™ (final concentration of 60 mM ethylenediaminetetraacetic acid and 1:60 Picogreen™) in a black plate with an opaque bottom. The fluorescence (emission wavelength 480 nm/excitation wavelength 520 nm) was measured using a Molecular Devices SpectraMax plate reader. Wells containing oligonucleotide but no TREX1 enzyme were used as negative controls. Wells containing oligonucleotide, TREX1 enzyme, and DMSO were used as positive controls.

The data is shown in Table 2 (IC₅₀ displayed in μM).

TABLE 2

| | TREX1/Human + |
|---|---|
| Ex. # | DTT IC₅₀ AVG |
| 1 | D |
| 2 | D |
| 3 | E |
| 4 | C |
| 5 | C |
| 6 | C |
| 7 | NT |
| 8 | NT |
| 9 | E |
| 10 | E |
| 11 | D |
| 12 | D |
| 13 | D |
| 14 | E |
| 15 | E |
| 16 | E |
| 17 | E |
| 18 | E |
| 19 | E |
| 20 | E |
| 21 | E |
| 22 | NT |
| 23 | C |
| 24 | E |
| 25 | E |
| 26 | E |
| 27 | D |
| 28 | D |
| 29 | E |
| 30 | D |
| 31 | C |
| 32 | D |
| 33 | D |
| 34 | D |
| 35 | E |
| 36 | E |
| 37 | E |
| 38 | E |
| 39 | D |
| 40 | E |
| 41 | E |
| 42 | D |
| 43 | D |
| 44 | E |
| 45 | E |
| 46 | D |
| 47 | D |
| 48 | E |
| 49 | E |
| 50 | C |
| 51 | E |
| 52 | E |
| 53 | E |
| 54 | D |
| 55 | E |
| 56 | E |
| 57 | E |
| 58 | E |
| 59 | D |
| 60 | D |
| 61 | E |
| 62 | C |
| 63 | D |
| 64 | D |
| 65 | E |
| 66 | D |
| 67 | E |
| 68 | E |
| 69 | E |
| 70 | E |
| 71 | D |
| 72 | D |
| 73 | E |
| 74 | D |
| 75 | E |
| 76 | D |
| 77 | D |
| 78 | D |
| 79 | E |
| 80 | E |
| 81 | E |
| 82 | E |
| 83 | D |
| 84 | E |
| 85 | C |
| 86 | C |
| 87 | E |
| 88 | E |
| 89 | E |
| 90 | D |
| 91 | D |
| 92 | E |
| 93 | E |
| 94 | D |
| 95 | D |
| 96 | D |
| 97 | D |
| 98 | C |
| 99 | C |
| 100 | E |
| 101 | D |
| 102 | E |
| 103 | E |
| 104 | E |
| 105 | E |
| 106 | C |
| 107 | C |
| 108 | C |
| 109 | D |
| 110 | E |
| 111 | C |
| 112 | E |
| 113 | E |
| 114 | D |
| 115 | D |
| 116 | C |
| 117 | D |
| 118 | D |
| 119 | D |
| 120 | D |
| 121 | E |
| 122 | D |
| 123 | D |
| 124 | C |
| 125 | E |
| 126 | E |
| 127 | D |
| 128 | D |
| 129 | C |
| 130 | D |
| 131 | D |
| 132 | D |
| 133 | E |
| 134 | E |
| 135 | C |
| 136 | E |
| 137 | C |
| 138 | E |
| 139 | C |
| 140 | D |
| 141 | E |
| 142 | E |
| 143 | E |
| 144 | D |
| 145 | E |
| 146 | C |
| 147 | E |
| 148 | C |
| 149 | D |
| 150 | C |
| 151 | D |
| 152 | E |
| 153 | D |
| 154 | C |
| 155 | D |
| 156 | D |
| 157 | C |
| 158 | C |
| 159 | C |
| 160 | E |
| 161 | E |
| 162 | E |
| 163 | B |
| 164 | C |
| 165 | D |
| 166 | D |
| 167 | C |
| 168 | B |
| 169 | B |
| 170 | D |
| 171 | E |
| 172 | E |
| 173 | D |
| 174 | D |
| 175 | D |
| 176 | B |
| 177 | B |
| 178 | C |
| 179 | B |
| 180 | A |
| 181 | A |
| 182 | B |
| 183 | D |
| 184 | A |
| 185 | C |
| 186 | C |
| 187 | C |
| 188 | C |
| 189 | C |
| 190 | B |
| 191 | D |
| 192 | A |
| 193 | B |
| 194 | A |
| 195 | B |
| 196 | E |
| 197 | B |
| 198 | A |
| 199 | C |
| 200 | C |
| 201 | D |
| 202 | E |
| 203 | A |
| 204 | A |
| 205 | A |
| 206 | B |
| 207 | A |
| 209 | B |
| 210 | B |
| 211 | A |
| 212 | A |
| 213 | A |
| 214 | B |
| 215 | A |
| 216 | D |
| 217 | D |
| 218 | A |
| 219 | B |
| 220 | B |
| 221 | C |
| 222 | A |
| 223 | B |
| 224 | A |
| 225 | C |
| 226 | C |
| 227 | B |
| 228 | D |
| 229 | C |
| 230 | B |
| 231 | E |
| 232 | B |
| 233 | E |
| 234 | C |
| 235 | B |
| 236 | B |
| 237 | A |
| 238 | A |
| 239 | E |
| 241 | NT |
| 242 | E |
| 243 | B |
| 244 | A |
| 245 | C |
| 246 | A |
| 247 | A |
| 248 | A |
| 249 | A |
| 250 | A |
| 251 | A |
| 252 | A |
| 253 | A |
| 254 | A |
| 255 | B |
| 256 | A |
| 257 | B |
| 258 | A |
| 259 | A |
| 260 | B |
| 261 | B |
| 262 | A |
| 263 | A |
| 264 | A |
| 265 | A |
| 266 | C |
| 267 | A |
| 268 | E |
| 269 | A |
| 270 | A |
| 271 | A |
| 272 | B |
| 273 | B |
| 274 | E |
| 275 | B |
| 276 | A |
| 277 | C |
| 278 | A |
| 279 | NT |
| 280 | B |
| 281 | B |
| 282 | NT |
| 283 | C |
| 284 | C |
| 285 | C |
| 286 | C |
| 287 | E |
| 288 | C |
| 289 | C |
| 290 | D |
| 291 | B |
| 292 | D |
| 293 | E |
| 294 | C |
| 295 | A |
| 296 | A |
| 297 | D |
| 298 | C |
| 299 | D |
| 300 | D |
| 301 | B |
| 302 | B |
| 303 | A |
| 304 | B |
| 305 | A |
| 306 | A |
| 307 | A |
| 308 | B |
| 309 | A |
| 310 | A |
| 311 | B |
| 312 | A |
| 313 | B |
| 314 | C |
| 314 | E |
| 315 | D |
| 316 | B |
| 317 | B |
| 318 | B |
| 319 | C |
| 320 | E |
| 321 | E |
| 322 | D |
| 323 | C |
| 324 | C |
| 325 | D |
| 326 | C |
| 327 | C |
| 328 | A |
| 329 | B |
| 330 | B |
| 331 | C |
| 332 | NT |
| 333 | E |
| 334 | B |
| 335 | B |

A is less than or equal to 100 nM;
B is more than 100 nM and less than or equal to 1 μM;
C is more than 1 μM and less than or equal to 10 μM;
D is more than 10 μM and less than or equal to 100 μM;
E is more than 100 μM.
NT is not tested

### Example B: Gel Assay

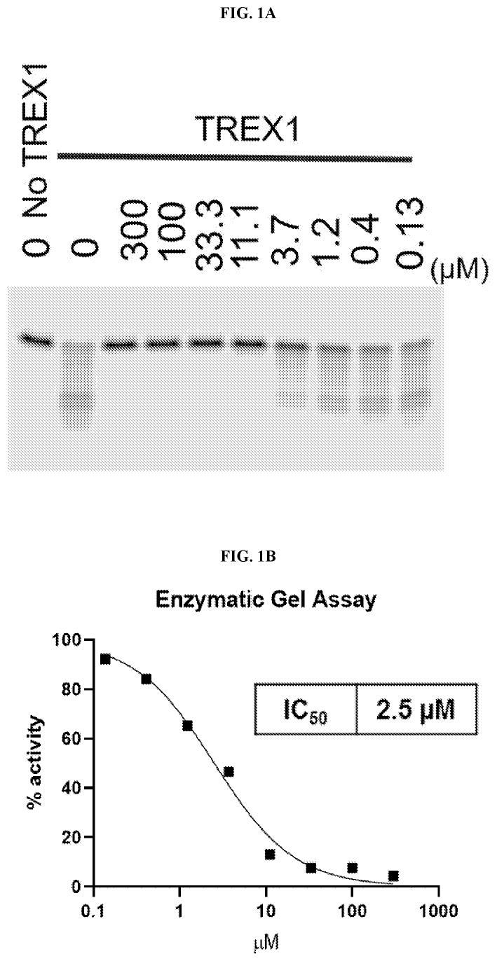

A gel assay was conducted as an orthogonal control to confirm observations of TREX1 inhibitors determined with the fluorescence-based assay. Briefly, compounds were diluted in DMSO at 50× the final concentration. Human TREX1 was diluted to 120 μM (2×concentration) in assay buffer (assay buffer: 20 mM Tris pH 7.7 (Life T-echnologies), 5 mM MgCl₂ (Sigma), 0.01% HSA (Sigma), 0.01% Brij-35 (ThermoFisher), 2 mM DTT (Sigma)). Oligonucleotide strand (IR-680-5′ACATTTCCCCGAAAAGTGCCACCCTTG-3′) (Custom probe made by Trilink) was diluted in the same assay buffer to a working concentration of 25 nM (final concentration of 12.5 nM). Example 107 was added to 60 nM human TREX1 enzyme in clear 96 well plates. The plate was then incubated at 25° C. for 30 minutes. Twenty five nM oligonucleotide strand was added to the plate, to a final concentration of 30 pM TREX1, 12.5 nM oligo, 1× compound and incubated at 25° C. Fifteen minutes later, the reaction was stopped by removing 10 μl of the reaction and adding it to 20 μl of a solution of 50 mM EDTA/1.5× loading dye buffer (6× Orange DNA loading dye; ThermoFisher) in a 96 well PCR plate. The plate was then incubated for 3 minutes at 70° C. and the reaction loaded on a Novex TBE polyacrylamide gel, 20% 10-well (ThermoFisher). The samples were run for 1 hour and a half at 150V using the Invitrogen mini gel box system. Gel was then visualized using Licor Odyssey, 700 nm wavelength with 0.5 offset. The top band was quantified using Image Studio (Licor). Controls were Oligonucleotide strand with no TREX1 enzyme and Oligonucleotide strand with TREX1 enzyme and DMSO (final DMSO concentration of 2%). Prism (GraphPad) was used to calculate IC₅₀. FIG. 1A: Scan of a 20% polyacrylamide gel with the product from the TREX1 enzymatic reaction in the presence of different doses of inhibitor. The top band intensity was measured using the Odyssey scanner (Li-Cor) and ImageStudio software (Li-Cor) and FIG. 1B: Graph representing the percent of top band intensity degradation as a measure of human TREX1 activity. GraphPad Prism 8 software was used to produce this graph and measure IC50. No TREX1 sample was used as the positive control and TREX1 with DMSO was as used as the negative control.

The examples and embodiments described herein are for illustrative purposes only and in some embodiments, various modifications or changes are to be included within the purview of disclosure and scope of the appended claims.

What is claimed is:

1. A compound of Formula (I), or a pharmaceutically acceptable salt, solvate, tautomer, or stereoisomer thereof: 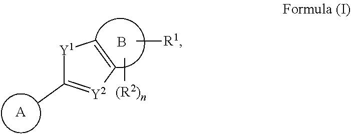
wherein:
Ring A is 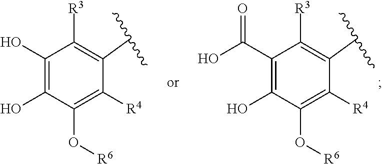
Ring B is phenyl or a 6-membered heteroaryl;
R¹ is hydrogen, deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{1a};
each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
Y¹ is O, S, or NR^{Y1};
Y² is N or CR^{Y2};
provided that when Y² is CR^{Y2}, Y¹ is not O;
R^{Y1} is hydrogen, —S(═O)R^{a}, —S(═O)₂R^{a}, —S(═O)₂NR^{W}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a}.
R^{Y2} is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y2a};
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R^{1a} is independently deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{1b};
or two R^{1a} on the same carbon are taken together to form an oxo;
each R¹⁰ is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{10a};
each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{11a};
each R¹² and R¹³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a};
or R¹² and R¹³ are taken together with the nitrogen atom to which they are attached to form a heterocycloalkyl optionally substituted with one or more R^{13a};
each R^{Y1a}, R^{Y2a}, R^{2a}, R^{3a}, R^{4a}, R^{6a}, R^{10a}, R^{11a}, R^{12a}, R^{13a}, and R^{1b} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{Y2a}, two R^{2a}, two R^{3a}, two R^{4a}, two R^{5a}, two R^{6a}, two R^{10a}, two R^{11a}, two R^{12a}, two R^{13a}, and two R^{1b} on the same carbon are taken together to form an oxo, a cycloalkyl, or a heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
provided that the compound of Formula (I) is not

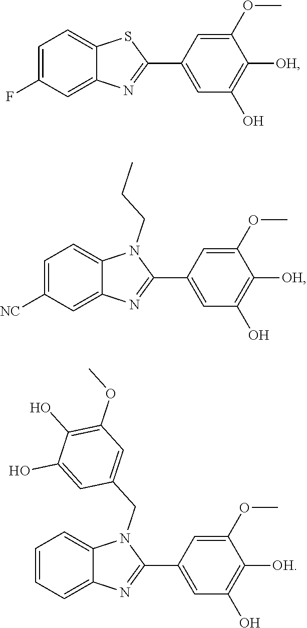 [SMILES: COc1cc(-c2nc3cc(F)ccc3s2)cc(O)c1O ; CCCn1c(-c2cc(O)c(O)c(OC)c2)nc2cc(C#N)ccc21 ; COc1cc(Cn2c(-c3cc(O)c(O)c(OC)c3)nc3ccccc32)cc(O)c1O]

2. The compound of claim 1, or a pharmaceutically acceptable salt, solvate, tautomer, or stereoisomer thereof, wherein the compound of Formula (I) is of Formula (Ia):

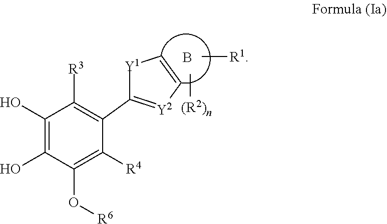

3. The compound of claim 1, or a pharmaceutically acceptable salt, solvate, tautomer, or stereoisomer thereof, wherein the compound of Formula (I) is of Formula (Ib):

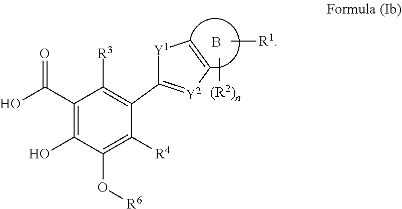

4. The compound of any one of claims 1-3, or a pharmaceutically acceptable salt, solvate, tautomer, or stereoisomer thereof, wherein:

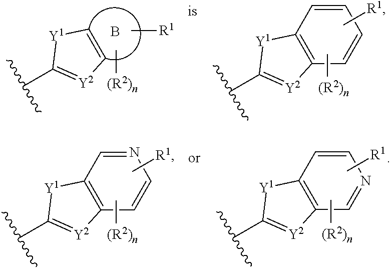

5. The compound of any one of claims 1-4, or a pharmaceutically acceptable salt, solvate, tautomer, or stereoisomer thereof, wherein:

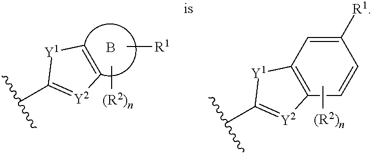

6. The compound of any one of claims 1-4, or a pharmaceutically acceptable salt, solvate, tautomer, or stereoisomer thereof, wherein:

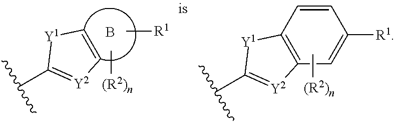

7. The compound of any one of claims 1-4, or a pharmaceutically acceptable salt, solvate, tautomer, or stereoisomer thereof, wherein:

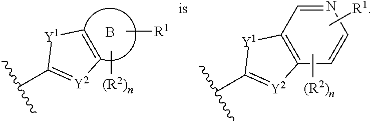

8. The compound of any one of claims 1-4, or a pharmaceutically acceptable salt, solvate, tautomer, or stereoisomer thereof, wherein:

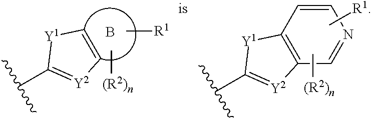

9. The compound of any one of claims 1-8, or a pharmaceutically acceptable salt, solvate, tautomer, or stereoisomer thereof, wherein:
Y¹ is S and Y² is N.

10. The compound of any one of claims 1-8, or a pharmaceutically acceptable salt, solvate, tautomer, or stereoisomer thereof, wherein:
Y¹ is O and Y² is N.

11. The compound of any one of claims 1-8, or a pharmaceutically acceptable salt, solvate, tautomer, or stereoisomer thereof, wherein:
Y¹ is NR^{Y1} and Y² is N.

12. The compound of any one of claims 1-8, or a pharmaceutically acceptable salt, solvate, tautomer, or stereoisomer thereof, wherein:
Y¹ is NR^{Y1} and Y² is CR^{Y2}.

13. The compound of any one of claims 1-8 or 12, or a pharmaceutically acceptable salt, solvate, tautomer, or stereoisomer thereof, wherein:
R^{Y1} is hydrogen, C₁-C₆alkyl, or heterocycloalkyl.

14. The compound of any one of claims 1-8 or 12 or 13, or a pharmaceutically acceptable salt, solvate, tautomer, or stereoisomer thereof, wherein:
R^{Y1} is hydrogen.

15. The compound of any one of claims 1-8 or 12 or 13, or a pharmaceutically acceptable salt, solvate, tautomer, or stereoisomer thereof, wherein:
R^{Y1} is heterocycloalkyl.

16. The compound of any one of claims 1-8 or 12-15, or a pharmaceutically acceptable salt, solvate, tautomer, or stereoisomer thereof, wherein:
R^{Y2} is hydrogen, deuterium, halogen, —CN, —OR^{b}, —NR^{c}R^{d}, C₁-C₆alkyl, C₁-C₆haloalkyl, C₁-C₆deuteroalkyl, cycloalkyl, heterocycloalkyl, aryl, or heteroaryl; wherein the alkyl, cycloalkyl, heterocycloalkyl, aryl, and heteroaryl is optionally substituted with one or more R^{Y2a}.

17. The compound of any one of claims 1-8 or 12-16, or a pharmaceutically acceptable salt, solvate, tautomer, or stereoisomer thereof, wherein:
R^{Y2} is hydrogen, halogen, C₁-C₆alkyl, or C₁-C₆haloalkyl.

18. The compound of any one of claims 1-8 or 12-17, or a pharmaceutically acceptable salt, solvate, tautomer, or stereoisomer thereof, wherein:
R^{Y2} is hydrogen.

19. The compound of any one of claims 1-18, or a pharmaceutically acceptable salt, solvate, tautomer, or stereoisomer thereof, wherein:
R³ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

20. The compound of any one of claims 1-19, or a pharmaceutically acceptable salt, solvate, tautomer, or stereoisomer thereof, wherein:
R³ is hydrogen or halogen.

21. The compound of any one of claims 1-20, or a pharmaceutically acceptable salt, solvate, tautomer, or stereoisomer thereof, wherein:
R³ is hydrogen.

22. The compound of any one of claims 1-21, or a pharmaceutically acceptable salt, solvate, tautomer, or stereoisomer thereof, wherein:
R⁴ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

23. The compound of any one of claims 1-22, or a pharmaceutically acceptable salt, solvate, tautomer, or stereoisomer thereof, wherein:
R⁴ is hydrogen or halogen.

24. The compound of any one of claims 1-23, or a pharmaceutically acceptable salt, solvate, tautomer, or stereoisomer thereof, wherein:
R⁴ is hydrogen.

25. The compound of any one of claims 1-24, or a pharmaceutically acceptable salt, solvate, tautomer, or stereoisomer thereof, wherein:
R⁶ is C₁-C₆alkyl.

26. The compound of any one of claims 1-25, or a pharmaceutically acceptable salt, solvate, tautomer, or stereoisomer thereof, wherein:
R⁶ is methyl.

27. The compound of any one of claims 1-26, or a pharmaceutically acceptable salt, solvate, tautomer, or stereoisomer thereof, wherein:
each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

28. The compound of any one of claims 1-27, or a pharmaceutically acceptable salt, solvate, tautomer, or stereoisomer thereof, wherein:
each R² is independently hydrogen, deuterium, halogen, —CN, —OR^{b}, —NR^{c}R^{d}, C₁-C₆alkyl, C₁-C₆haloalkyl, or C₁-C₆deuteroalkyl.

29. The compound of any one of claims 1-28, or a pharmaceutically acceptable salt, solvate, tautomer, or stereoisomer thereof, wherein:
each R² is independently hydrogen, halogen, C₁-C₆alkyl, or C₁-C₆haloalkyl.

30. The compound of any one of claims 1-29, or a pharmaceutically acceptable salt, solvate, tautomer, or stereoisomer thereof, wherein:
each R² is independently hydrogen or halogen.

31. The compound of any one of claims 1-30, or a pharmaceutically acceptable salt, solvate, tautomer, or stereoisomer thereof, wherein:
each R² is hydrogen.

32. The compound of any one of claims 1-31, or a pharmaceutically acceptable salt, solvate, tautomer, or stereoisomer thereof, wherein:
n is 1 or 2.

33. The compound of any one of claims 1-32, or a pharmaceutically acceptable salt, solvate, tautomer, or stereoisomer thereof, wherein:
n is 1.

34. The compound of any one of claims 1-33, or a pharmaceutically acceptable salt, solvate, tautomer, or stereoisomer thereof, wherein:
R¹ is hydrogen, deuterium, halogen, —CN, —OR¹¹, —NR¹²R¹³, —C(═O)R¹⁰, —C(═O)OR¹¹, —C(═O)NR¹²R¹³, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein the alkyl, cycloalkyl, heterocycloalkyl, aryl, and heteroaryl is optionally substituted with one or more R^{1a}.

35. The compound of any one of claims 1-34, or a pharmaceutically acceptable salt, solvate, tautomer, or stereoisomer thereof, wherein:
R¹ is hydrogen, deuterium, halogen, —CN, —OR¹¹, —NR¹²R¹³, —C(═O)R¹⁰, —C(═O)OR¹¹, —C(═O)NR¹²R¹³, C₁-C₆alkyl, C₁-C₆haloalkyl, or C₁-C₆deuteroalkyl.

36. The compound of any one of claims 1-35, or a pharmaceutically acceptable salt, solvate, tautomer, or stereoisomer thereof, wherein:
R¹ is hydrogen, halogen, C₁-C₆alkyl, or C₁-C₆haloalkyl.

37. The compound of any one of claims 1-36, or a pharmaceutically acceptable salt, solvate, tautomer, or stereoisomer thereof, wherein:
R¹ is hydrogen or halogen.

38. The compound of any one of claims 1-37, or a pharmaceutically acceptable salt, solvate, tautomer, or stereoisomer thereof, wherein:
R¹ is hydrogen.

39. The compound of any one of claims 1-34, or a pharmaceutically acceptable salt, solvate, tautomer, or stereoisomer thereof, wherein:
R¹ is cycloalkyl, heterocycloalkyl, aryl, or heteroaryl; wherein the cycloalkyl, heterocycloalkyl, aryl, and heteroaryl is optionally substituted with one or more R^{1a}.

40. The compound of any one of claims 1-34 or 39, or a pharmaceutically acceptable salt, solvate, tautomer, or stereoisomer thereof, wherein:
R¹ is heterocycloalkyl optionally substituted with one or more R^{1a}.

41. The compound of any one of claims 1-34 or 39 or 40, or a pharmaceutically acceptable salt, solvate, tautomer, or stereoisomer thereof, wherein:
R¹ is heterocycloalkyl optionally substituted with one or more R^{1a}; wherein the heterocycloalkyl is piperidinyl or piperazinyl.

42. The compound of any one of claims 1-34 or 39-41, or a pharmaceutically acceptable salt, solvate, tautomer, or stereoisomer thereof, wherein:
R¹ is piperidinyl optionally substituted with one or more R^{1a}.

43. The compound of any one of claims 1-34 or 39-41, or a pharmaceutically acceptable salt, solvate, tautomer, or stereoisomer thereof, wherein:
R¹ is piperazinyl optionally substituted with one or more R^{1a}.

44. The compound of any one of claims 1-34 or 39-43, or a pharmaceutically acceptable salt, solvate, tautomer, or stereoisomer thereof, wherein:
each R^{1a} is independently deuterium, halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, —C(═O)OR¹¹, —C(═O)NR¹²R¹³, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more R^{1b}; or two R^{1a} on the same carbon are taken together to form an oxo.

45. The compound of any one of claims 1-34 or 39-44, or a pharmaceutically acceptable salt, solvate, tautomer, or stereoisomer thereof, wherein:
each R^{1a} is independently halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, C₁-C₆alkyl, C₁-C₆haloalkyl, or heteroaryl; wherein each alkyl and heteroaryl is independently optionally substituted with one or more R^{1b}; or two R^{1a} on the same carbon are taken together to form an oxo.

46. The compound of any one of claims 1-34 or 39-45, or a pharmaceutically acceptable salt, solvate, tautomer, or stereoisomer thereof, wherein:
each R^{1a} is independently —C(═O)R¹⁰.

47. The compound of any one of claims 1-34 or 39-46, or a pharmaceutically acceptable salt, solvate, tautomer, or stereoisomer thereof, wherein:
each R¹⁰ is independently C₁-C₆alkyl, C₂-C₆alkenyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl;
wherein each alkyl, alkenyl, cycloalkyl, heterocycloalkyl, aryl, and heteroaryl is independently optionally substituted with one or more R^{10a}.

48. The compound of any one of claims 1-34 or 39-47, or a pharmaceutically acceptable salt, solvate, tautomer, or stereoisomer thereof, wherein:
each R¹⁰ is independently C₁-C₆alkyl, C₂-C₆alkenyl, C₁-C₆aminoalkyl, aryl, or heteroaryl;
wherein each alkyl, alkenyl, aryl, and heteroaryl is independently optionally substituted with one or more R^{10a}.

49. The compound of any one of claims 1-33 or 38-47, or a pharmaceutically acceptable salt, solvate, tautomer, or stereoisomer thereof, wherein:
each R¹⁰ is independently aryl optionally substituted with one or more R^{10a}.

50. The compound of any one of claims 1-33 or 38-48, or a pharmaceutically acceptable salt, solvate, tautomer, or stereoisomer thereof, wherein:
each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl.

51. The compound of any one of claims 1-34 or 39-50, or a pharmaceutically acceptable salt, solvate, tautomer, or stereoisomer thereof, wherein:
each R^{10a} is independently halogen, —OR^{b}, —C(═O)OR^{b}, or C₁-C₆alkyl.

52. A compound of Formula (II) or (III), or a pharmaceutically acceptable salt, solvate, tautomer, or stereoisomer thereof: 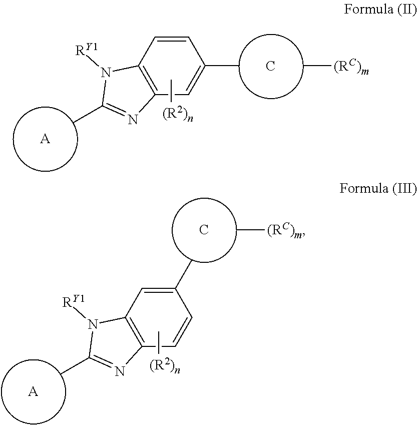
wherein:
Ring A is 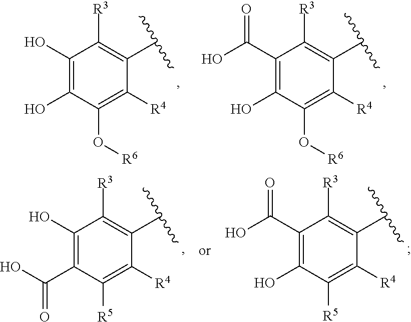
Ring C is cycloalkyl or heterocycloalkyl;
each R^{C} is independently deuterium, halogen, —CN, —OR¹¹, —SR¹¹, —S(═O)R¹⁰, —S(═O)₂R¹⁰, —NO₂, —NR¹²R¹³, —NHS(═O)₂R¹⁰, —S(═O)₂NR¹²R¹³, —C(═O)R¹⁰, —OC(═O)R¹⁰, —C(═O)OR¹¹, —OC(═O)OR¹¹, —C(═O)NR¹²R¹³, —OC(═O)NR¹²R¹³, —NR¹¹C(═O)NR¹²R¹³, —NR¹¹C(═O)R¹⁰, —NR¹¹C(═O)OR¹¹, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{Ca};
or two R^{C} on the same carbon are taken together to form an oxo;
m is 1-3,
each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a};
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or R⁴ and R⁵ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R¹⁰ is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{10a};
each R¹¹ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{11a};
each R¹² and R¹³ is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{12a};
or R¹² and R¹³ are taken together with the nitrogen atom to which they are attached to form a heterocycloalkyl optionally substituted with one or more R^{13a}.
each R^{Y1a}, R^{2a}, R^{3a}, R^{4a}, R^{5a}, R^{6a}, R^{10a}, R^{11a}, R^{12a}, R^{13a}, and R^{Ca} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{2a}, two R^{3a}, two R^{4a}, two R^{5a}, two R^{6a}, two R^{10a}, two R^{11a}, two R^{12a}, two R^{13a}, and two R^{Ca} on the same carbon are taken together to form an oxo, a cycloalkyl, or heterocycloalkyl;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

53. The compound of claim 52, or a pharmaceutically acceptable salt, solvate, tautomer, or stereoisomer thereof, wherein the compound of Formula (II) is of Formula (IIa):

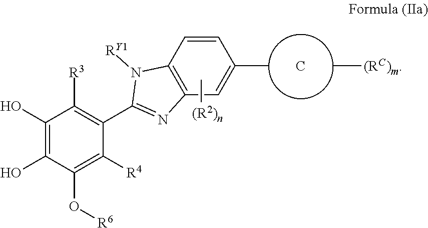

54. The compound of claim 52, or a pharmaceutically acceptable salt, solvate, tautomer, or stereoisomer thereof, wherein the compound of Formula (II) is of Formula (IIb):

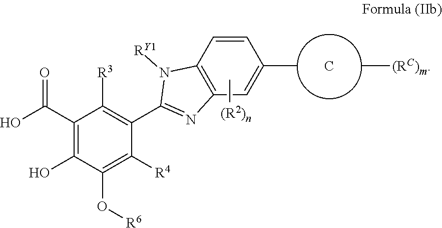

55. The compound of claim 52, or a pharmaceutically acceptable salt, solvate, tautomer, or stereoisomer thereof, wherein the compound of Formula (II) is of Formula (IIc):

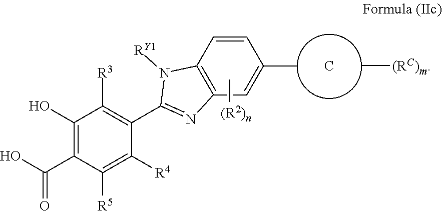

56. The compound of claim 51, or a pharmaceutically acceptable salt, solvate, tautomer, or stereoisomer thereof, wherein the compound of Formula (II) is of Formula (IId):

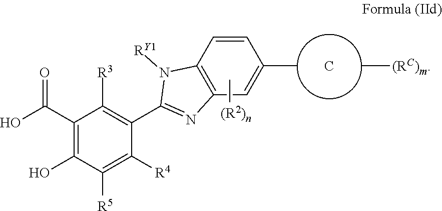

57. The compound of claim 51, or a pharmaceutically acceptable salt, solvate, tautomer, or stereoisomer thereof, wherein the compound of Formula (III) is of Formula (IIIa):

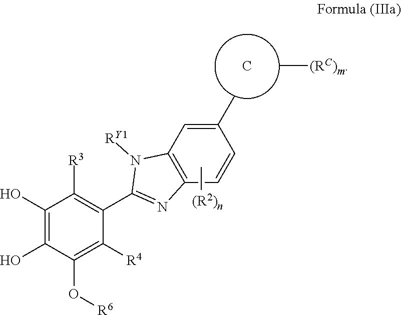

58. The compound of claim 51, or a pharmaceutically acceptable salt, solvate, tautomer, or stereoisomer thereof, wherein the compound of Formula (III) is of Formula (IIIb):

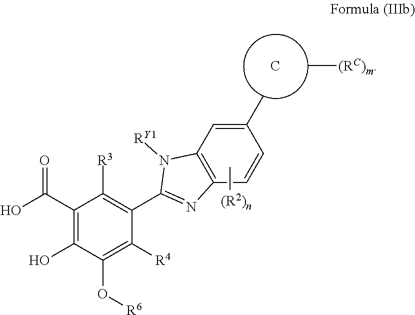

59. The compound of claim 51, or a pharmaceutically acceptable salt, solvate, tautomer, or stereoisomer thereof, wherein the compound of Formula (III) is of Formula (IIIc):

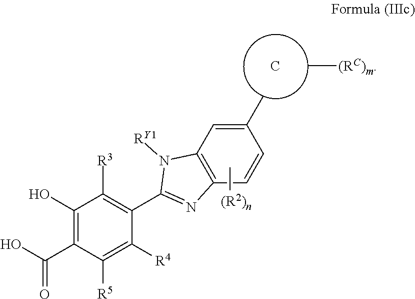

60. The compound of claim 51, or a pharmaceutically acceptable salt, solvate, tautomer, or stereoisomer thereof, wherein the compound of Formula (III) is of Formula (IIId):

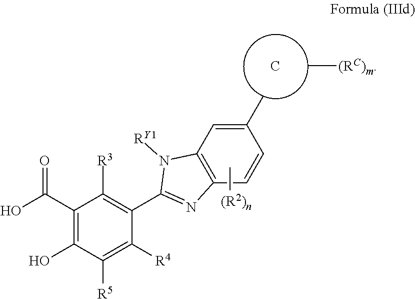

61. The compound of any one of claims 52-60, or a pharmaceutically acceptable salt, solvate, tautomer, or stereoisomer thereof, wherein:
R^{Y1} is hydrogen, C₁-C₆alkyl, or heterocycloalkyl.

62. The compound of any one of claims 52-61, or a pharmaceutically acceptable salt, solvate, tautomer, or stereoisomer thereof, wherein:
R^{Y1} is hydrogen.

63. The compound of any one of claims 52-62, or a pharmaceutically acceptable salt, solvate, tautomer, or stereoisomer thereof, wherein:
R_{Y1} is heterocycloalkyl.

64. The compound of any one of claims 52-63, or a pharmaceutically acceptable salt, solvate, tautomer, or stereoisomer thereof, wherein:
R³ is hydrogen, deuterium, halogen, —CN, —OR^{b}, —NR^{c}R^{d}, —C(═O)R, —C(═O)OR^{b}, —C(═O)NR^{c}R^{d}, C₁-C₆alkyl, C₁-C₆haloalkyl, or C₁-C₆deuteroalkyl.

65. The compound of any one of claims 52-64, or a pharmaceutically acceptable salt, solvate, tautomer, or stereoisomer thereof, wherein:
R³ is hydrogen or halogen.

66. The compound of any one of claims 52-65, or a pharmaceutically acceptable salt, solvate, tautomer, or stereoisomer thereof, wherein:
R³ is hydrogen.

67. The compound of any one of claims 52-66, or a pharmaceutically acceptable salt, solvate, tautomer, or stereoisomer thereof, wherein:
R⁴ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

68. The compound of any one of claims 52-67, or a pharmaceutically acceptable salt, solvate, tautomer, or stereoisomer thereof, wherein:
R⁴ is hydrogen or halogen.

69. The compound of any one of claims 52-68, or a pharmaceutically acceptable salt, solvate, tautomer, or stereoisomer thereof, wherein:
R⁴ is hydrogen.

70. The compound of any one of claims 52-69, or a pharmaceutically acceptable salt, solvate, tautomer, or stereoisomer thereof, wherein:
R⁵ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

71. The compound of any one of claims 52-70, or a pharmaceutically acceptable salt, solvate, tautomer, or stereoisomer thereof, wherein:
R⁵ is hydrogen or halogen.

72. The compound of any one of claims 52-71, or a pharmaceutically acceptable salt, solvate, tautomer, or stereoisomer thereof, wherein:
R⁵ is hydrogen.

73. The compound of any one of claims 52-72, or a pharmaceutically acceptable salt, solvate, tautomer, or stereoisomer thereof, wherein:
R⁶ is C₁-C₆alkyl.

74. The compound of any one of claims 52-73, or a pharmaceutically acceptable salt, solvate, tautomer, or stereoisomer thereof, wherein:
R⁶ is methyl.

75. The compound of any one of claims 52-74, or a pharmaceutically acceptable salt, solvate, tautomer, or stereoisomer thereof, wherein:
each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

76. The compound of any one of claims 52-75, or a pharmaceutically acceptable salt, solvate, tautomer, or stereoisomer thereof, wherein:
each R² is independently hydrogen, deuterium, halogen, —CN, —OR^{b}, —NR^{c}R^{d}, C₁-C₆alkyl, C₁-C₆haloalkyl, or C₁-C₆deuteroalkyl.

77. The compound of any one of claims 52-76, or a pharmaceutically acceptable salt, solvate, tautomer, or stereoisomer thereof, wherein:
each R² is independently hydrogen, halogen, C₁-C₆alkyl, or C₁-C₆haloalkyl.

78. The compound of any one of claims 52-77, or a pharmaceutically acceptable salt, solvate, tautomer, or stereoisomer thereof, wherein:
each R² is independently hydrogen or halogen.

79. The compound of any one of claims 52-78, or a pharmaceutically acceptable salt, solvate, tautomer, or stereoisomer thereof, wherein:
each R² is hydrogen.

80. The compound of any one of claims 52-79, or a pharmaceutically acceptable salt, solvate, tautomer, or stereoisomer thereof, wherein:
n is 1 or 2.

81. The compound of any one of claims 52-79, or a pharmaceutically acceptable salt, solvate, tautomer, or stereoisomer thereof, wherein:
n is 1.

82. The compound of any one of claims 52-81, or a pharmaceutically acceptable salt, solvate, tautomer, or stereoisomer thereof, wherein:
Ring C is heterocycloalkyl.

83. The compound of any one of claims 52-81, or a pharmaceutically acceptable salt, solvate, tautomer, or stereoisomer thereof, wherein:
Ring C is cycloalkyl.

84. The compound of any one of claims 52-83, or a pharmaceutically acceptable salt, solvate, tautomer, or stereoisomer thereof, wherein:
each R^{C} is independently deuterium, halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, —C(═O)OR¹¹, —C(═O)NR¹²R¹³, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one or more R^{Ca}; or two R^{C} on the same carbon are taken together to form an oxo.

85. The compound of any one of claims 52-83, or a pharmaceutically acceptable salt, solvate, tautomer, or stereoisomer thereof, wherein:
each R^{C} is independently halogen, —CN, —OR¹¹, —S(═O)₂R¹⁰, —NR¹²R¹³, —NHS(═O)₂R¹⁰, —S(═O)₂NR¹²R¹³, —C(═O)R¹⁰, C₁-C₆alkyl, C₁-C₆haloalkyl, or heteroaryl; wherein each alkyl and heteroaryl is independently optionally substituted with one or more R^{Ca}; or two R^{C} on the same carbon are taken together to form an oxo.

86. The compound of any one of claims 52-85, or a pharmaceutically acceptable salt, solvate, tautomer, or stereoisomer thereof, wherein:
each R^{C} is independently —C(═O)R¹⁰.

87. The compound of any one of claims 52-86, or a pharmaceutically acceptable salt, solvate, tautomer, or stereoisomer thereof, wherein:
each R¹⁰ is independently C₁-C₆alkyl, C₂-C₆alkenyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl; wherein each alkyl, alkenyl, cycloalkyl, heterocycloalkyl, aryl, and heteroaryl is independently optionally substituted with one or more R^{10a}.

88. The compound of any one of claims 52-87, or a pharmaceutically acceptable salt, solvate, tautomer, or stereoisomer thereof, wherein:
each R¹⁰ is independently C₁-C₆alkyl, C₂-C₆alkenyl, C₁-C₆aminoalkyl, aryl, or heteroaryl; wherein each alkyl, alkenyl, aryl, and heteroaryl is independently optionally substituted with one or more R^{10a}.

89. The compound of any one of claims 52-88, or a pharmaceutically acceptable salt, solvate, tautomer, or stereoisomer thereof, wherein:
each R¹⁰ is independently aryl optionally substituted with one or more R^{10a}.

90. The compound of any one of claims 52-89, or a pharmaceutically acceptable salt, solvate, tautomer, or stereoisomer thereof, wherein:
each R^{10a} is independently deuterium, halogen, —CN, —OR^{b}, —NR^{c}R^{d}, —C(═O)R^{a}, —C(═O)OR^{b}, —C(═O)NR^{c}R^{d}, C₁-C₆alkyl, or C₁-C₆haloalkyl.

91. The compound of any one of claims 52-90, or a pharmaceutically acceptable salt, solvate, tautomer, or stereoisomer thereof, wherein:
each R^{10a} is independently halogen, —OR^{b}, —C(═O)OR^{b}, or C₁-C₆alkyl.

92. A compound of Formula (IV) and (V), or a pharmaceutically acceptable salt, solvate, tautomer, or stereoisomer thereof: 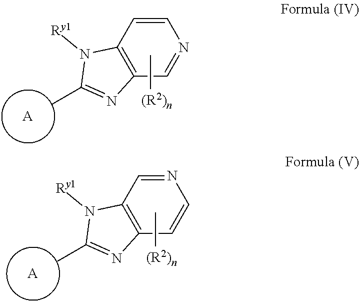
wherein
Ring A is 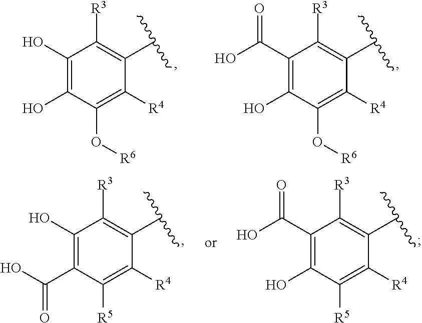
each R² is independently hydrogen, deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
n is 1-3;
R^{Y1} is hydrogen, —S(═O)R^{a}, —S(═O)₂R^{a}, —S(═O)₂NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{Y1a};
R³ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{3a};
R⁴ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{4a};
R⁵ is hydrogen, deuterium, halogen, —CN, —OR^{b}, —NO₂, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{5a};
R⁶ is —C(═O)R^{a}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{6a};
or R⁴ and R⁶ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or R⁴ and R⁵ are taken together to form a heterocycloalkyl optionally substituted with deuterium, halogen, —CN, —OR^{b}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
each R^{Y1a}, R^{2a}, R^{3a}, R^{4a}, R^{5a}, R^{6a}, R^{10a}, R^{11a}, R^{12a}, R^{13a}, and R^{Ca} is independently deuterium, halogen, —CN, —OR^{b}, —SR^{b}, —S(═O)R^{a}, —S(═O)₂R^{a}, —NO₂, —NR^{c}R^{d}, —NHS(═O)₂R^{a}, —S(═O)₂NR^{c}R^{d}, —C(═O)R^{a}, —OC(═O)R^{a}, —C(═O)OR^{b}, —OC(═O)OR^{b}, —C(═O)NR^{c}R^{d}, —OC(═O)NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl;
or two R^{Y1a}, two R^{2a}, two R^{3a}, two R^{4a}, two R^{5a}, and two R^{6a} on the same carbon are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl; and
each R^{c} and R^{d} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl;
or R^{c} and R^{d} are taken together with the nitrogen atom to which they are attached to form a heterocycloalkyl optionally substituted with one or more oxo, deuterium, halogen, —CN, —OH, —OMe, —NH₂, —C(═O)Me, —C(═O)OH, —C(═O)OMe, C₁-C₆alkyl, or C₁-C₆haloalkyl.

93. The compound of claim 92, or a pharmaceutically acceptable salt, solvate, tautomer, or stereoisomer thereof, wherein the compound of Formula (IV) is of Formula (IVa):

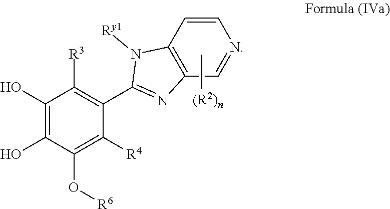

94. The compound of claim 92, or a pharmaceutically acceptable salt, solvate, tautomer, or stereoisomer thereof, wherein the compound of Formula (IV) is of Formula (IVb):

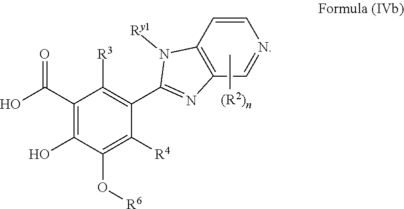

95. The compound of claim 92, or a pharmaceutically acceptable salt, solvate, tautomer, or stereoisomer thereof, wherein the compound of Formula (IV) is of Formula (IVc):

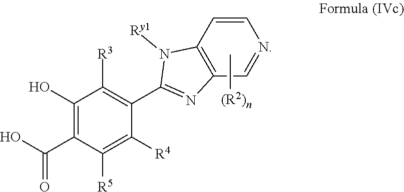

96. The compound of claim 92, or a pharmaceutically acceptable salt, solvate, tautomer, or stereoisomer thereof, wherein the compound of Formula (IV) is of Formula (IVd):

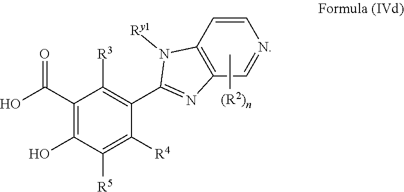

97. The compound of claim 92, or a pharmaceutically acceptable salt, solvate, tautomer, or stereoisomer thereof, wherein the compound of Formula (V) is of Formula (Va):

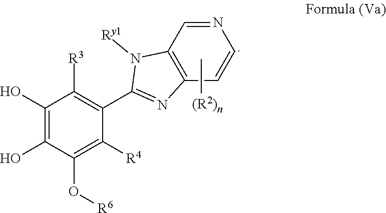

98. The compound of claim 92, or a pharmaceutically acceptable salt, solvate, tautomer, or stereoisomer thereof, wherein the compound of Formula (V) is of Formula (Vb):

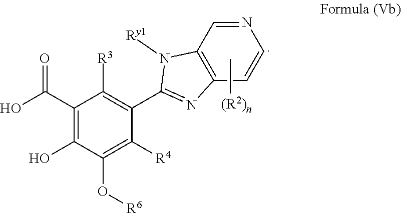

99. The compound of claim 92, or a pharmaceutically acceptable salt, solvate, tautomer, or stereoisomer thereof, wherein the compound of Formula (V) is of Formula (Vc):

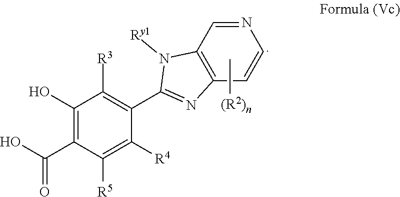

100. The compound of claim 92, or a pharmaceutically acceptable salt, solvate, tautomer, or stereoisomer thereof, wherein the compound of Formula (V) is of Formula (Vd):

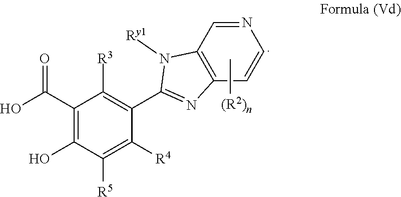

101. The compound of any one of claims 92-100, or a pharmaceutically acceptable salt, solvate, tautomer, or stereoisomer thereof, wherein:
R^{Y1} is hydrogen, C₁-C₆alkyl, or heterocycloalkyl.

102. The compound of any one of claims 92-101, or a pharmaceutically acceptable salt, solvate, tautomer, or stereoisomer thereof, wherein:
R^{Y1} is hydrogen.

103. The compound of any one of claims 92-102, or a pharmaceutically acceptable salt, solvate, tautomer, or stereoisomer thereof, wherein:
R^{Y1} is heterocycloalkyl.

104. The compound of any one of claims 92-103, or a pharmaceutically acceptable salt, solvate, tautomer, or stereoisomer thereof, wherein:
R³ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

105. The compound of any one of claims 92-104, or a pharmaceutically acceptable salt, solvate, tautomer, or stereoisomer thereof, wherein:
R³ is hydrogen or halogen.

106. The compound of any one of claims 92-105, or a pharmaceutically acceptable salt, solvate, tautomer, or stereoisomer thereof, wherein:
R³ is hydrogen.

107. The compound of any one of claims 92-106, or a pharmaceutically acceptable salt, solvate, tautomer, or stereoisomer thereof, wherein:
R⁴ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

108. The compound of any one of claims 92-107, or a pharmaceutically acceptable salt, solvate, tautomer, or stereoisomer thereof, wherein:
R⁴ is hydrogen or halogen.

109. The compound of any one of claims 92-108, or a pharmaceutically acceptable salt, solvate, tautomer, or stereoisomer thereof, wherein:
R⁴ is hydrogen.

110. The compound of any one of claims 92-109, or a pharmaceutically acceptable salt, solvate, tautomer, or stereoisomer thereof, wherein:
R⁵ is hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

111. The compound of any one of claims 92-110, or a pharmaceutically acceptable salt, solvate, tautomer, or stereoisomer thereof, wherein:
R⁵ is hydrogen or halogen.

112. The compound of any one of claims 92-111, or a pharmaceutically acceptable salt, solvate, tautomer, or stereoisomer thereof, wherein:
R⁵ is hydrogen.

113. The compound of any one of claims 92-112, or a pharmaceutically acceptable salt, solvate, tautomer, or stereoisomer thereof, wherein:
R⁶ is C₁-C₆alkyl.

114. The compound of any one of claims 92-113, or a pharmaceutically acceptable salt, solvate, tautomer, or stereoisomer thereof, wherein:
R⁶ is methyl.

115. The compound of any one of claims 92-114, or a pharmaceutically acceptable salt, solvate, tautomer, or stereoisomer thereof, wherein:
each R² is independently hydrogen, deuterium, halogen, —CN, —OR^{b}, —NR^{c}R^{d}, —C(═O)R^{a}, —C(═O)OR^{b}, —C(═O)NR^{c}R^{d}, C₁-C₆alkyl, C₁-C₆haloalkyl, or C₁-C₆deuteroalkyl.

116. The compound of any one of claims 92-115, or a pharmaceutically acceptable salt, solvate, tautomer, or stereoisomer thereof, wherein:
each R² is independently hydrogen, deuterium, halogen, —CN, —OR^{b}, —NR^{c}R^{d}, C₁-C₆alkyl, C₁-C₆haloalkyl, or C₁-C₆deuteroalkyl.

117. The compound of any one of claims 92-116, or a pharmaceutically acceptable salt, solvate, tautomer, or stereoisomer thereof, wherein:
each R² is independently hydrogen, halogen, C₁-C₆alkyl, or C₁-C₆haloalkyl.

118. The compound of any one of claims 92-117, or a pharmaceutically acceptable salt, solvate, tautomer, or stereoisomer thereof, wherein:
each R² is independently hydrogen or halogen.

119. The compound of any one of claims 92-118, or a pharmaceutically acceptable salt, solvate, tautomer, or stereoisomer thereof, wherein:
each R² is hydrogen.

120. The compound of any one of claims 92-119, or a pharmaceutically acceptable salt, solvate, tautomer, or stereoisomer thereof, wherein:
n is 1 or 2.

121. The compound of any one of claims 92-120, or a pharmaceutically acceptable salt, solvate, tautomer, or stereoisomer thereof, wherein:
n is 1.

122. The compound of any one of claims 1-121, or a pharmaceutically acceptable salt, solvate, tautomer, or stereoisomer thereof, wherein:
R^{Y1} is cycloalkyl or heterocycloalkyl; wherein the cycloalkyl and heterocycloalkyl is optionally substituted with one or more R^{Y1a}.

123. The compound of any one of claims 1-122, or a pharmaceutically acceptable salt, solvate, tautomer, or stereoisomer thereof, wherein:
R^{Y1} is cycloalkyl optionally substituted with one or more R^{Y1a}.

124. The compound of any one of claims 1-122, or a pharmaceutically acceptable salt, solvate, tautomer, or stereoisomer thereof, wherein:
R^{Y1} is a bicyclic cycloalkyl optionally substituted with one or more R^{Y1a}.

125. The compound of any one of claims 1-122, or a pharmaceutically acceptable salt, solvate, tautomer, or stereoisomer thereof, wherein:
R^{Y1} is heterocycloalkyl optionally substituted with one or more R^{Y1a}.

126. The compound of any one of claims 1-122, or a pharmaceutically acceptable salt, solvate, tautomer, or stereoisomer thereof, wherein:
R^{Y1} is

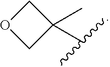 [SMILES: *C1(C)COC1]

127. The compound of any one of claims 1-122, or a pharmaceutically acceptable salt, solvate, tautomer, or stereoisomer thereof, wherein:
R_{Y1} is

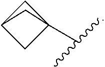 [SMILES: *C12CC(C1)C2]

128. A compound selected from the group consisting of a compound found in table 1, or a pharmaceutically acceptable salt, solvate, tautomer, or stereoisomer thereof.

129. A pharmaceutical composition comprising the compound of any one of claims 1-128, or a pharmaceutically acceptable salt, solvate, tautomer, or stereoisomer thereof, and a pharmaceutically acceptable excipient.

130. A method of treating cancer in a subject in need thereof, the method comprising administering the compound of any one of claims 1-128, or a pharmaceutically acceptable salt, solvate, tautomer, or stereoisomer thereof.

131. The method of claim 130, wherein the cancer is characterized by a deficiency in one or more DNA repair pathways.

132. The method of claim 130, wherein the DNA repair deficiency is a deficiency in the base excision repair (“BER”) pathway, the Fanconi anaemia-mediated repair (“FA”) pathway, the homologous recombination (“HR”) pathway, the nucleotide excision repair (“NER”) pathway, the non-homologous end joining (“NHEJ”) pathway, the mismatch repair (“MMR”) pathway, the RecQ-mediated repair (“RecQ”) pathway, or the double-stranded breaks (“DSB”) pathway.

133. The method of claim 131 or claim 132, wherein the DNA repair deficiency is a deficiency in the homologous recombination (“HR”) pathway.

134. The method of any one of claims 131-133, wherein the DNA repair deficiency is a BRCA1 mutation.

135. The method of any one of claims 131-134, further comprising administering a DNA repair inhibitor.

136. The method of claim 135, wherein the DNA repair inhibitor is a poly ADP ribose polymerase (“PARP”) inhibitor.

137. The method of any one of claims 131-134, further comprising administering an alkylating agent.

138. The method of claim 137, wherein the alkylating agent is cyclophosphamide, chlormethine, uramustine, melphalan, chlorambucil, ifosfamide, bendamustine, carmustine, lomustine, nimustine, fotemustine, streptozocin, or busulfan.

139. The method of any one of claims 131-134, further comprising administering a DNA damaging agent.

140. The method of claim 139, wherein the DNA damaging agent is camptothecin, etoposide, oxaliplatin, cisplatin, or doxorubicin.

141. The method of any one of claims 131-134, wherein the compound is administered in conjunction with high-dose radiotherapy.

142. The method of claim 141, wherein the high-dose radiotherapy is administered as a single dose and/or hypofractionated.

143. The method of any one of claims 131-140, wherein the compound is administered in conjunction with Stereotactic Body Radiation Therapy (SBRT).
